(19) United States
(12) Patent Application Publication
(10) Pub. No.: US 2023/0346885 A1
(43) Pub. Date: Nov. 2, 2023

(54) Peptides

(71) Applicant: Immunocore Limited, Abingdon (GB)

(72) Inventors: Alex Powlesland, Abingdon (GB); Alfred Lim, Abingdon (GB); Alina Popa, Abingdon (GB); Andrew Brooks, Abingdon (GB); Benjamin Oestringer, Abingdon (GB); Chandramouli Reddy Chillakuri, Abingdon (GB); Christopher Sayer, Abingdon (GB); Dhaval Jaykant Sangani, Abingdon (GB); Elena Galfre, Abingdon (GB); Fiona Chester, Abingdon (GB); Garret Keating, Abingdon (GB); Graham Anthony Hood, Abingdon (GB); Izabela Bombik, Abingdon (GB); Johanne Pentier, Abingdon (GB); Maurits Kleijnen, Abingdon (GB); Meidai Sun, Abingdon (GB); Nicole Mai, Abingdon (GB); Philip Addis, Abingdon (GB); Pietro Dellacristina, Abingdon (GB); Sarah Gilgunn, Abingdon (GB); Victoria Arena De Souza, Abingdon (GB)

(21) Appl. No.: 18/064,180

(22) Filed: Dec. 9, 2022

Related U.S. Application Data
(63) Continuation-in-part of application No. 18/049,198, filed on Oct. 24, 2022.
Application No. 18/049,198 is a continuation of application No. 17/831,193, filed on Jun. 2, 2022, now abandoned.
Application No. 17/831,193 is a division of application No. 15/777,144, filed on May 17, 2018, now Pat. No. 10,792,333.
Application No. 15/777,144 is a continuation-in-part of application No. 17/099,242, filed on Nov. 16, 2020.
Application No. 17/099,242 is a continuation of application No. 15/777,163, filed on May 17, 2018, now abandoned.
Application No. 15/777,163 is a continuation-in-part of application No. 17/031,691, filed on Sep. 24, 2020, now abandoned.
Application No. 17/031,691 is a division of application No. 16/097,582, filed on Oct. 29, 2018, now abandoned.
Application No. 16/097,582 is a continuation-in-part of application No. 17/087,363, filed on Nov. 2, 2020.
Application No. 17/087,363 is a continuation of application No. 15/777,149, filed on May 17, 2018, now abandoned.
Application No. 15/777,149 is a continuation-in-part of application No. 17/017,444, filed on Sep. 10, 2020.
Application No. 17/017,444 is a continuation of application No. 16/097,587, filed on Oct. 29, 2018, now abandoned.
Application No. 16/097,587 is a continuation-in-part of application No. 16/985,061, filed on Aug. 4, 2020.
Application No. 16/985,061 is a continuation of application No. 15/777,180, filed on May 17, 2018, now abandoned.
Application No. 15/777,180 is a continuation-in-part of application No. 16/988,431, filed on Aug. 7, 2020.
Application No. 16/988,431 is a continuation of application No. 15/777,874, filed on May 21, 2018, now abandoned.
Application No. 15/777,874 is a continuation-in-part of application No. 17/104,677, filed on Nov. 25, 2020, now abandoned.
Application No. 17/104,677 is a continuation of application No. 15/777,875, filed on May 21, 2018, now abandoned.
Application No. 15/777,875 is a continuation-in-part of application No. 17/104,668, filed on Nov. 25, 2020, now abandoned.
Application No. 17/104,668 is a continuation of application No. 15/777,877, filed on May 21, 2018, now abandoned.
Application No. 15/777,877 is a continuation-in-part of application No. 17/104,415, filed on Nov. 25, 2020, now abandoned.
Application No. 17/104,415 is a continuation of application No. 15/777,613, filed on May 18, 2018, now abandoned.
Application No. 15/777,613 is a continuation-in-part of application No. 16/998,945, filed on Aug. 20, 2020.
Application No. 16/998,945 is a continuation of application No. 15/777,155, filed on May 17, 2018, now abandoned.
Application No. 15/777,155 is a continuation-in-part of application No. 17/083,158, filed on Oct. 28, 2020, now abandoned.
Application No. 17/083,158 is a division of application No. 15/777,621, filed on May 18, 2018, now Pat. No. 10,851,366.
Application No. 15/777,621 is a continuation-in-part of application No. 16/988,425, filed on Aug. 7, 2020.
Application No. 16/988,425 is a continuation of application No. 15/777,629, filed on May 18, 2018, now abandoned.
Application No. 15/777,629 is a continuation-in-part of application No. 17/013,228, filed on Sep. 4, 2020.
Application No. 17/013,228 is a continuation of application No. 15/777,882, filed on May 21, 2018, now abandoned.
Application No. 15/777,882 is a continuation-in-part of application No. 17/243,932, filed on Apr. 29, 2021.
Application No. 17/243,932 is a continuation of application No. 15/777,880, filed on May 21, 2018, now abandoned.
Application No. 15/777,880 is a continuation-in-part of application No. 17/010,653, filed on Sep. 2, 2020.
Application No. 17/010,653 is a continuation of application No. 15/777,885, filed on May 21, 2018, now abandoned.
Application No. 15/777,885 is a continuation-in-part of application No. 17/938,323, filed on Oct. 5, 2022.
Application No. 17/938,323 is a continuation of application No. 17/092,715, filed on Nov. 9, 2020, now abandoned.
Application No. 17/092,715 is a continuation of application No. 16/084,883, filed on Sep. 13, 2018, now abandoned.
Application No. 16/084,883 is a continuation-in-part of application No. 16/985,058, filed on Aug. 4, 2020.
Application No. 16/985,058 is a continuation of application No. 15/777,888, filed on May 21, 2018, now abandoned.
Application No. 15/777,888 is a continuation-in-part of application No. 17/017,457, filed on Sep. 10, 2020.
Application No. 17/017,457 is a continuation of application No. 15/777,892, filed on May 21, 2018, now abandoned.
Application No. 15/777,892 is a continuation-in-part of application No. 16/994,399, filed on Aug. 14, 2020.
Application No. 16/994,399 is a continuation of application No. 15/778,175, filed on May 22, 2018, now abandoned.
Application No. 15/778,175 is a continuation-in-part of application No. 17/099,267, filed on Nov. 16, 2020.
Application No. 17/099,267 is a continuation of application No. 15/778,177, filed on May 22, 2018, now abandoned.
Application No. 15/778,177 is a continuation-in-part of application No. 17/105,010, filed on Nov. 25, 2020, now abandoned.
Application No. 17/105,010 is a continuation of application No. 15/778,179, filed on May 22, 2018, now abandoned.
Application No. 15/778,179 is a continuation-in-part of application No. 16/938,263, filed on Jul. 24, 2020.
Application No. 16/938,263 is a continuation of application No. 15/778,198, filed on May 22, 2018, now abandoned.
Application No. 15/778,198 is a continuation-in-part of application No. 17/099,286, filed on Nov. 16, 2020.
Application No. 17/099,286 is a continuation of application No. 15/778,204, filed on May 22, 2018, now abandoned.
Application No. 15/778,204 is a continuation-in-part of application No. 17/963,988, filed on Oct. 11, 2022.
Application No. 17/963,988 is a continuation of application No. 17/092,970, filed on Nov. 9, 2020, now abandoned.
Application No. 17/092,970 is a continuation of application No. 15/778,199, filed on May 22, 2018, now abandoned.
Application No. 15/778,199 is a continuation-in-part of application No. 18/046,148, filed on Oct. 12, 2022.
Application No. 18/046,148 is a continuation of application No. 16/988,523, filed on Aug. 7, 2020, now abandoned.
Application No. 16/988,523 is a division of application No. 15/778,206, filed on May 22, 2018, now Pat. No. 10,980,893.
Application No. 15/778,206 is a continuation-in-part of application No. 17/811,040, filed on Jul. 6, 2022, now abandoned.
Application No. 17/811,040 is a continuation of application No. 16/347,384, filed on May 3, 2019, now abandoned.

(30) Foreign Application Priority Data
Nov. 23, 2015 (GB) 1520536.2
Nov. 23, 2015 (GB) 1520541.2
Nov. 23, 2015 (GB) 1520542.0
Nov. 23, 2015 (GB) 1520543.8
Nov. 23, 2015 (GB) 1520544.6
Nov. 23, 2015 (GB) 1520545.3
Nov. 23, 2015 (GB) 1520546.1
Nov. 23, 2015 (GB) 1520548.7
Nov. 23, 2015 (GB) 1520550.3
Nov. 23, 2015 (GB) 1520557.8
Nov. 23, 2015 (GB) 1520558.6
Nov. 23, 2015 (GB) 1520562.8
Nov. 23, 2015 (GB) 1520563.6
Nov. 23, 2015 (GB) 1520564.4
Nov. 23, 2015 (GB) 1520566.9
Nov. 23, 2015 (GB) 1520567.7
Nov. 23, 2015 (GB) 1520568.5
Nov. 23, 2015 (GB) 1520570.1
Nov. 23, 2015 (GB) 1520583.4
Nov. 23, 2015 (GB) 1520589.1
Nov. 23, 2015 (GB) 1520595.8
Nov. 23, 2015 (GB) 1520603.0
Mar. 16, 2016 (GB) 1604468.7
Apr. 29, 2016 (GB) 1607534.3
Apr. 29, 2016 (GB) 1607535.0
Nov. 7, 2016 (GB) 1618769.2
Nov. 23, 2016 (GB) 1520592.5

(51) Int. Cl. A61K 38/17; A61K 38/04; C07K 14/74; C07K 14/47; C07K 7/06; A61P 35/00

(52) U.S. Cl. CPC A61K 38/1774; A61K 38/04; C07K 14/70539; C07K 14/47; C07K 7/06; A61P 35/00; A61K 38/00

(57) ABSTRACT

The present invention relates to novel peptides derived from various target proteins, complexes comprising such peptides bound to recombinant MHC molecules, and cells presenting said peptide in complex with MHC molecules. Also provided by the present invention are binding moieties that bind to the peptides and/or complexes of the invention. Such moieties are useful for the development of immunotherapeutic reagents for the treatment of diseases such as cancer.

### CROSS-REFERENCE TO RELATED APPLICATIONS

This application is a continuation-in-part of co-pending U.S. application Ser. No. 18/049,198, filed Oct. 24, 2022, which is a continuation of U.S. application Ser. No. 17/831,193, filed Jun. 2, 2022, which is a divisional of U.S. application Ser. No. 15/777,144, filed May 17, 2018, now U.S. Pat. No. 10,792,333, which is the National Stage of International Application No. PCT/GB2016/053643, filed Nov. 23, 2016, which claims priority benefit of GB Application No. 1520568.5, filed Nov. 23, 2015, each of which is hereby incorporated by reference in its entirety.

This application is a continuation-in-part of co-pending of U.S. application Ser. No. 17/099,242, filed Nov. 16, 2020, which is a continuation of U.S. application Ser. No. 15/777,163, filed May 17, 2018, now abandoned, which is the National Stage of International Application No. PCT/GB2016/053661, filed Nov. 23, 2016, which claims priority benefit of GB Application No. 1520592.5, filed Nov. 23, 2015, each of which is hereby incorporated by reference in its entirety.

This application is a continuation-in-part of co-pending U.S. application Ser. No. 17/031,691, filed Sep. 24, 2020, which is a divisional of U.S. application Ser. No. 16/097,582, filed Oct. 29, 2018, now abandoned, which is the National Stage of International Application No. PCT/GB2017/051192, filed Apr. 28, 2017, which claims priority benefit of GB Application No. 1607535.0, filed Apr. 29, 2016, each of which is hereby incorporated by reference in its entirety.

This application is a continuation-in-part of co-pending U.S. application Ser. No. 17/087,363, filed Nov. 2, 2020, which is a continuation of U.S. application Ser. No. 15/777,149, filed May 17, 2018, now abandoned, which is the National Stage of International Application No. PCT/GB2016/053670, filed Nov. 23, 2016, which claims priority benefit of GB Application No. 1520564.4, filed Nov. 23, 2015, each of which is hereby incorporated by reference in its entirety.

This application is a continuation-in-part of co-pending U.S. application Ser. No. 17/017,444, filed Se. 10, 2020, which is a continuation of U.S. application Ser. No. 16/097,587, filed Oct. 29, 2018, now abandoned, which is the National Stage of International Application No. PCT/GB2017/051193, filed Apr. 28, 2017, which claims priority benefit of GB Application No. 1607534.3, filed Apr. 29, 2016, each of which is hereby incorporated by reference in its entirety.

This application is a continuation-in-part of co-pending U.S. application Ser. No. 16/985,061, filed Aug. 4, 2020, which is a continuation of U.S. application Ser. No. 15/777,180, filed May 17, 2018, now abandoned, which is the National Stage of International Application No. PCT/GB2016/053664, filed Nov. 23, 2016, which claims priority benefit of GB Application No. 1520563.6, filed Nov. 23, 2015, each of which is hereby incorporated by reference in its entirety.

This application is a continuation-in-part of co-pending U.S. application Ser. No. 16/988,431, filed Aug. 7, 2020, which is a continuation of U.S. application Ser. No. 15/777,874, filed May 21, 2018, now abandoned, which is the National Stage of International Application No. PCT/GB2016/053645, filed Nov. 23, 2016, which claims priority benefit of GB Application No. 1520583.4, filed Nov. 23, 2015, each of which is hereby incorporated by reference in its entirety.

This application is a continuation-in-part of co-pending U.S. application Ser. No. 17/104,677, filed Nov. 25, 2020, which is a continuation of U.S. application Ser. No. 15/777,875, filed May 21, 2018, now abandoned, which is the National Stage of International Application No. PCT/GB2016/053660, filed Nov. 23, 2016, which claims priority benefit of GB Application No. 1520557.8, filed Nov. 23, 2015, each of which is hereby incorporated by reference in its entirety.

This application is a continuation-in-part of co-pending U.S. application Ser. No. 17/104,668, filed Nov. 25, 2020, which is a continuation of U.S. application Ser. No. 15/777,877, filed May 21, 2018, now abandoned, which is the National Stage of International Application No. PCT/GB2016/053664, filed Nov. 23, 2016, which claims priority benefit of GB Application No. 1520558.6, filed Nov. 23, 2015, each of which is hereby incorporated by reference in its entirety.

This application is a continuation-in-part of co-pending U.S. application Ser. No. 17/104,415, filed Nov. 25, 2020, which is a continuation of U.S. application Ser. No. 15/777,613, filed May 17, 2018, now abandoned, which is the National Stage of International Application No. PCT/GB2016/053665, filed Nov. 23, 2016, which claims priority benefit of GB Application No. 1520570.1, filed Nov. 23, 2015, each of which is hereby incorporated by reference in its entirety.

This application is a continuation-in-part of co-pending U.S. application Ser. No. 16/998,945, filed Aug. 20, 2020, which is a continuation of U.S. application Ser. No. 15/777,155, filed May 17, 2018, now abandoned, which is the National Stage of International Application No. PCT/GB2016/053654, filed Nov. 23, 2016, which claims priority benefit of GB Application No. 1520566.9, filed Nov. 23, 2015, each of which is hereby incorporated by reference in its entirety.

This application is a continuation-in-part of co-pending U.S. application Ser. No. 17/083,158, filed Oct. 28, 2020, which is a divisional of U.S. application Ser. No. 15/777,621, filed May 18, 2018, now U.S. Pat. No. 10,851,366, which is the National Stage of International Application No. PCT/GB2016/053659, filed November 23, 2016, which claims priority benefit of GB Application No. 1520589.1, filed Nov. 23, 2015, each of which is hereby incorporated by reference in its entirety.

This application is a continuation-in-part of co-pending U.S. application Ser. No. 16/988,425, filed Aug. 7, 2020, which is a continuation of U.S. application Ser. No. 15/777,629, filed May 18, 2018, now abandoned, which is the National Stage of International Application No. PCT/GB2016/053658, filed Nov. 23, 2016, which claims priority benefit of GB Application No. 1520544.6, filed Nov. 23, 2015, each of which is hereby incorporated by reference in its entirety.

This application is a continuation-in-part of co-pending U.S. application Ser. No. 17/013,228, filed Sep. 4, 2020, which is a continuation of U.S. application Ser. No. 15/777,882, filed May 21, 2018, now abandoned, which is the National Stage of International Application No. PCT/GB2016/053656, filed Nov. 23, 2016, which claims priority benefit of GB Application No. 1520541.2, filed Nov. 23, 2015, each of which is hereby incorporated by reference in its entirety.

This application is a continuation-in-part of co-pending U.S. application Ser. No. 17/243,932, filed Apr. 29, 2021, which is a continuation of U.S. application Ser. No. 15/777,880, filed May 21, 2018, now abandoned, which is the National Stage of International Application No. PCT/GB2016/053648, filed Nov. 23, 2016, which claims priority benefit of GB Application No. 1520562.8, filed Nov. 23, 2015, each of which is hereby incorporated by reference in its entirety.

This application is a continuation-in-part of co-pending U.S. application Ser. No. 17/010,653, filed Sep. 2, 2020, which is a continuation of U.S. application Ser. No. 15/777,885, filed May 21, 2018, now abandoned, which is the National Stage of International Application No. PCT/GB2016/053647, filed Nov. 23, 2016, which claims priority benefit of GB Application No. 1520567.7, filed Nov. 23, 2015, each of which is hereby incorporated by reference in its entirety.

This application is a continuation-in-part of co-pending U.S. application Ser. No. 17/938,323, filed Oct. 5, 2022, which is a continuation of U.S. application Ser. No. 17/092,715, filed Nov. 9, 2020, now abandoned, which is a continuation U.S. application Ser. No. 16/084,883, filed Sep. 13, 2018, now abandoned, which is the National Stage of International Application No. PCT/GB2017/050732, filed Mar. 16, 2017, which claims priority benefit of GB Application No. 1604468.7, filed Mar. 16, 2016, each of which is hereby incorporated by reference in its entirety.

This application is a continuation-in-part of co-pending U.S. application Ser. No. 16/985,058, filed Aug. 4, 2020, which is a continuation of U.S. application Ser. No. 15/777,888, filed May 21, 2018, now abandoned, which is the National Stage of International Application No. PCT/GB2016/053642, filed Nov. 23, 2016, which claims priority benefit of GB Application No. 1520542.0, filed Nov. 23, 2015, each of which is hereby incorporated by reference in its entirety.

This application is a continuation-in-part of co-pending U.S. application Ser. No. 17/017,457, filed Sep. 10, 2020, which is a continuation of U.S. application Ser. No. 15/777,892, filed May 21, 2018, now abandoned, which is the National Stage of International Application No. PCT/GB2016/053640, filed Nov. 23, 2016, which claims priority benefit of GB Application No. 1520548.7, filed Nov. 23, 2015, each of which is hereby incorporated by reference in its entirety.

This application is a continuation-in-part of co-pending U.S. application Ser. No. 16/994,399, filed Aug. 14, 2020, which is a continuation of U.S. application Ser. No. 15/778,175, filed May 22, 2018, now abandoned, which is the National Stage of International Application No. PCT/GB2016/053653, filed Nov. 23, 2016, which claims priority benefit of GB Application No. 1520546.1, filed Nov. 23, 2015, each of which is hereby incorporated by reference in its entirety.

This application is a continuation-in-part of co-pending U.S. application Ser. No. 17/099,267, filed Nov. 16, 2020, which is a continuation of U.S. application Ser. No. 15/778,177, filed May 22, 2018, now abandoned, which is the National Stage of International Application No. PCT/GB2016/053641, filed Nov. 23, 2016, which claims priority benefit of GB Application No. 1520545.3, filed November 23, 2015, each of which is hereby incorporated by reference in its entirety.

This application is a continuation-in-part of co-pending U.S. application Ser. No. 17/105,010, filed Nov. 25, 2020, which is a continuation of U.S. application Ser. No. 15/778,179, filed May 22, 2018, now abandoned, which is the National Stage of International Application No. PCT/GB2016/053666, filed Nov. 23, 2016, which claims priority benefit of GB Application No. 1520543.8, filed Nov. 23, 2015, each of which is hereby incorporated by reference in its entirety.

This application is a continuation-in-part of co-pending U.S. application Ser. No. 16/938,263, filed Jul. 24, 2020, which is continuation of U.S. application Ser. No. 15/778,198, filed May 22, 2018, now abandoned, which is the National Stage of International Application No. PCT/GB2016/053651, filed Nov. 23, 2016, which claims priority benefit of GB Application No. 1520603.0, filed Nov. 23, 2015, each of which is hereby incorporated by reference in its entirety.

This application is a continuation-in-part of co-pending U.S. application Ser. No. 17/099286, filed Nov. 16, 2020, which is continuation of U.S. application Ser. No. 15/778,204, filed May 22, 2018, now abandoned, which is the National Stage of International Application No. PCT/GB2016/053655, filed Nov. 23, 2016, which claims priority benefit of GB Application No. 1520536.2, filed Nov. 23, 2015, each of which is hereby incorporated by reference in its entirety.

This application is a continuation-in-part of co-pending U.S. application Ser. No. 17/963,988, filed Oct. 11, 2022, which is a continuation of U.S. application Ser. No. 17/092,970, filed Nov. 9, 2020, now abandoned, which is a continuation of U.S. application Ser. No. 15/778,199, filed May 22, 2018, now abandoned, which is the National Stage of International Application No. PCT/GB2016/053650, filed Nov. 23, 2016, which claims priority benefit of GB Application No. 1520595.8, filed Nov. 23, 2015, each of which is hereby incorporated by reference in its entirety.

This application is a continuation-in-part of co-pending U.S. application Ser. No. 18/046,148, filed Oct. 12, 2022, which is a continuation of U.S. application Ser. No. 16/988,523 filed Aug. 7, 2020, now abandoned, which is a divisional of U.S. application Ser. No. 15/778,206, filed May 22, 2018, now U.S. Pat. No. 10,980,893, which is the National Stage of International Application No. PCT/GB2016/053646, filed Nov. 23, 2016, which claims priority benefit of GB Application No. 1520550.3, filed Nov. 23, 2015, each of which is hereby incorporated by reference in its entirety.

This application is a continuation-in-part of co-pending U.S. application Ser. No. 17/811,040, filed Jul. 6, 2022, which is a continuation of U.S. application Ser. No. 16/347,384, filed May 3, 2019, now abandoned, which is the National Stage of International Application No. PCT/GB2017/053350, filed Nov. 7, 2017, which claims priority benefit of GB Application No. 1618769.2, filed Nov. 7, 2016, each of which is hereby incorporated by reference in its entirety.

### SEQUENCE LISTING

The instant application contains a Sequence Listing which has been submitted via Patent Center and is hereby incorporated by reference in its entirety. Said XML copy, created on Dec. 6, 2022, is named 54425US_Sequence Listing.xml, and is 571,575 bytes in size.

### BRIEF DESCRIPTION OF THE FIGURES

FIGS. 1A-1JN shows the respective fragmentation spectra for the peptides of SEQ ID NOS: 1 to 274, eluted from cells. A table highlighting the matching ions is shown below each spectrum.

FIGS. 2A-BF shows ELISA plates demonstrating specific TCRs can be isolated against 58 of the peptides in complex with HLA.

FIG. 3 shows RT-PCR analysis of XAGE1 expression in lung tumour samples and normal tissue samples.

The present invention relates to novel tumour-associated antigenic peptides derived from various proteins, complexes comprising such peptides bound to recombinant MHC molecules, and cells presenting said peptide in complex with MHC molecules. Also provided by the present invention are binding moieties that bind to the peptides and/or complexes of the invention. Such moieties are useful for the development of immunotherapeutic reagents for the treatment of diseases such as cancer.

T cells are a key part of the cellular arm of the immune system. They specifically recognise peptide fragments that are derived from intracellular proteins and presented in complex with Major Histocompatibility Complex (MHC) molecules on the surface of antigen presenting cells (APCs). In humans, MHC molecules are known as human leukocyte antigens (HLA), and both terms are used synonymously herein. MHC molecules have a binding groove in which the peptide fragments bind. Recognition of particular peptide-MHC antigens is mediated by a corresponding T cell receptor (TCR). Tumour cells express various tumour associated antigens (TAA) and peptides derived from these antigens may be displayed on the tumour cell surface. Detection of a MHC class I-presented TAA-derived peptide by a CD8+ T cell bearing the corresponding T cell receptor, leads to targeted killing of the tumour cell. However, as a consequence of the selection processes which occur during T cell maturation in the thymus, there is a scarcity of T cells (and TCRs) in the circulating repertoire, which recognise TAA-derived peptides with a sufficiently high level of affinity. Therefore tumour cells often escape detection.

The identification of particular TAA-derived peptides presented by MHC molecules on tumour cells enables the development of novel immunotherapeutic reagents designed to specifically target and destroy said tumour cells. Such reagents may be moieties that bind to the TAA-derived peptide and/or complexes of peptide and MHC and they typically function by inducing a T cell response.

For example, such reagents may be based, exclusively, or in part, on T cells, or T cell receptors (TCRs), or antibodies. The identification of suitable TAAs for therapeutic targeting requires careful consideration in order to mitigate off-tumour on-target toxicity in a clinical setting. TAAs that are suitable as targets for immunotherapeutic intervention should show a sufficient difference in expression levels between tumour tissue and normal, healthy tissues; in other words there should be a suitable therapeutic window, which will enable targeting of tumour tissue and minimise targeting healthy tissues. Ideally TAAs are highly expressed in tumour tissue and have limited or no expression in normal healthy tissue. Typically, a person skilled in the art would use protein expression data to identify whether a therapeutic window exists for a given TAA. Higher protein expression being indicative of higher levels of peptide-MHC presented peptide on the cell surface. The inventors of the present application have found that differences in RNA expression, rather than protein expression is a more reliable indicator of pMHC levels and consequently the therapeutic window.

It is therefore desirable to provide peptides derived from TAAs with a suitable therapeutic window, based on RNA expression, MHC complexes thereof and binding moieties that can be used for the development of new cancer therapies. Furthermore, it is desirable that said peptides are not identical to, or highly similar to, any other MHC restricted peptide, derived from an alternative protein(s), and presented by MHC on the surface of non-cancerous cells. The existence of such peptide mimics increase the risk of in vivo toxicity for targeted cancer therapies.

In silico algorithms, such as SYFPETHEI (Rammensee, et al., Immunogenetics. 1999 November; 50(3-4):213-9 (access via www.syfpeithi.de) and BIMAS (Parker, et al., J. Immunol. 1994 Jan. 1; 152(1):163-75 (access via http://www-bimas.cit.nih.gov/molbio/hla_bind/)) are available to predict the amino acid sequences of MHC-presented peptides derived from proteins. However, these methods are known to generate a high proportion of false positives (since they simply define the likelihood of a given peptide being able to bind a given MHC and do not account for intracellular processing). Therefore, it is not possible to accurately predict whether a given peptide-MHC is actually presented by tumour cells. Direct experimental data is typically required.

ACTL8 (also known as CT57and having Uniprot accession number Q9H568) belongs to the cancer testis family of germline encoded tumour antigens (Chen et al. Proc Natl Acad Sci U S A. 2005 May 31; 102(22):7940-5). ACTL8 is proposed to play a role in epithelial cell differentiation. Expression of ACTL8 is restricted to testis and pancreas, with weak expression in placenta; expression is upregulated in various tumour types (Yao et al. Cancer immunology research 2.4: 371-379 (2014)). The inventors have found that ACTL8 has a particularly suitable therapeutic window based on RNA expression. Furthermore the inventors have identified novel peptides derived from ACTL8 that are presented on the cell surface in complex with MHC. These peptides are particularly useful for the development of reagents that can targets cells expressing ACTL8 and for the treatment of cancers, including breast, colon and oesophageal cancers.

ASCL2 (also known as achaete-scute homolog 2 or Ash-2 or bHLHa45 and having Uniprot accession number Q99929) functions as a transcription factor. ASLC2 has been implicated in various cancers (Zhu et al. PLoS One. 2012; 7(2):e32170; Hu et al. J Clin Pathol. 2015. pii: jclinpath-2015-203025; Kwon et al. Cancer Sci. 2013 March; 104(3):391-7). ASCL2 is therefore a particularly attractive target for therapeutic intervention. The inventors have found novel peptides derived from ACTL8 that are presented on the cell surface in complex with MHC. These peptides are particularly useful for the development of reagents that can targets cells expressing ACTL8 and for the treatment of cancers, including colon and oesophageal cancers.

BRDT (also known as Bromodomain testis-specific protein, or Cancer/testis antigen 9 or RING3-like protein, and having Uniprot accession number: Q58F21) is a testis-specific chromatin protein that is involved in spermatogenesis and is a member of the RING3 family of transcriptional regulators (Jones et al., (1997) Genomics 45(3):529-34; Zheng et al., (2005) Int J Mol Med 15(2):315-21). Expression of BRDT has been reported in cancers including non-small cell lung cancer and squamous cell carcinomas of the head and neck and oesophagus (Scanlan et al., (2000) Cancer Lett 150(2):155-64; WO9904265) . The inventors have found that BRDThas a particularly suitable therapeutic window based on RNA expression. Furthermore the inventors have identified novel peptides derived from BRDT that are presented on the cell surface in complex with MHC. These peptides are particularly useful for the development of reagents that can targets cells expressing BRDT and for the treatment of cancers, including non-small cell lung cancer and squamous cell carcinomas of the head and neck and oesophagus

CALHM3 (also known as FAM26A, and having Uniprot accession number: Q86XJ0) is a multi-transmembrane domain protein predicted to function as a pore-forming subunit of a voltage-gated ion channel. A polymorphism in the related CALHM family member 1 has been associated with the development of Alzheimer's disease (Dreses-Werringloer et al., Cell. 2008 Jun. 27; 133(7): 1149-1161) and genetic variability in CALHM family genes has been linked to sporadic Creutzfeldt-Jakob disease (Calero et al., Prion. 2012 September-October; 6(4):407-12). CALHM3 is expressed at high levels in tumour tissues, while its expression is low or absent from normal tissue (for example see WO2005030250 and WO2006100089); therefore, CALHM3 is a particularly attractive target for therapeutic intervention. The inventors have found that CALMH3 has a particularly suitable therapeutic window based on RNA expression. The inventors have found peptides derived from CALHM3 that are presented on the cell surface in complex with MHC. These peptides are particularly useful for the development of reagents that can target cancer cells expressing CALHM3 and are particularly useful for the treatment of various cancers including oesophageal and ovarian.

CLDN6 (also known Claudin-6 or Skullin, and having Uniprot accession number: P56747), is a member of the claudin family of cell adhesion molecules involved in the formation of tight junctions Turksen (2013) Tissue Barriers1(3):e26750. Turksen et al., (2001) Dev Dyn 222(2):292-300. Expression of CLDN6 has been reported in a number of cancers including ovarian, lung, gastric and breast cancers (WO2015150327; Kwon et al., (2013) Int J Mol Sci. 14(9):18148-80; Lal-Nag et al., (2012) Oncogenesis 1:e33; Wang et al., (2013) Diagn Pathol 8:190; Ushiku et al., (2012) Histopathology 61(6):1043-56). The inventors have found that CLDN6 has a particularly suitable therapeutic window based on RNA expression. Furthermore the inventors have identified novel peptides derived from CLDN6 that are presented on the cell surface in complex with MHC. These peptides are particularly useful for the development of reagents that can targets cells expressing CLDN6 and for the treatment of cancers, including ovarian, lung, gastric and breast cancers.

CT45A1 (also known as cancer/testis antigen family 45 member A1 and having Uniprot accession number Q5HYN5) belongs to the cancer/testis family of germline encoded tumour antigens. Expression of CT45A1 has been reported in cancer cells, while expression in normal tissue is restricted to testis (Chen et al. Proc Natl Acad Sci U S A. 2005 May 31; 102(22):7940-5; Chen et al. Cancer Immunol Res. 2014 May; 2(5):480-6; Chen et al. Int J Cancer. 2009 Jun. 15; 124(12):2893-8; WO2006029176). CT45A1 is therefore a particularly attractive target for therapeutic intervention. The inventors have found novel peptides derived from CT45A1 that are presented on the cell surface in complex with MHC. These peptides are particularly useful for the development of reagents that can targets cells expressing CT45A1 and for the treatment of cancers, including non small cell lung cancer (squamous) and oesophageal cancer.

DCAF4L2 (also known as DDB1- and CUL4-associated factor 4-like protein 2 or WD repeat-containing protein 21C and having Uniprot accession number Q8NA75) is a cell cycle protein. DCAF4L2 is an ideal target for immunotherapeutic applications. The inventors have found novel peptides derived from DCAF4L2 that are presented on the cell surface in complex with MHC. These peptides are particularly useful for the development of reagents that can target cells expressing DCAF4L2 and for the treatment of cancers, including non small cell lung cancer and liver cancer.

HOXB13 (also known as homeobox protein Hox-B13 and having Uniprot accession number Q92826) is a sequence-specific transcription factor. HOXB13 has been associated with cancer (Xu et al. Hum Genet. 2013 January; 132(1):5-14). HOXB13 is an ideal target for immunotherapeutic applications. The inventors have found novel peptides derived from HOXB13 that are presented on the cell surface in complex with MHC. These peptides are particularly useful for the development of reagents that can targets cells expressing HOXB13 and for the treatment of cancers, including prostate cancer.

HTR3A (also known as 5-hydroxytryptamine receptor 3A and having Uniprot accession number P46098) is a serotonin receptor and ligand gated ion channel. HTR3A has been linked to cancer (WO2006021343). HTR3A is an ideal target for immunotherapeutic applications. The inventors have found novel peptides derived from HTR3A that are presented on the cell surface in complex with MHC. These peptides are particularly useful for the development of reagents that can targets cells expressing HTR3A and for the treatment of cancers, including ovarian cancer.

KLK3 (also known as kallikrein-3 or PSA or seminin and having Uniprot accession number P07288) is a serine endopeptidase. Expression of KLK3 has been linked with prostate cancer (Hong et al. Biomed Res Int. 2014; 2014:526341). KLK3 is an ideal target for immunotherapeutic applications. The inventors have found novel peptides derived from KLK3 that are presented on the cell surface in complex with MHC. These peptides are particularly useful for the development of reagents that can targets cells expressing KLK3 and for the treatment of cancers, including prostate cancer.

KLK4 (also known as serine protease 17, and having Uniprot accession number: Q9Y5K2) is believed to play a role in enamel formation (Hart et al., J. Med. Genet. 41:545-549(2004)). KLK4 is known to have a role in a number of diseases including cancer. KLK4 expression has been reported in breast and prostate cancers (Klokk et al., Cancer Res. 67:5221-5230(2007); Mange et al., Biochem. Biophys. Res. Commun. 375:107-112(2008); Papachristopoulou et al., Thromb. Haemost. 101:381-387(2009); Gao et al., Prostate 67:348-360(2007)). Thus, KLK4 is a particularly attractive target for therapeutic intervention (for examples see, WO2005083110; WO02077243; WO2011009173; WO2015014820). The inventors have found novel peptides from KLK4 that are presented on the cell surface in complex with MHC. These peptides are particularly useful for the development of reagents that can target cells expressing KLK4 and for the treatment of cancers, in particular prostate cancer.

LGSN (also known as lengsin or glutamate-ammonia ligase domain-containing protein 1 or lens glutamine synthase-like and having Uniprot accession number Q5TDP6) is a ligase involved in terminal differentiation in the lens. LGSN has been associated with cancer (Nakatsugawa et al. Cancer Sci. 2009 August; 100(8):1485-93). LGSN is an ideal target for immunotherapeutic applications. The inventors have found novel peptides derived from LGSN that are presented on the cell surface in complex with MHC. These peptides are particularly useful for the development of reagents that can targets cells expressing LGSN and for the treatment of cancers, including non small cell lung cancer (adenocarcinoma).

MAGEB2 (also known as melanoma-associated antigen B2 or CT3.2 or DAME and having Uniprot accession number 015479) belongs to the cancer/testis family of germline encoded tumour antigens. MAGE B2 is may function to enhance the ubiquitin ligase activity of RING-type zinc finger-containing E3 ubiquitin-protein ligases (Doyle et al. Mol Cell. 2010 Sep. 24; 39(6):963-74). Expression of MAGEB2 has been reported in various tumours, while expression in normal tissues is restricted to placenta and testis (Lurquin et al. Genomics. 1997 Dec. 15; 46(3):397-408). MAGEB2 is therefore a particularly attractive target for therapeutic intervention. The inventors have found novel peptides derived from MAGEB2 that are presented on the cell surface in complex with MHC. These peptides are particularly useful for the development of reagents that can targets cells expressing MAGEB2 and for the treatment of cancers, including non small cell lung cancer (squamous), oesophageal, liver and head&neck cancers.

MAGEC1 (also known as melanoma-associated antigen C1 or CT7.1, and having Uniprot accession number O60732) belongs to the cancer/testis family of germline encoded tumour antigens. Expression of MAGEC1 has been reported in various tumours, while expression in normal tissues is restricted to testis (Chen et al. Proc Natl Acad Sci U S A. 2005 May 31; 102(22):7940-5; Lucas et al. Cancer Res. 1998 Feb. 15; 58(4):743-52; Zimmerman et al. Virchows Arch. 2013 May; 462(5):565-74; Jungbluth et al. Int J Cancer. 2002 Jun. 20; 99(6):839-45; WO9954738).MAGEC1 is therefore a particularly attractive target for therapeutic intervention. The inventors have found novel peptides derived from MAGEC1 that are presented on the cell surface in complex with MHC. These peptides are particularly useful for the development of reagents that can targets cells expressing MAGEC1 and for the treatment of cancers, including non small cell lung cancer (squamous and adenocarcinoma), liver and ovarian cancers.

NPSR1 (having Uniprot accession number B7ZMA2) belongs to the family of G protein coupled receptors and has neuropeptide receptor activity. NPRS1 is reported to play a role in cancer (EP1365030; Pulkkinen et al. Virchows Archiv 465.2: 173-183 (2014)) and therefore is a suitable target for therapeutic intervention. The inventors have found that NPSR1 is a particularly attractive target for cancer therapy having expression in cancer cells including colon and oesophageal and low expression in normal tissues. The inventors have found novel peptides derived from NPSR1 that are presented on the cell surface in complex with MHC. These peptides are particularly useful for the development of reagents that can targets cells expressing NPSR1 and for the treatment of cancers, including colon and oesophageal cancers.

NR0B1 (also known as nuclear receptor subfamily 0 group B member 1 or DSS-AHC critical region on the X chromosome protein 1 or nuclear receptor DAX-1 and having Uniprot accession number P51843) is a nuclear receptor involved in transcriptional regulation. NR0B1 expression has been lined with cancer (Nakamura et al. Endocr J. 2009; 56(1):39-44; Oda et al. Am J Pathol. 2009 September; 175(3):1235-45). NR0B1 is an ideal target for immunotherapeutic applications. The inventors have found novel peptides derived from NR0B1 that are presented on the cell surface in complex with MHC. These peptides are particularly useful for the development of reagents that can targets cells expressing NR0B1 and for the treatment of cancers, including non small cell lung cancer (adenocarcinoma and squamous).

PAEP (also known as Progesterone-associated endometrial protein, or Glycodelin, or Placental protein 14, or Pregnancy-associated endometrial alpha-2 globulin, and having Uniprot accession number: P09466), is a glycoprotein with Immunosupressive functions, expressed in the endometrium during the menstrual cycle and in the first semester of pregnancy. Expression of PAEP has been reported in a number of cancers including endometrial, ovarian and non-small cell lung cancer (Lenhard et al., (2013) BMC Cancer; 13:616; Tsviliana et al., (2010) Anticancer Res. 5:1637-40; Schneider et al., (2015) Clin Cancer Res; 21(15):3529-40) . The inventors have found that PAEP has a particularly suitable therapeutic window. Furthermore the inventors have identified novel peptides derived from PAEP that are presented on the cell surface in complex with MHC. These peptides are particularly useful for the development of reagents that can targets cells expressing PAEP and for the treatment of cancers, including lung adenocarcinoma, ovarian cancer, mesothelioma, endometrial cancers and cutaneous melanoma.

PAGE2 (also known as P antigen family member 2 or GAGEC2 and having Uniprot accession number Q7Z2X7) belongs to the GAGE family of germline encoded tumour antigens. Expression of PAGE2 has been reported in various tumours, while expression in normal tissues is restricted to testis (Yilmaz-Ozcan et al. PLoS One. 2014 Sep. 17; 9(9):e107905; Gjerstorff Tissue Antigens. 2008 March; 71(3):187-92). PAGE2 is an ideal target for immunotherapeutic applications (WO200402907 and Li et al Clin Exp Immunol. May 2005; 140(2): 310-319). The inventors have found novel peptides derived from PAGE2 that are presented on the cell surface in complex with MHC. These peptides are particularly useful for the development of reagents that can targets cells expressing PAGE2 and for the treatment of cancers, including non small cell lung cancer (squamous) and liver cancer.

PAGE5 (also known as P antigen family member 5 or CT16.1 or GAGEE and having Uniprot accession number Q96GU1) belongs to GAGE family of germline encoded tumour antigens. Expression of PAGE2 has been reported in various tumours, while expression in normal tissues is restricted to testis (Nylund et al. PLoS One. 2012; 7(9):e45382; Gjerstorff Tissue Antigens. 2008 March; 71(3):187-92). PAGE5 is an ideal target for immunotherapeutic applications (WO200402907 and Li et al Clin Exp Immunol. May 2005; 140(2): 310-319). The inventors have found novel peptides derived from PAGE5 that are presented on the cell surface in complex with MHC. These peptides are particularly useful for the development of reagents that can targets cells expressing PAGE5 and for the treatment of cancers, including liver cancer.

PIWIL1 (also known as piwi-like protein 1 or HIWI and having Uniprot accession number Q96J94) is associated with meiotic division and plays a central role during spermatogenesis. Expression of PIWIL1 has been reported in various tumours, while expression in normal tissues is restricted to testis (He et al. BMC Cancer. 2009 Dec. 8; 9:426; Grochola et al. Br J Cancer. 2008 Oct. 7; 99(7):1083-8; Taubert et al. Oncogene. 2007 Feb. 15; 26(7):1098-100; Li et al. Oncol Rep. 2010 April; 23(4):1063-8; Zeng et al. Chin Med J (Engl). 2011 July; 124(14):2144-9; WO2000032039). PIWIL1 is an ideal target for immunotherapeutic applications. The inventors have found novel peptides derived from PIWIL1 that are presented on the cell surface in complex with MHC. These peptides are particularly useful for the development of reagents that can targets cells expressing PIWIL1 and for the treatment of cancers, including colon and oesophageal cancers.

RLN1 (also known as prorelaxin H1 and having Uniprot accession number P04808) is an ovarian hormone that plays a role in pregnancy. Expression of RLN1 has been associated with prostate cancer (Feng et al. Clin Cancer Res. 2007 Mar. 15; 13(6):1695-702; Feng et al. Ann N Y Acad Sci. 2009 April; 1160:379-80; WO2000055174; US20040006205). RLN1 is an ideal target for immunotherapeutic applications. The inventors have found novel peptides derived from RLN1 that are presented on the cell surface in complex with MHC. These peptides are particularly useful for the development of reagents that can targets cells expressing RLN1 and for the treatment of cancers, including prostate cancer.

SAGE1 (also known as sarcoma antigen 1 or CT14 and having Uniprot accession number Q9NXZ1) belongs to the cancer/testis family of germline encoded tumour antigens. Expression of SAGE1 has been reported in various tumours while expression in normal tissues is restricted to testis Martelange et al. Cancer Res. 2000 Jul. 15; 60(14):3848-55; US20120238012). SAGE1 is an ideal target for immunotherapeutic applications. The inventors have found novel peptides derived from SAGE1 that are presented on the cell surface in complex with MHC. These peptides are particularly useful for the development of reagents that can targets cells expressing SAGE1 and for the treatment of cancers, including acute myeloid leukemia, non small cell lung cancer (squamous) and head and neck cancer.

SLC30A8 (also known as Solute carrier family 30 member 8 or Zinc transporter 8 and having Uniprot accession number Q81WU4) is a zinc transporter protein which may have a role in insulin maturation and storage (Chimienti et al. J Cell Sci. 2006 Oct. 15; 119(Pt 20):4199-206). SLC30A8 is an ideal target for immunotherapeutic applications. The inventors have found novel peptides derived from SLC30A8 that are presented on the cell surface in complex with MHC. These peptides are particularly useful for the development of reagents that can target cells expressing SLC30A8 and for the treatment of cancers, including breast cancer.

SLC45A2 (also known as solute carrier family 45 member 2 or melanoma antigen AIM1 and having Uniprot accession number Q9UMX9) is a melanocyte differentiation antigen. Expression of SLC45A2 has been associated with melanoma (Fernandez et al. Hum Mutat. 2008 September; 29(9):1161-7; WO2009065944). SLC45A2 is an ideal target for immunotherapeutic applications. The inventors have found novel peptides derived from SLC45A2 that are presented on the cell surface in complex with MHC. These peptides are particularly useful for the development of reagents that can targets cells expressing SLC45A2 and for the treatment of cancers, including uveal melanoma.

SMC1B (also known as structural maintenance of chromosomes protein 1B or SMC-1-beta and having Uniprot accession number Q8NDV3) is a DNA binding protein involved in meiotic division. Expression of SMC1B has been associated with cancer (Ansari et al. J Cancer Res Clin Oncol. 2015 February; 141(2):369-80). SMC1 B is an ideal target for immunotherapeutic applications. The inventors have found novel peptides derived from SMC1B that are presented on the cell surface in complex with MHC. These peptides are particularly useful for the development of reagents that can target cells expressing SMC1B and for the treatment of cancers, including non small cell lung cancer (adenocarcinoma and squamous), breast cancer and liver cancer.

TRPM1 (also known as transient receptor potential cation channel subfamily M member 1 or LTrpC1 or Melastatin-1 and having Uniprot accession number Q7Z4N2) is a cation channel expressed in the retina. Expression of TRMP1 has been linked with melanoma (Fang et al. Biochem Biophys Res Commun. 2000 Dec. 9; 279(1):53-61). TRPM1 is an ideal target for immunotherapeutic applications. The inventors have found novel peptides derived from TRPM1 that are presented on the cell surface in complex with MHC. These peptides are particularly useful for the development of reagents that can targets cells expressing TRPM1 and for the treatment of cancers, including uveal melanoma.

XAGE1 (also known as G antigen family D member 2 (GAGED2); Uniprot accession number: Q9HD64) is a human X-linked gene belonging to the XAGE family of cancer testis (CT) tumour antigens (Liu et al., Cancer Res. 2000 Sep. 1; 60(17):4752-5; Zendman et al., Int J Cancer. 2002 May 20; 99(3):361-9). High level expression of XAGE1 has been detected in normal testis and in various cancers including, Ewings sarcoma, melanoma, lung and breast cancers (Liu et al., Cancer Res. 2000 Sep. 1; 60(17):4752-5; Zendman et al., Int J Cancer. 2002 May 20; 99(3):361-9; Zendman et al. Int J Cancer. 2002 Jan. 10; 97(2):195-204; Egland et al., Mol Cancer Ther. 2002 May; 1(7):441-50). Four transcript variants of XAGE1 have been identified, with XAGE1 b identified as a dominant antigen in a non-small cell lung cancer and other tumours (Nakagawa et al. (2005) Clin Cancer Res. 1; 11(15):5496-503; Sato et al (2007) Cancer Immun. 5; 7:5). Spontaneous immune responses against XAGE1 have been reported (Ohue et al. (2012) Int J Cancer. 1; 131(5):E649-58; Morishita et al (2007) Microbiol Immunol. 51(8):755-62). Like other CT antigens the restricted expression of XAGE1 in normal tissue makes it a particularly attractive target for therapeutic intervention.

In a first aspect, the invention provides a peptide comprising, consisting essentially of, or consisting of (a) the amino acid sequence of any one of SEQ ID NOS: 1-274, or
(b) the amino acid sequence of any one of SEQ ID NOs: 1-274 with the exception of 1, 2 or 3 amino acid substitutions, and/or 1, 2 or 3 amino acid insertions, and/or 1, 2 or 3 amino acid deletions,
   wherein the peptide forms a complex with a Major Histocompatibility Complex (MHC) molecule.

The inventors have found that peptides of the invention are presented by MHC on the surface of tumour cells. Accordingly, the peptides of the invention, as well as moieties that bind the peptide-MHC complexes, can be used to develop therapeutic reagents.

In a preferred embodiment the peptides have the following sequences:

| Target Protein | Peptides |
|---|---|
| ACTL8 | Seq ID NOs: 1-6 |
| ASCL2 | Seq ID NOs: 7-21 |
| BRDT | Seq ID NOs: 22-24 |
| CALHM3 | Seq ID NOs: 25-29 |
| CLDN6 | Seq ID NOs: 30-34 |
| CT45A1 | Seq ID NOs: 35-39 |
| DCAF4L2 | Seq ID NOs: 40-42 |
| HOXB13 | Seq ID NOs: 43-45 |
| HTR3A | Seq ID NOs: 46-51 |
| KLK3 | Seq ID NOs: 52 |
| KLK4 | Seq ID NOs: 53-58 |
| LGSN | Seq ID NOs: 59-61 |
| MAGEB2 | Seq ID NOs: 62-66 |
| MAGEC1 | Seq ID NOs: 67-112 |
| NPSR1 | Seq ID NOs: 113-122 |
| NR0B1 | Seq ID NOs: 123-141 |
| PAEP | Seq ID NOs: 142-149 |
| PAGE2 | Seq ID NOs: 150-160 |
| PAGE5 | Seq ID NOs: 161-169 |
| PIWIL1 | Seq ID NOs: 170-186 |
| RLN1 | Seq ID NOs: 187-191 |
| SAGE1 | Seq ID NOs: 192-198 |
| SLC30A8 | Seq ID NOs: 199-205 |
| SLC45A2 | Seq ID NOs: 206-242 |
| SMC1B | Seq ID NOs: 243-256 |
| TRPM1 | Seq ID NOs: 257-272 |
| XAGE1 | Seq ID NOs: 272-274 |

As is known in the art the ability of a peptide to form an immunogenic complex with a given MHC type, and thus activate T cells, is determined by the stability and affinity of the peptide-MHC interaction (van der Burg et al. J Immunol. 1996 May 1; 156(9):3308-14). The skilled person can, for example, determine whether or not a given polypeptide forms a complex with an MHC molecule by determining whether the MHC can be refolded in the presence of the polypeptide using the process set out in Example 2. If the polypeptide does not form a complex with MHC then MHC will not refold. Refolding is commonly confirmed using an antibody that recognises MHC in a folded state only. Further details can be found in Garboczi et al., Proc Natl Acad Sci USA. 1992 Apr. 15; 89(8):3429-33. Alternatively, the skilled person may determine the ability of a peptide to stabilise MHC on the surface of TAP-deficient cell lines such as T2 cells, or other biophysical methods to determine interaction parameters (Harndahl et al. J Biomol Screen. 2009 February; 14(2):173-80).

Preferably, peptides of the invention are from about 8 to about 16 amino acids in length, and are most preferably 8, 9, or 10 or 11 or 12 amino acids in length.

The peptides of the invention may consist or consist essentially of the amino acid sequences provided in SEQ ID NOs: 1-274.

The amino acid residues comprising the peptides of the invention may be chemically modified. Examples of chemical modifications include those corresponding to post translational modifications for example phosphorylation, acetylation and deamidation (Engelhard et al., Curr Opin Immunol. 2006 February; 18(1):92-7). Chemical modifications may not correspond to those that may be present in vivo. For example, the N or C terminal ends of the peptide may be modified improve the stability, bioavailability and or affinity of the peptides (see for example, Brinckerhoff et al Int J Cancer. 1999 Oct. 29; 83(3):326-34). Further examples of non-natural modifications include incorporation of non-encoded α-amino acids, photoreactive cross-linking amino acids, N-methylated amino acids, and β-amino acids, backbone reduction, retroinversion by using d-amino acids, N-terminal methylation and C-terminal amidation and pegylation.

Amino acid substitution means that an amino acid residue is substituted for a replacement amino acid residue at the same position. Inserted amino acid residues may be inserted at any position and may be inserted such that some or all of the inserted amino acid residues are immediately adjacent one another or may be inserted such that none of the inserted amino acid residues is immediately adjacent another inserted amino acid residue. One, two or three amino acids may be deleted from the sequence of SEQ ID NOs: 1-274. Each deletion can take place at any position of SEQ ID NOs: 1-274.

In some embodiments, the polypeptide of the invention may comprise one, two or three additional amino acids at the C-terminal end and/or at the N-terminal end of the sequence of SEQ ID NOs: 1-274. A polypeptide of the invention may comprise the amino acid sequence of SEQ ID NOs: 1-274 with the exception of one amino acid substitution and one amino acid insertion, one amino acid substitution and one amino acid deletion, or one amino acid insertion and one amino acid deletion. A polypeptide of the invention may comprise the amino acid sequence of SEQ ID NOs: 1-274, with the exception of one amino acid substitution, one amino acid insertion and one amino acid deletion.

Inserted amino acids and replacement amino acids may be naturally occurring amino acids or may be non-naturally occurring amino acids and, for example, may contain a non-natural side chain, and/or be linked together via non-native peptide bonds. Such altered peptide ligands are discussed further in Douat-Casassus et al., J. Med. Chem, 2007 Apr. 5; 50(7):1598-609 and Hoppes et al., J. Immunol 2014 Nov. 15; 193(10):4803-13 and references therein). If more than one amino acid residue is substituted and/or inserted, the replacement/inserted amino acid residues may be the same as each other or different from one another. Each replacement amino acid may have a different side chain to the amino acid being replaced.

Amino acid substitutions may be conservative, by which it is meant the substituted amino acid has similar chemical properties to the original amino acid. A skilled person would understand which amino acids share similar chemical properties. For example, the following groups of amino acids share similar chemical properties such as size, charge and polarity: Group 1 Ala, Ser, Thr, Pro, Gly; Group 2 asp, asn, glu, gln; Group 3 His, Arg, Lys; Group 4 Met, Leu, Ile, Val, Cys; Group 5 Phe Thy Trp.

Preferably, polypeptides of the invention bind to MHC in the peptide binding groove of the MHC molecule. Generally the amino acid modifications described above will not impair the ability of the peptide to bind MHC. In a preferred embodiment, the amino acid modifications improve the ability of the peptide to bind MHC. For example, mutations may be made at positions which anchor the peptide to MHC. Such anchor positions and the preferred residues at these locations are known in the art, particularly for peptides which bind HLA-A*02 (see, e. g. Parkhurst et al., J. Immunol. 1996 Sep. 15; 157(6):2539-48 and Parker et al. J Immunol. 1992 Dec. 1; 149(11):3580-7). Amino acids residues at position 2, and at the C terminal end, of the peptide are considered primary anchor positions. Preferred anchor residues may be different for each HLA type. The preferred amino acids in position 2 for HLA-A*02 are Leu, Ile, Val or Met. At the C terminal end, a valine or leucine is favoured.

A peptide of the invention may be used to elicit an immune response. If this is the case, it is important that the immune response is specific to the intended target in order to avoid the risk of unwanted side effects that may be associated with an “off target” immune response. Therefore, it is preferred that the amino acid sequence of a peptide of the invention does not match the amino acid sequence of a peptide from any other protein(s), in particular, that of another human protein. A person of skill in the art would understand how to search a database of known protein sequences to ascertain whether a peptide according to the invention is present in another protein.

Peptides of the invention may be conjugated to additional moieties such as carrier molecules or adjuvants for use as vaccines (for specific examples see Liu et al. Bioconjug Chem. 2015 May 20; 26(5): 791-801 and references therein). The peptides may be biotinylated or include a tag, such as a His tag. Examples of adjuvants used in cancer vaccines include microbes, such as the bacterium

Bacillus Calmette-Guérin (BCG), and/or substances produced by bacteria, such as Detox B (an oil droplet emulsion of monophosphoryl lipid A and mycobacterial cell wall skeleton). KLH (keyhole limpet hemocyanin) and bovine serum albumin are examples of suitable carrier proteins used in vaccine compositions Alternatively or additionally, the peptide may attached, covalently or otherwise, to proteins such as MHC molecules and/or antibodies (for example , see King et al. Cancer Immunol Immunother. 2013 June; 62(6):1093-105). Alternatively or additionally the peptides may be encapsulated into liposomes (for example see Adamina et al Br J Cancer. 2004 Jan. 12; 90(1):263-9). Such modified peptides may not correspond to any molecule that exists in nature.

Peptides of the invention can be synthesised easily by Merrifield synthesis, also known as solid phase synthesis, or any other peptide synthesis methodology. GMP grade peptide is produced by solid-phase synthesis techniques by Multiple Peptide Systems, San Diego, CA. As such, the peptides may be immobilised, for example to a solid support such as a bead. Alternatively, the peptide may be recombinantly produced, if so desired, in accordance with methods known in the art. Such methods typically involve the use of a vector comprising a nucleic acid sequence encoding the peptide to be expressed, to express the polypeptide in vivo; for example, in bacteria, yeast, insect or mammalian cells. Alternatively, in vitro cell-free systems may be used. Such systems are known in the art and are commercially available for example from Life Technologies, Paisley, UK. The peptides may be isolated and/or may be provided in substantially pure form. For example, they may be provided in a form which is substantially free of other peptides or proteins.

In a second aspect the invention provides a complex of the peptide of the first aspect and an MHC molecule. Preferably, the peptide is bound to the peptide binding groove of the MHC molecule. The MHC molecule may be MHC class I. The MHC class I molecule may be selected from HLA-A*02, HLA-A*01, HLA-A*03, HLA-A11, HLA-A23, HLA-A24, HLA-B*07, HLA-B*08, HLA-B40, HLA-B44, HLA-B15, HLA-C*04, HLA*C*03 HLA-C*07. As is known to those skilled in the art there are allelic variants of the above HLA types, all of which are encompassed by the present invention. A full list of HLA alleles can be found on the EMBL Immune Polymorphism Database (www.ebi.ac.uk/ipd/imgt/hla/allele.html; Robinson et al. Nucleic Acids Research (2015) 43:D423-431). The MHC molecule may be HLA-A*02.

The complex of the invention may be isolated and/or in a substantially pure form. For example, the complex may be provided in a form which is substantially free of other peptides or proteins. It should be noted that in the context of the present invention, the term “MHC molecule” includes recombinant MHC molecules, non-naturally occurring MHC molecules and functionally equivalent fragments of MHC, including derivatives or variants thereof, provided that peptide binding is retained. For example, MHC molecules may be fused to a therapeutic moiety, attached to a solid support, in soluble form, attached to a tag, biotinylated and/or in multimeric form. The peptide may be covalently attached to the MHC.

Methods to produce soluble recombinant MHC molecules with which peptides of the invention can form a complex are known in the art. Suitable methods include, but are not limited to, expression and purification from E. coli cells or insect cells. A suitable method is provided in Example 2 herein. Alternatively, MHC molecules may be produced synthetically, or using cell free systems.

Polypeptides and/or polypeptide-MHC complexes of the invention may be associated (covalently or otherwise) with a moiety capable of eliciting a therapeutic effect. Such a moiety may be a carrier protein which is known to be immunogenic. KLH (keyhole limpet hemocyanin) and bovine serum albumin are examples of suitable carrier proteins used in vaccine compositions. Alternatively, the peptides and/or peptide-MHC complexes of the invention may be associated with a fusion partner.

Fusion partners may be used for detection purposes, or for attaching said peptide or MHC to a solid support, or for MHC oligomerisation. The MHC complexes may incorporate a biotinylation site to which biotin can be added, for example, using the BirA enzyme (O'Callaghan et al., 1999 Jan. 1; 266(1):9-15). Other suitable fusion partners include, but are not limited to, fluorescent, or luminescent labels, radiolabels, nucleic acid probes and contrast reagents, antibodies, or enzymes that produce a detectable product. Detection methods may include flow cytometry, microscopy, electrophoresis or scintillation counting. Fusion partners may include cytokines, such as interleukin 2, interferon alpha, and granulocyte-macrophage colony-stimulating factor.

Peptide-MHC complexes of the invention may be provided in soluble form, or may be immobilised by attachment to a suitable solid support. Examples of solid supports include, but are not limited to, a bead, a membrane, sepharose, a magnetic bead, a plate, a tube, a column. Peptide-MHC complexes may be attached to an ELISA plate, a magnetic bead, or a surface plasmon reasonance biosensor chip. Methods of attaching peptide-MHC complexes to a solid support are known to the skilled person, and include, for example, using an affinity binding pair, e.g. biotin and streptavidin, or antibodies and antigens. In a preferred embodiment peptide-MHC complexes are labelled with biotin and attached to streptavidin-coated surfaces.

Peptide-MHC complexes of the invention may be in multimeric form, for example, dimeric, or tetrameric, or pentameric, or octomeric, or greater. Examples of suitable methods for the production of multimeric peptide MHC complexes are described in Greten et al., Clin. Diagn. Lab. Immunol. 2002 March; 9(2):216-20 and references therein. In general, peptide-MHC multimers may be produced using peptide-MHC tagged with a biotin residue and complexed through fluorescent labelled streptavidin. Alternatively, multimeric peptide-MHC complexes may be formed by using immunoglobulin as a molecular scaffold. In this system, the extracellular domains of MHC molecules are fused with the constant region of an immunoglobulin heavy chain separated by a short amino acid linker. Peptide-MHC multimers have also been produced using carrier molecules such as dextran (WO02072631). Multimeric peptide MHC complexes can be useful for improving the detection of binding moieties, such as T cell receptors, which bind said complex, because of avidity effects.

The polypeptides of the invention may be presented on the surface of a cell in complex with MHC. Thus, the invention also provides a cell presenting on its surface a complex of the invention. Such a cell may be a mammalian cell, preferably a cell of the immune system, and in particular a specialised antigen presenting cell such as a dendritic cell or a B cell. Other preferred cells include T2 cells (Hosken, et al., Science. 1990 Apr. 20; 248(4953):367-70). Cells presenting the polypeptide or complex of the invention may be isolated, preferably in the form of a population, or provided in a substantially pure form. Said cells may not naturally present the complex of the invention, or alternatively said cells may present the complex at a level higher than they would in nature. Such cells may be obtained by pulsing said cells with the polypeptide of the invention. Pulsing involves incubating the cells with the polypeptide for several hours using polypeptide concentrations typically ranging from 10⁻⁵ to 10^{31 12} M. Said cells may additionally be transduced with HLA molecules, such as HLA-A*02 to further induce presentation of the peptide. Cells may be produced recombinantly. Cells presenting peptides of the invention may be used to isolate T cells and T cell receptors (TCRs) which are activated by, or bind to, said cells, as described in more detail below.

In a third aspect, the invention provides a nucleic acid molecule comprising a nucleic acid sequence encoding the polypeptide of the first aspect of the invention. The nucleic acid may be cDNA. The nucleic acid molecule may consist essentially of a nucleic acid sequence encoding the peptide of the first aspect of the invention or may encode only the peptide of the invention, i.e. encode no other peptide or polypeptide.

Such a nucleic acid molecule can be synthesised in accordance with methods known in the art. Due to the degeneracy of the genetic code, one of ordinary skill in the art will appreciate that nucleic acid molecules of different nucleotide sequence can encode the same amino acid sequence.

In a fourth aspect, the invention provides a vector comprising a nucleic acid sequence according to the third aspect of the invention. The vector may include, in addition to a nucleic acid sequence encoding only a peptide of the invention, one or more additional nucleic acid sequences encoding one or more additional peptides. Such additional peptides may, once expressed, be fused to the N-terminus or the C-terminus of the peptide of the invention. In one embodiment, the vector includes a nucleic acid sequence encoding a peptide or protein tag such as, for example, a biotinylation site, a FLAG-tag, a MYC-tag, an HA-tag, a GST-tag, a Strep-tag or a poly-histidine tag.

Suitable vectors are known in the art as is vector construction, including the selection of promoters and other regulatory elements, such as enhancer elements. The vector utilised in the context of the present invention desirably comprises sequences appropriate for introduction into cells. For instance, the vector may be an expression vector, a vector in which the coding sequence of the polypeptide is under the control of its own cis-acting regulatory elements, a vector designed to facilitate gene integration or gene replacement in host cells, and the like.

In the context of the present invention, the term “vector” encompasses a DNA molecule, such as a plasmid, bacteriophage, phagemid, virus or other vehicle, which contains one or more heterologous or recombinant nucleotide sequences (e.g., an above-described nucleic acid molecule of the invention, under the control of a functional promoter and, possibly, also an enhancer) and is capable of functioning as a vector in the sense understood by those of ordinary skill in the art. Appropriate phage and viral vectors include, but are not limited to, lambda (X) bacteriophage, EMBL bacteriophage, simian virus 40, bovine papilloma virus, Epstein-Barr virus, adenovirus, herpes virus, vaccinia virus, Moloney murine leukemia virus, Harvey murine sarcoma virus, murine mammary tumor virus, lentivirus and Rous sarcoma virus.

In a fifth aspect, the invention provides a cell comprising the vector of the fourth aspect of the invention. The cell may be an antigen presenting cell and is preferably a cell of the immune system. In particular, the cell may be a specialised antigen presenting cell such as a dendritic cell or a B cell. The cell may be a mammalian cell.

Peptides and complexes of the invention can be used to identify and/or isolate binding moieties that bind specifically to the peptide and/or the complex of the invention. Such binding moieties may be used as immunotherapeutic reagents and may include antibodies and TCRs.

In a sixth aspect, the invention provides a binding moiety that binds the polypeptide of the invention.

Preferably the binding moiety binds the peptide when said peptide is in complex with MHC. In the latter instance, the binding moiety may bind partially to the MHC, provided that it also binds to the peptide. The binding moiety may bind only the peptide, and that binding may be specific. The binding moiety may bind only the peptide MHC complex and that binding may be specific.

When used with reference to binding moieties that bind the complex of the invention, “specific” is generally used herein to refer to the situation in which the binding moiety does not show any significant binding to one or more alternative polypeptide-MHC complexes other than the polypeptide-MHC complex of the invention. TCRs that bind to one or more, and in particular several, antigens presented by cells that are not the intended target of the TCR, pose an increased risk of toxicity when administered in vivo because of potential off target reactivity. Such highly cross-reactive TCRs are not suitable for therapeutic use.

The binding moiety may be a T cell receptor (TCR). TCRs are described using the International Immunogenetics (IMGT) TCR nomenclature, and links to the IMGT public database of TCR sequences. The unique sequences defined by the IMGT nomenclature are widely known and accessible to those working in the TCR field. For example, they can be found in the “T cell Receptor Factsbook”, (2001) LeFranc and LeFranc, Academic Press, ISBN 0-12-441352-8; Lefranc, (2011), Cold Spring Harb Protoc 2011(6): 595-603; Lefranc, (2001), Curr Protoc Immunol Appendix 1: Appendix 1O; Lefranc, (2003), Leukemia 17(1): 260-266, and on the IMGT website (www.IMGT.org)

The TCRs of the invention may be in any format known to those in the art. For example, the TCRs may be αβ heterodimers, or αα or ββ homodimers.

Alpha-beta heterodimeric TCRs have an alpha chain and a beta chain. Broadly, each chain comprises variable, joining and constant region, and the beta chain also usually contains a short diversity region between the variable and joining regions, but this diversity region is often considered as part of the joining region. Each variable region comprises three hypervariable CDRs (Complementarity Determining Regions) embedded in a framework sequence; CDR3 is believed to be the main mediator of antigen recognition. There are several types of alpha chain variable (Vα) regions and several types of beta chain variable (Vβ) regions distinguished by their framework, CDR1 and CDR2 sequences, and by a partly defined CDR3 sequence.

The TCRs of the invention may not correspond to TCRs as they exist in nature. For example, they may comprise alpha and beta chain combinations that are not present in a natural repertoire. Alternatively or additionally they may be soluble, and/or the alpha and/or beta chain constant domain may be truncated relative to the native/naturally occurring TRAC/TRBC sequences such that, for example, the C terminal transmembrane domain and intracellular regions are not present. Such truncation may result in removal of the cysteine residues from TRAC/TRBC that form the native interchain disulphide bond.

In addition the TRAC/TRBC domains may contain modifications. For example, the alpha chain extracellular sequence may include a modification relative to the native/naturally occurring TRAC whereby amino acid T48 of TRAC, with reference to IMGT numbering, is replaced with C48. Likewise, the beta chain extracellular sequence may include a modification relative to the native/naturally occurring TRBC1 or TRBC2 whereby S57 of TRBC1 or TRBC2, with reference to IMGT numbering, is replaced with C57. These cysteine substitutions relative to the native alpha and beta chain extracellular sequences enable the formation of a non-native interchain disulphide bond which stabilises the refolded soluble TCR, i.e. the TCR formed by refolding extracellular alpha and beta chains (WO 03/020763). This non-native disulphide bond facilitates the display of correctly folded TCRs on phage. (Li et al., Nat Biotechnol. 2005 March; 23(3):349-54). In addition the use of the stable disulphide linked soluble TCR enables more convenient assessment of binding affinity and binding half-life. Alternative positions for the formation of a non-native disulphide bond are described in WO 03/020763. These include Thr 45 of exon 1 of TRAC*01 and Ser 77 of exon 1 of TRBC1*01 or TRBC2*01; Tyr 10 of exon 1 of TRAC*01 and Ser 17 of exon 1 of TRBC1*01 or TRBC2*01; Thr 45 of exon 1 of TRAC*01 and Asp 59 of exon 1 of TRBC1*01 or TRBC2*01; and Ser 15 of exon 1 of TRAC*01 and Glu 15 of exon 1 of TRBC1*01 or TRBC2*01. TCRs with a non-native disulphide bond may be full length or may be truncated.

TCRs of the invention may be in single chain format (such as those described in WO9918129). Single chain TCRs include αβ TCR polypeptides of the type: Vα-L-Vβ, Vβ-L-Vα, Vα-Cα-L-Vβ, Vα-L-Vβ-Cβ or Vα-Cα-L-Vβ-Cβ, optionally in the reverse orientation, wherein Vα and Vβ are TCR α and β variable regions respectively, Cα and Cβ are TCR α and β constant regions respectively, and L is a linker sequence. Single chain TCRs may contain a non-native disulphide bond. The TCR may be in a soluble form (i.e. having no transmembrane or cytoplasmic domains), or may contain full length alpha and beta chains. The TCR may be provided on the surface of a cell, such as a T cell.

TCRs of the invention may be engineered to include mutations. Methods for producing mutated high affinity TCR variants such as phage display and site directed mutagenesis and are known to those in the art (for example see WO 04/044004 and Li et al., Nat Biotechnol 2005 March; 23(3):349-54).). Preferably, mutations to improve affinity are made within the variable regions of alpha and/or beta chains. More preferably mutations to improve affinity are made within the CDRs. There may be between 1 and 15 mutations in the alpha and or beta chain variable regions.

TCRs of the invention may also be may be labelled with an imaging compound, for example a label that is suitable for diagnostic purposes. Such labelled high affinity TCRs are useful in a method for detecting a TCR ligand selected from CD1-antigen complexes, bacterial superantigens, and MHC-peptide/superantigen complexes, which method comprises contacting the TCR ligand with a high affinity TCR (or a multimeric high affinity TCR complex) which is specific for the TCR ligand; and detecting binding to the TCR ligand. In multimeric high affinity TCR complexes such as those described in Zhu et al., J. Immunol. 2006 Mar. 1; 176(5):3223-32, (formed, for example, using biotinylated heterodimers) fluorescent streptavidin (commercially available) can be used to provide a detectable label. A fluorescently-labelled multimer is suitable for use in FACS analysis, for example to detect antigen presenting cells carrying the peptide for which the high affinity TCR is specific.

A TCR of the present invention (or multivalent complex thereof) may alternatively or additionally be associated with (e.g. covalently or otherwise linked to) a therapeutic agent which may be, for example, a toxic moiety for use in cell killing, or an immunostimulating agent such as an interleukin or a cytokine. A multivalent high affinity TCR complex of the present invention may have enhanced binding capability for a TCR ligand compared to a non-multimeric wild-type or high affinity T cell receptor heterodimer. Thus, the multivalent high affinity TCR complexes according to the invention are particularly useful for tracking or targeting cells presenting particular antigens in vitro or in vivo, and are also useful as intermediates for the production of further multivalent high affinity TCR complexes having such uses. The high affinity TCR or multivalent high affinity TCR complex may therefore be provided in a pharmaceutically acceptable formulation for use in vivo.

High affinity TCRs of the invention may be used in the production of soluble bi-specific reagents. A preferred embodiment is a reagent which comprises a soluble TCR, fused via a linker to an anti-CD3 specific antibody fragment. Further details including how to produce such reagents are described in WO10/133828.

In a further aspect, the invention provides nucleic acid encoding the TCR of the invention, a TCR expression vector comprising nucleic acid encoding a TCR of the invention, as well as a cell harbouring such a vector. The TCR may be encoded either in a single open reading frame or two distinct open reading frames. Also included in the scope of the invention is a cell harbouring a first expression vector which comprises nucleic acid encoding an alpha chain of a TCR of the invention, and a second expression vector which comprises nucleic acid encoding a beta chain of a TCR of the invention. Alternatively, one vector may encode both an alpha and a beta chain of a TCR of the invention.

A further aspect of the invention provides a cell displaying on its surface a TCR of the invention. The cell may be a T cell, or other immune cell. The T cell may be modified such that it does not correspond to a T cell as it exists in nature. For example, the cell may be transfected with a vector encoding a TCR of the invention such that the T cell expresses a further TCR in addition to the native TCR. Additionally or alternatively the T cell may be modified such that it is not able to present the native TCR. There are a number of methods suitable for the transfection of T cells with DNA or RNA encoding the TCRs of the invention (see for example Robbins et al., J. Immunol. 2008 May 1; 180(9):6116-31). T cells expressing the TCRs of the invention are suitable for use in adoptive therapy-based treatment of diseases such as cancers. As will be known to those skilled in the art there are a number of suitable methods by which adoptive therapy can be carried out (see for example Rosenberg et al., Nat Rev Cancer. 2008 April; 8(4):299-308).

The TCRs of the invention intended for use in adoptive therapy are generally glycosylated when expressed by the transfected T cells. As is well known, the glycosylation pattern of transfected TCRs may be modified by mutations of the transfected gene (Kuball J et al., J Exp Med. 2009 Feb. 16; 206(2):463-75).

Examples of TCR variable region amino acid sequences that are able to specifically recognise peptides of the invention are provided in Example 3. TCRs having 90, 91, 92, 93, 94, 95, 96, 97, 98 or 99% identity to the sequences provided are also contemplated by the invention. TCRs with the same alpha and beta chain usage are also included in the invention.

The binding moiety of the invention may be an antibody. The term “antibody” as used herein refers to immunoglobulin molecules and immunologically active portions of immunoglobulin molecules, i.e., molecules that contain an antigen binding site that specifically binds an antigen, whether natural or partly or wholly synthetically produced. The term “antibody” includes antibody fragments, derivatives, functional equivalents and homologues of antibodies, humanised antibodies, including any polypeptide comprising an immunoglobulin binding domain, whether natural or wholly or partially synthetic and any polypeptide or protein having a binding domain which is, or is homologous to, an antibody binding domain. Chimeric molecules comprising an immunoglobulin binding domain, or equivalent, fused to another polypeptide are therefore included. Cloning and expression of chimeric antibodies are described in EP-A-0120694 and EP-A-0125023. A humanised antibody may be a modified antibody having the variable regions of a non-human, e.g. murine, antibody and the constant region of a human antibody. Methods for making humanised antibodies are described in, for example, U.S. Pat. No. 5,225,539. Examples of antibodies are the immunoglobulin isotypes (e.g., IgG, IgE, IgM, IgD and IgA) and their isotypic subclasses; fragments which comprise an antigen binding domain such as Fab, scFv, Fv, dAb, Fd; and diabodies. Antibodies may be polyclonal or monoclonal. A monoclonal antibody may be referred to herein as “mab”.

It is possible to take an antibody, for example a monoclonal antibody, and use recombinant DNA technology to produce other antibodies or chimeric molecules which retain the specificity of the original antibody. Such techniques may involve introducing DNA encoding the immunoglobulin variable region, or the complementary determining regions (CDRs), of an antibody to the constant regions, or constant regions plus framework regions, of a different immunoglobulin (see, for instance, EP-A-184187, GB 2188638A or EP-A-239400). A hybridoma (or other cell that produces antibodies) may be subject to genetic mutation or other changes, which may or may not alter the binding specificity of antibodies produced.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature. 1989 Oct. 12; 341(6242):544-6) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., Science. 1988 Oct. 21; 242(4877):423-6; Huston et al., Proc Natl Acad Sci U S A. 1988 August; 85(16):5879-83); (viii) bispecific single chain Fv dimers (PCT/US92/09965) and (ix) “diabodies”, multivalent or multispecific fragments constructed by gene fusion (WO94/13804; P. Hollinger et al., Proc Natl Acad Sci U S A. 1993 Jul. 15; 90(14):6444-8). Diabodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associate with each other to form an antigen binding site: antigen binding sites are formed by the association of the first domain of one polypeptide within the multimer with the second domain of another polypeptide within the multimer (WO94/13804). Where bispecific antibodies are to be used, these may be conventional bispecific antibodies, which can be manufactured in a variety of ways (Hollinger & Winter, Curr Opin Biotechnol. 1993 August; 4(4):446-9), e.g. prepared chemically or from hybrid hybridomas, or may be any of the bispecific antibody fragments mentioned above. It may be preferable to use scFv dimers or diabodies rather than whole antibodies. Diabodies and scFv can be constructed without an Fc region, using only variable domains, potentially reducing the effects of anti-idiotypic reaction. Other forms of bispecific antibodies include the single chain “Janusins” described in Traunecker et al., EMBO J. 1991 December; 10(12):3655-9). Bispecific diabodies, as opposed to bispecific whole antibodies, may also be useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied and an antibody of appropriate specificity selected. An “antigen binding domain” is the part of an antibody which comprises the area which specifically binds to and is complementary to part or all of an antigen. Where an antigen is large, an antibody may only bind to a particular part of the antigen, which part is termed an epitope. An antigen binding domain may be provided by one or more antibody variable domains. An antigen binding domain may comprise an antibody light chain variable region (VL) and an antibody heavy chain variable region (VH).

The binding moiety may be an antibody-like molecule that has been designed to specifically bind a peptide—MHC complex of the invention. Of particular preference are TCR-mimic antibodies, such as, for example those described in WO2007143104 and Sergeeva et al., Blood. 2011 Apr. 21; 117(16):4262-72 and/or Dahan and Reiter. Expert Rev Mol Med. 2012 Feb. 24; 14:e6.

Also encompassed within the present invention are binding moieties based on engineered protein scaffolds. Protein scaffolds are derived from stable, soluble, natural protein structures which have been modified to provide a binding site for a target molecule of interest. Examples of engineered protein scaffolds include, but are not limited to, affibodies, which are based on the Z-domain of staphylococcal protein A that provides a binding interface on two of its a-helices (Nygren, FEBS J. 2008 June; 275(11):2668-76); anticalins, derived from lipocalins, that incorporate binding sites for small ligands at the open end of a beta-barrel fold (Skerra, FEBS J. 2008 June; 275(11):2677-83), nanobodies, and DARPins. Engineered protein scaffolds are typically targeted to bind the same antigenic proteins as antibodies, and are potential therapeutic agents. They may act as inhibitors or antagonists, or as delivery vehicles to target molecules, such as toxins, to a specific tissue in vivo (Gebauer and Skerra, Curr Opin Chem Biol. 2009 June; 13(3):245-55). Short peptides may also be used to bind a target protein. Phylomers are natural structured peptides derived from bacterial genomes. Such peptides represent a diverse array of protein structural folds and can be used to inhibit/disrupt protein-protein interactions in vivo (Watt, Nat Biotechnol. 2006 February; 24(2):177-83)].

In another aspect, the invention further provides a peptide of the invention, a nucleic acid molecule of the invention, a vector of the invention, a cell of the invention or a binding moiety of the invention for use in medicine. The peptide, complex, nucleic acid, vector, cell or binding moiety may be used for in the treatment or prevention of cancer, in particular, breast, colon and oesophageal cancers

In a further aspect, the invention provides a pharmaceutical composition comprising a peptide of the invention, a nucleic acid molecule of the invention, a vector of the invention, a cell of the invention or a binding moiety of the invention together with a pharmaceutically acceptable carrier. This pharmaceutical composition may be in any suitable form, (depending upon the desired method of administering it to a patient). It may be provided in unit dosage form, will generally be provided in a sealed container and may be provided as part of a kit. Such a kit would normally (although not necessarily) include instructions for use. It may include a plurality of said unit dosage forms. Suitable compositions and methods of administration are known to those skilled in the art, for example see, Johnson et al., Blood. 2009 Jul. 16; 114(3):535-46, with reference to clinical trial numbers NCI-07-C-0175 and NCI-07-C-0174. Cells in accordance with the invention will usually be supplied as part of a sterile, pharmaceutical composition which will normally include a pharmaceutically acceptable carrier. For example, T cells transfected with TCRs of the invention may be provided in pharmaceutical composition together with a pharmaceutically acceptable carrier. The pharmaceutically acceptable carrier may be a cream, emulsion, gel, liposome, nanoparticle or ointment.

The pharmaceutical composition may be adapted for administration by any appropriate route such as a parenteral (including subcutaneous, intramuscular, or intravenous), enteral (including oral or rectal), inhalation or intranasal routes. Such compositions may be prepared by any method known in the art of pharmacy, for example by mixing the active ingredient with the carrier(s) or excipient(s) under sterile conditions.

Dosages of the substances of the present invention can vary between wide limits, depending upon the disease or disorder to be treated (such as cancer, viral infection or autoimmune disease), the age and condition of the individual to be treated, etc. For example, a suitable dose range for a reagent comprising a soluble TCR fused to an anti-CD3 domain may be between 25 ng/kg and 50 μg/kg. A physician will ultimately determine appropriate dosages to be used.

The polypeptide of the invention may be provided in the form of a vaccine composition. The vaccine composition may be useful for the treatment or prevention of cancer. All such compositions are encompassed in the present invention. As will be appreciated, vaccines may take several forms (Schlom, J Natl Cancer Inst. 2012 Apr. 18; 104(8):599-613). For example, the peptide of the invention may be used directly to immunise patients (Salgaller, Cancer Res. 1996 Oct. 15; 56(20):4749-57 and Marchand, Int J Cancer. 1999 Jan. 18; 80(2):219-30). The vaccine composition may include additional peptides such that the peptide of the invention is one of a mixture of peptides. Adjuvants may be added to the vaccine composition to augment the immune response

Alternatively the vaccine composition may take the form of an antigen presenting cell displaying the peptide of the invention in complex with MHC. Preferably the antigen presenting cell is an immune cell, more preferably a dendritic cell. The peptide may be pulsed onto the surface of the cell (Thurner, J Exp Med. 1999 Dec. 6; 190(11):1669-78), or nucleic acid encoding for the peptide of the invention may be introduced into dendritic cells (for example by electroporation. Van Tendeloo, Blood. 2001 Jul. 1; 98(1):49-56).

The polypeptides, complexes, nucleic acid molecules, vectors, cells and binding moieties of the invention may be non-naturally occurring and/or purified and/or engineered and/or recombinant and/or isolated and/or synthetic.

The invention also provides a method of identifying a binding moiety that binds a complex of the invention, the method comprising contacting a candidate binding moiety with the complex and determining whether the candidate binding moiety binds the complex. Methods to determine binding to polypeptide-MHC complexes are well known in the art. Preferred methods include, but are not limited to, surface plasmon resonance, or any other biosensor technique, ELISA, flow cytometry, chromatography, microscopy. Alternatively, or in addition, binding may be determined by functional assays in which a biological response is detected upon binding, for example, cytokine release or cell apoptosis.

The candidate binding moiety may be a binding moiety of the type already described, such as a TCR or an antibody. Said binding moiety may be obtained using methods that are known in the art.

For example, antigen binding T cells and TCRs have traditionally been are isolated from fresh blood obtained from patients or healthy donors. Such a method involves stimulating T cells using autologous DCs, followed by autologous B cells, pulsed with the polypeptide of the invention. Several rounds of stimulation may be carried out, for example three or four rounds. Activated T cells may then be tested for specificity by measuring cytokine release in the presence of T2 cells pulsed with the peptide of the invention (for example using an IFNγ ELISpot assay). Activated cells may then be sorted by fluorescence-activated cell sorting (FACS) using labelled antibodies to detect intracellular cytokine production (e.g. IFNγ), or expression of a cell surface marker (such as CD137). Sorted cells may be expanded and further validated, for example, by ELISpot assay and/or cytotoxicity against target cells and/or staining by peptide-MHC tetramer. The TCR chains from validated clones may then be amplified by rapid amplification of cDNA ends (RACE) and sequenced.

Alternatively, TCRs and antibodies may be obtained from display libraries in which the peptide MHC complex of the invention is used to pan the library. The production of antibody libraries using phage display is well known in the art, for example see Aitken, Antibody phage display: Methods and Protocols (2009, Humana, New York). TCRs can be displayed on the surface of phage particles and yeast particles for example, and such libraries have been used for the isolation of high affinity variants of TCR derived from T cell clones (as described in WO04044004 and Li et al. Nat Biotechnol. 2005 March; 23(3):349-54 and WO9936569). It has been demonstrated more recently that TCR phage libraries can be used to isolate TCRs with novel antigen specificity. Such libraries are typically constructed with alpha and beta chain sequences corresponding to those found in a natural repertoire. However, the random combination of these alpha and beta chain sequences, which occurs during library creation, produces a repertoire of TCRs not present in nature (as described in WO2015/136072, PCT/EP2016/071757, PCT/EP2016/071761, PCT/EP2016/071762, PCT/EP2016/071765, PCT/EP2016/071767, PCT/EP2016/071768, PCT/EP2016/071771 and PCT/EP2016/071772).

In a preferred embodiment, the peptide-MHC complex of the invention may be used to screen a library of diverse TCRs displayed on the surface of phage particles. The TCRs displayed by said library may not correspond to those contained in a natural repertoire, for example, they may contain alpha and beta chain pairing that would not be present in vivo, and or the TCRs may contain non-natural mutations and or the TCRs may be in soluble form. Screening may involve panning the phage library with peptide-MHC complexes of the invention and subsequently isolating bound phage. For this purpose peptide-MHC complexes may be attached to a solid support, such as a magnet bead, or column matrix and phage bound peptide MHC complexes isolated, with a magnet, or by chromatography, respectively. The panning steps may be repeated several times for example three or four times. Isolated phage may be further expanded in E. coli cells. Isolated phage particles may be tested for specific binding peptide-MHC complexes of the invention. Binding can be detected using techniques including, but not limited to, ELISA, or SPR for example using a BiaCore instrument. The DNA sequence of the T cell receptor displayed by peptide-MHC binding phage can be further identified by standard PCR methods.

Preferred or optional features of each aspect of the invention are as for each of the other aspects mutatis mutandis. The prior art documents mentioned herein are incorporated by reference to the fullest extent permitted by law.

The present invention will be further illustrated in the following Examples and Figures which are given for illustration purposes only and are not intended to limit the invention in any way.

### EXAMPLES

### Example 1—Identification of Target-Derived Peptides by Mass Spectrometry

Presentation of HLA-restricted peptides from each of the target proteins on the surface of tumour cell lines was investigated using mass spectrometry.

### Method

Immortalised cell lines obtained from commercial sources were maintained and expanded under standard conditions.

Class I HLA complexes were purified by immunoaffinity using commercially available anti-HLA antibodies BB7.1 (anti-HLA-B*07), BB7.2 (anti-HLA-A*02) and W6/32 (anti-Class 1). Briefly, cells were lysed in buffer containing non-ionic detergent NP-40 (0.5% v/v) at 5×10⁷ cells per ml and incubated at 4° C. for 1 h with agitation/mixing. Cell debris was removed by centrifugation and supernatant pre-cleared using proteinA-Sepharose. Supernatant was passed over 5 ml of resin containing 8 mg of anti-HLA antibody immobilised on a proteinA-Sepharose scaffold. Columns were washed with low salt and high salt buffers and complexes eluted in acid. Eluted peptides were separated from HLA complexes by reversed phase chromatography using a solid phase extraction cartridge (Phenomenex). Bound material was eluted from the column and reduced in volume using a vacuum centrifuge.

Peptides were separated by high pressure liquid chromatography (HPLC) on a Dionex Ultimate 3000 system using a C18 column (Phenomenex). Peptides were loaded in 98% buffer A (0.1% aqueous trifluoroacetic acid (TFA)) and 2% buffer B (0.1% TFA in acetonitrile). Peptides were eluted using a stepped gradient of B (2-60%) over 20 min. Fractions were collected at one minute intervals and lyophilised.

Peptides were analysed by nanoLCMS/MS using a Dionex Ultimate 3000 nanoLC coupled to either AB Sciex Triple TOF 5600 or Thermo Orbitrap Fusion mass spectrometers. Both machines were equipped with nanoelectrospray ion sources. Peptides were loaded onto an Acclaim PepMap 100 trap column (Dionex) and separated using an Acclaim PepMap RSLC column (Dionex). Peptides were loaded in mobile phase A (0.5% formic acid: water) and eluted using a gradient of buffer B (acetonitrile:0.5% formic acid) directly into the nanospray ionisation source.

For peptide identification the mass spectrometer was operated using an information dependent acquisition (IDA) workflow. Information acquired in these experiments was used to search the Uniprot database of human proteins for peptides consistent with the fragmentation patterns seen, using Protein pilot software (Ab Sciex) and PEAKS software (Bioinformatics solutions). Peptides identified are assigned a score by the software, based on the match between the observed and expected fragmentation patterns.

### Results

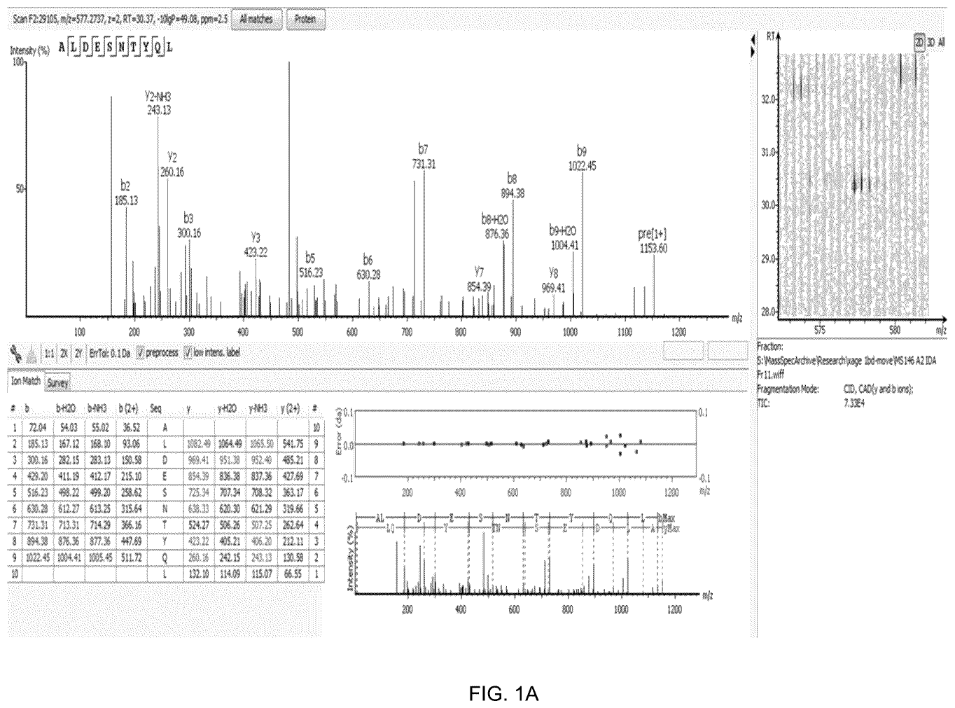

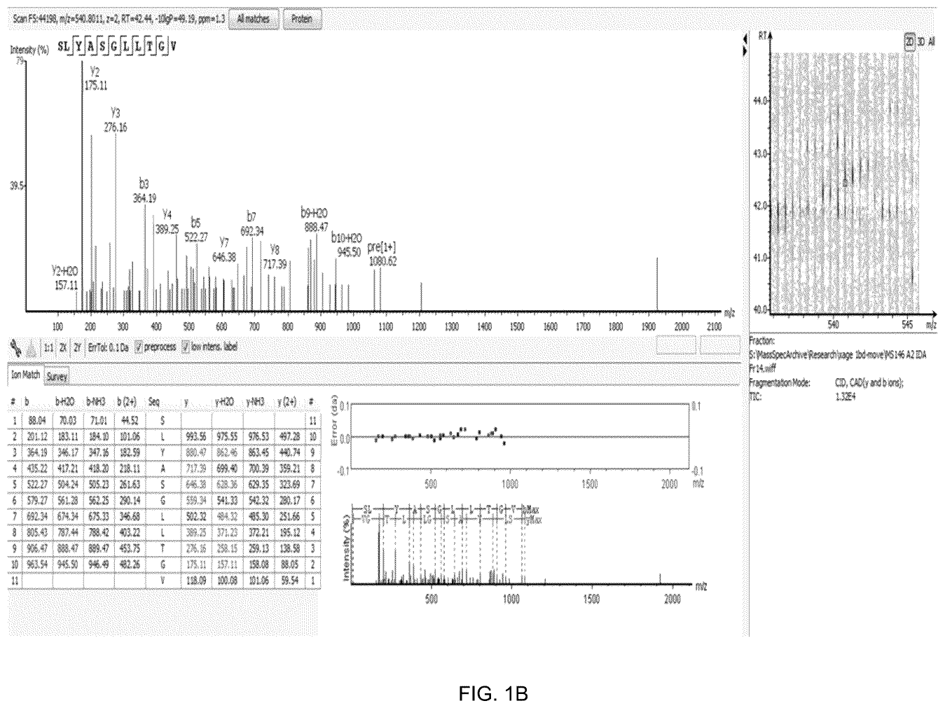

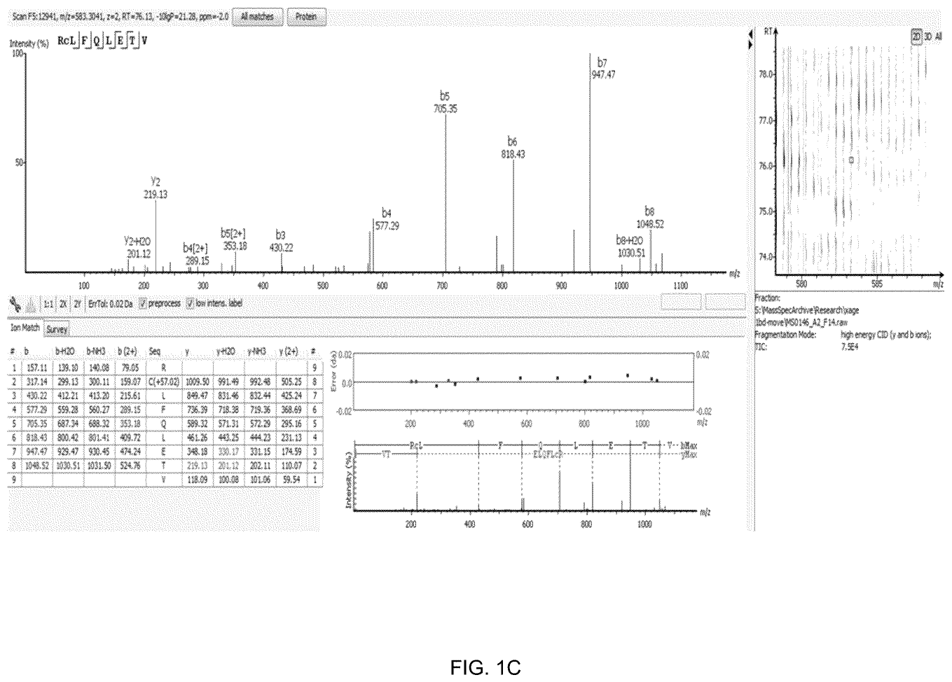

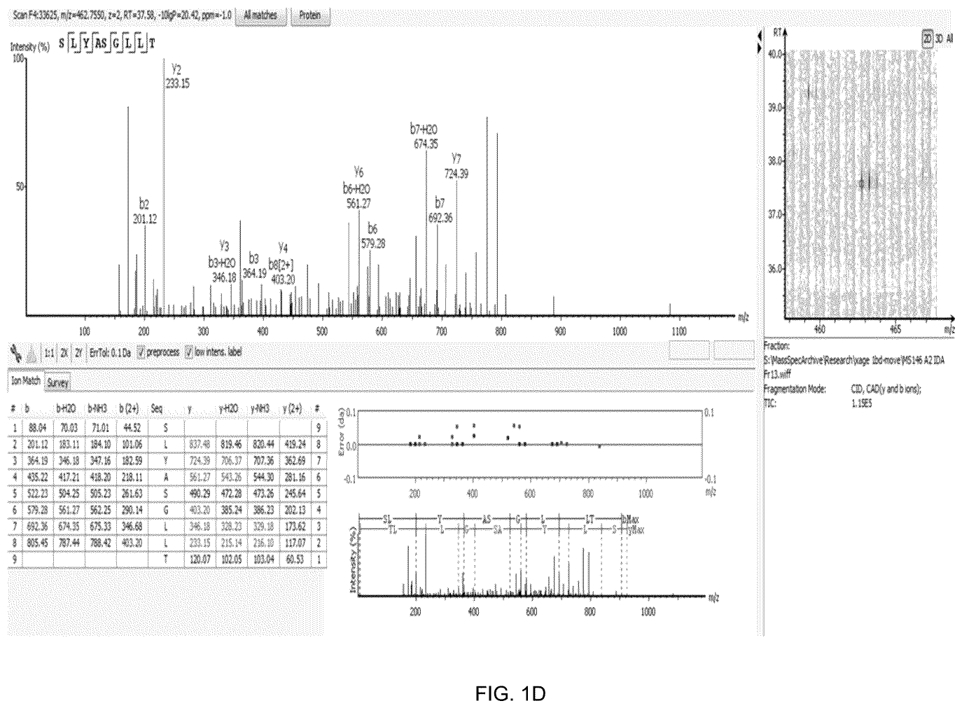

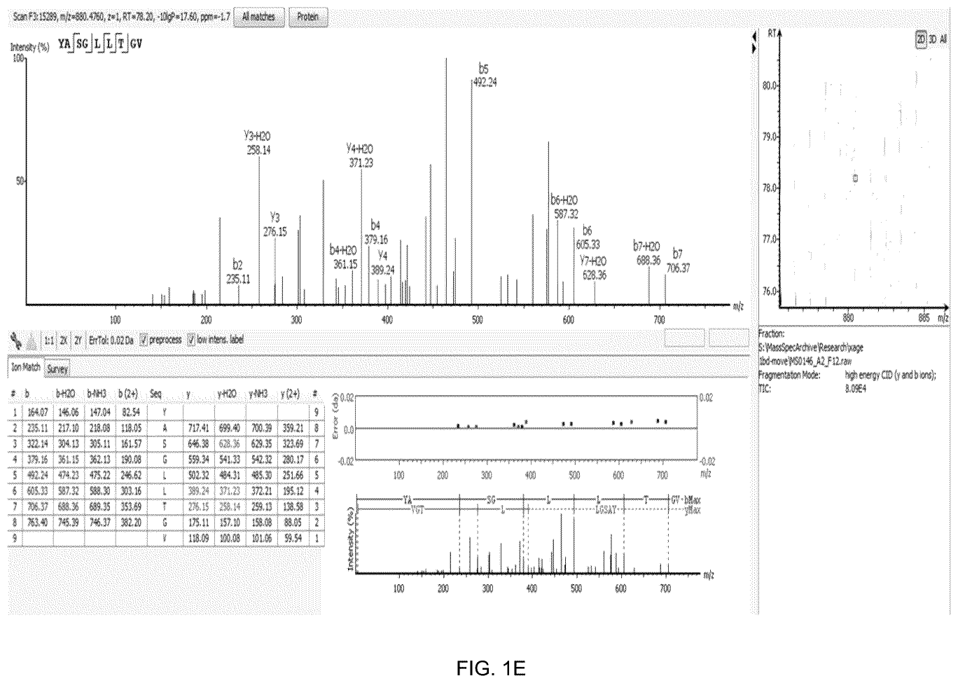

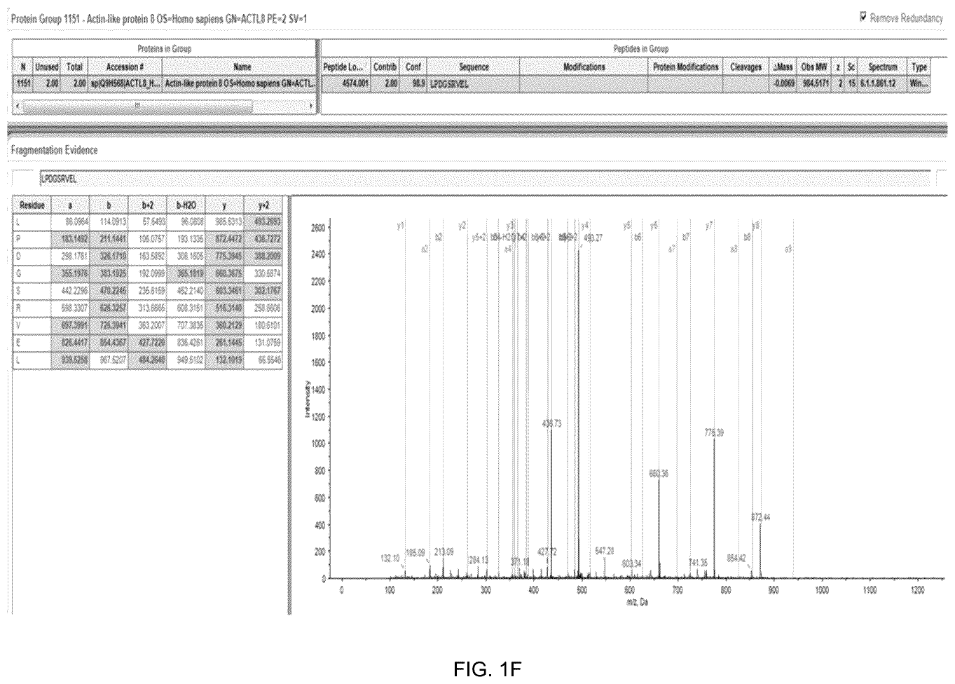

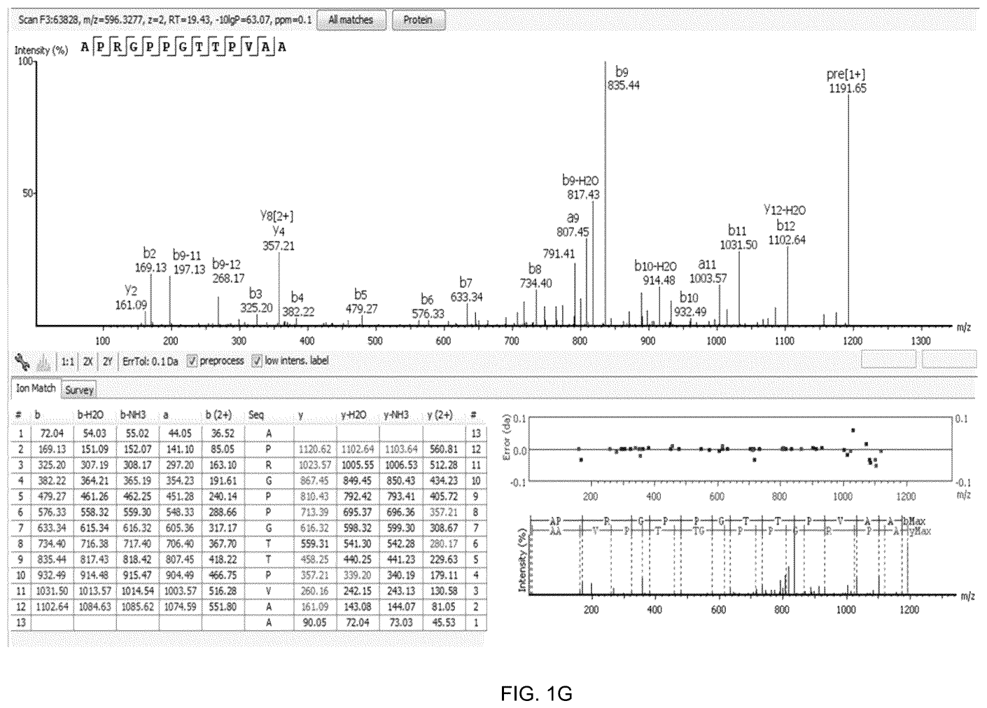

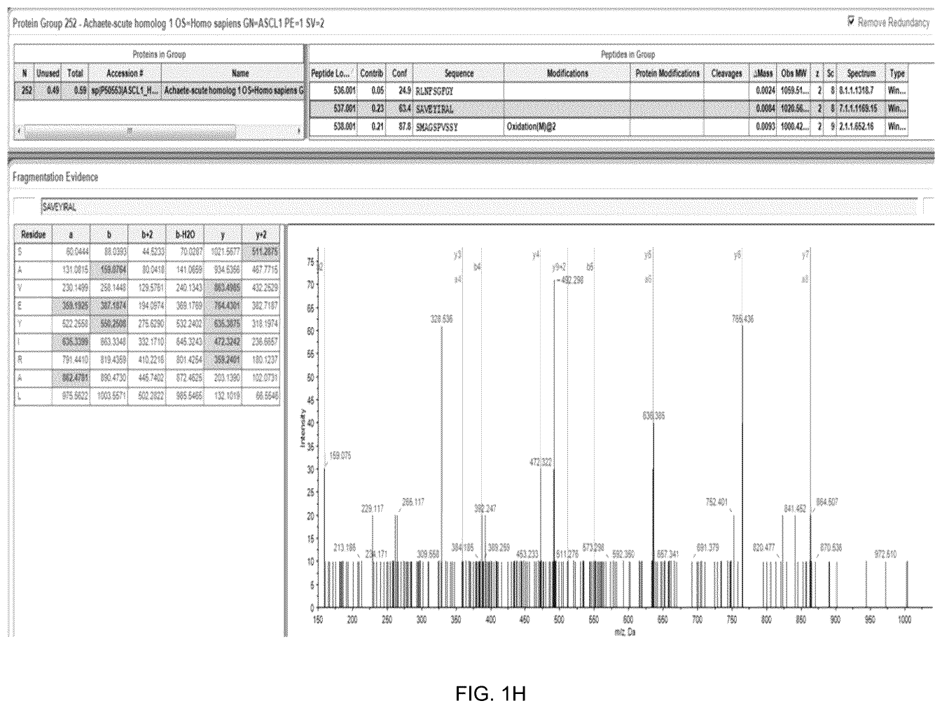

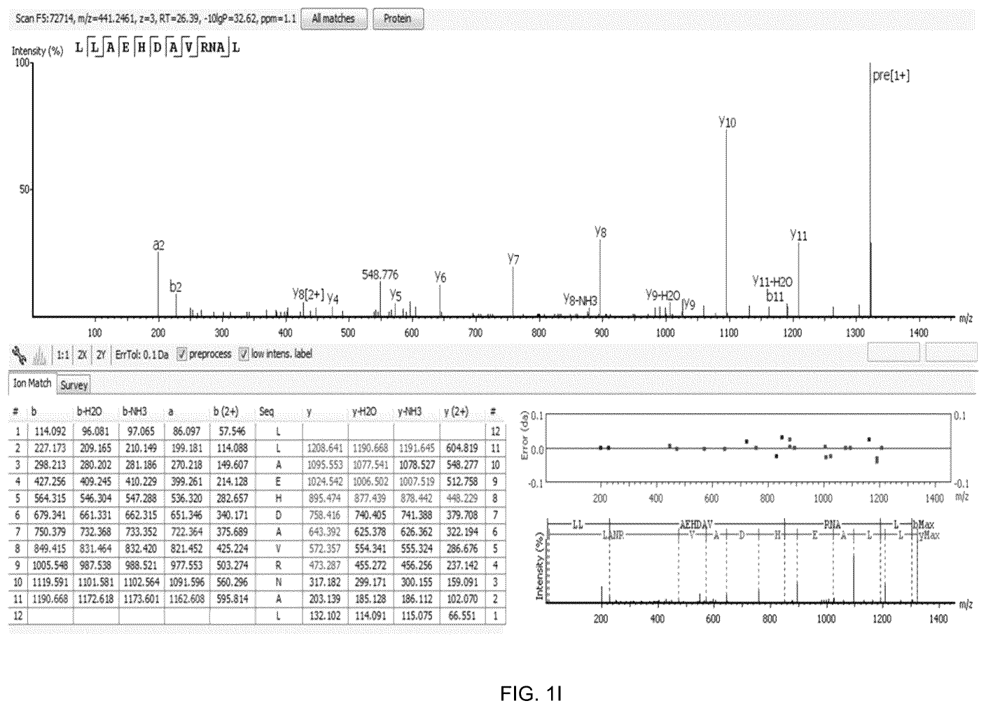

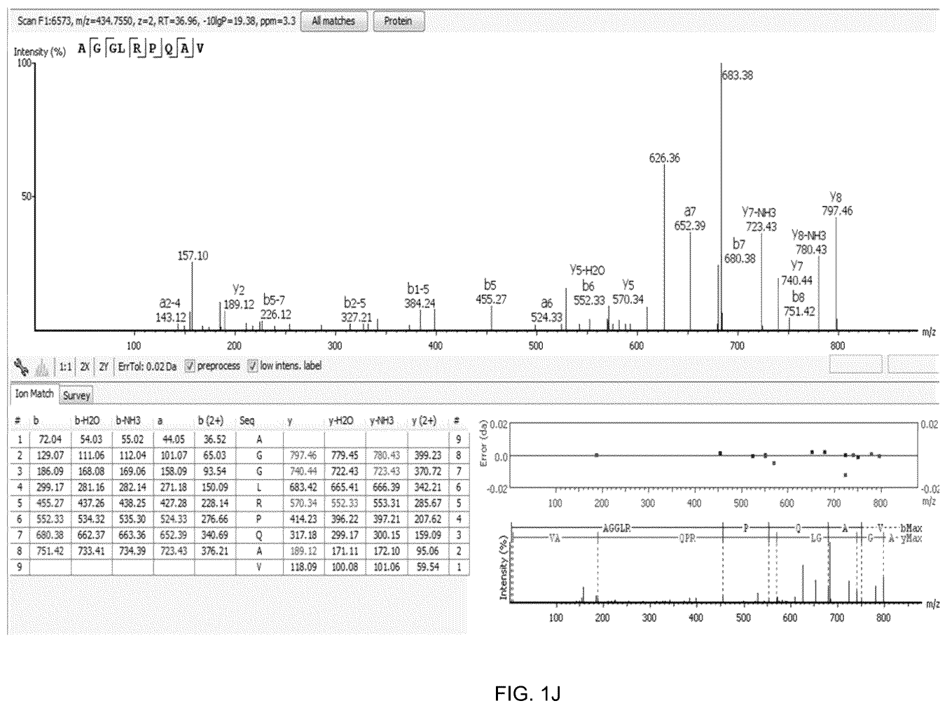

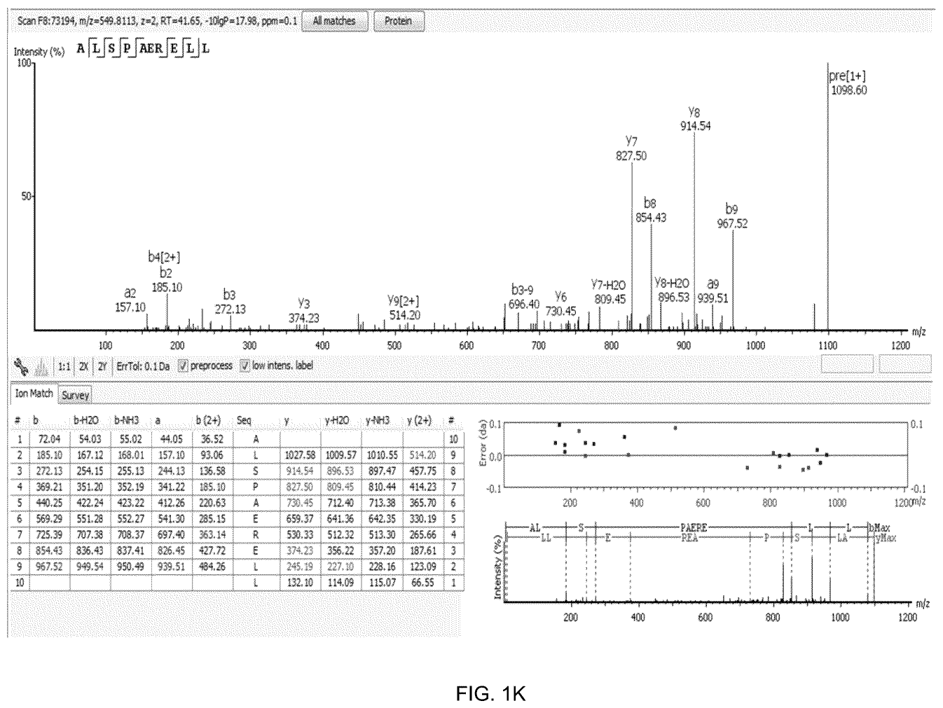

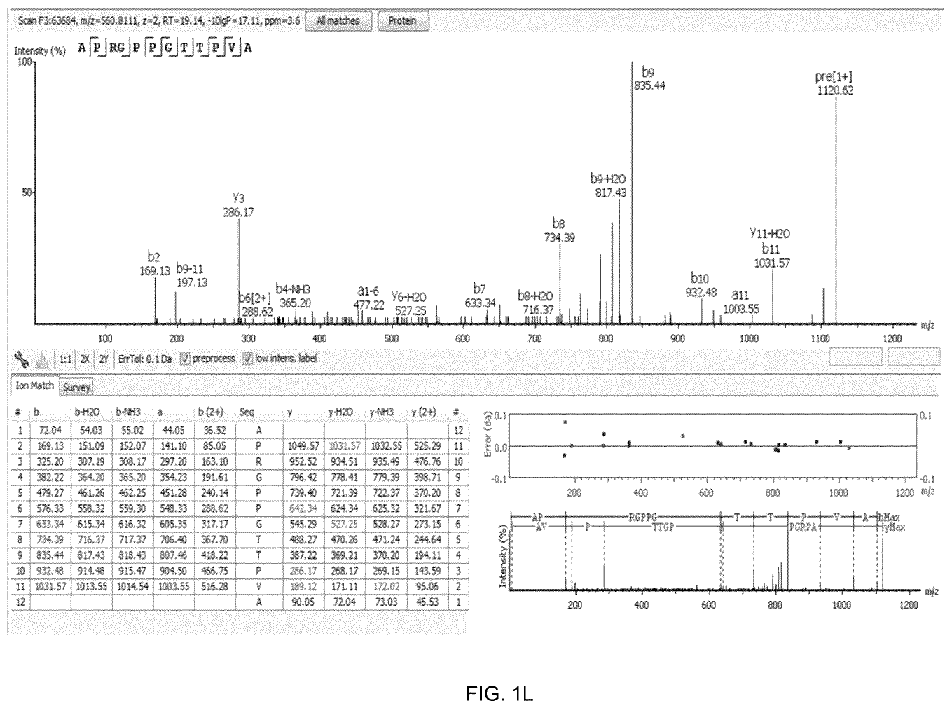

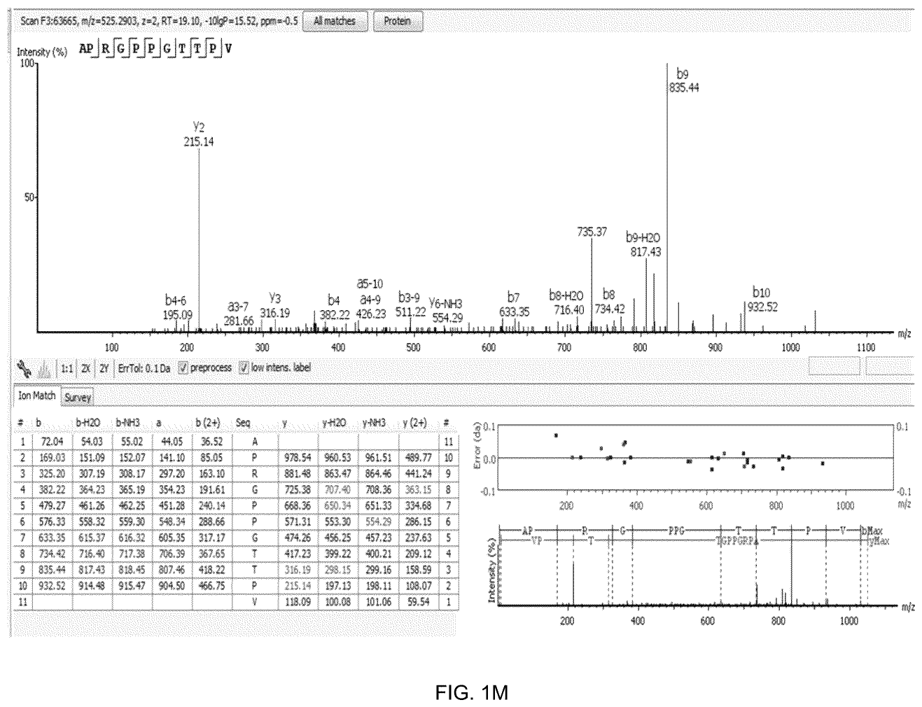

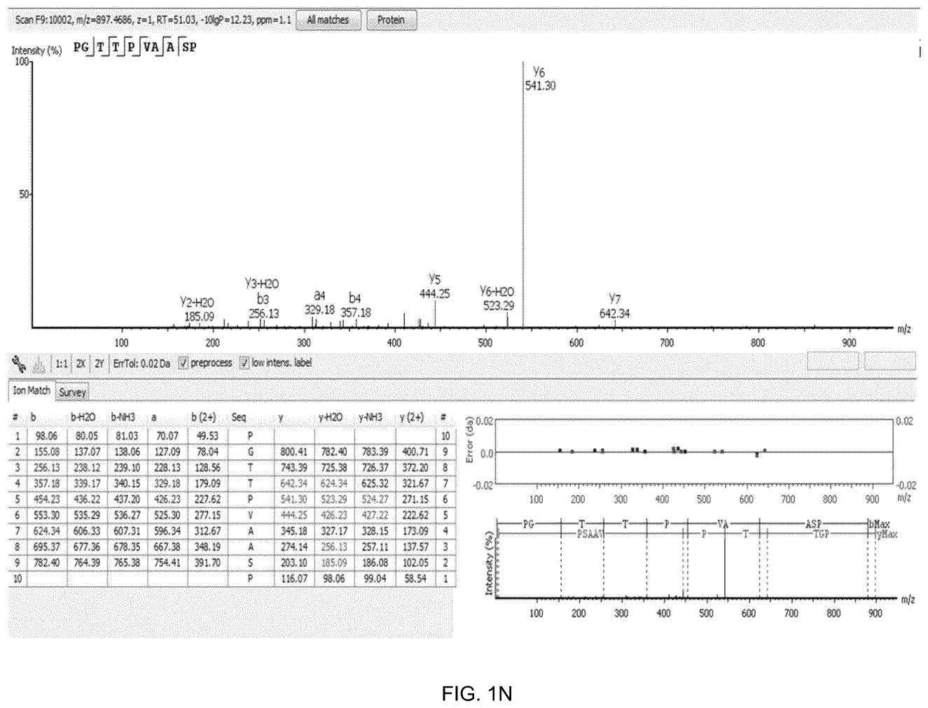

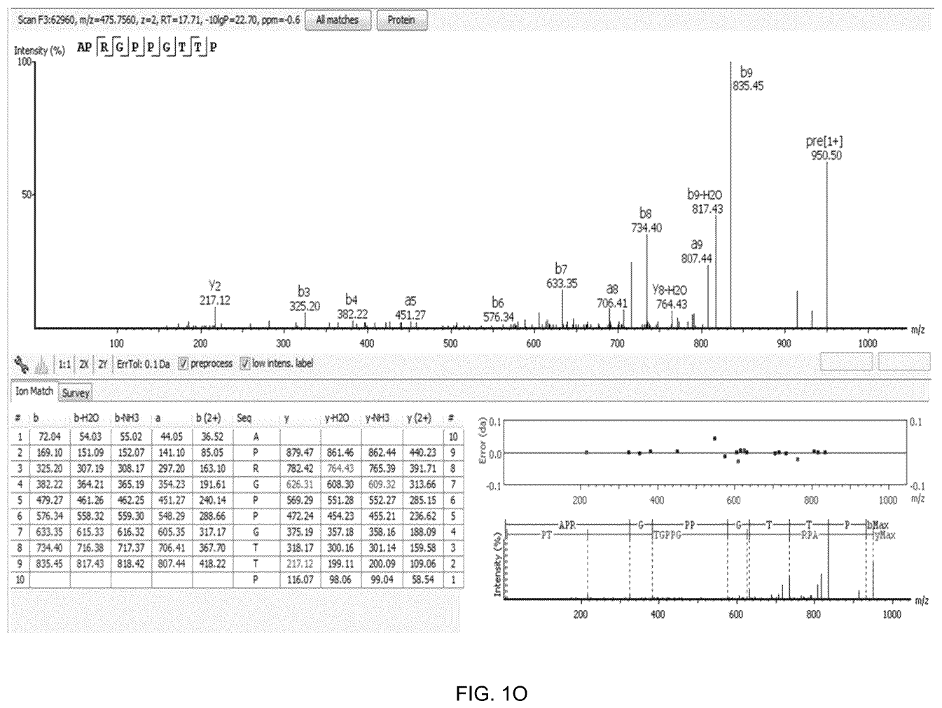

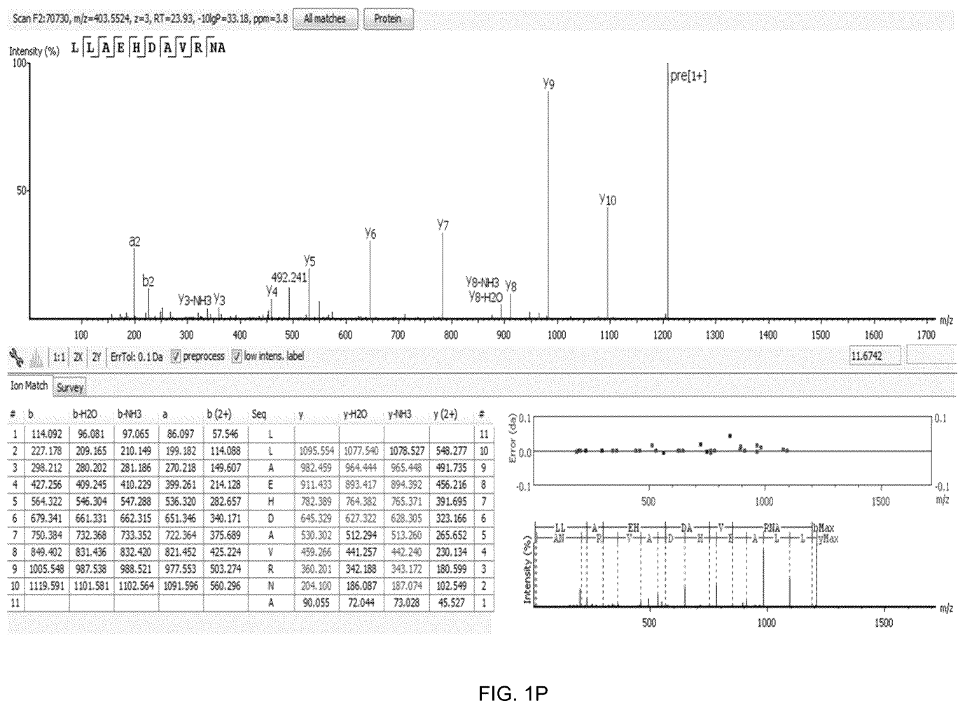

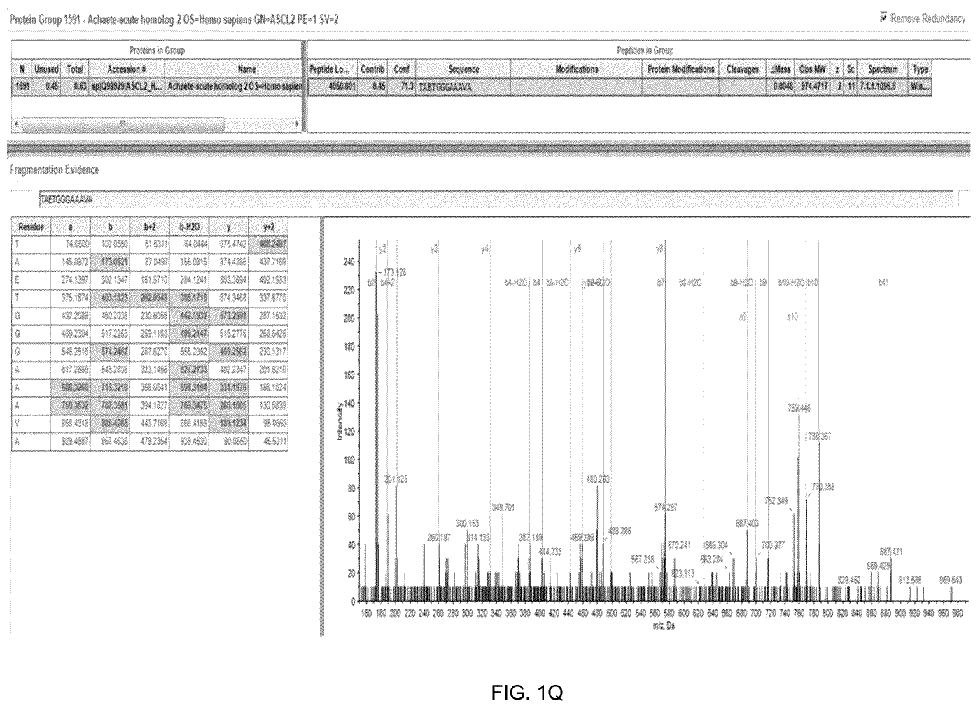

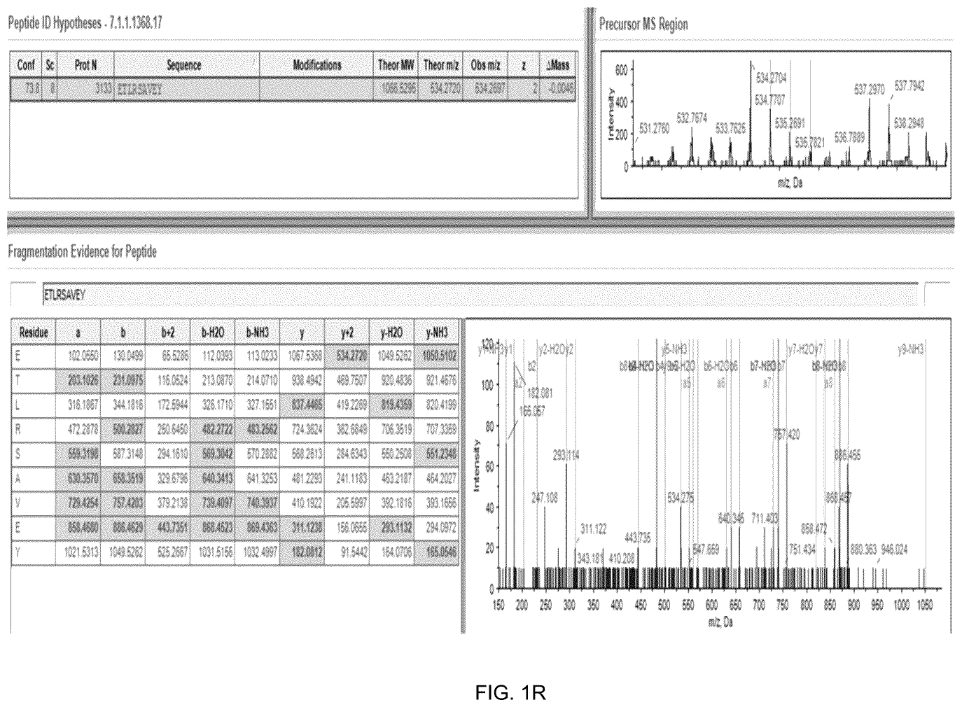

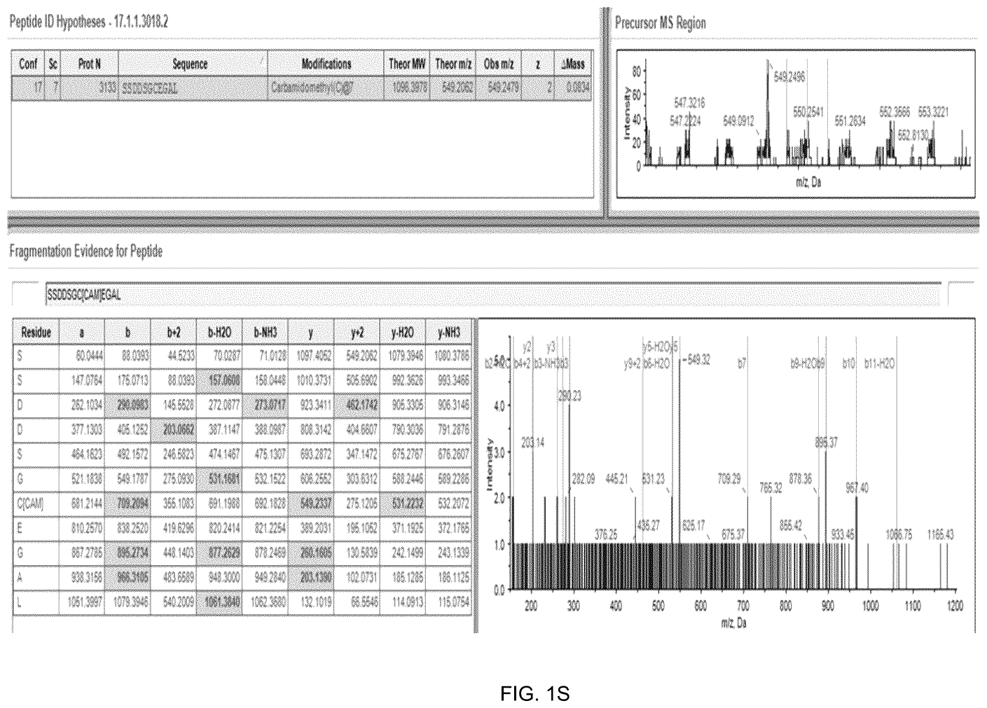

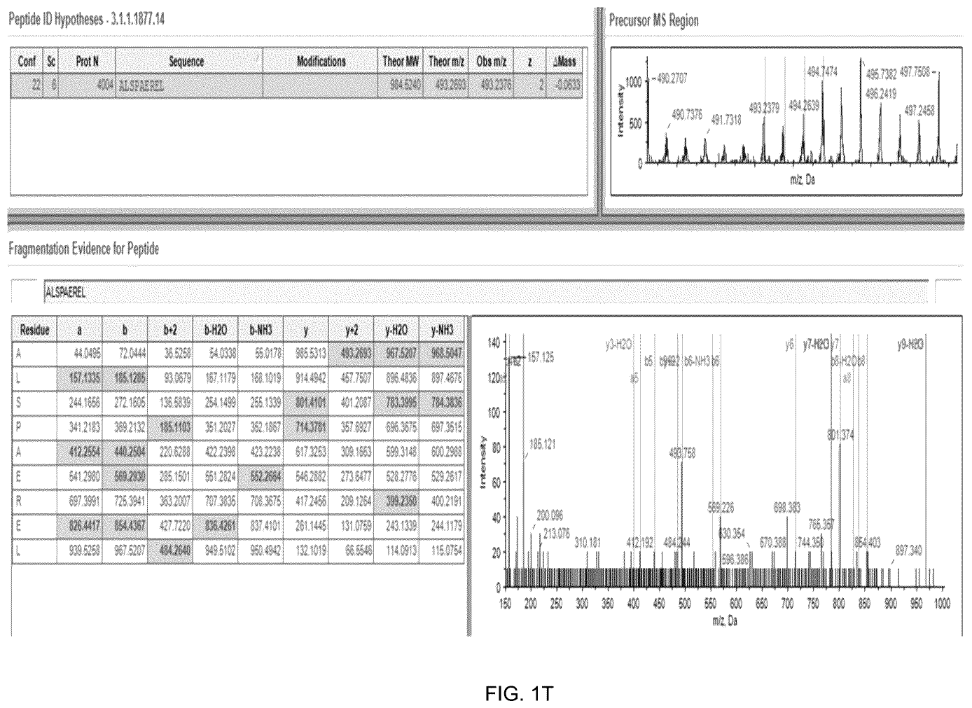

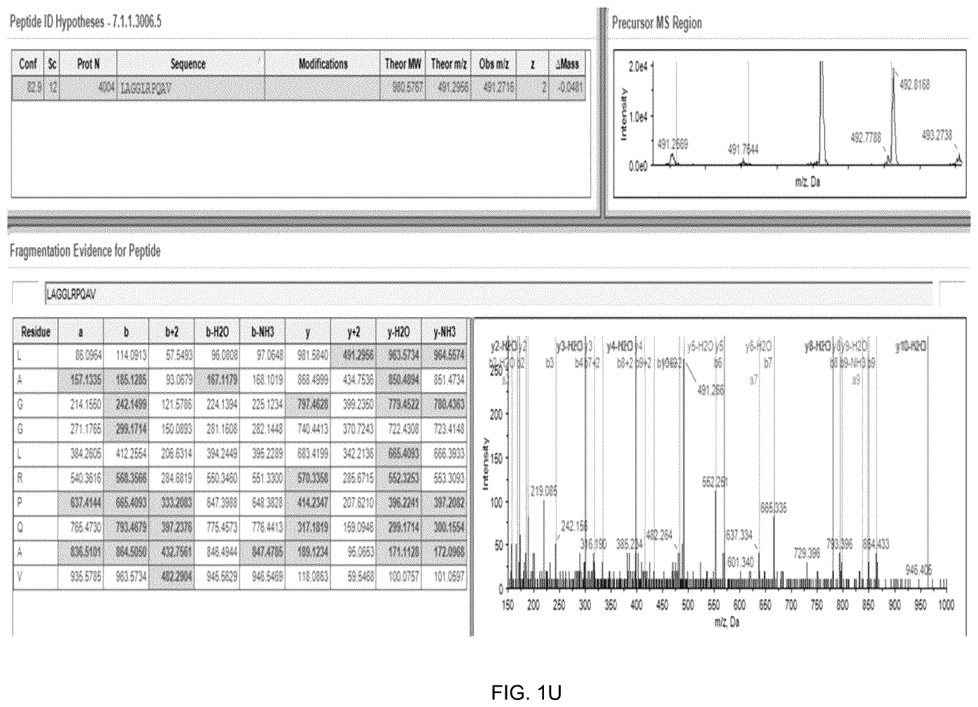

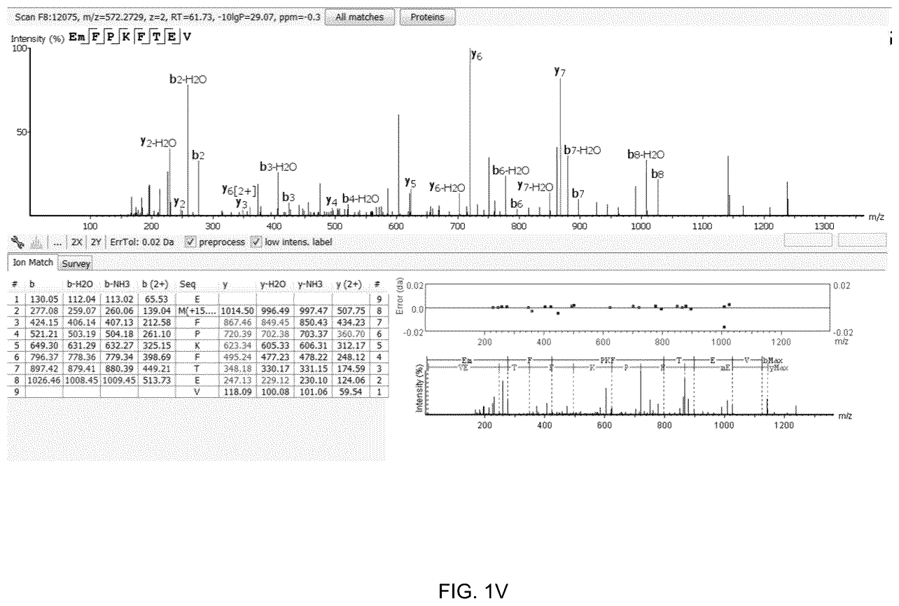

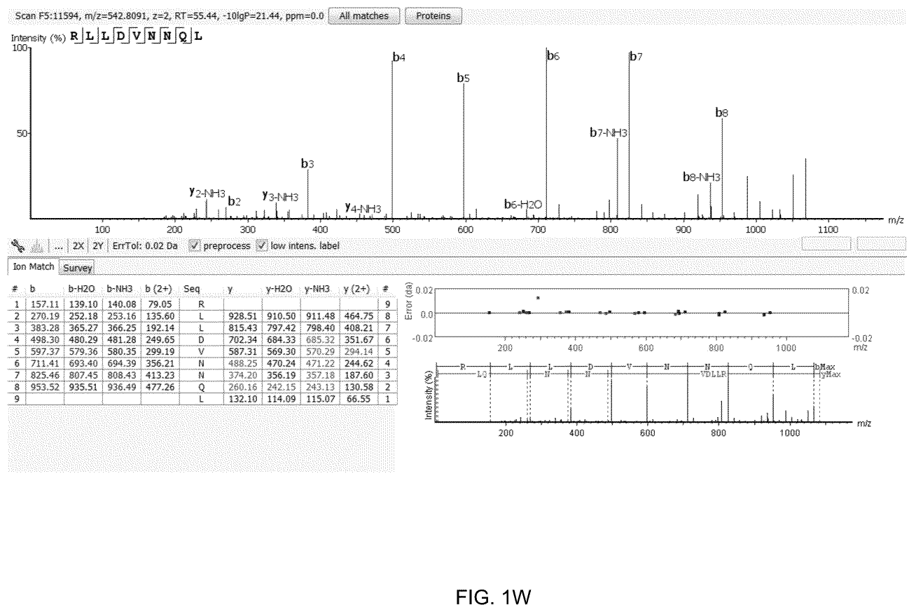

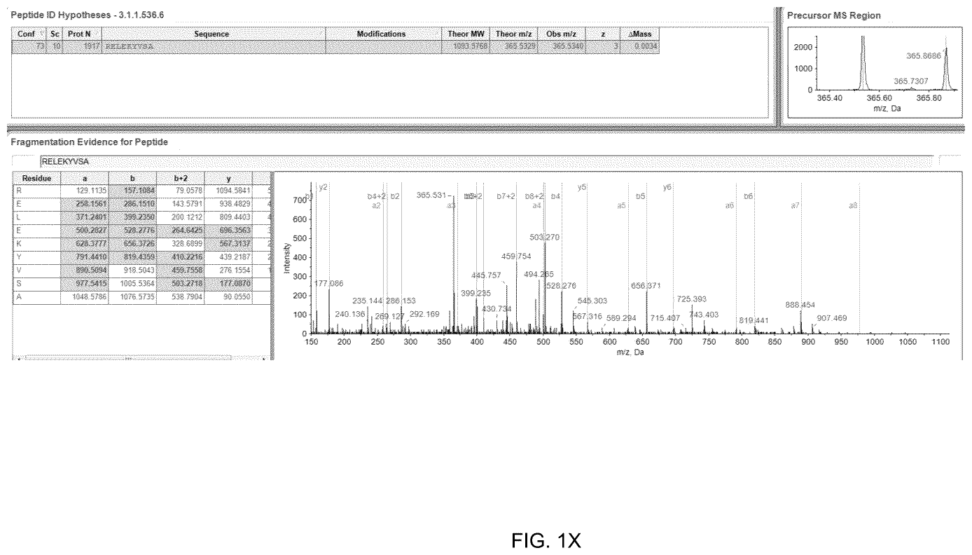

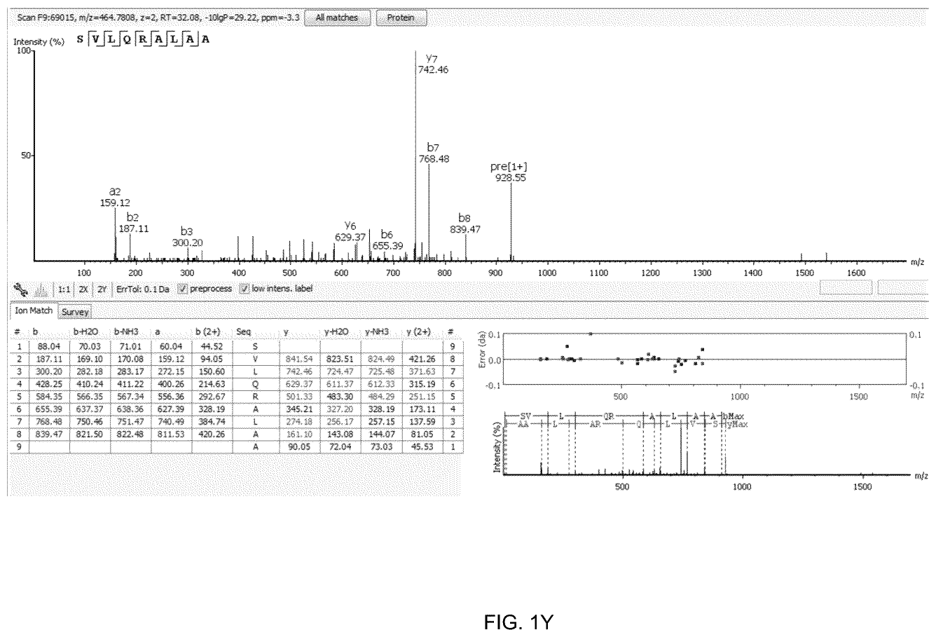

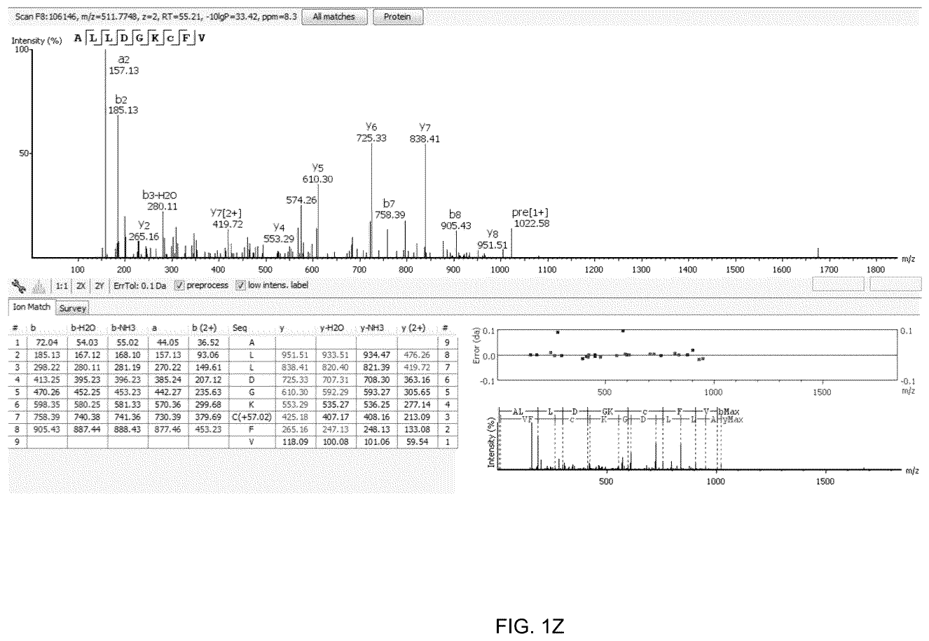

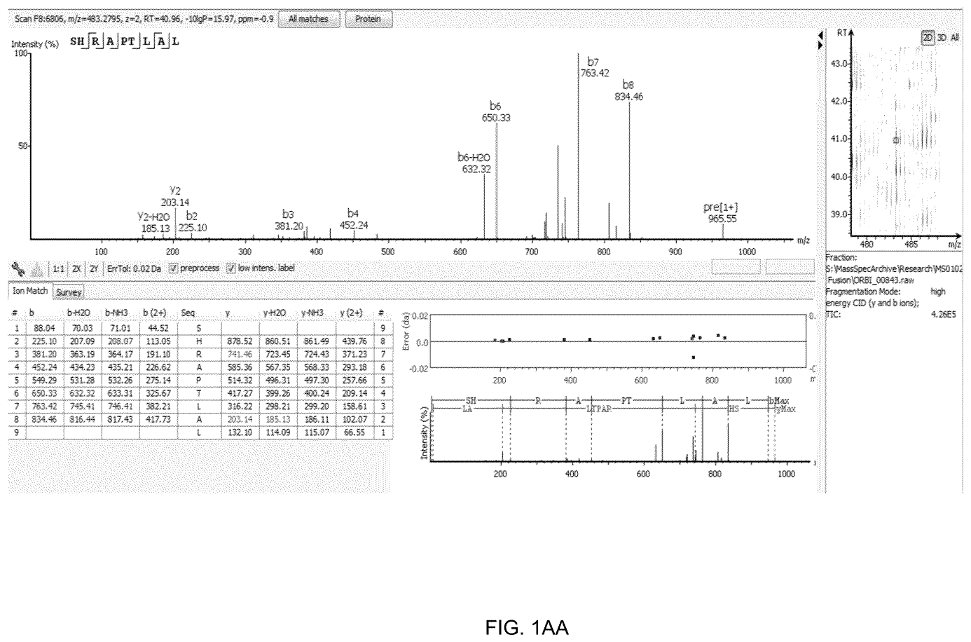

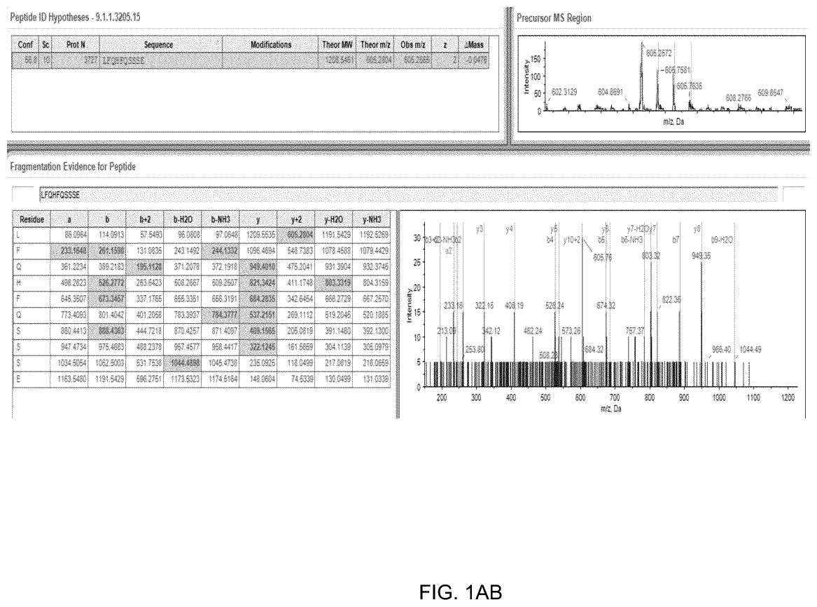

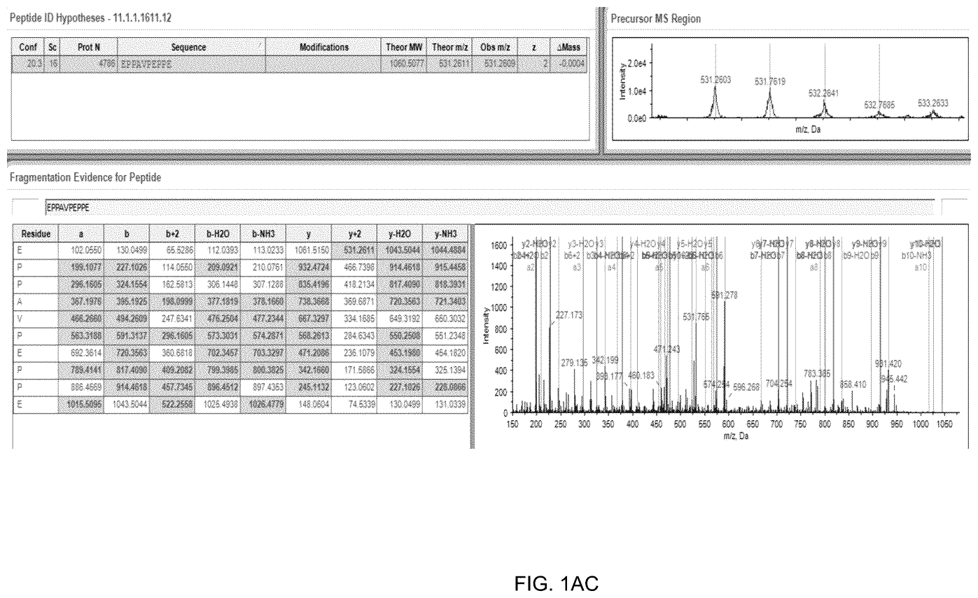

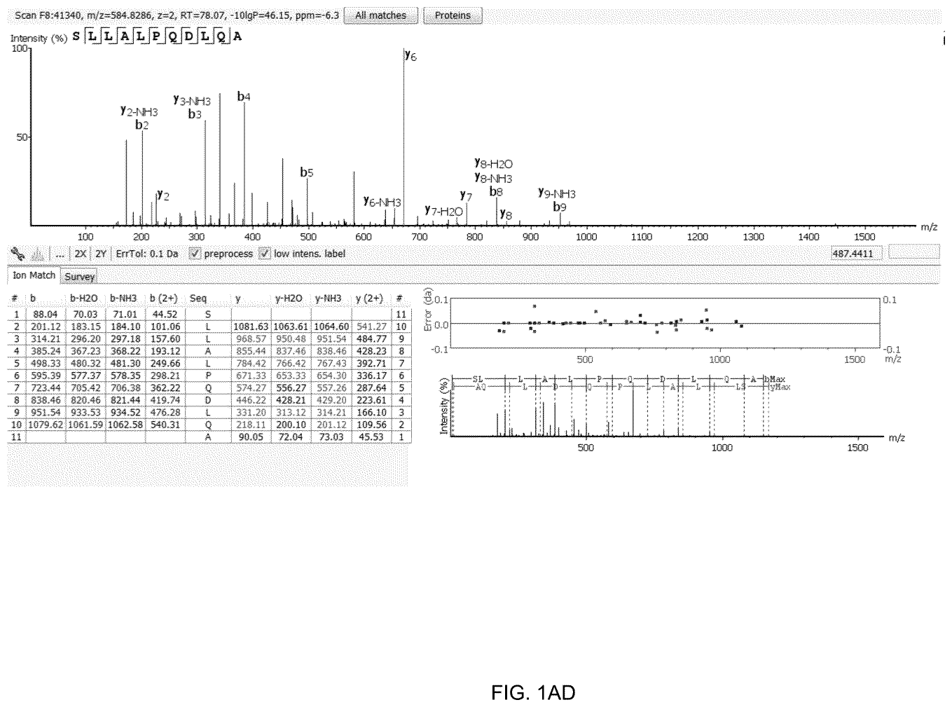

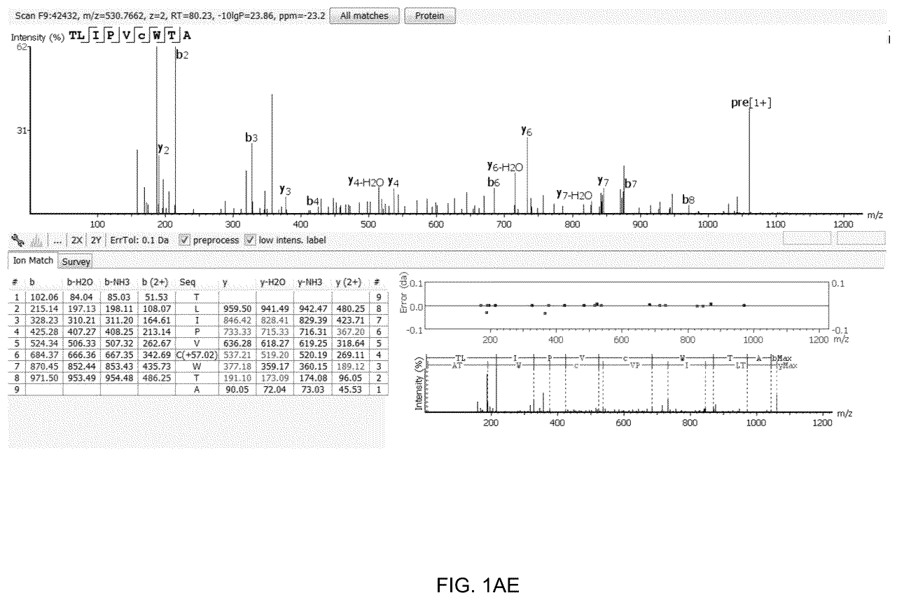

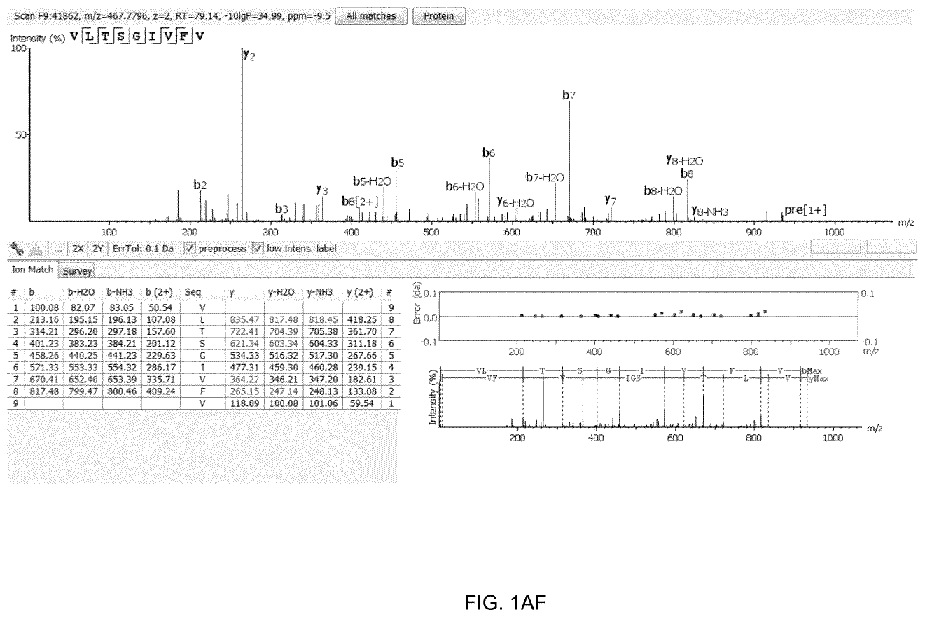

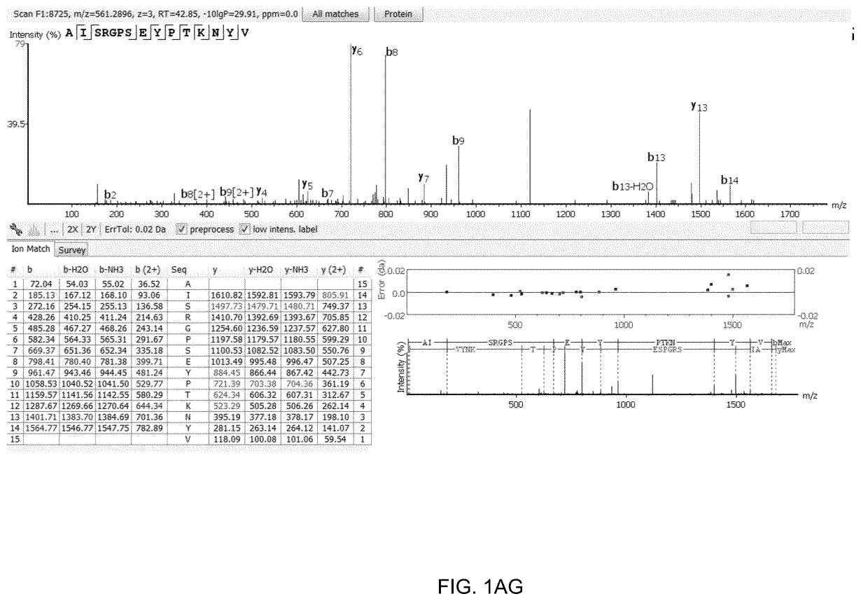

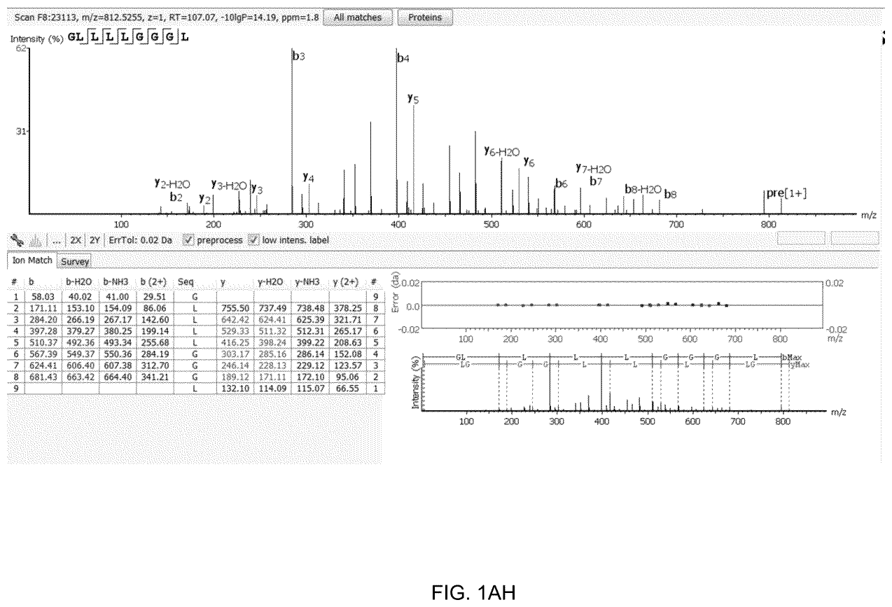

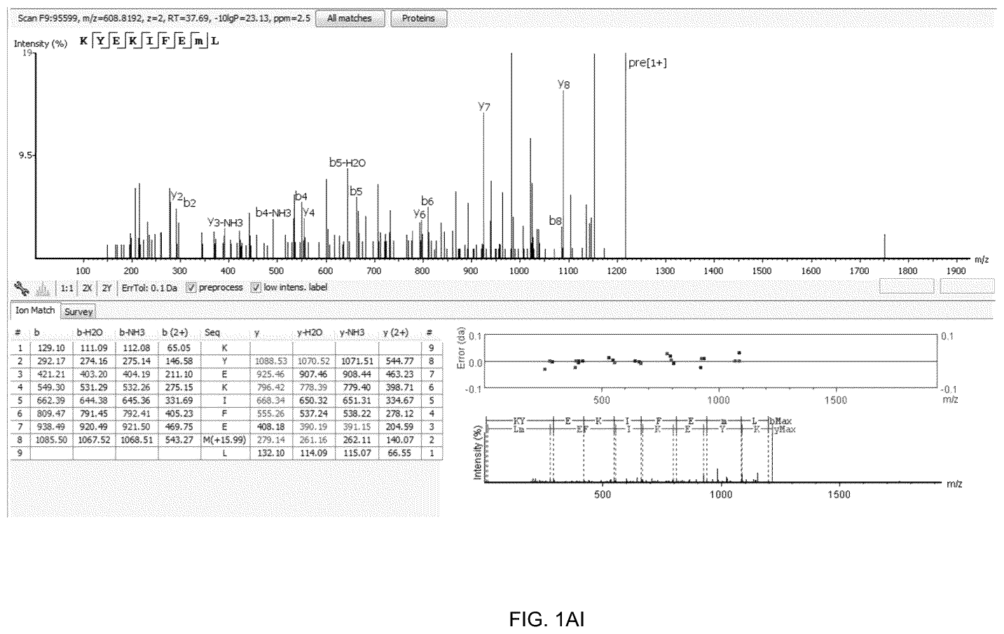

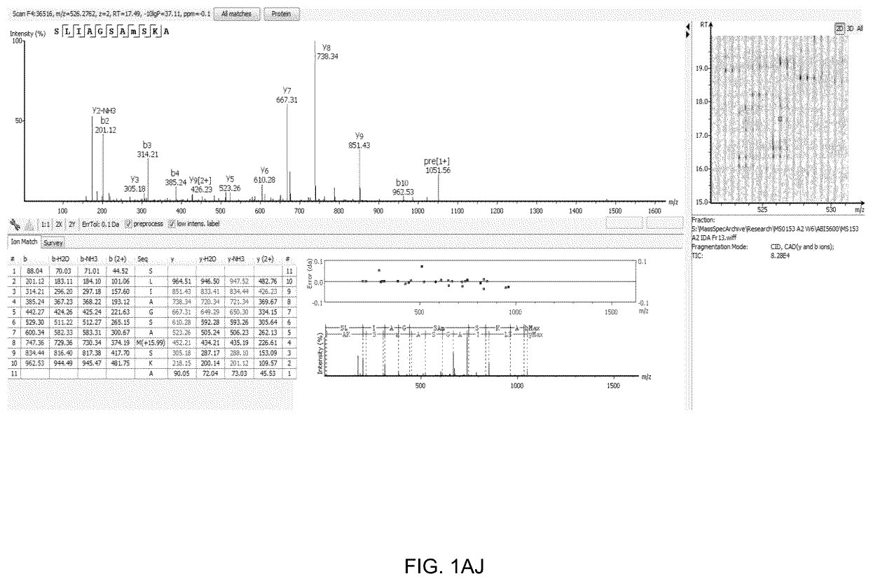

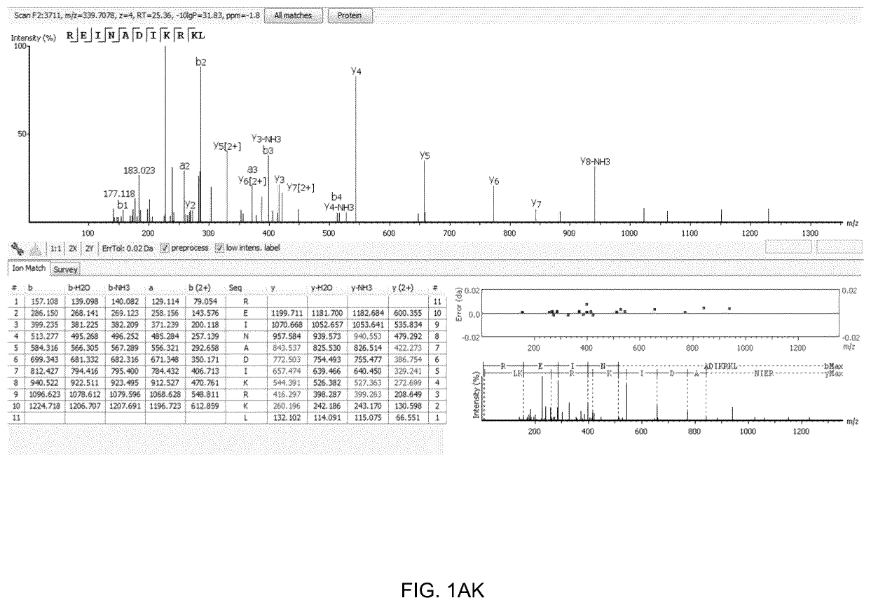

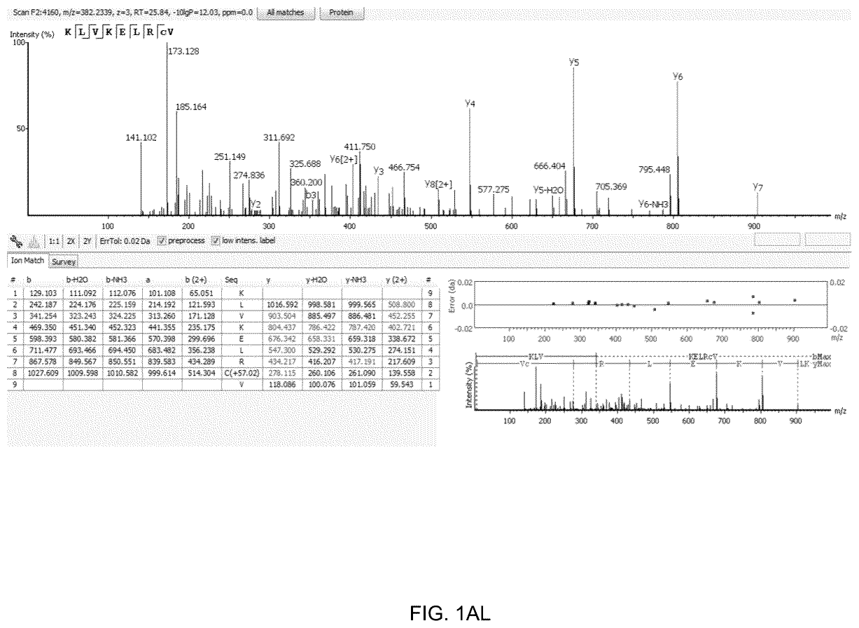

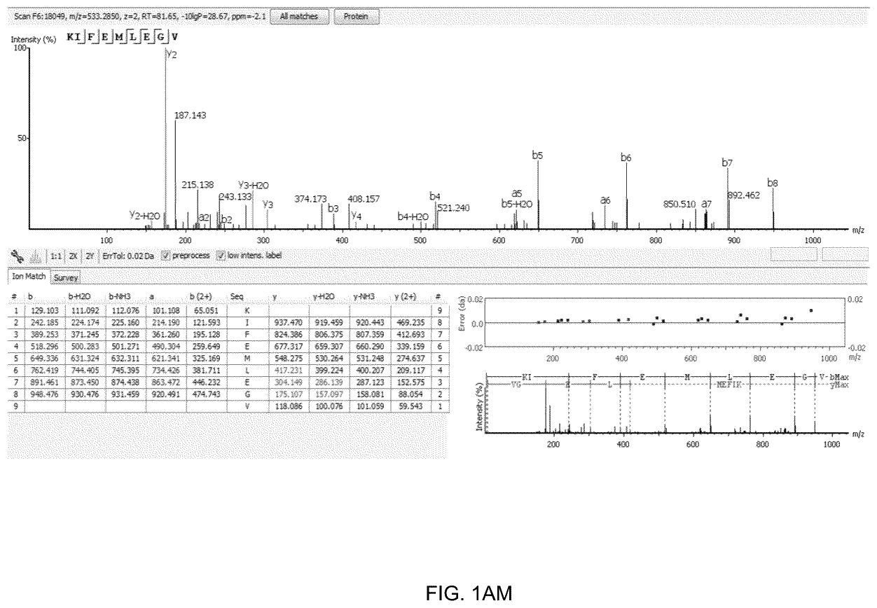

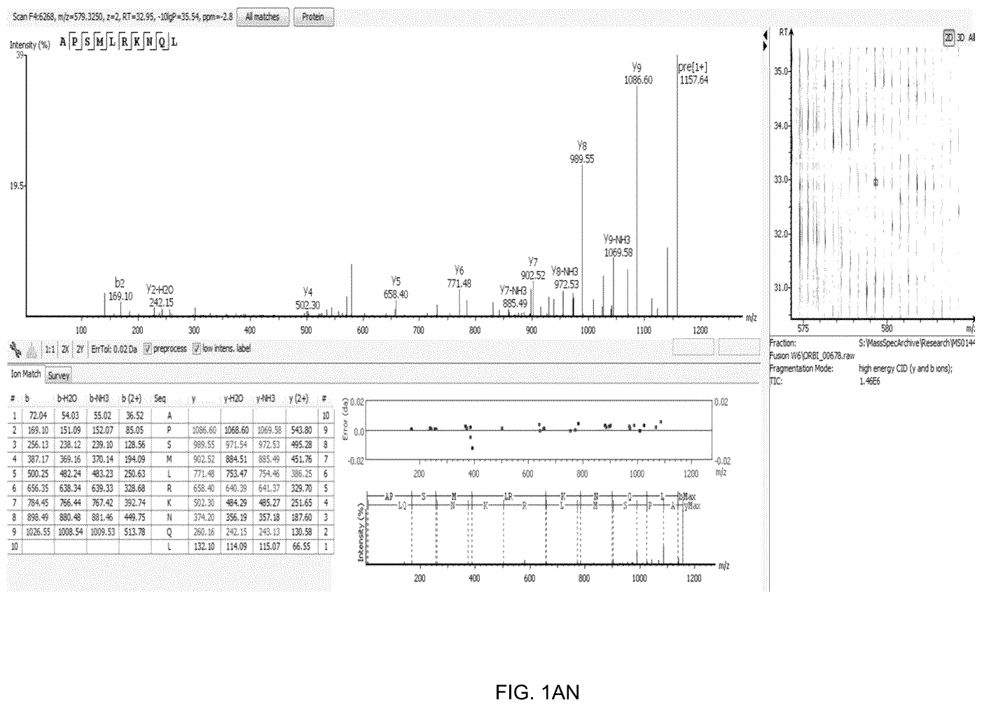

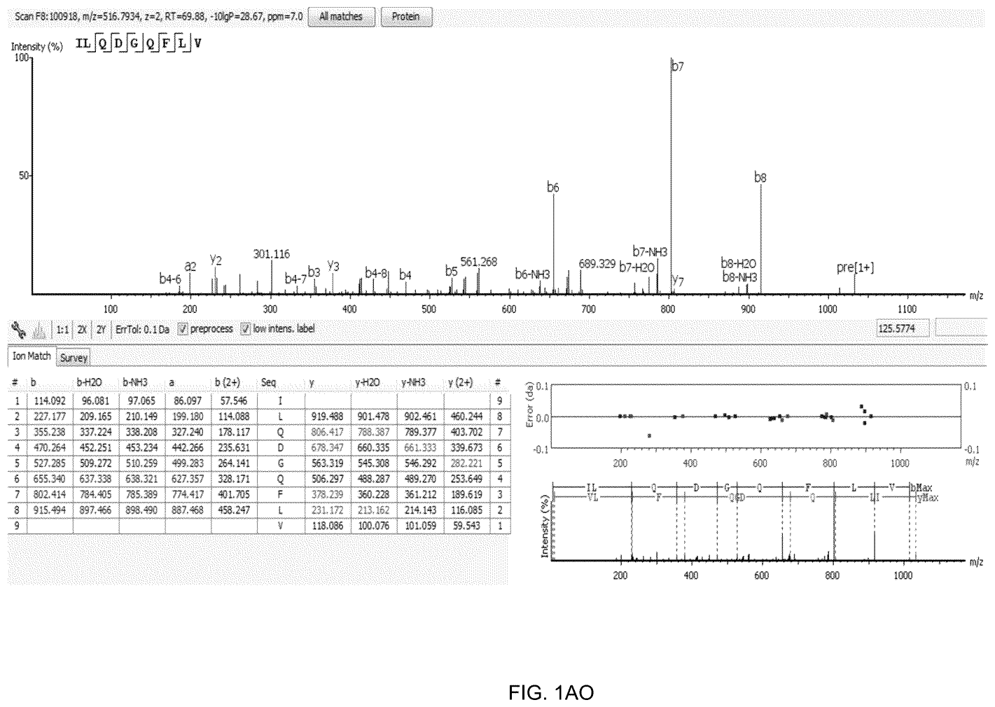

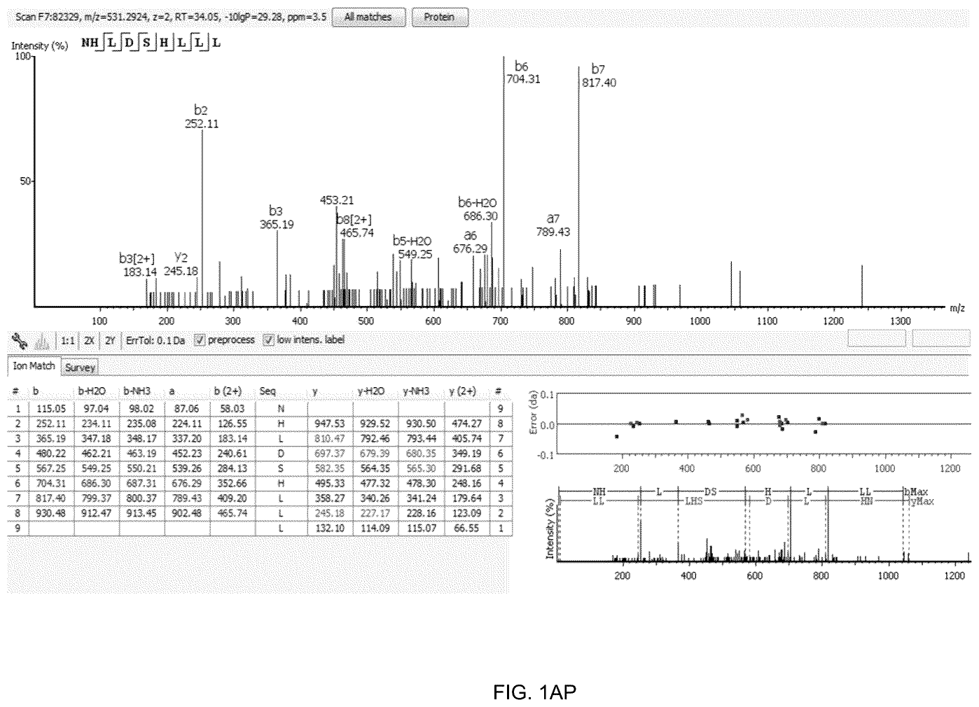

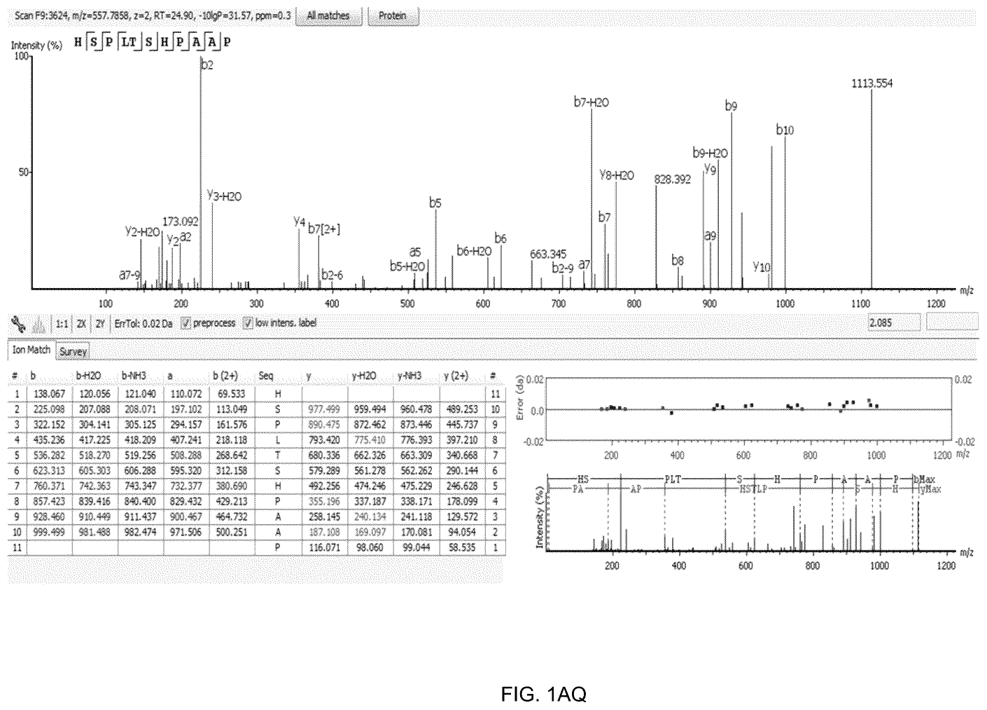

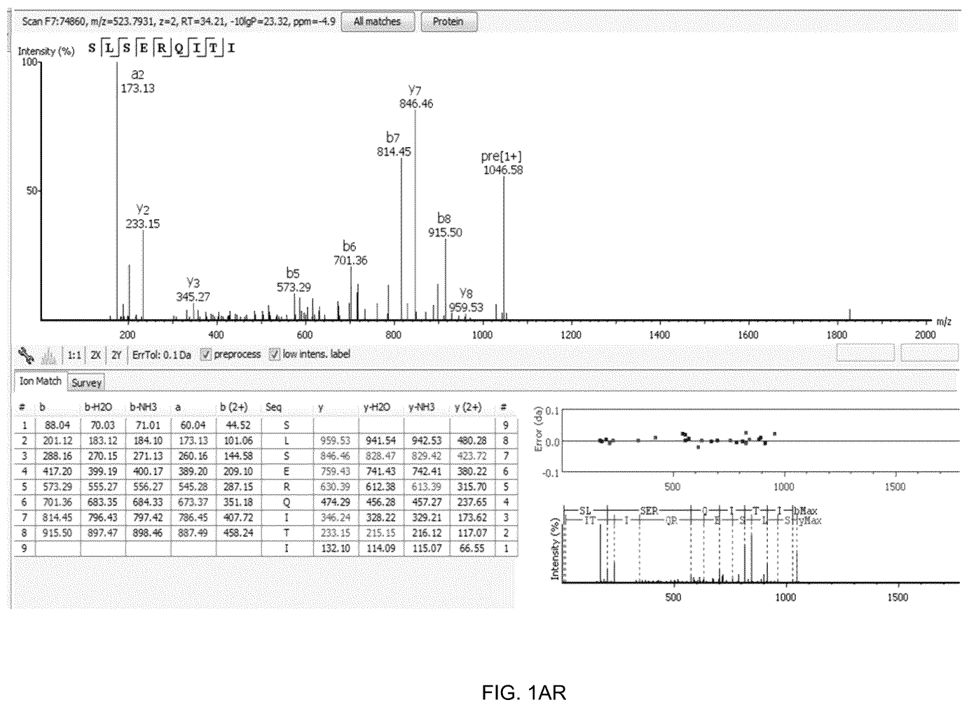

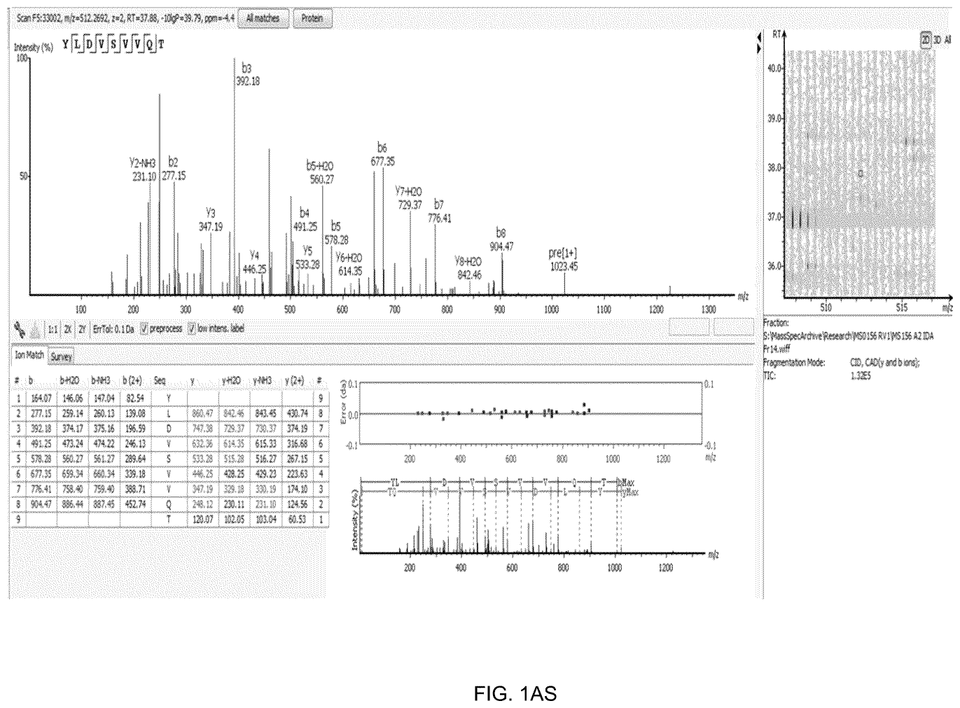

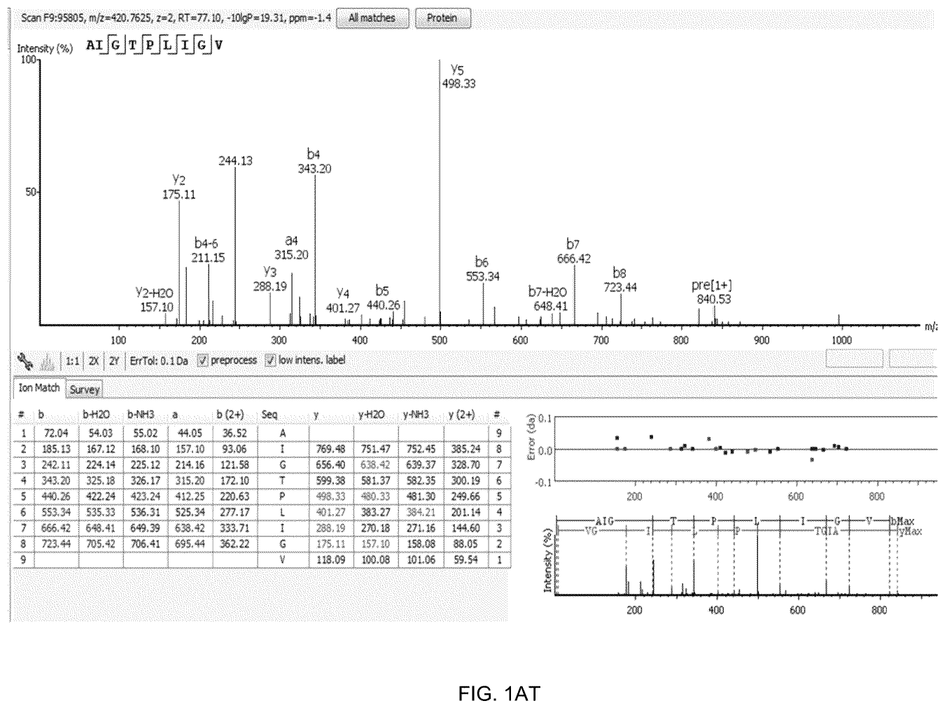

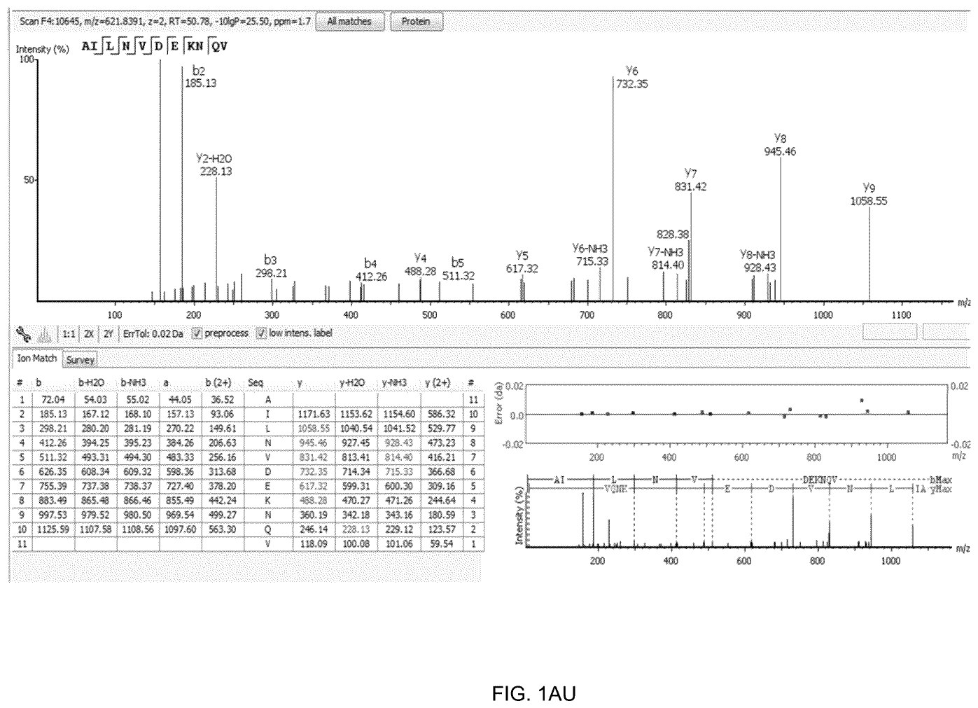

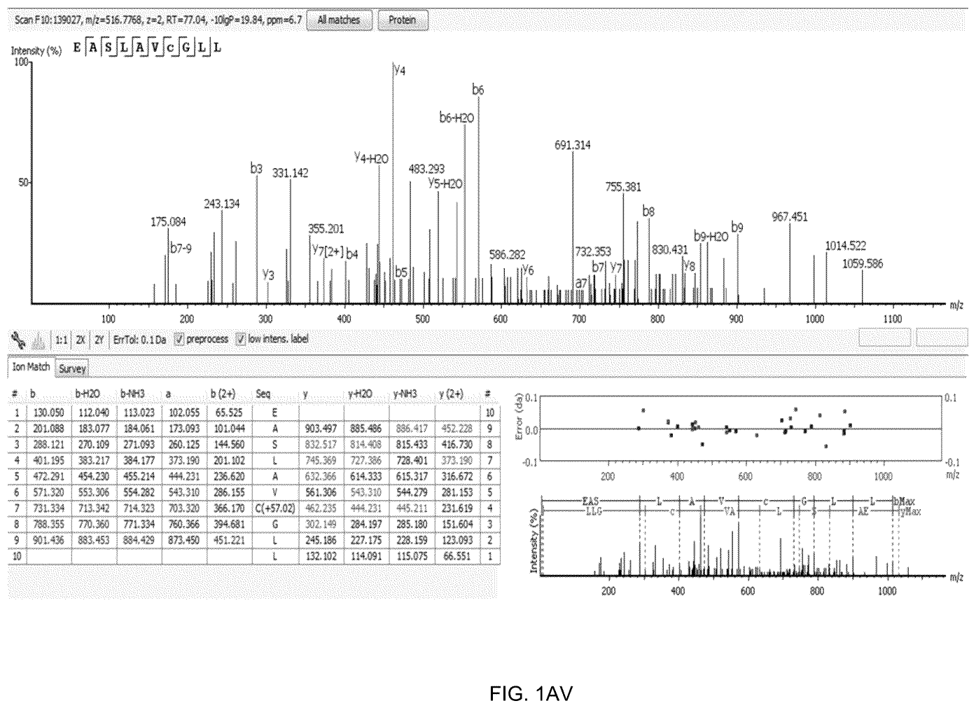

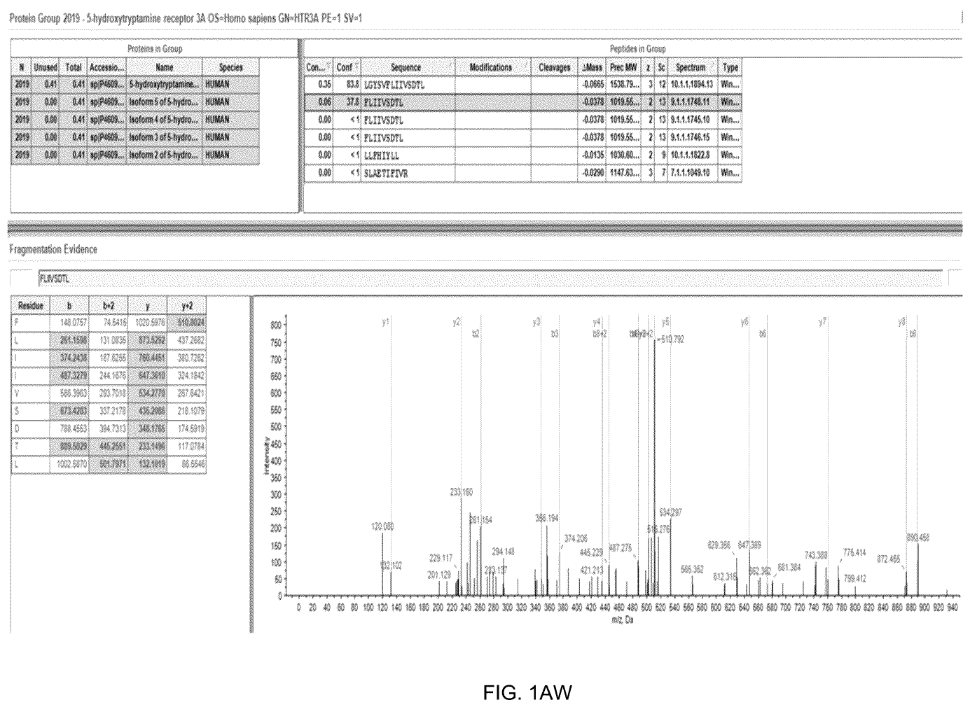

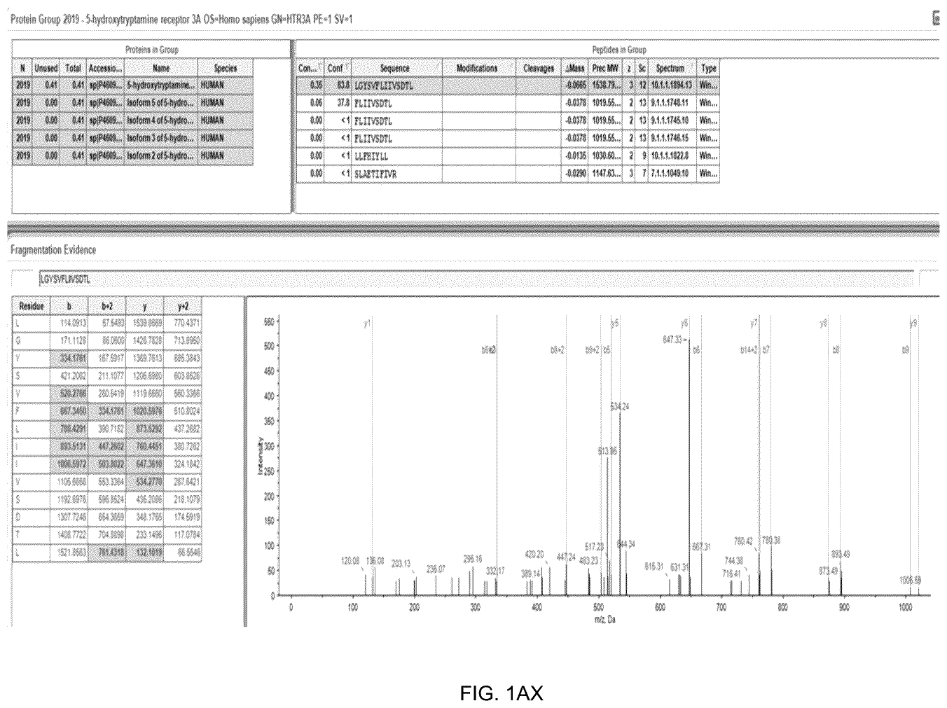

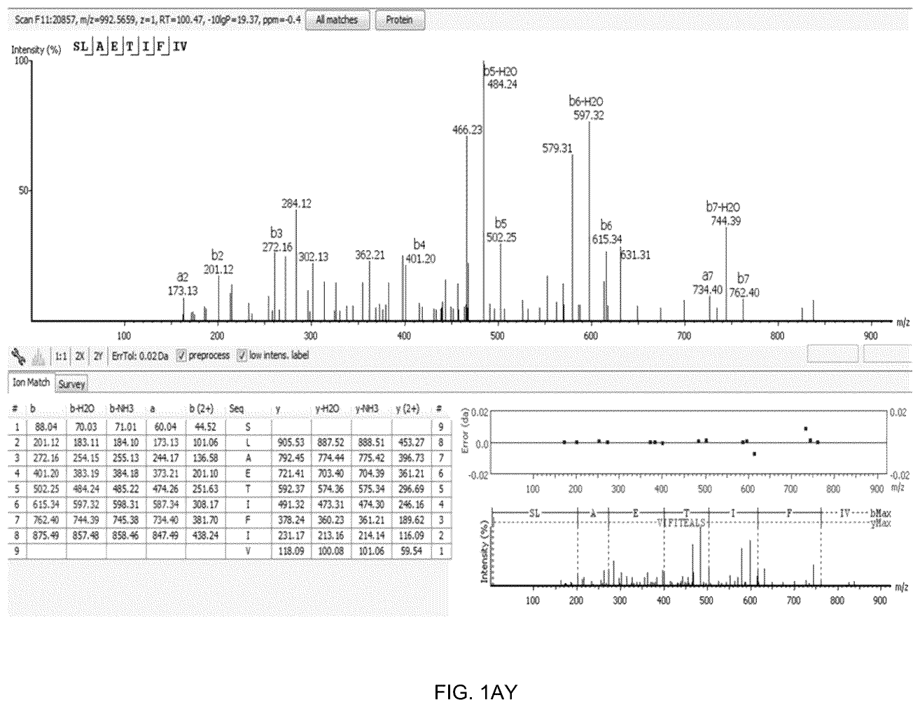

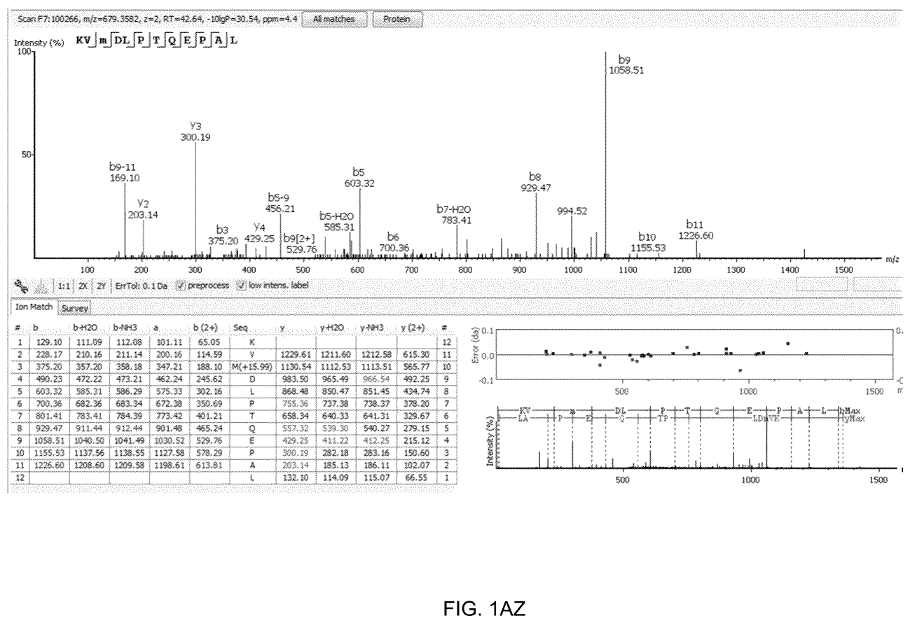

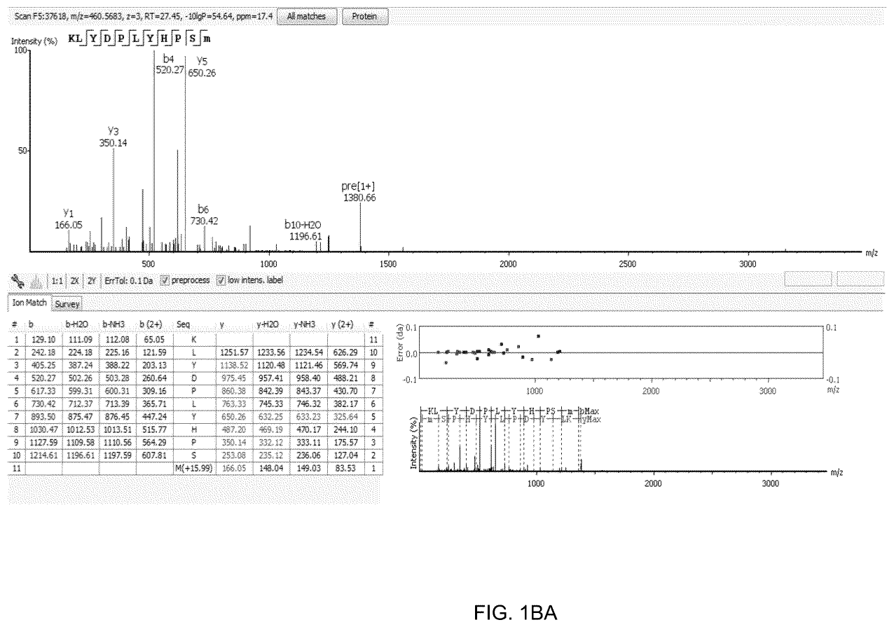

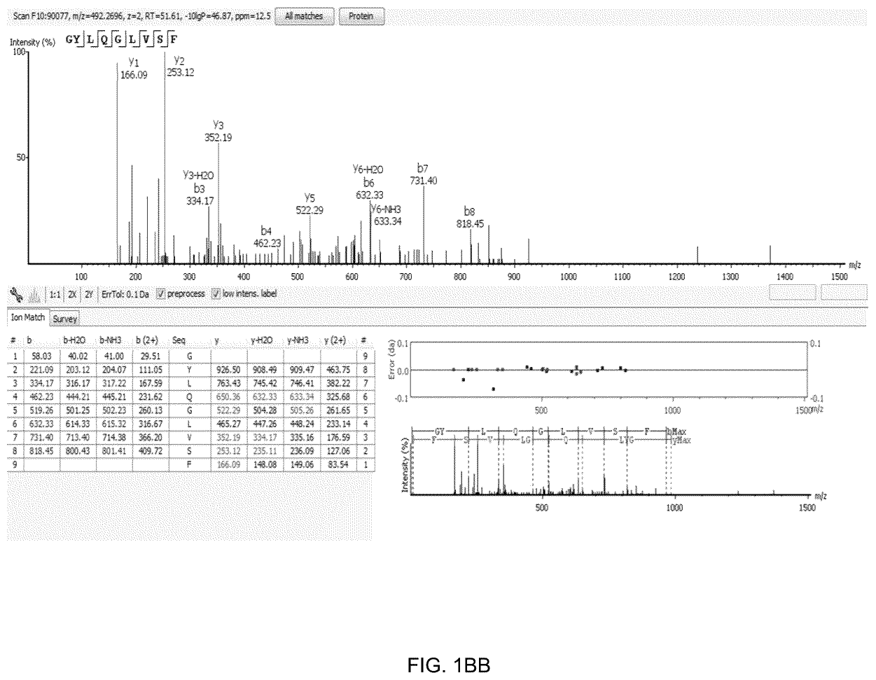

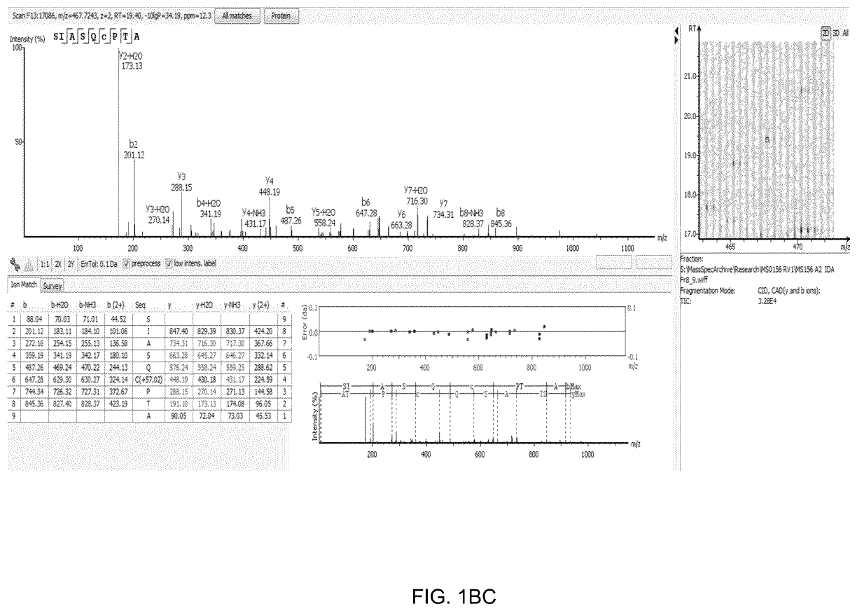

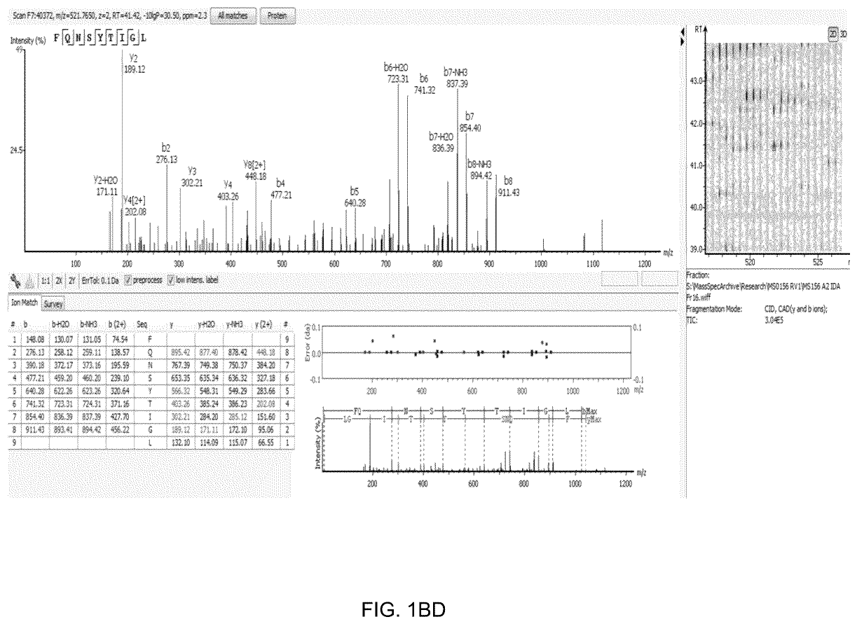

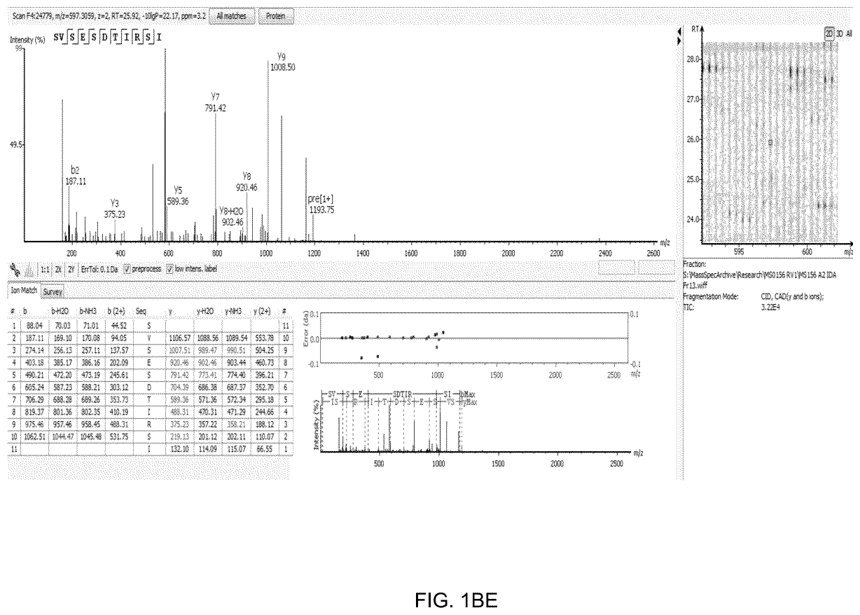

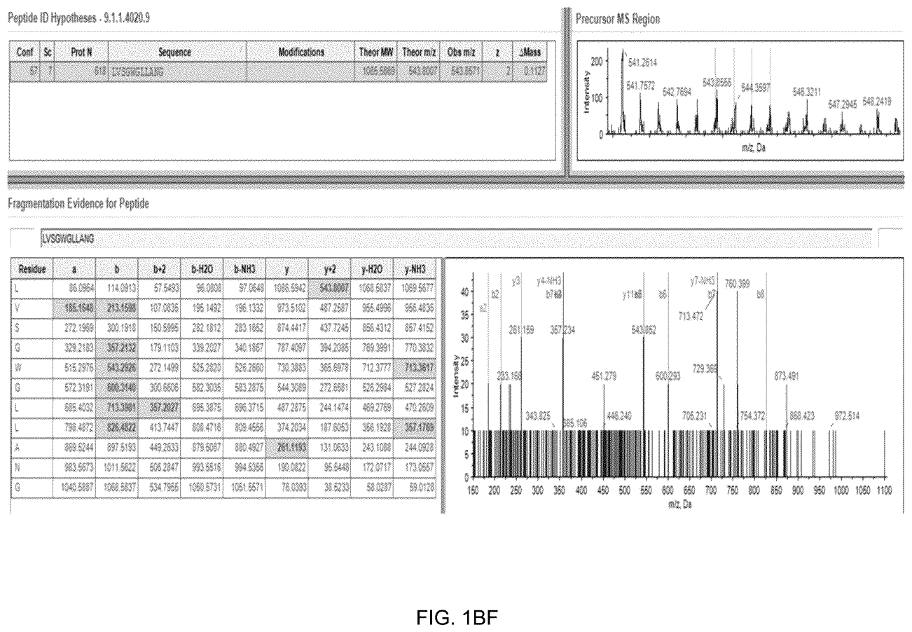

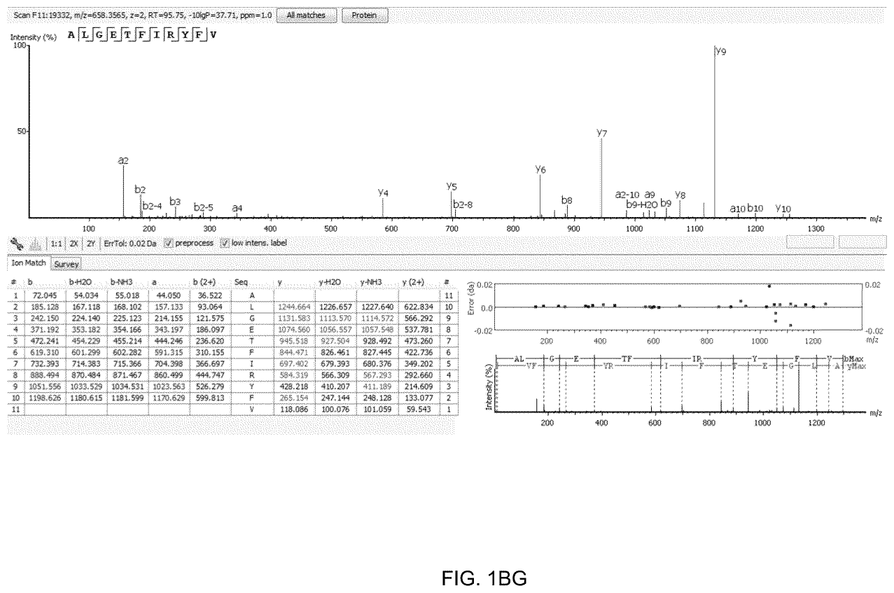

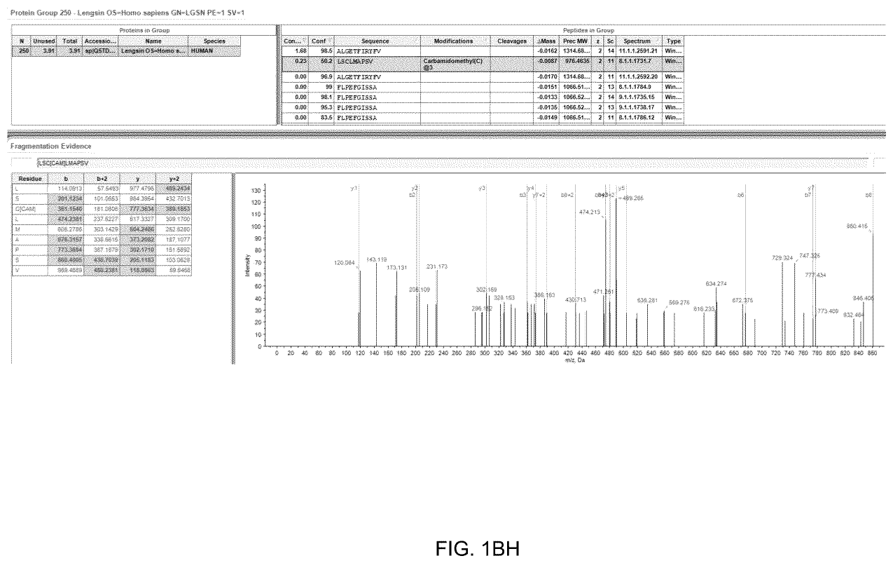

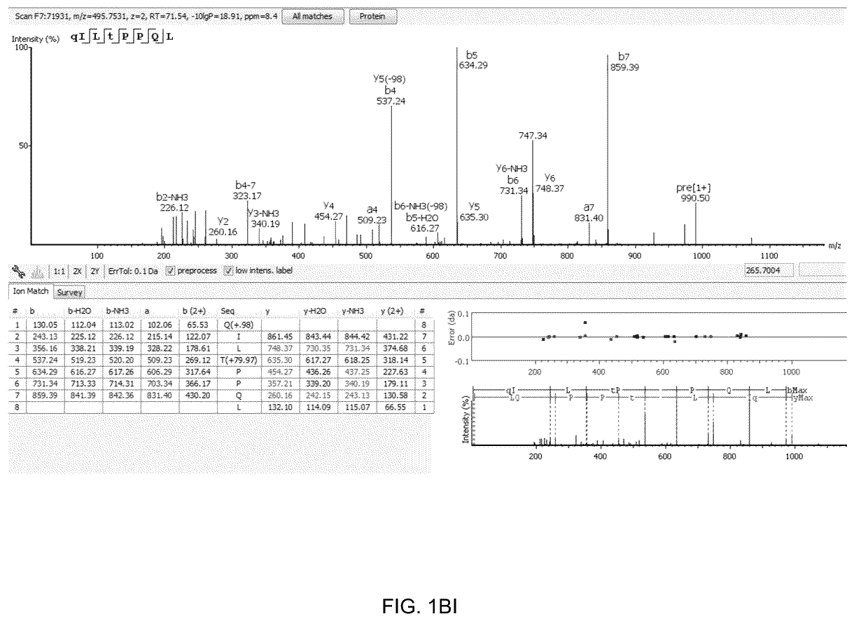

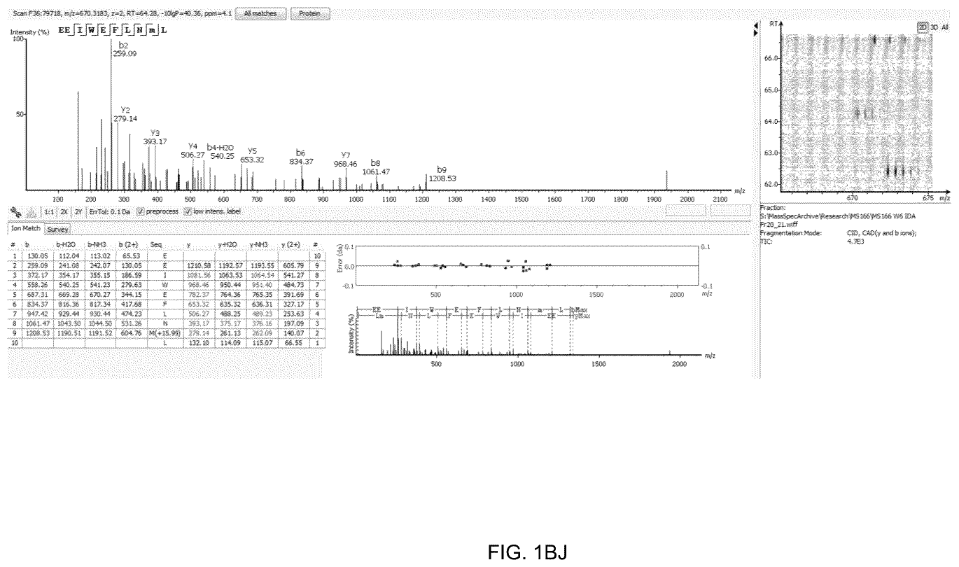

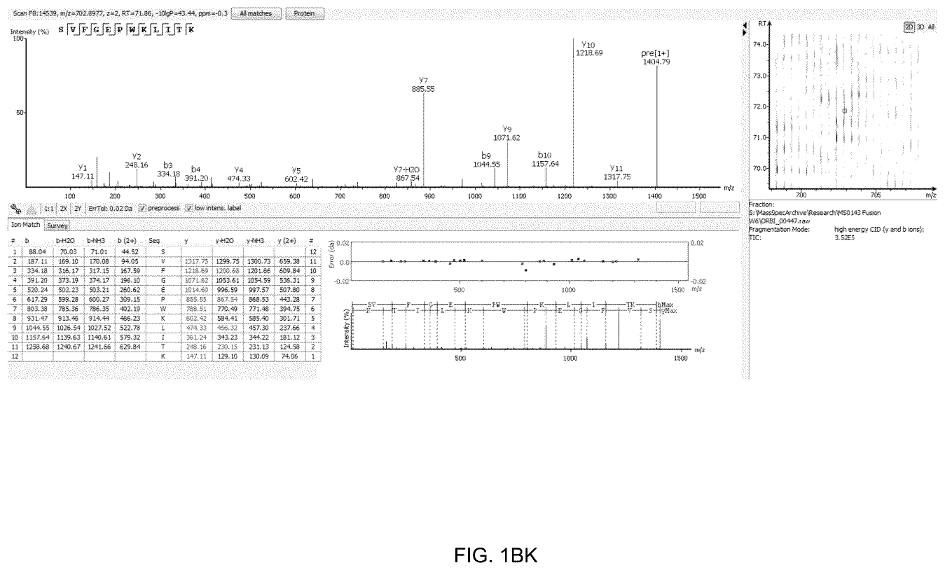

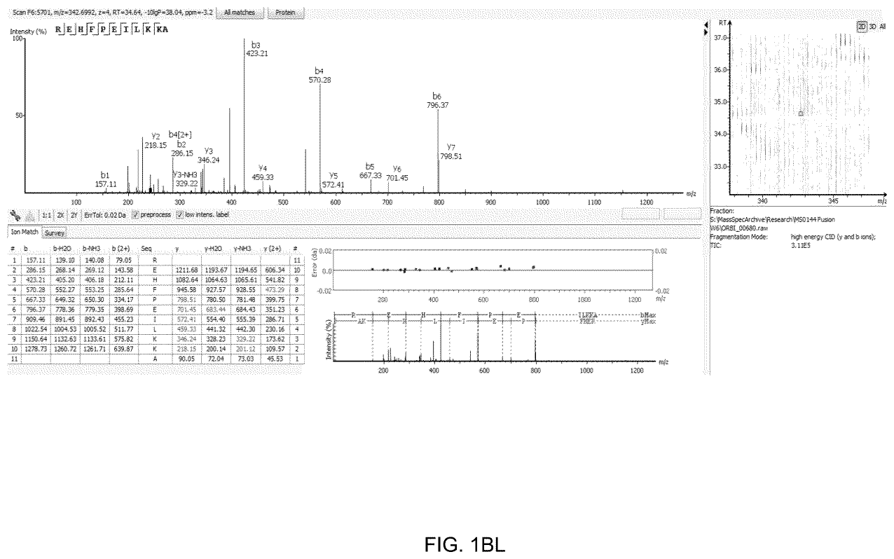

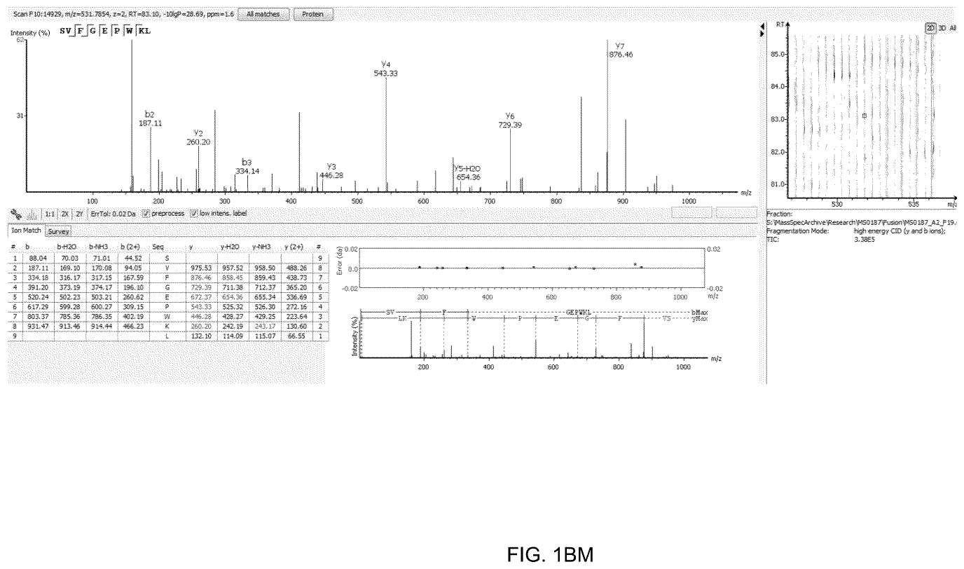

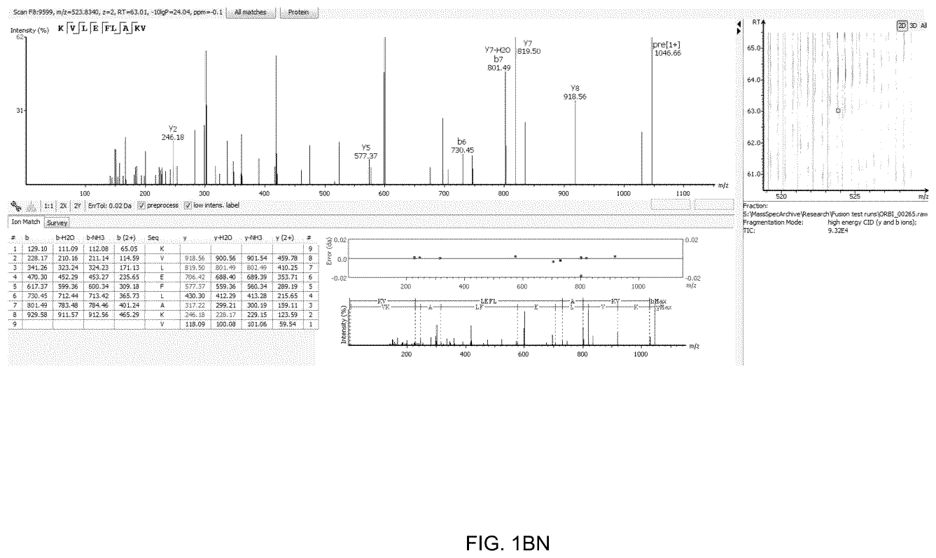

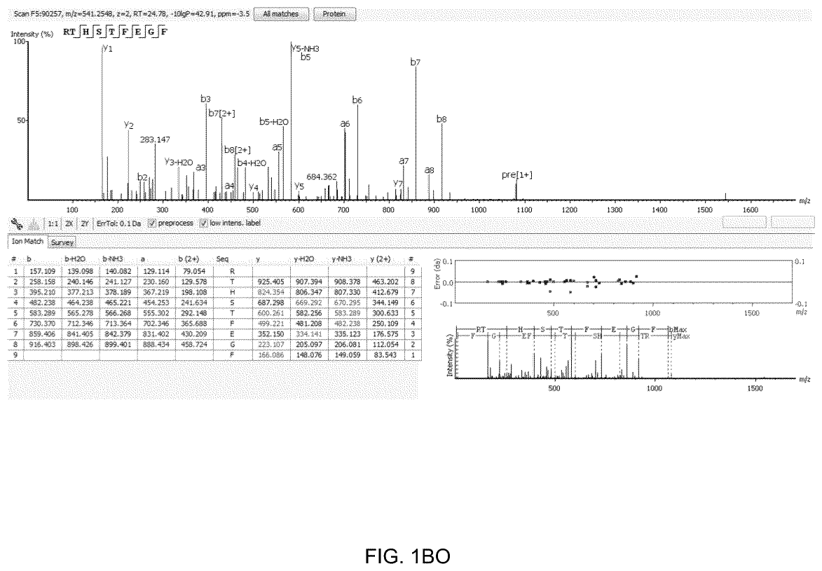

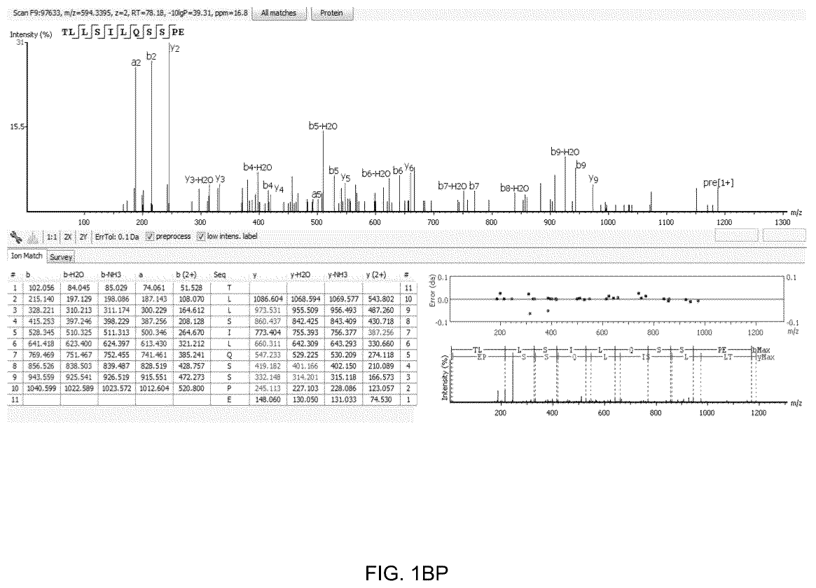

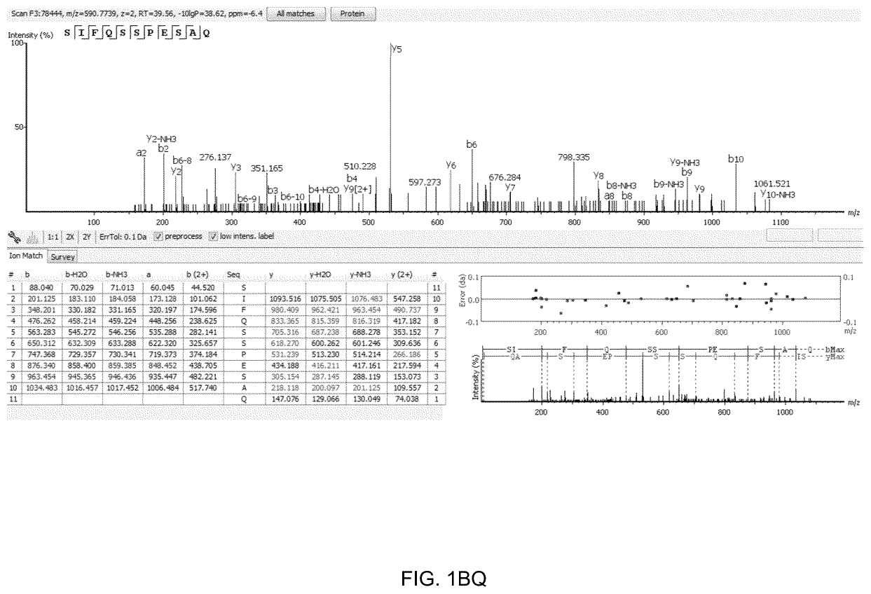

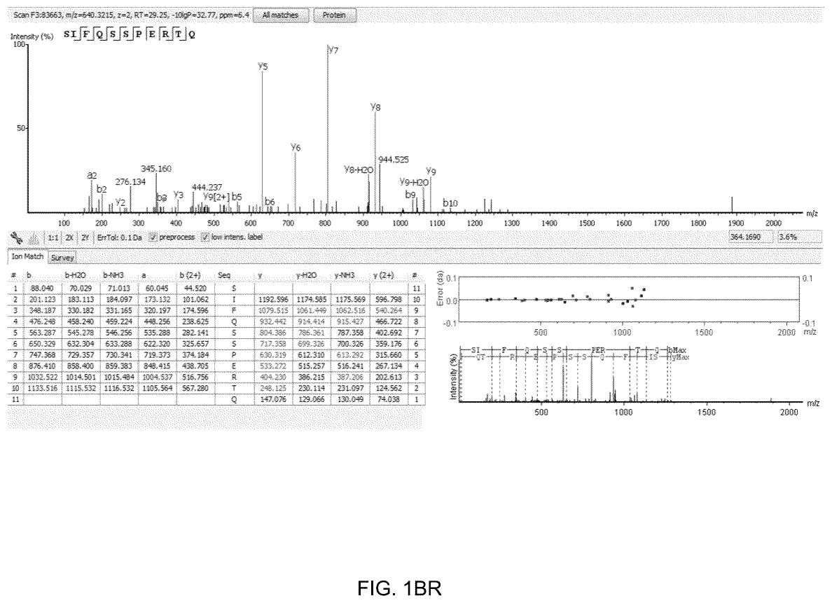

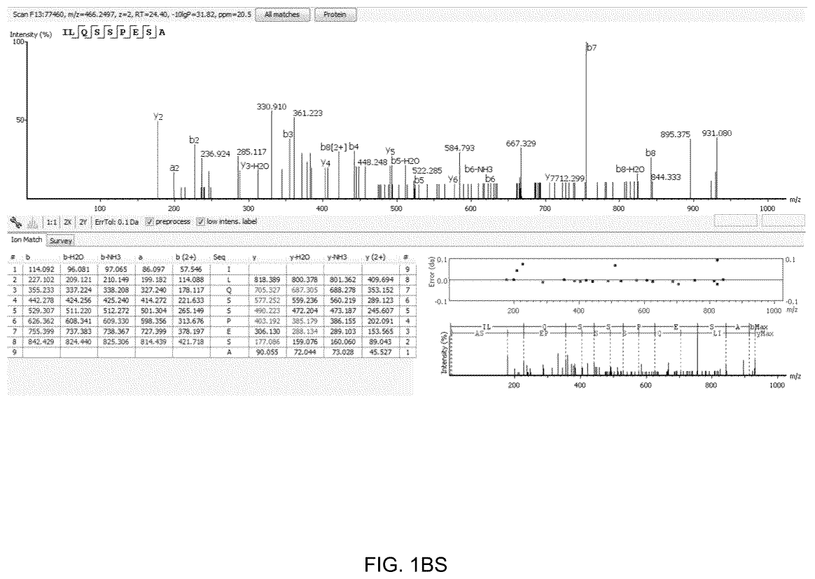

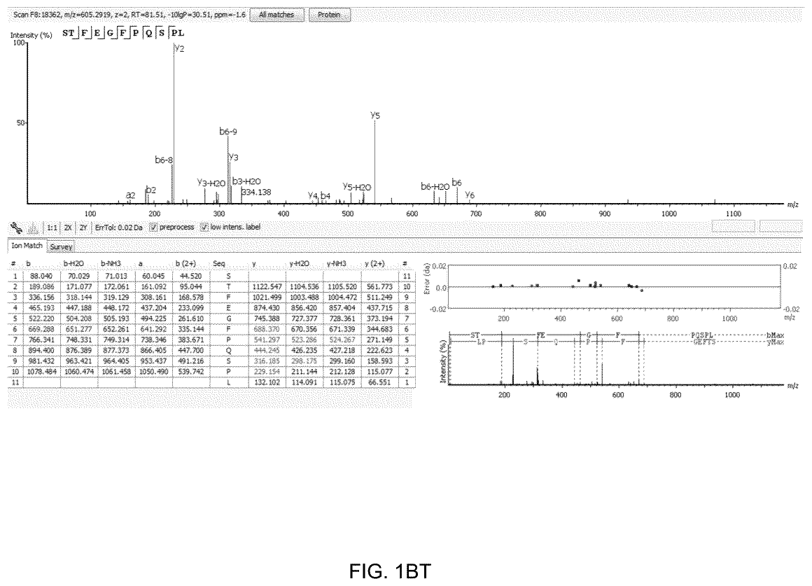

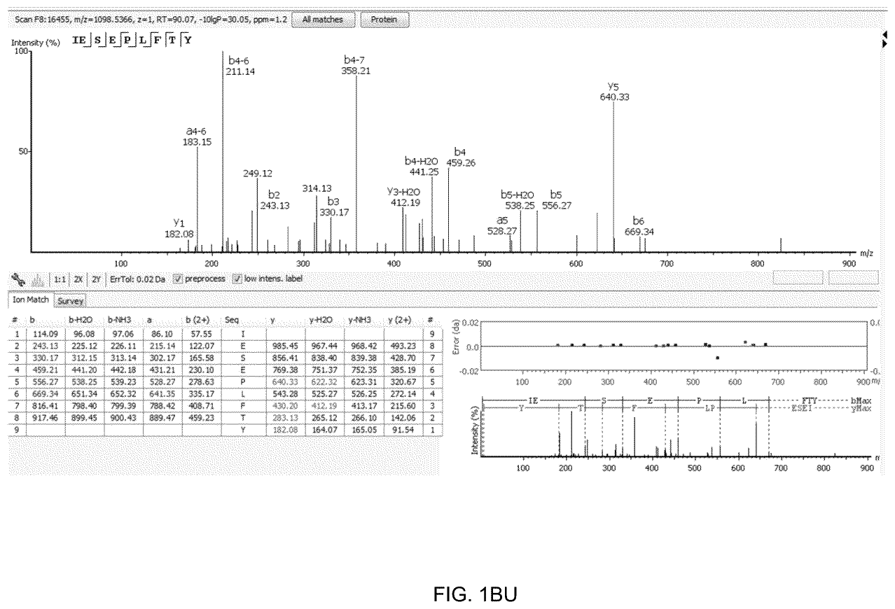

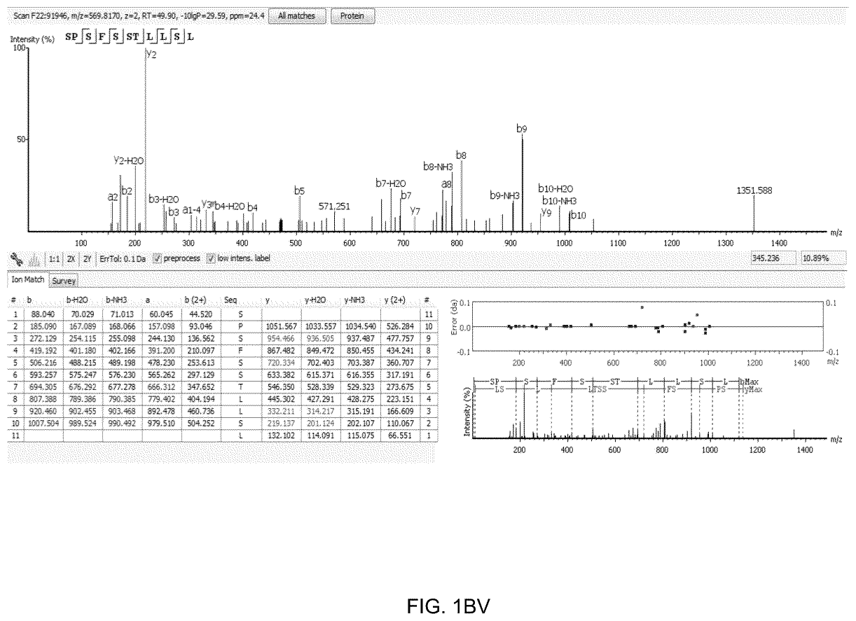

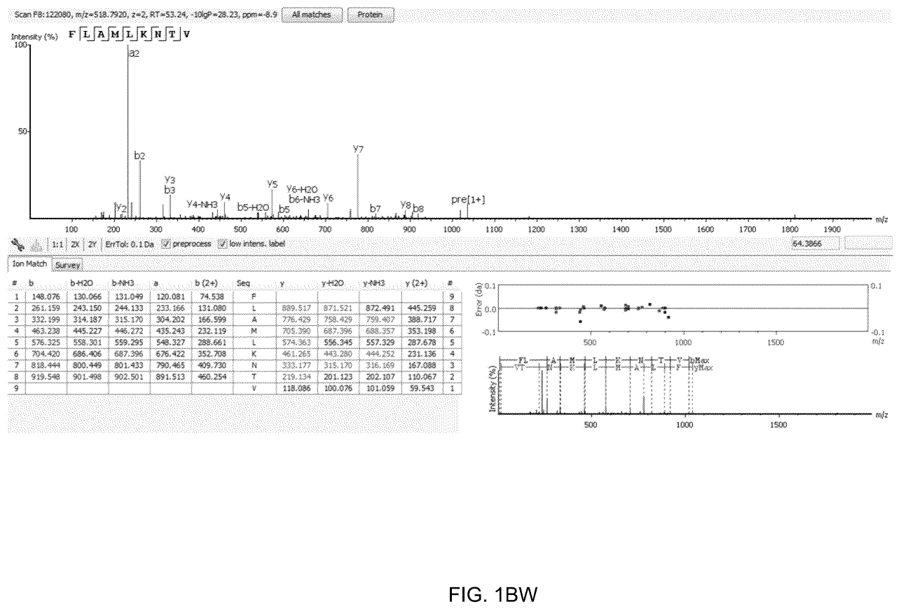

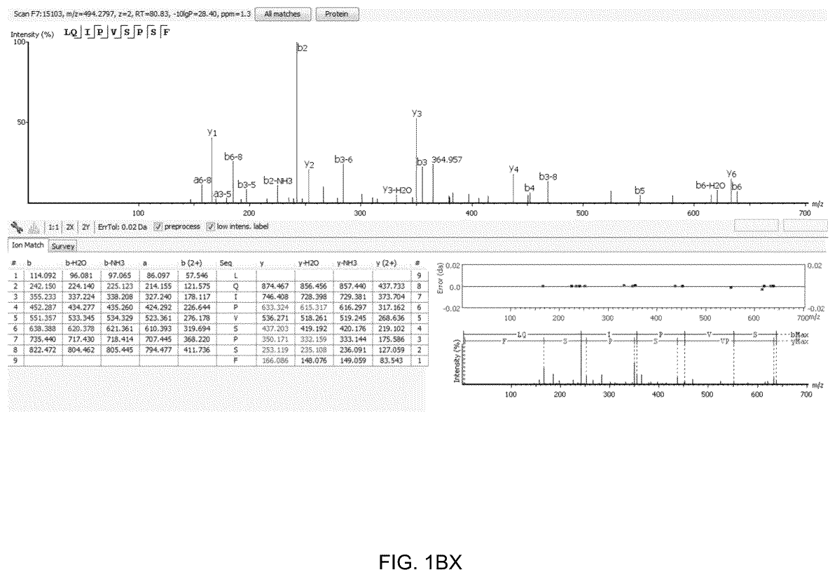

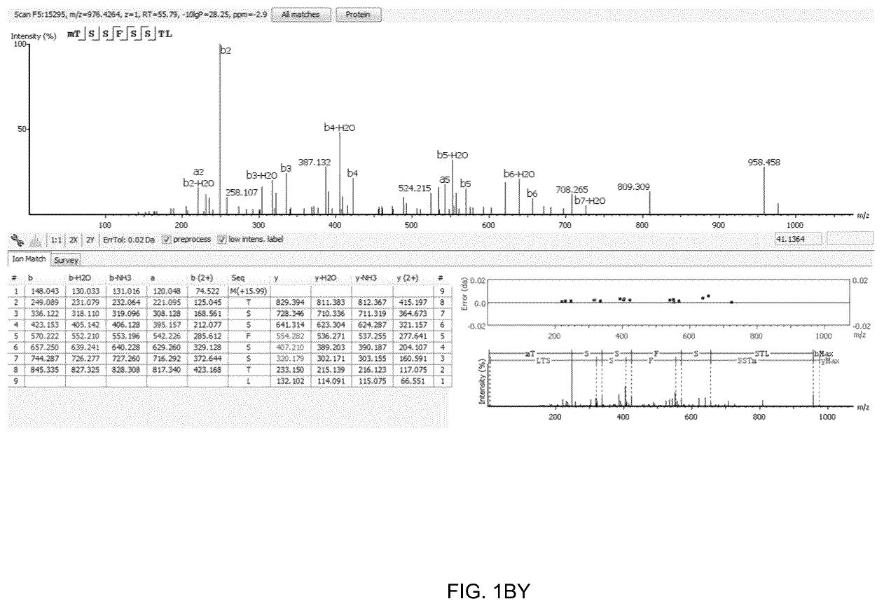

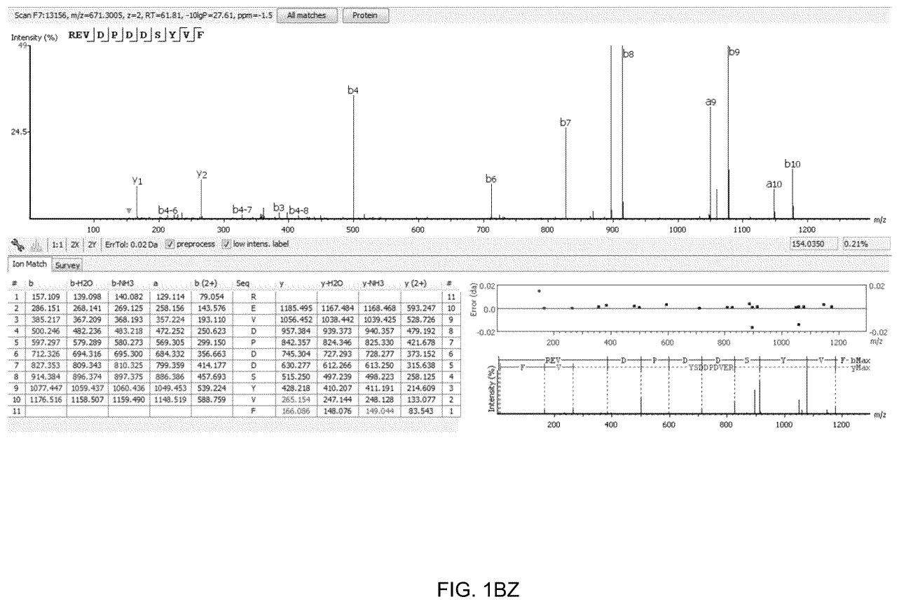

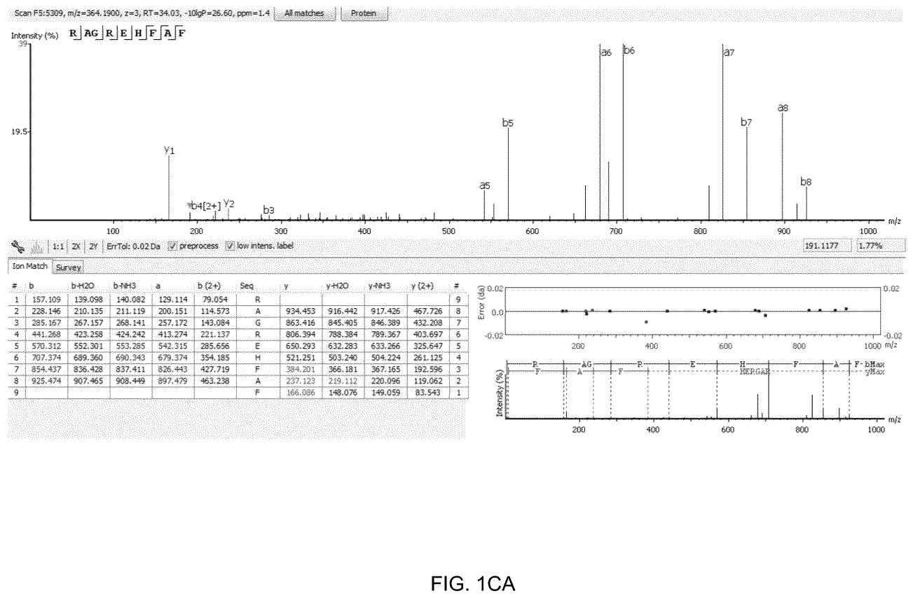

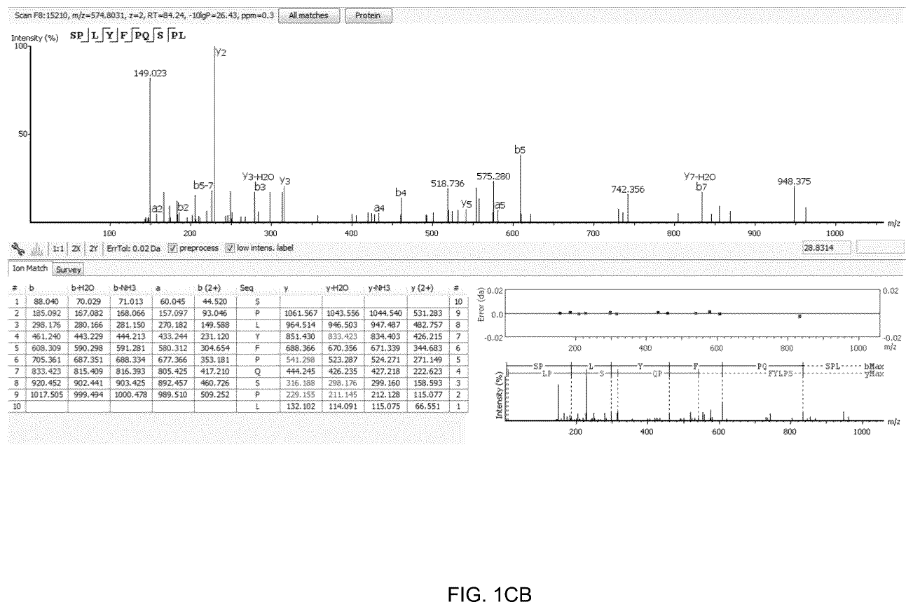

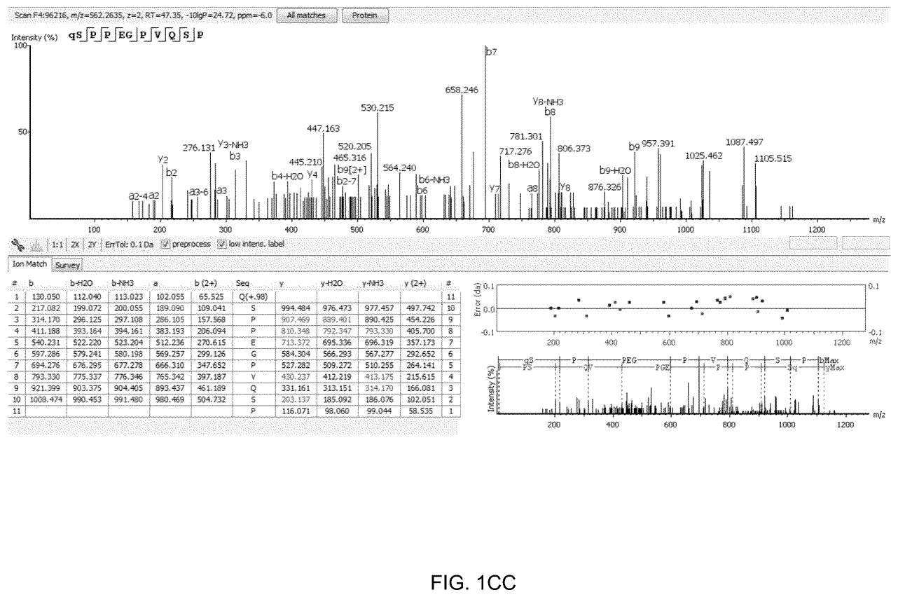

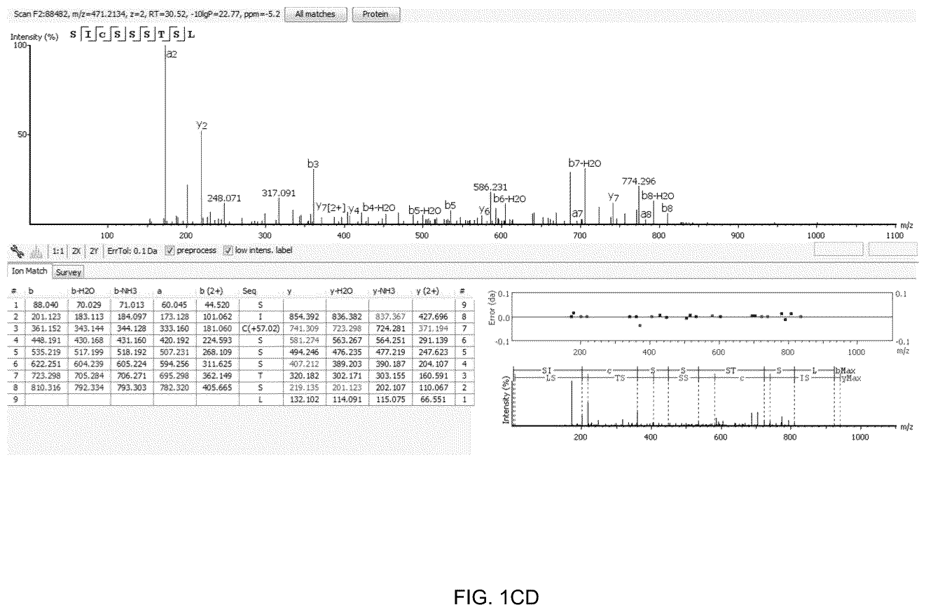

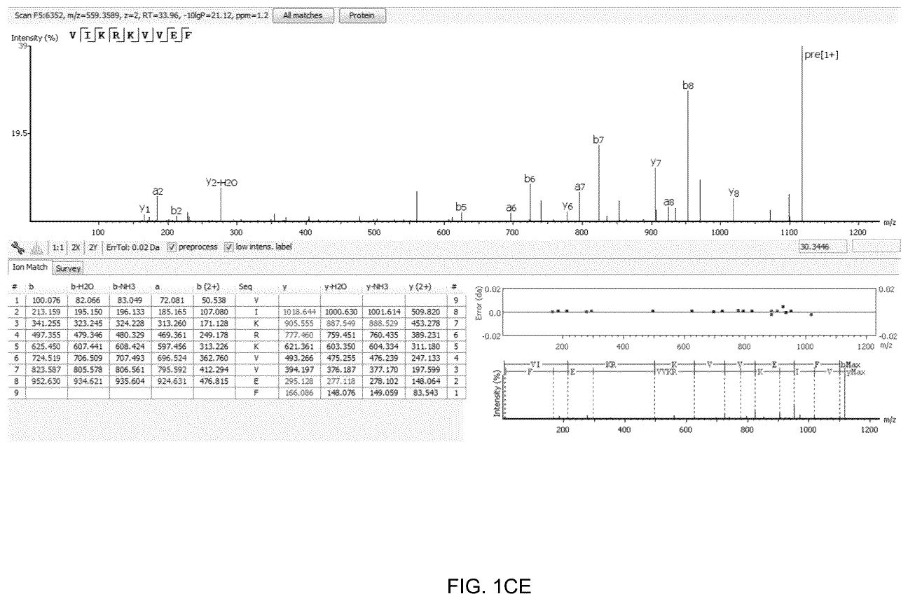

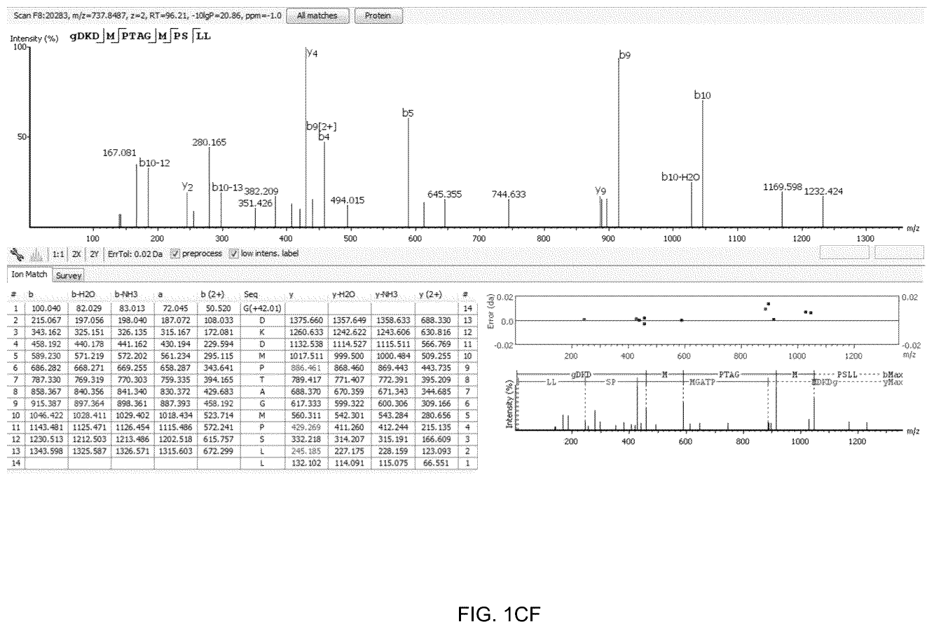

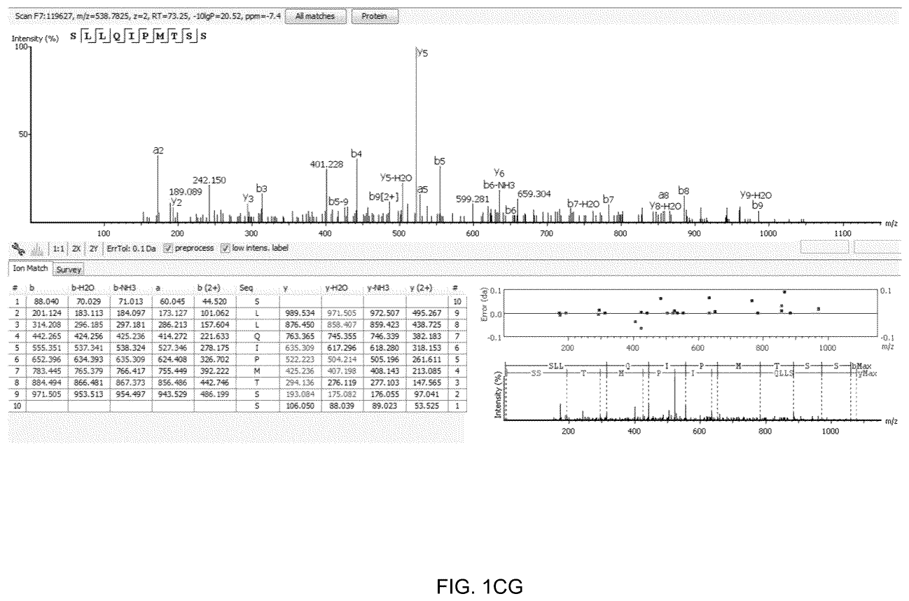

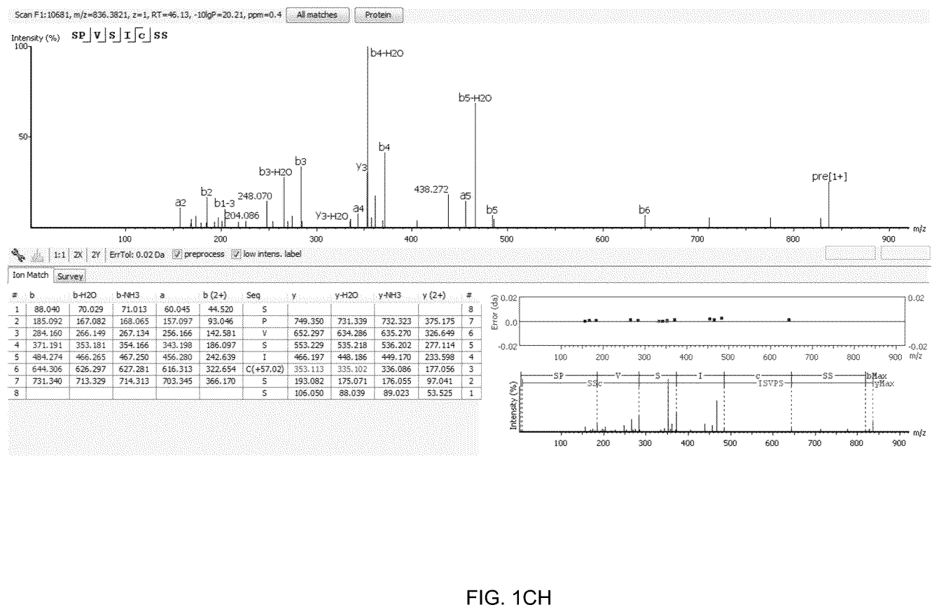

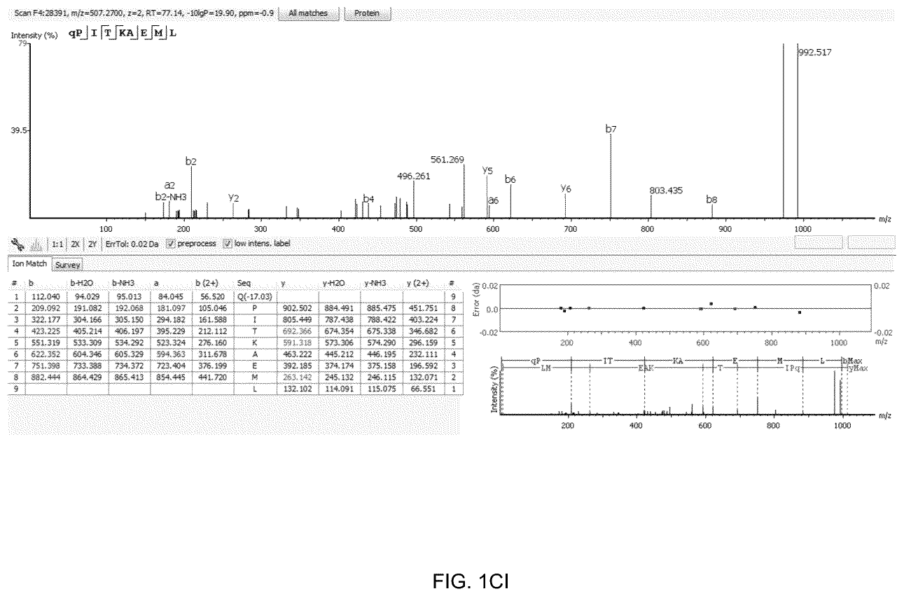

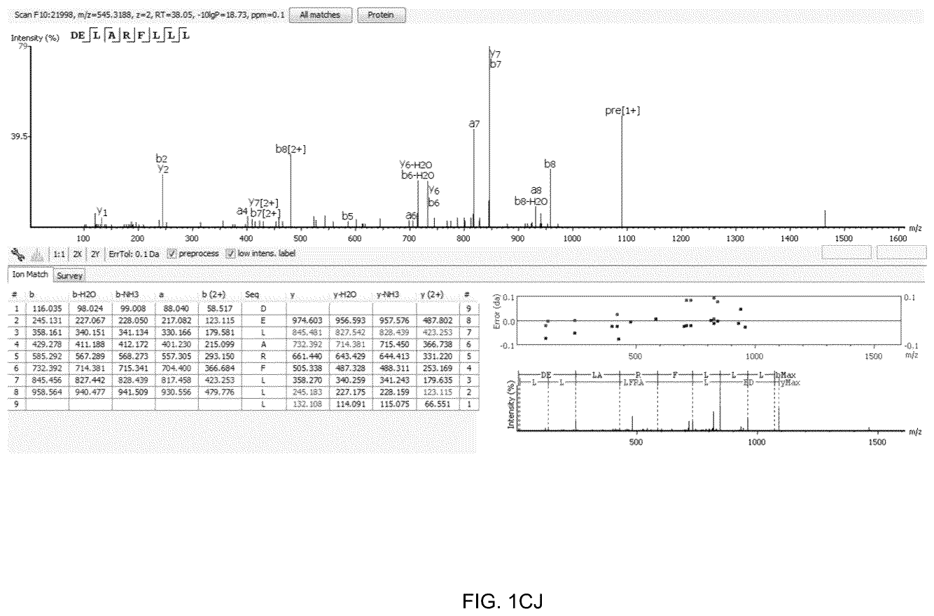

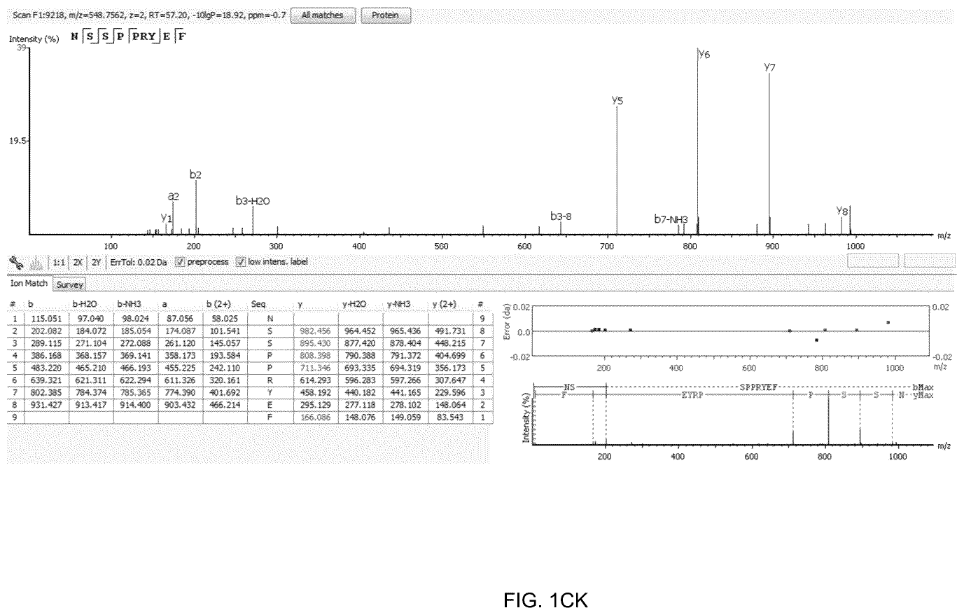

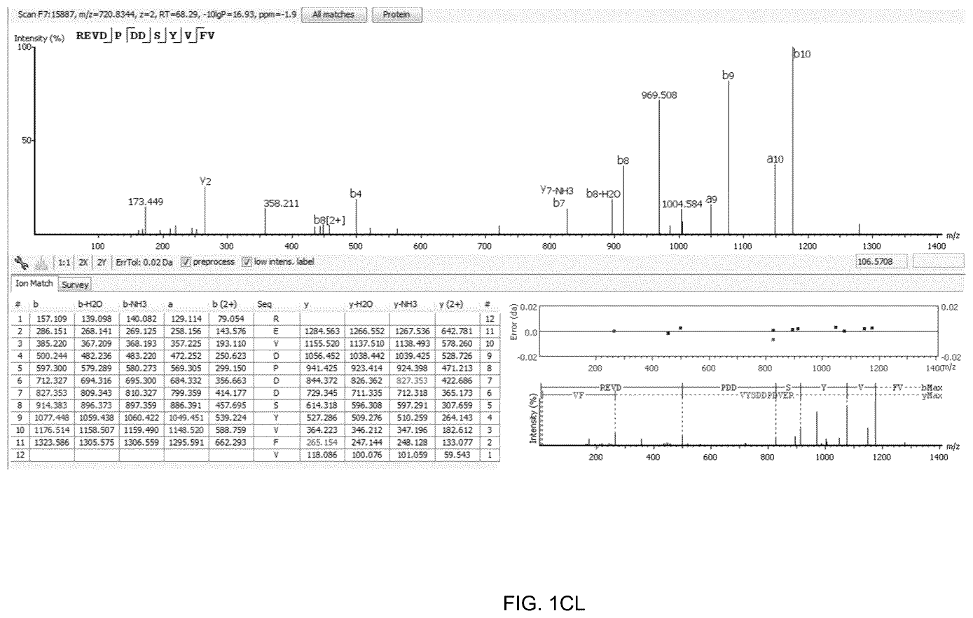

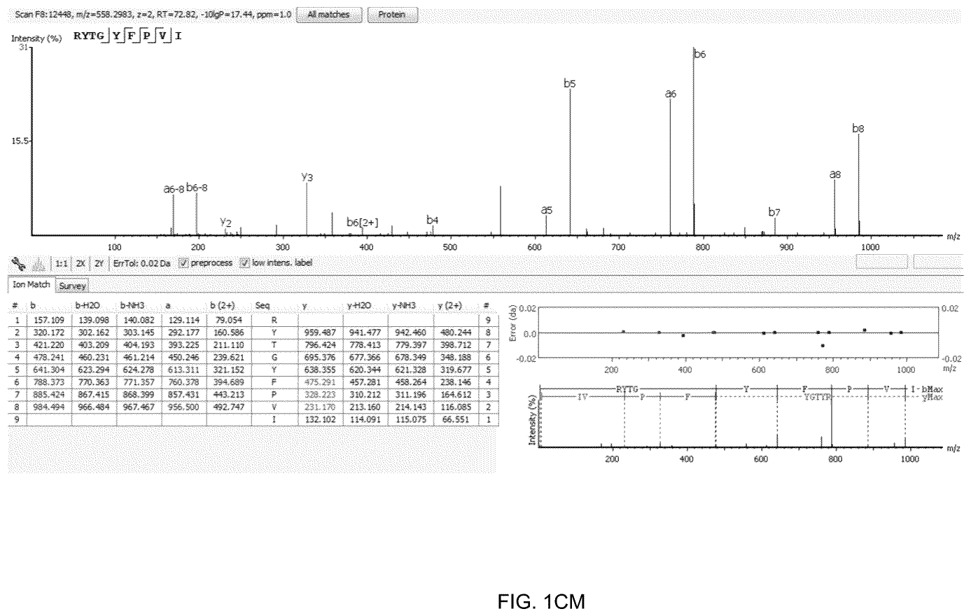

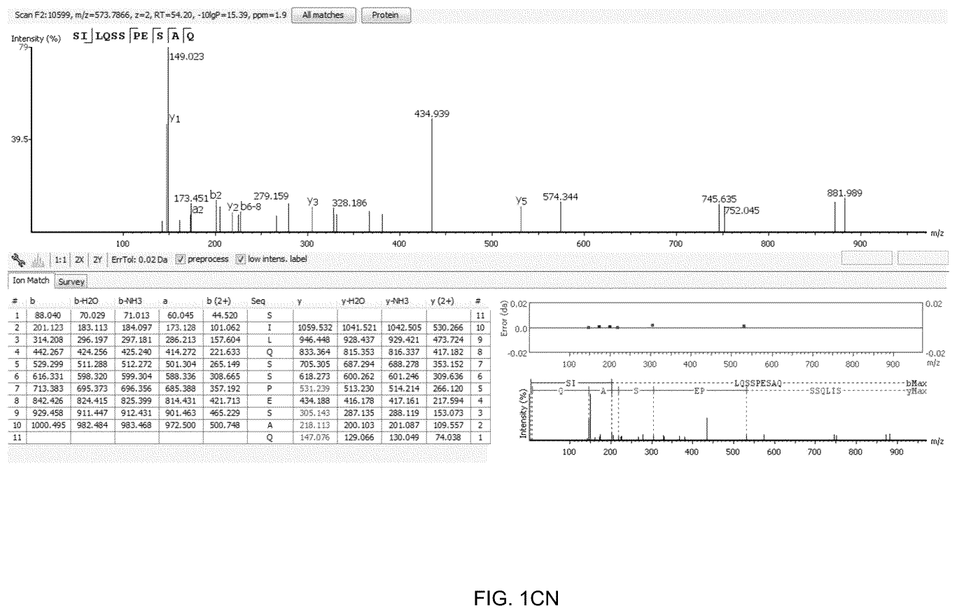

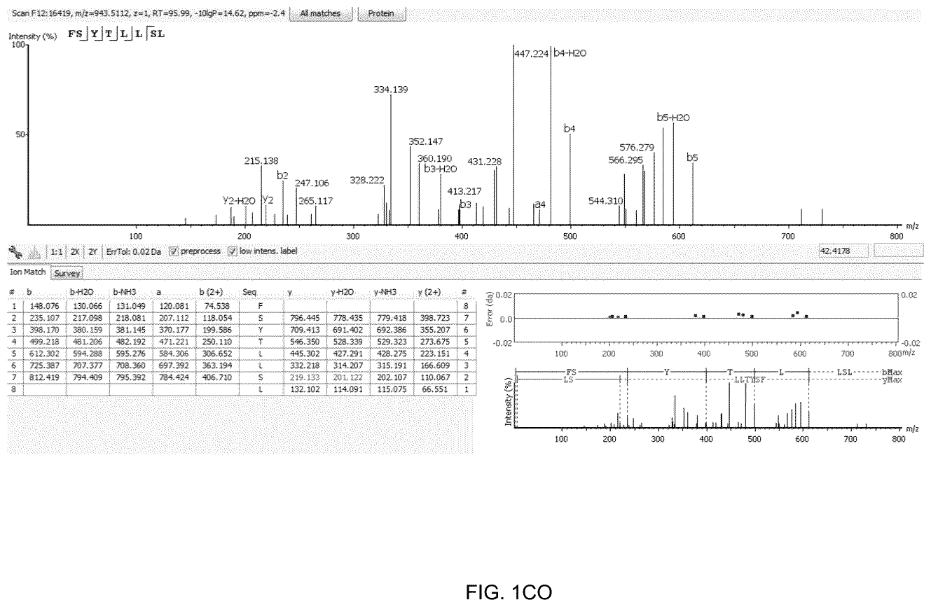

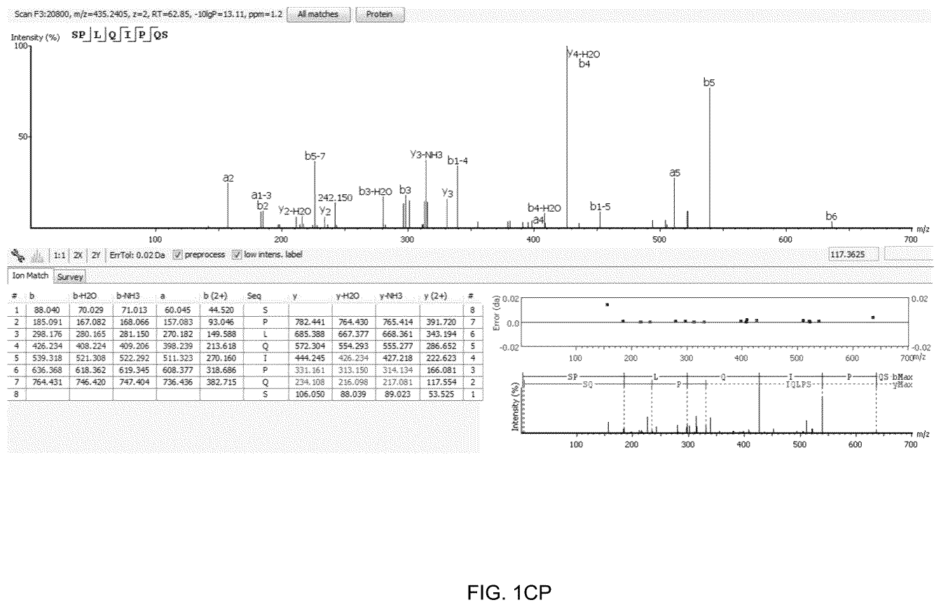

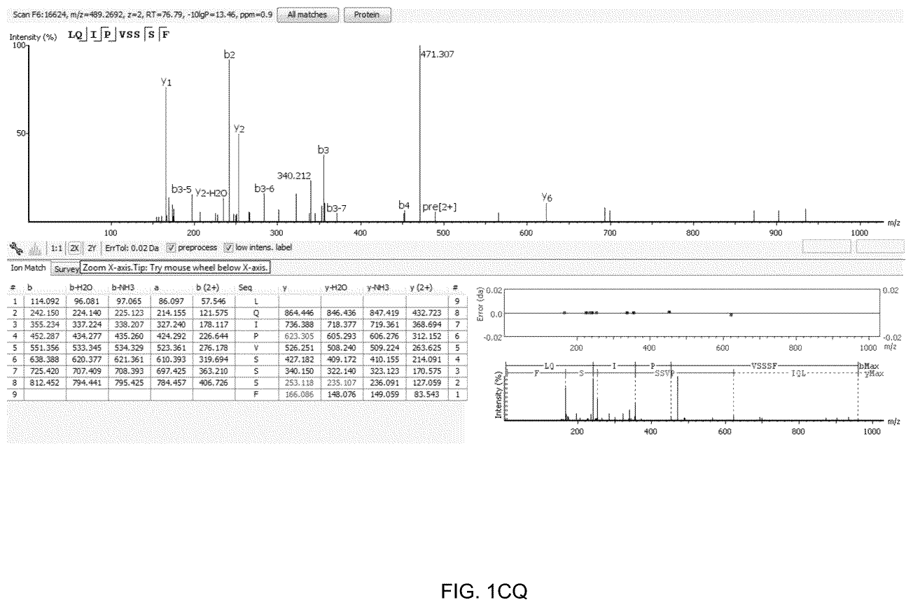

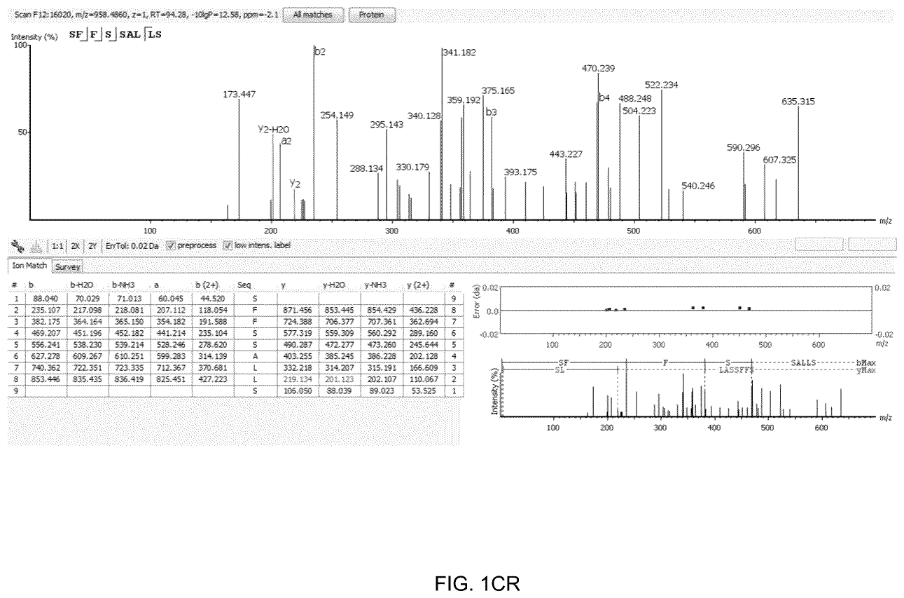

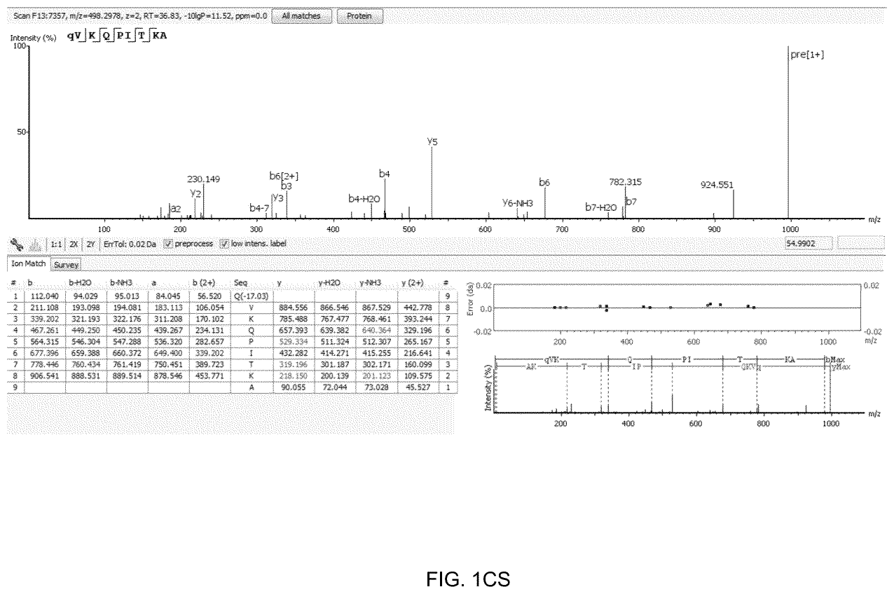

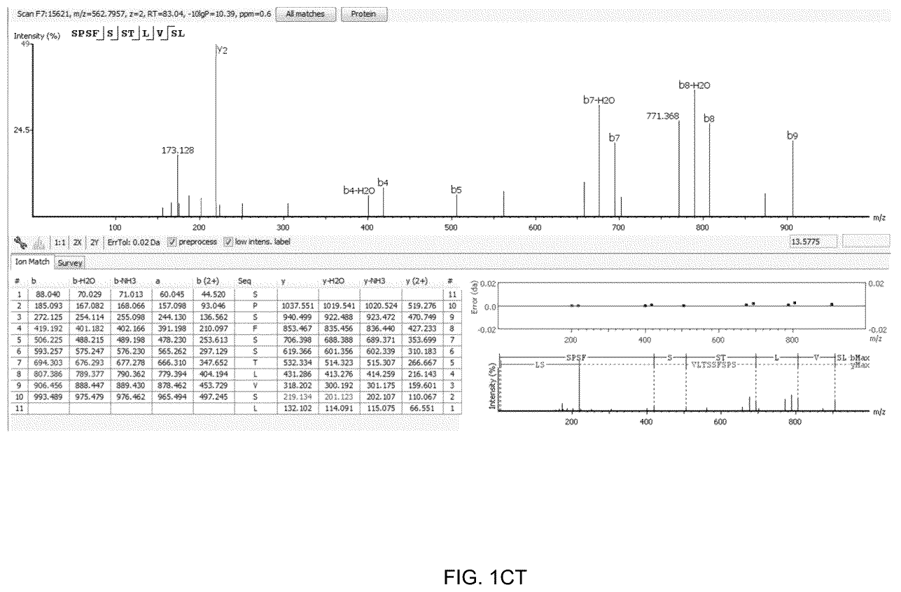

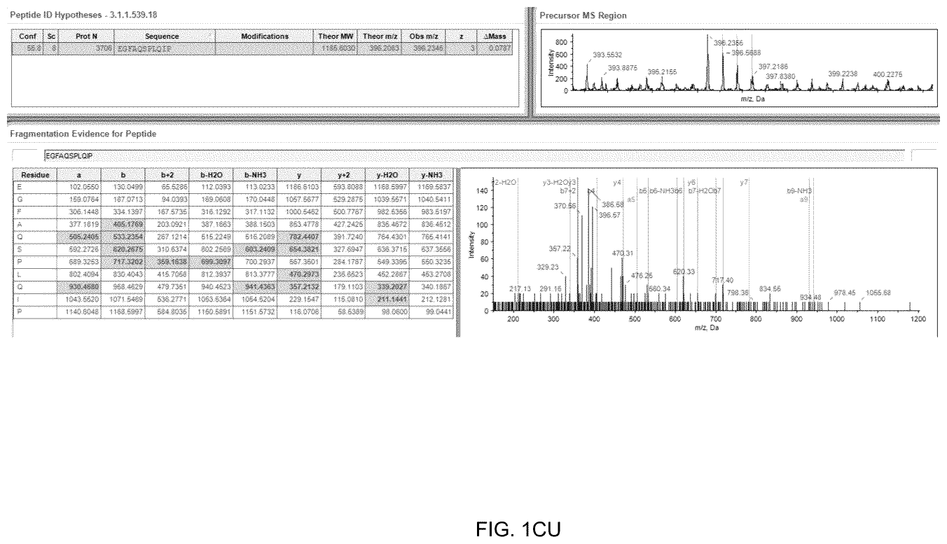

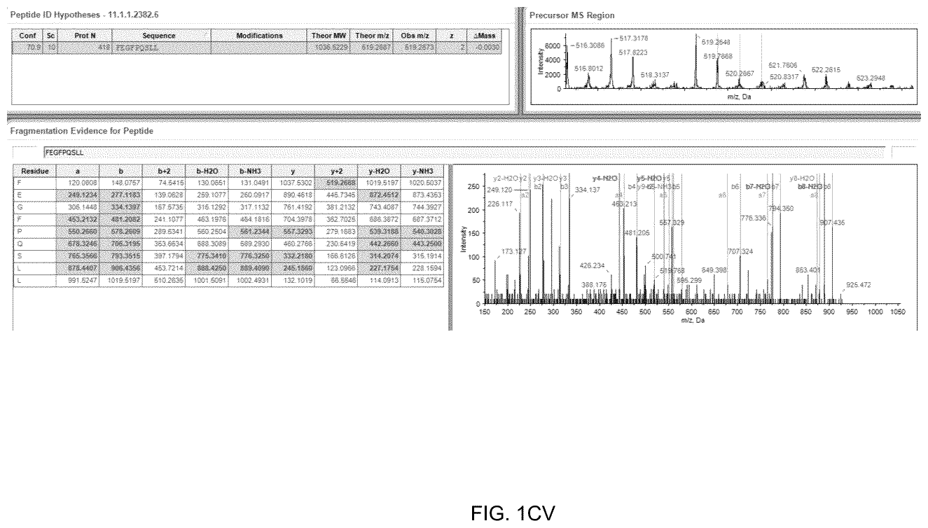

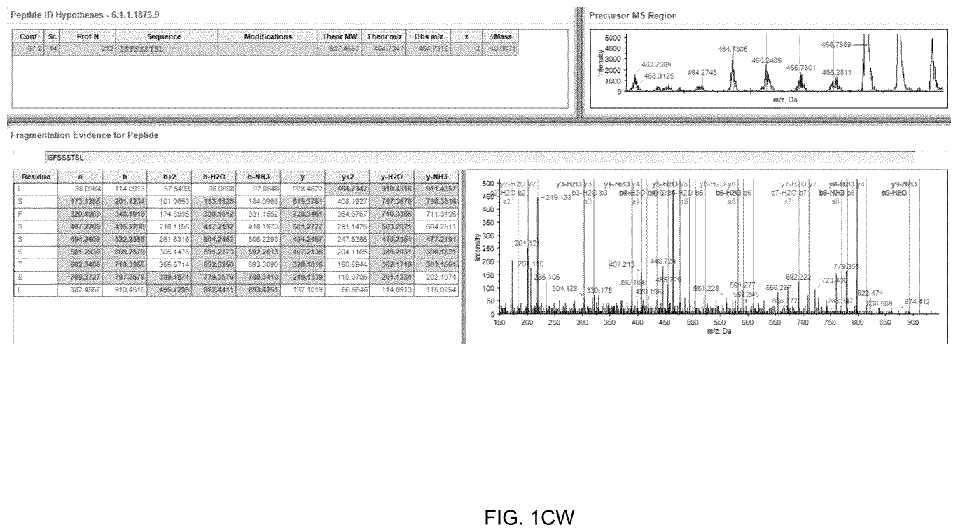

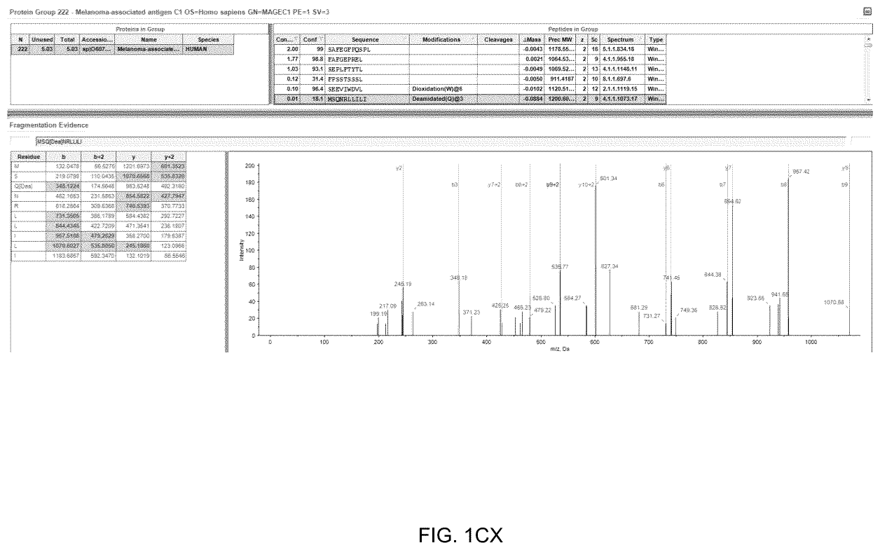

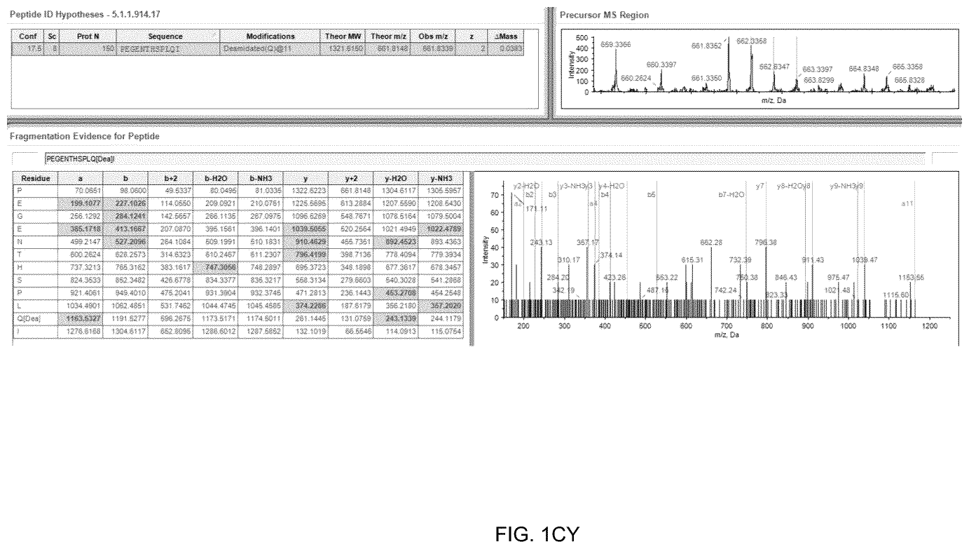

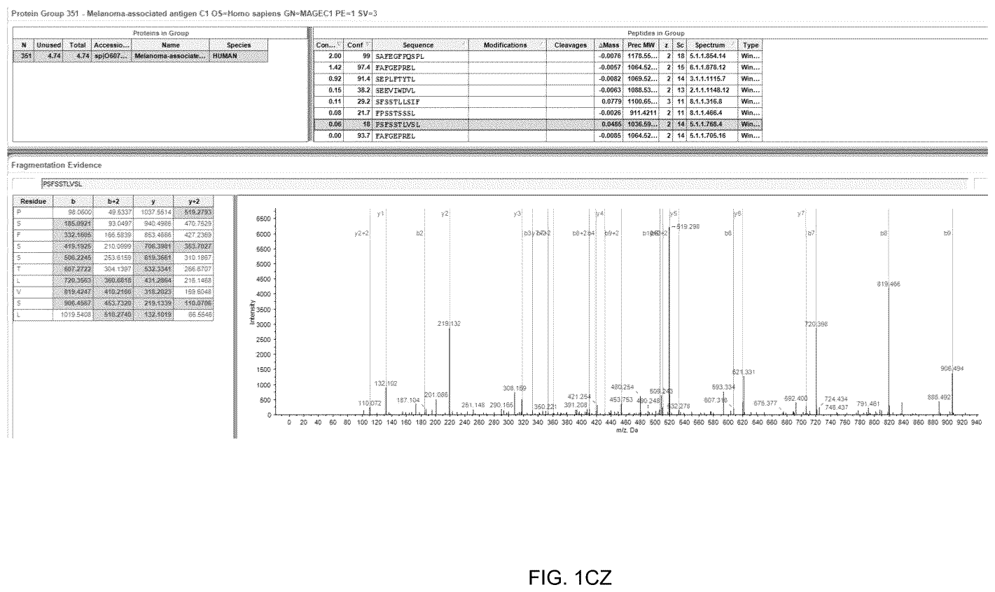

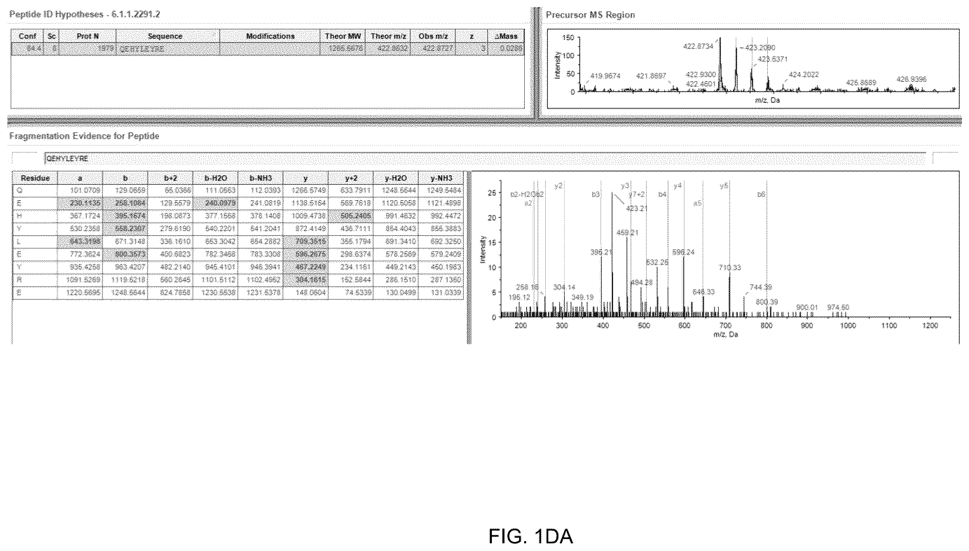

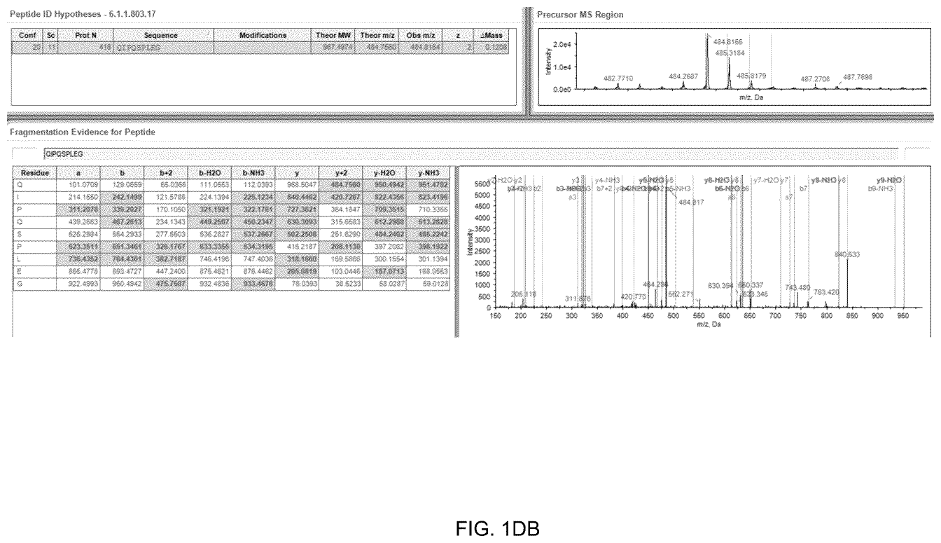

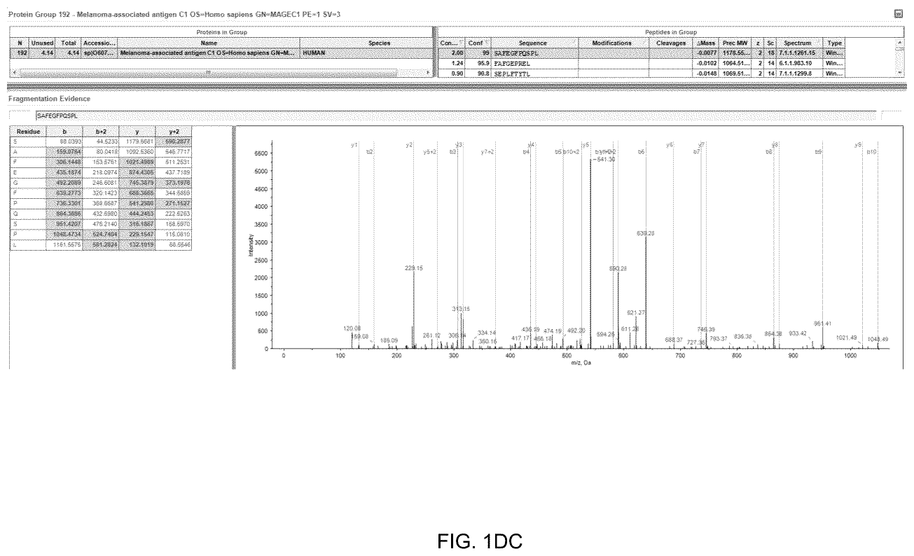

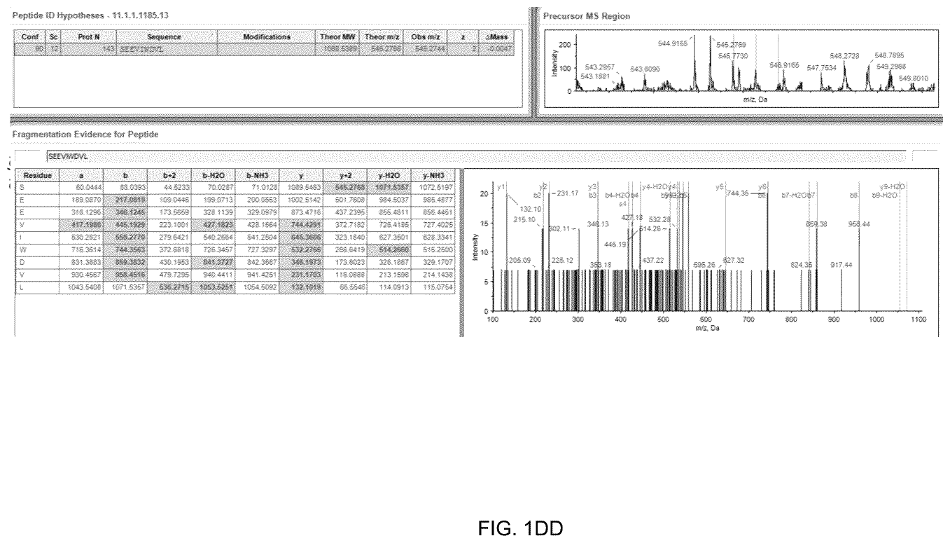

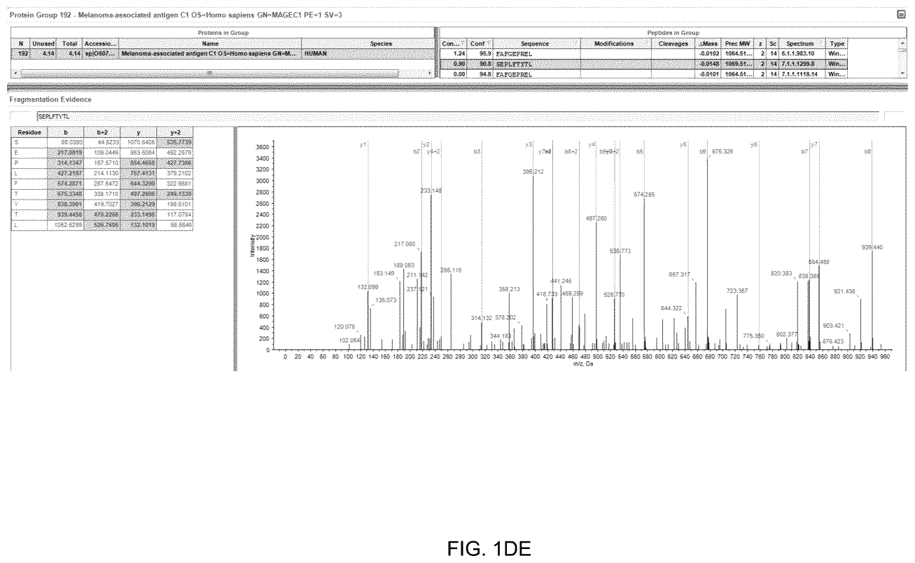

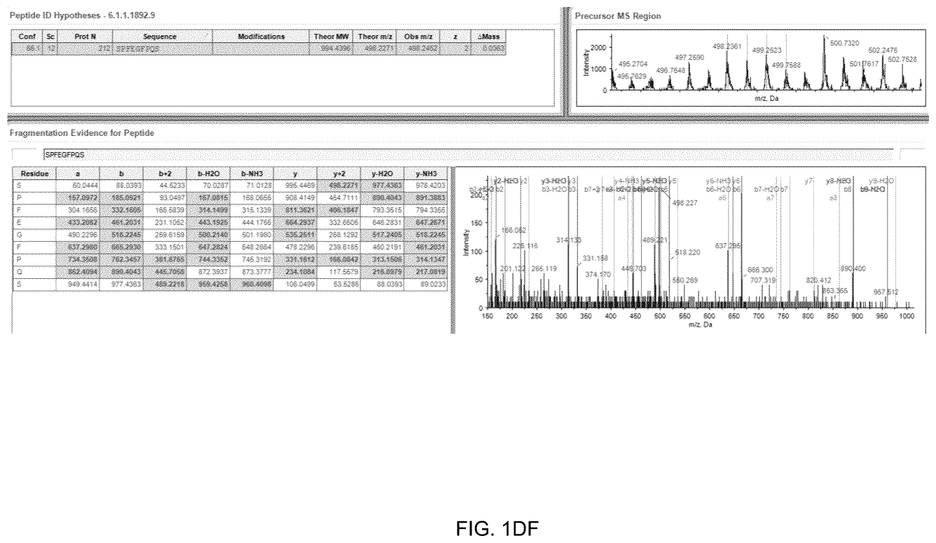

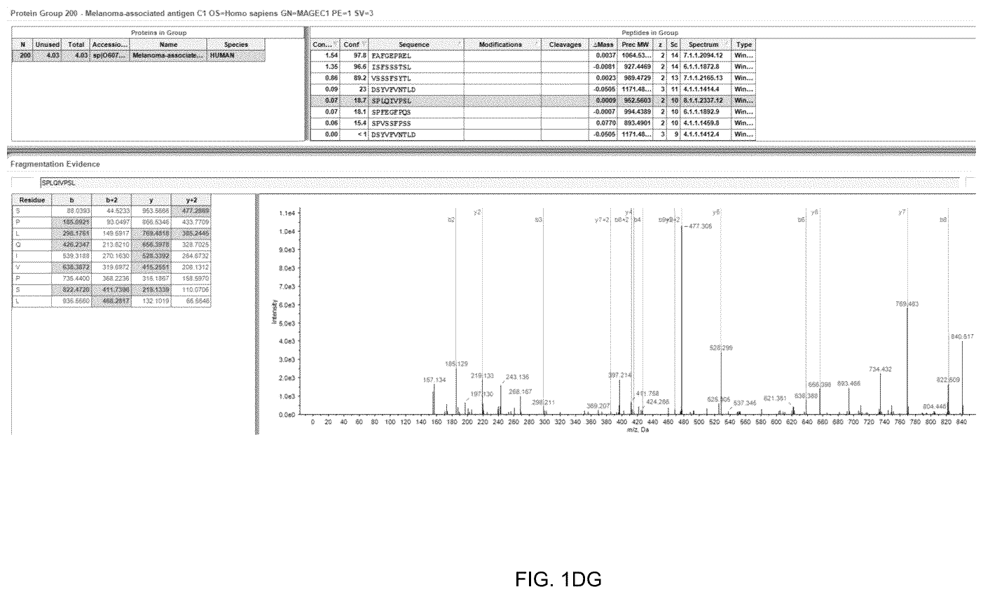

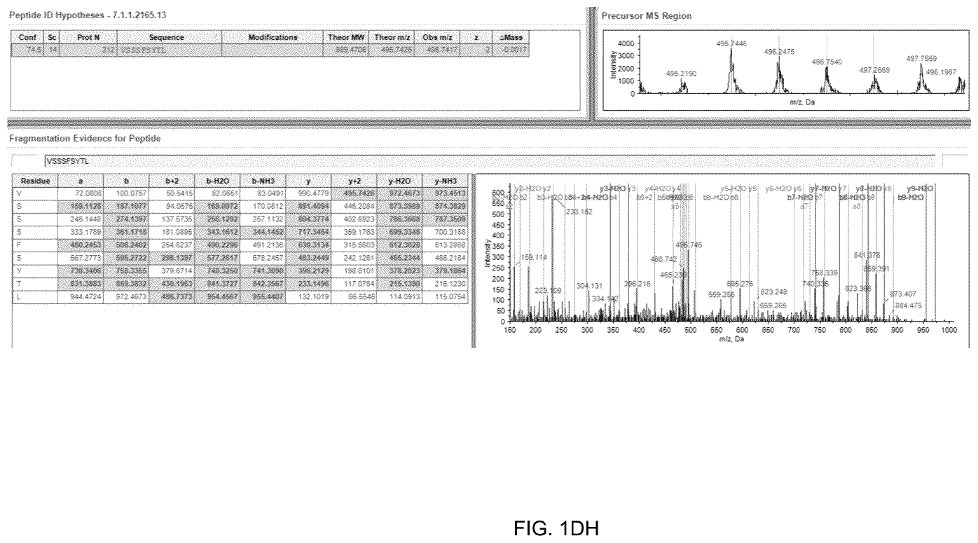

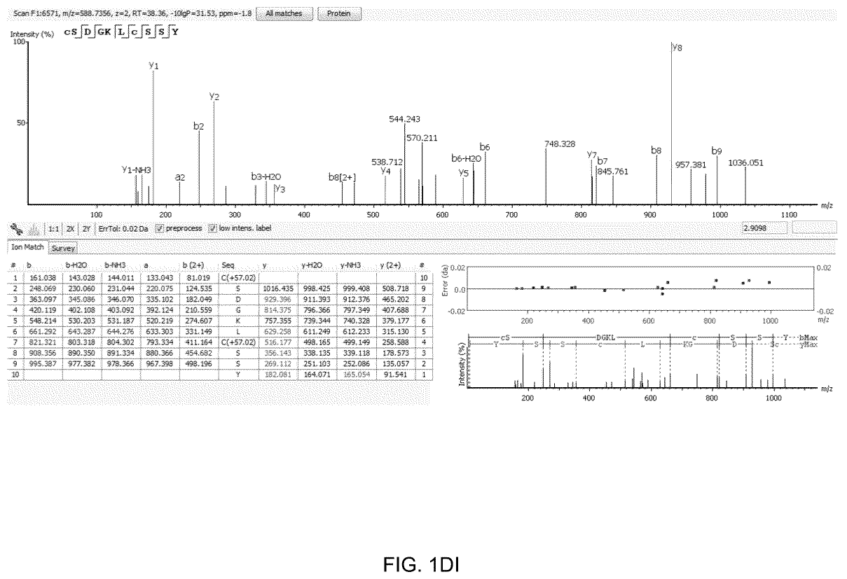

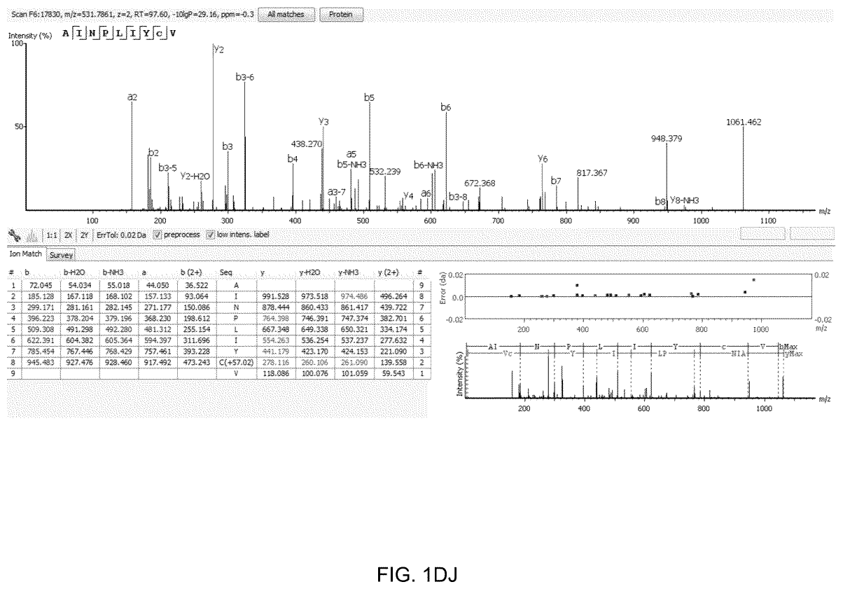

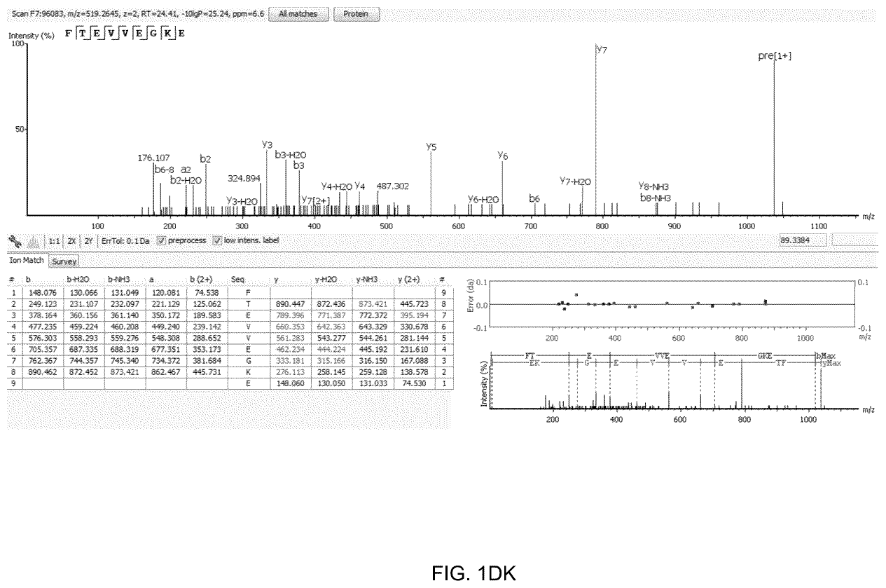

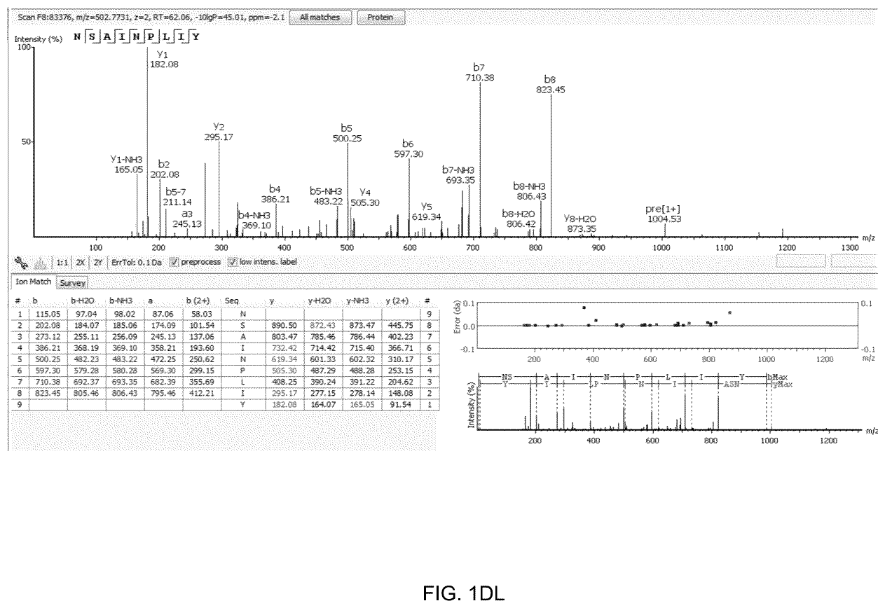

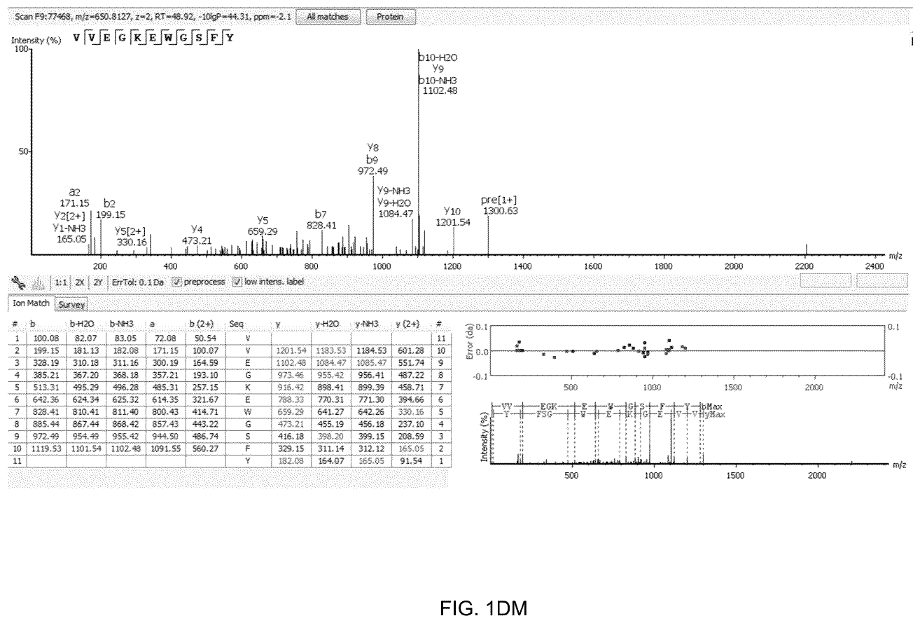

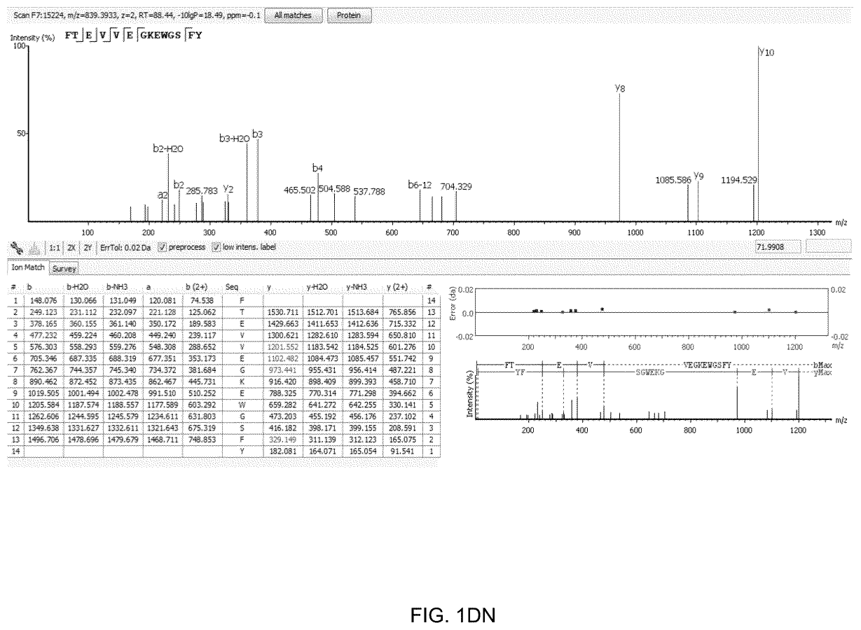

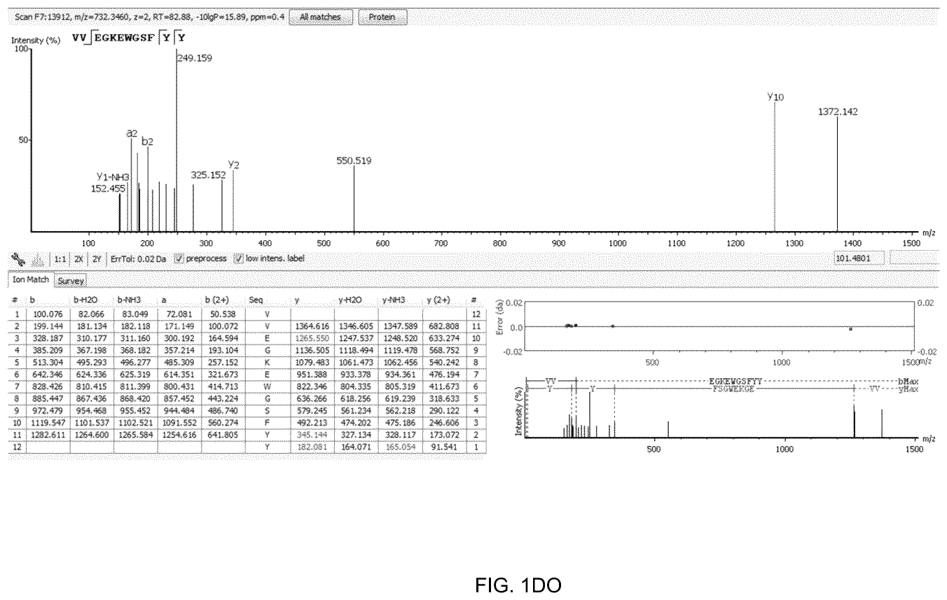

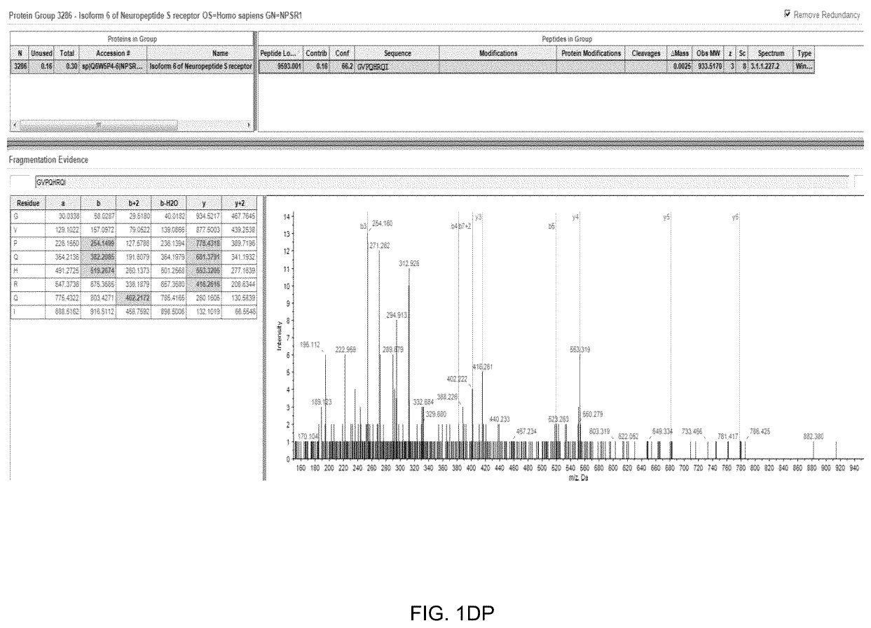

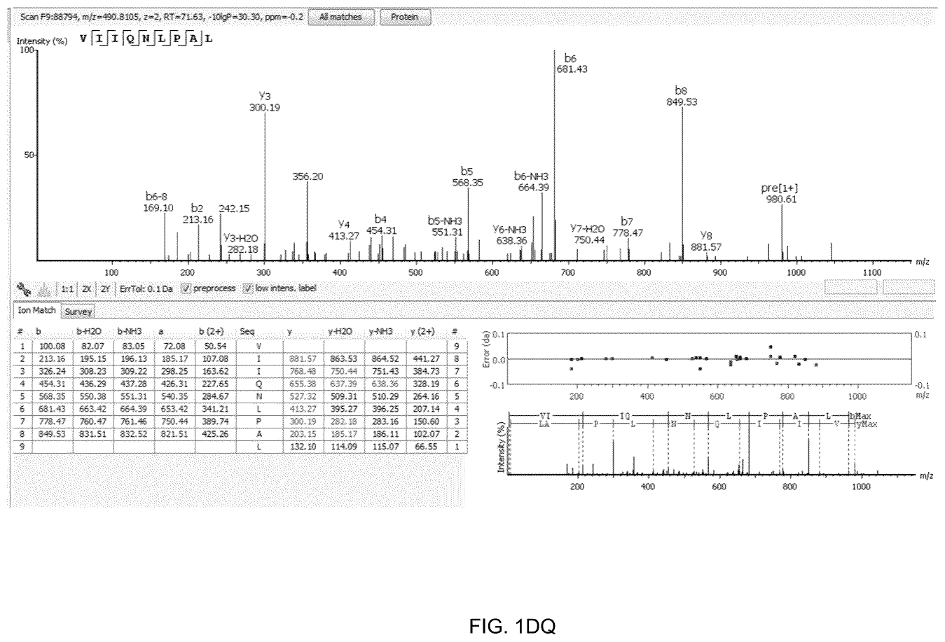

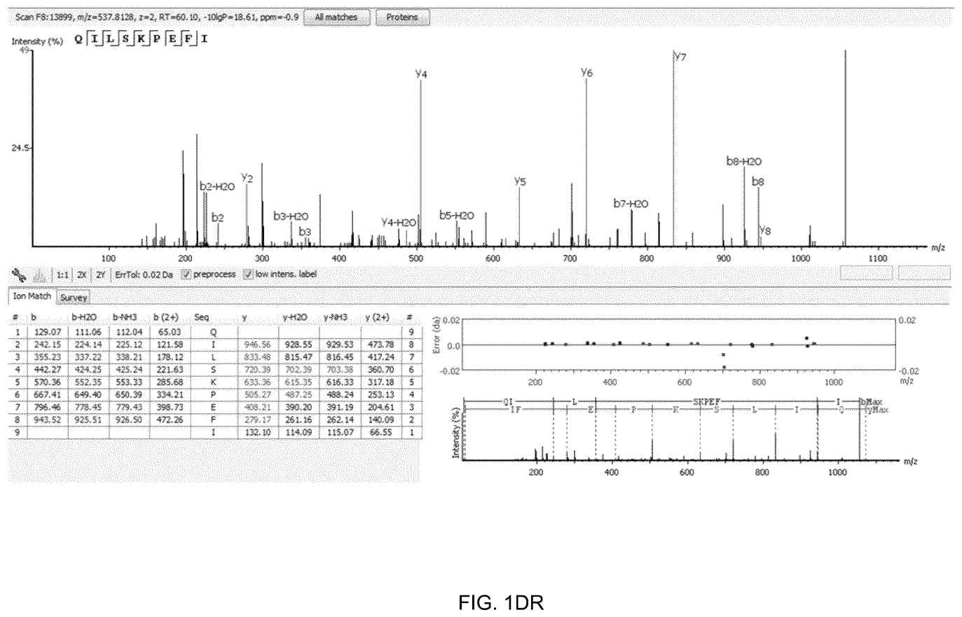

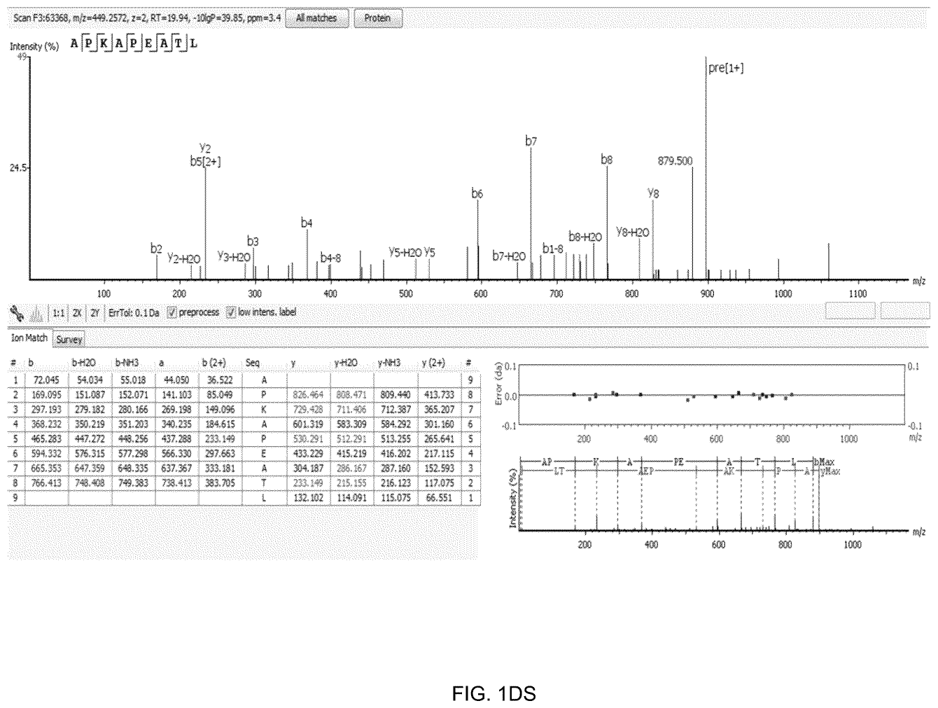

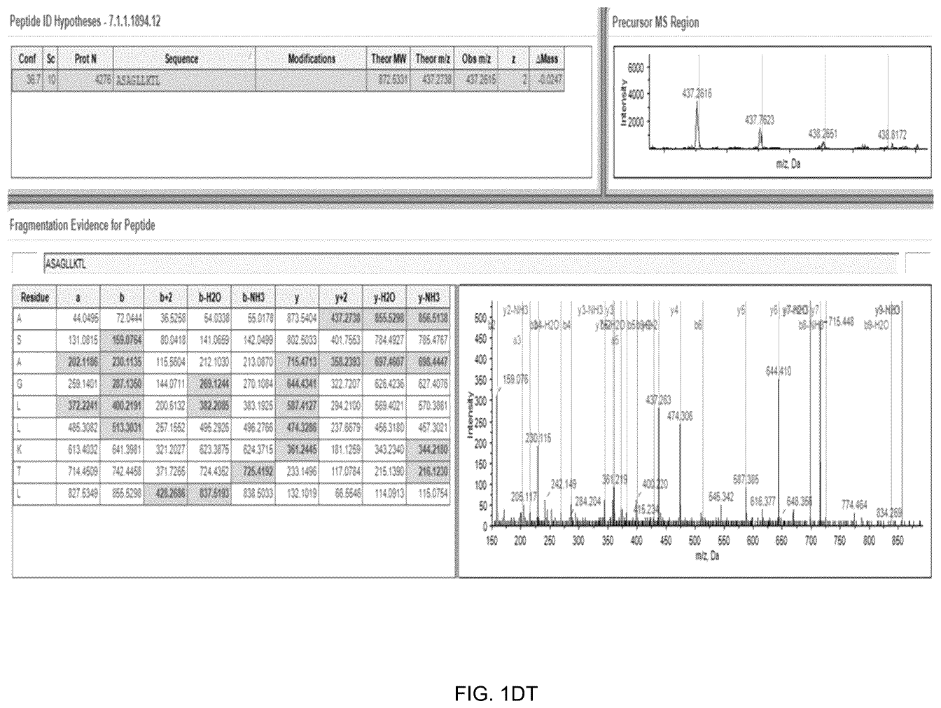

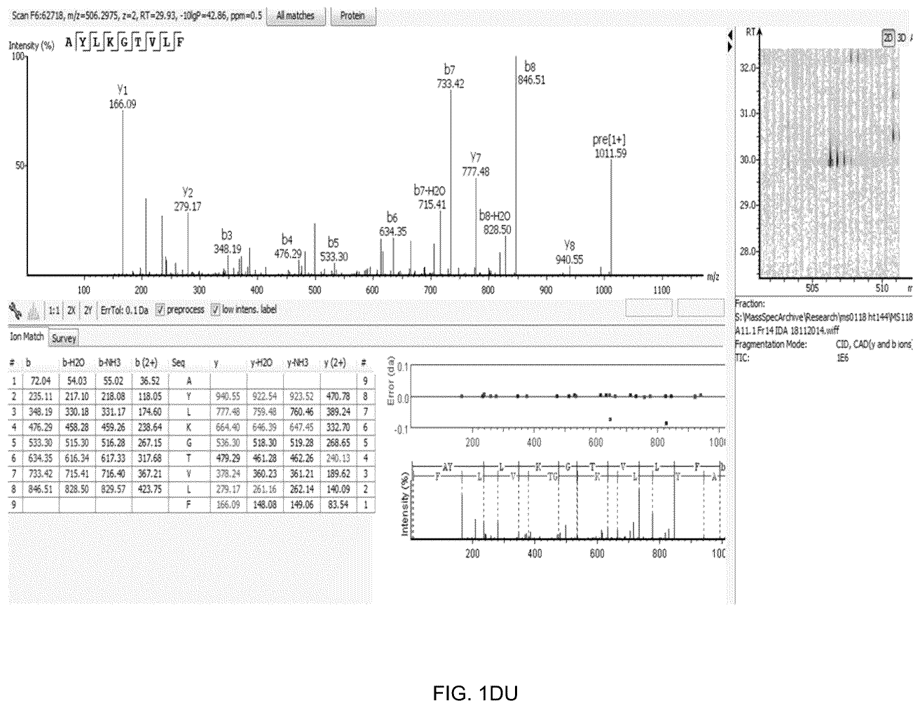

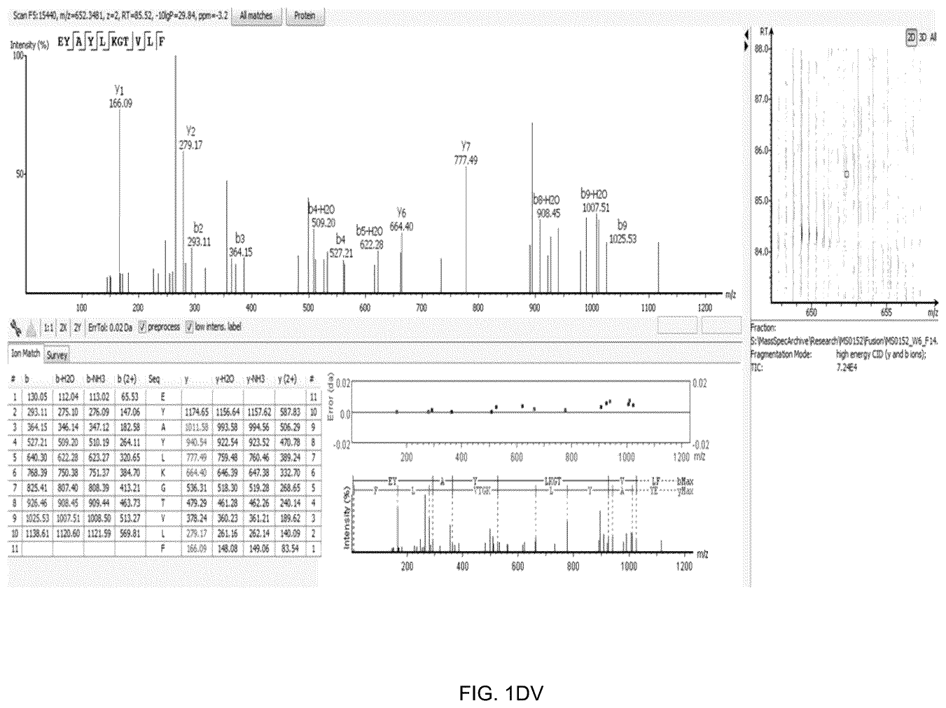

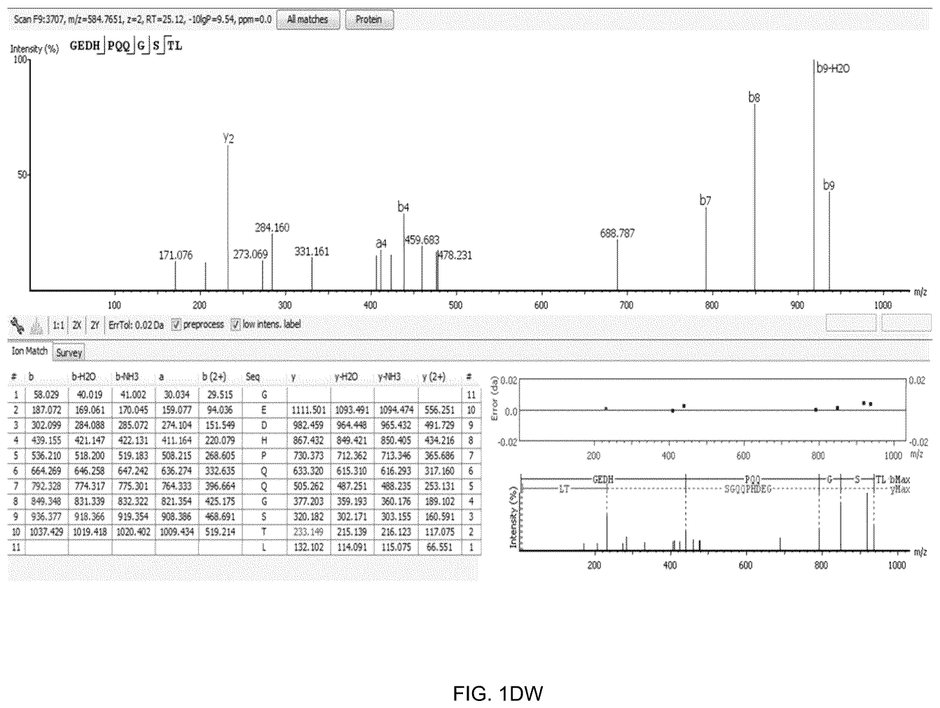

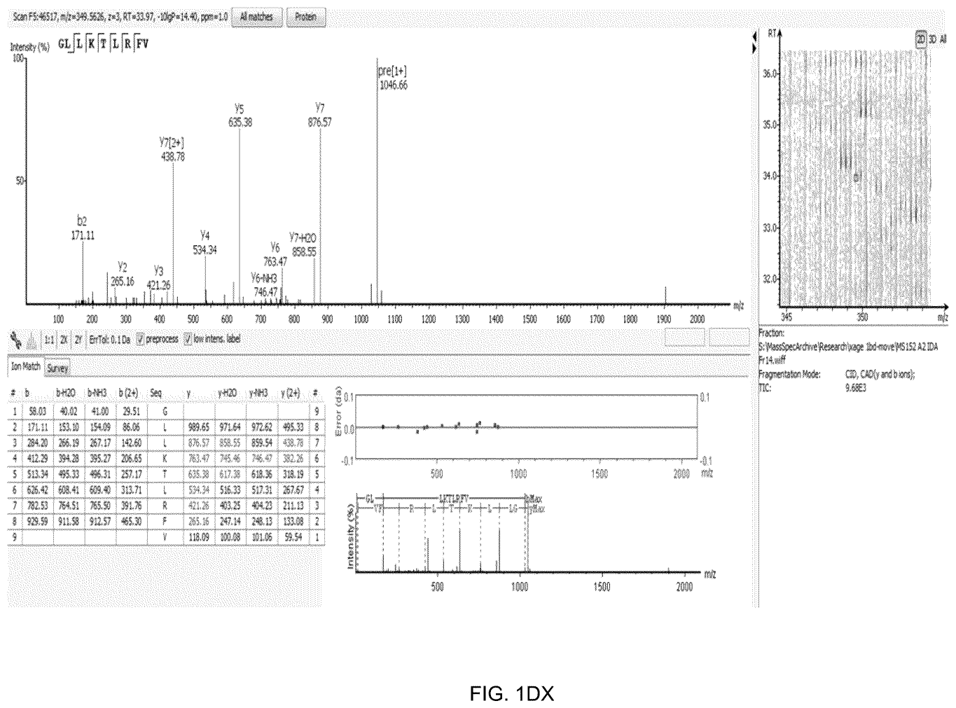

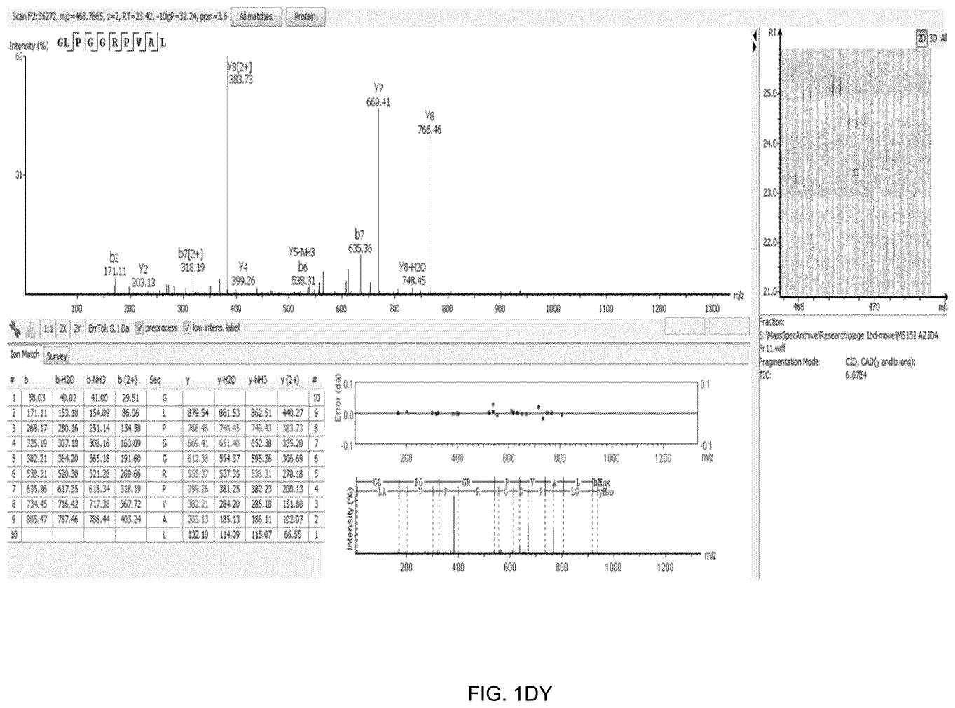

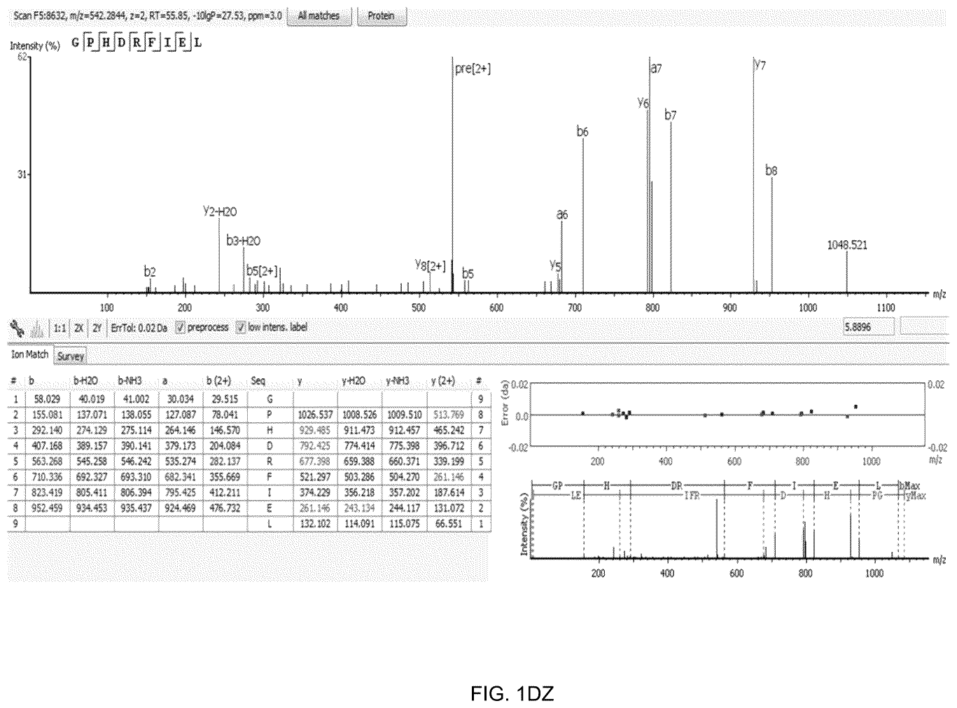

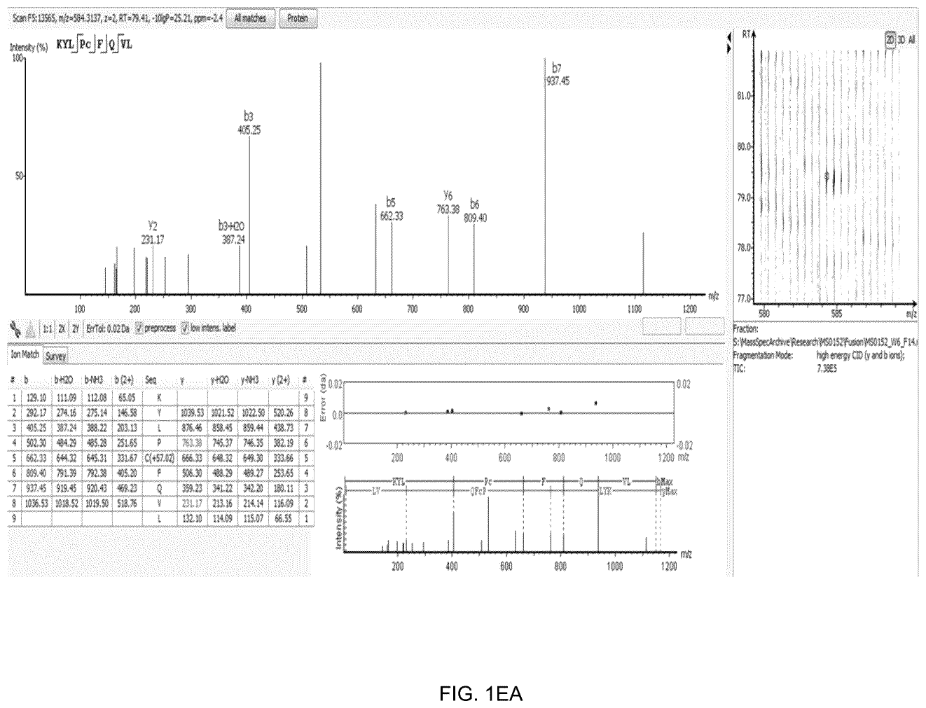

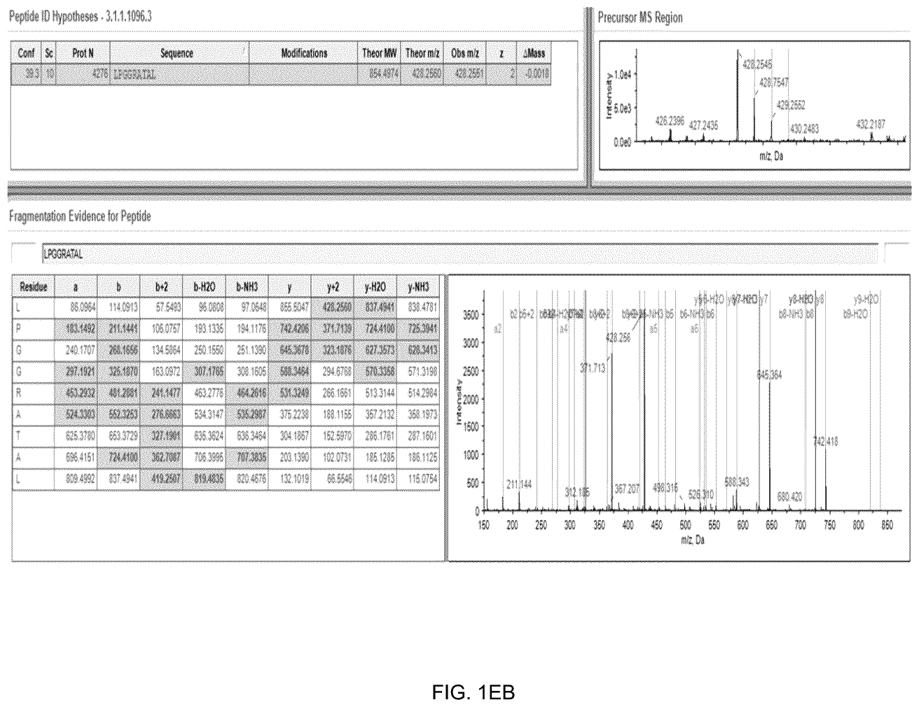

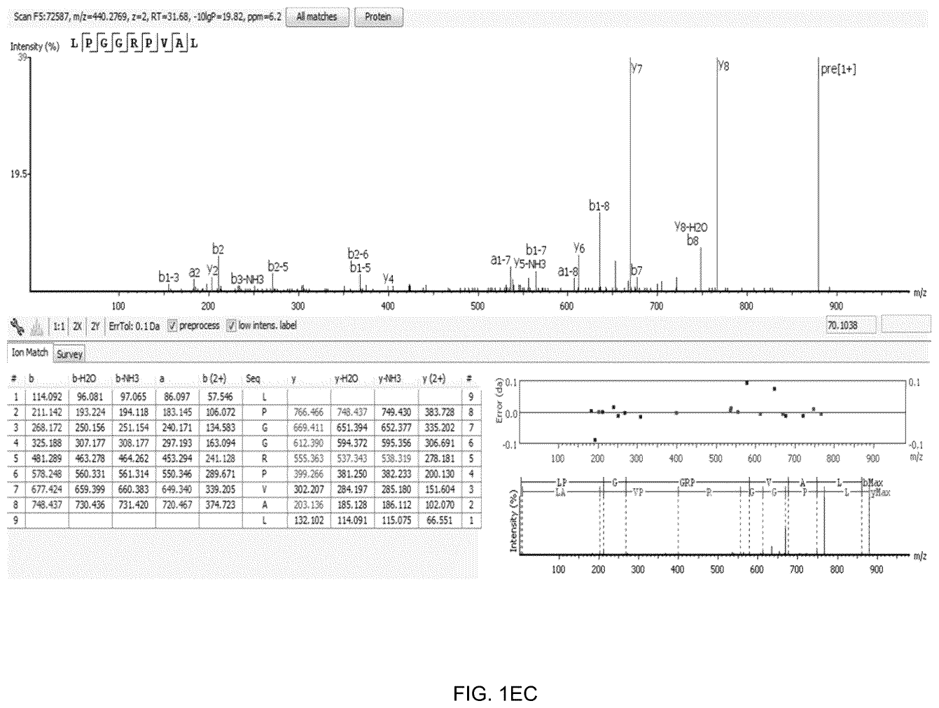

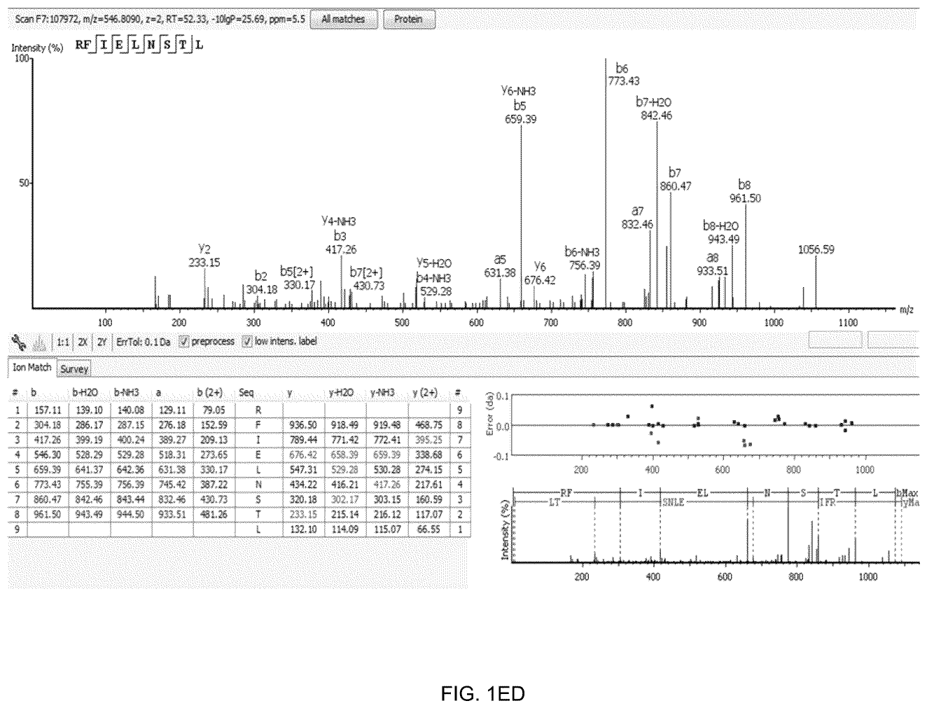

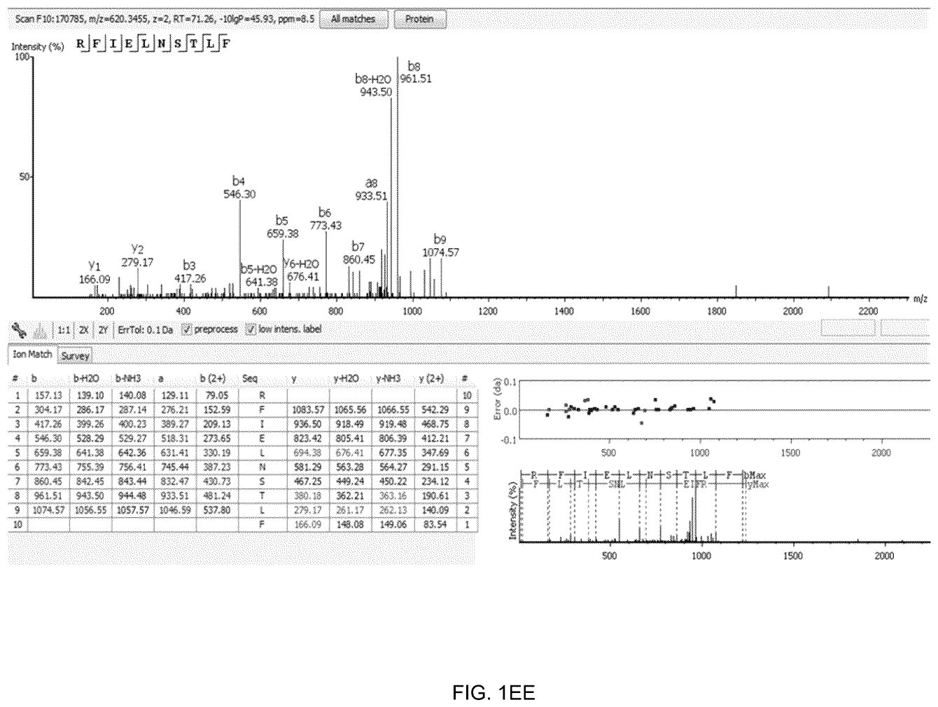

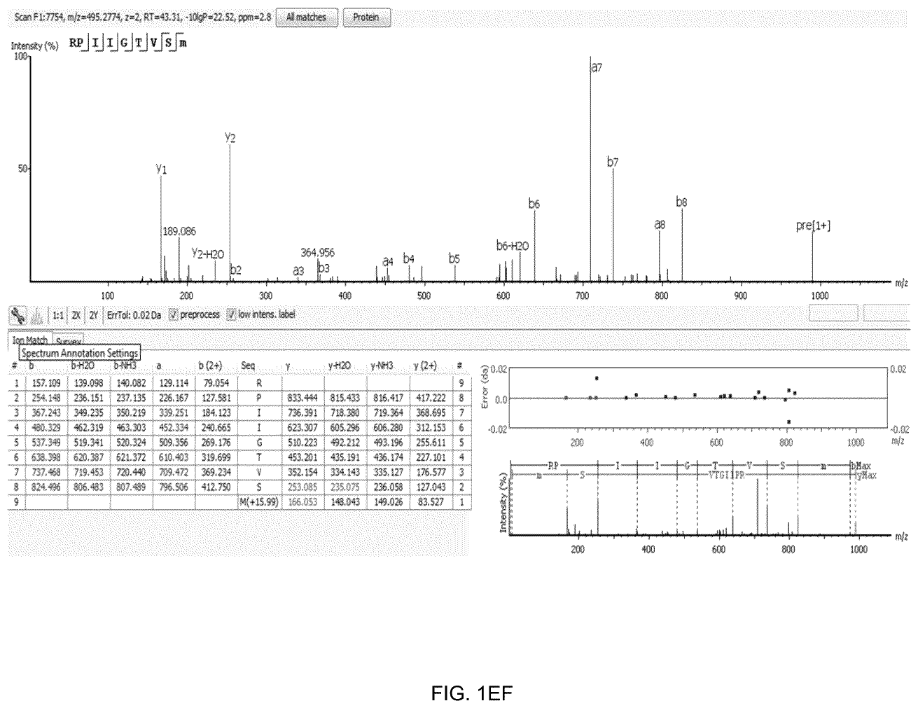

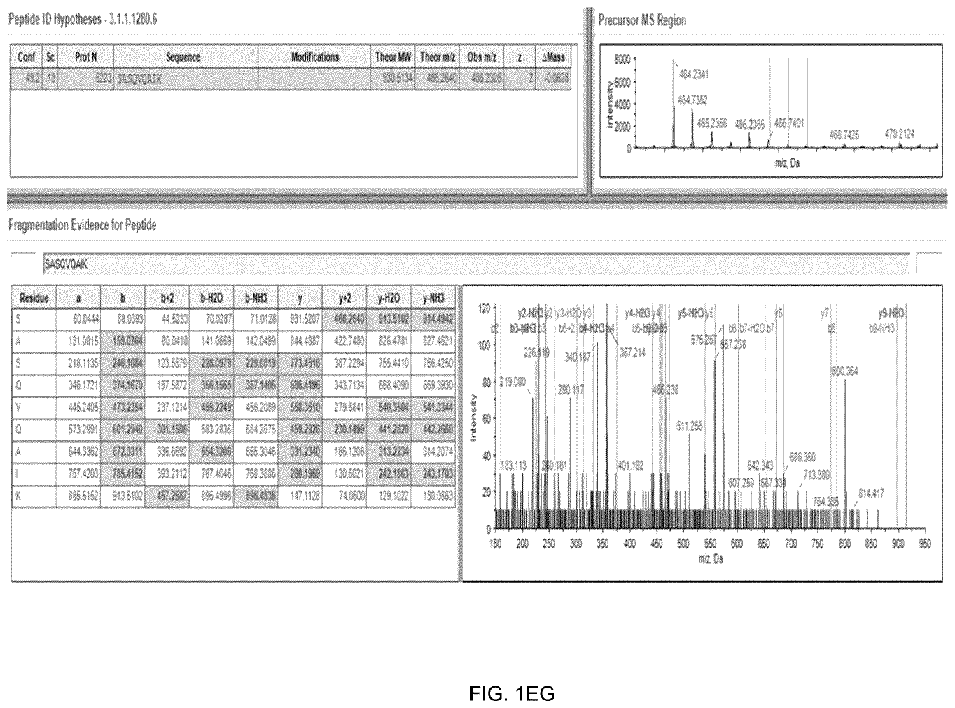

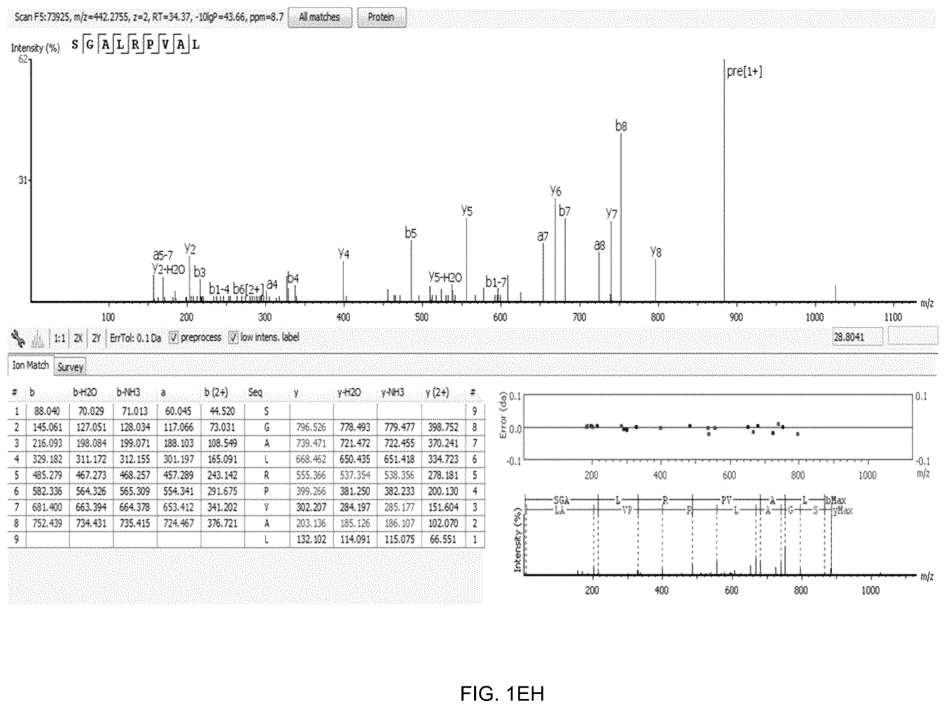

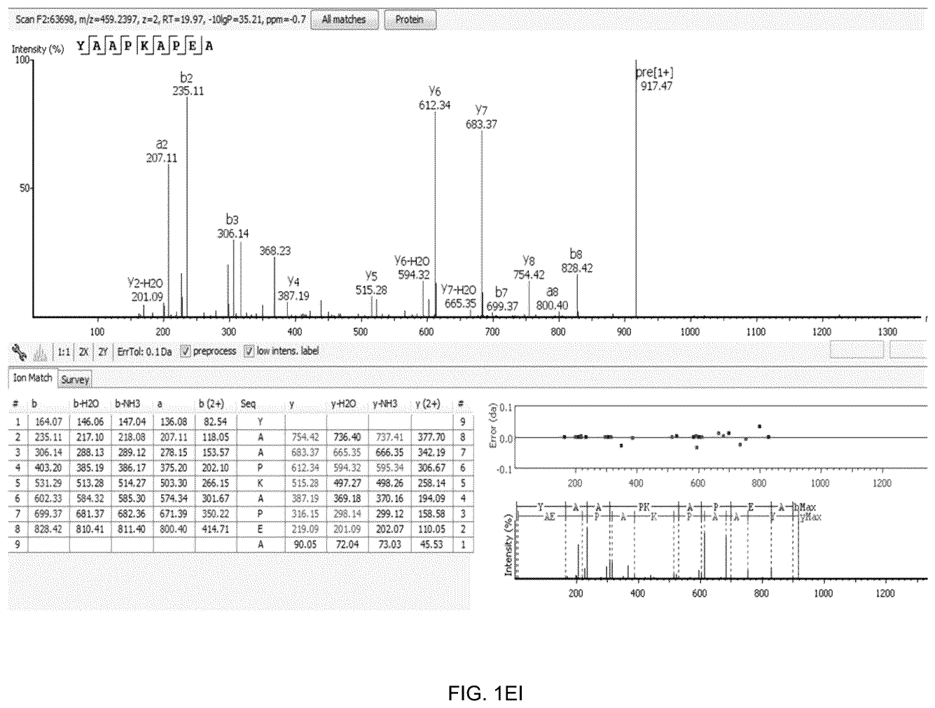

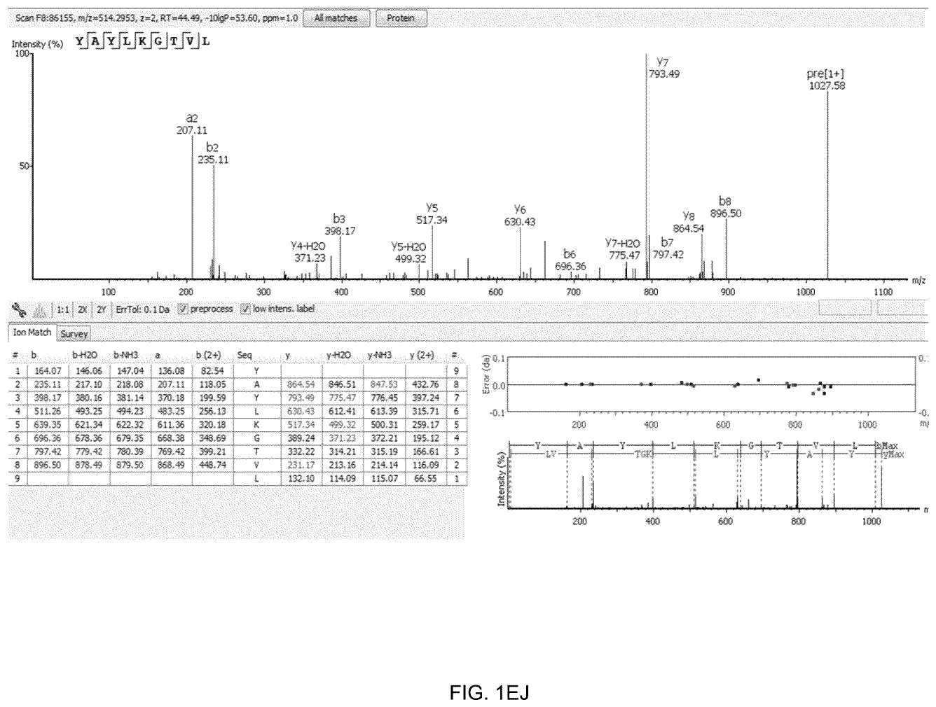

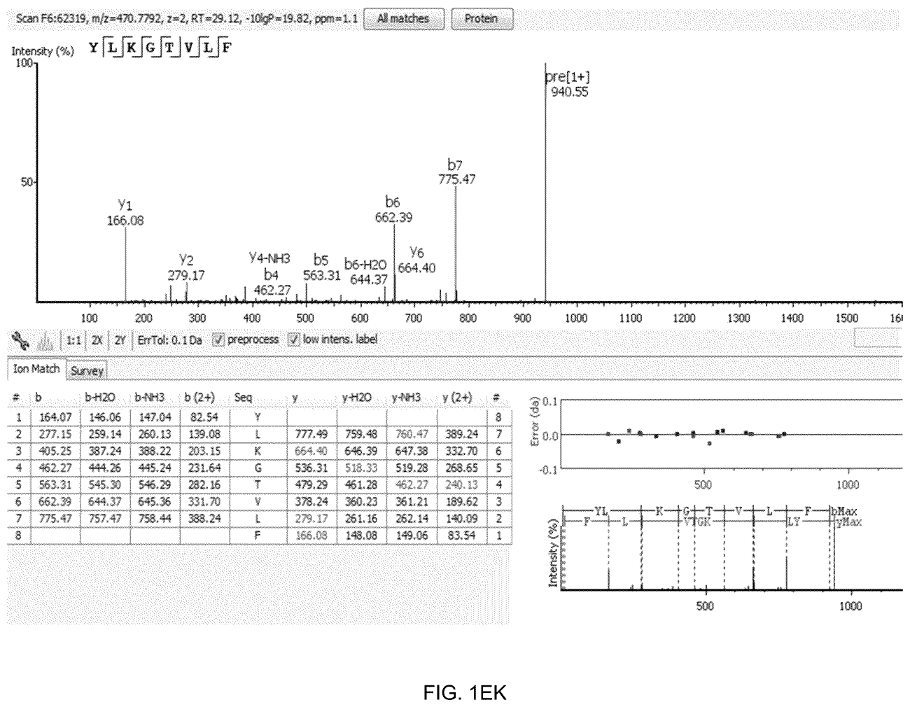

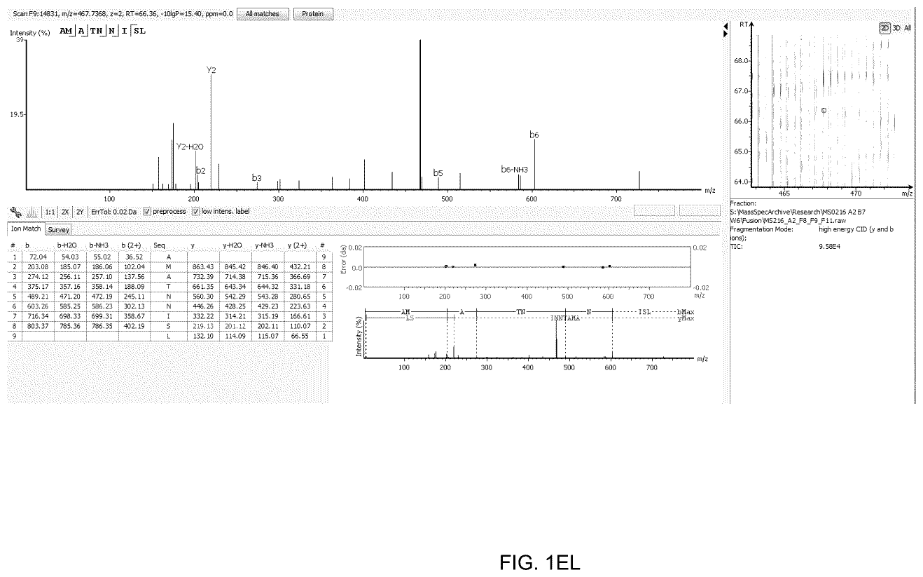

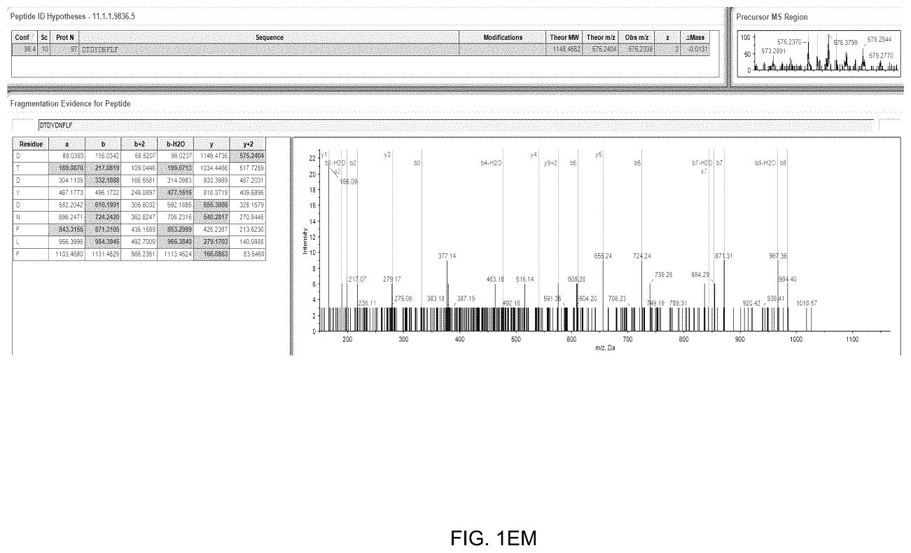

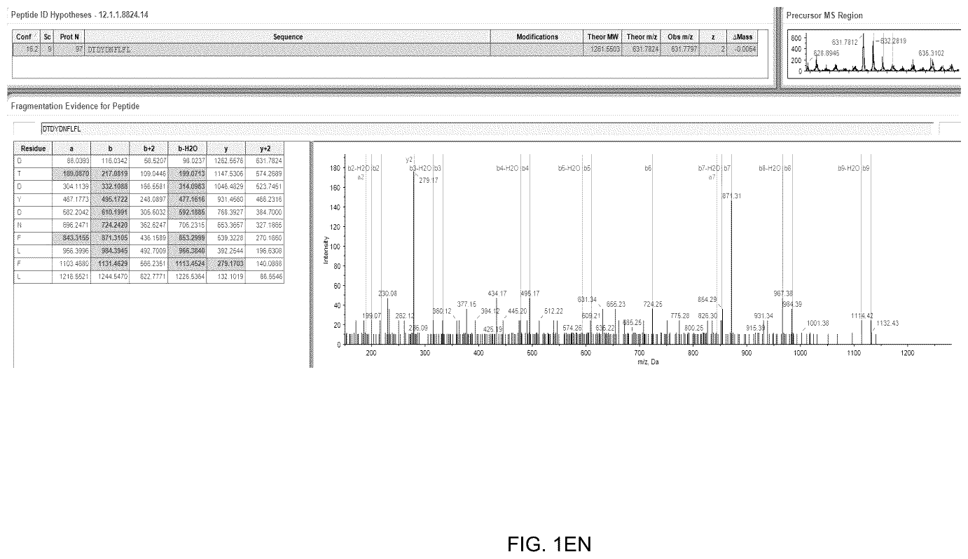

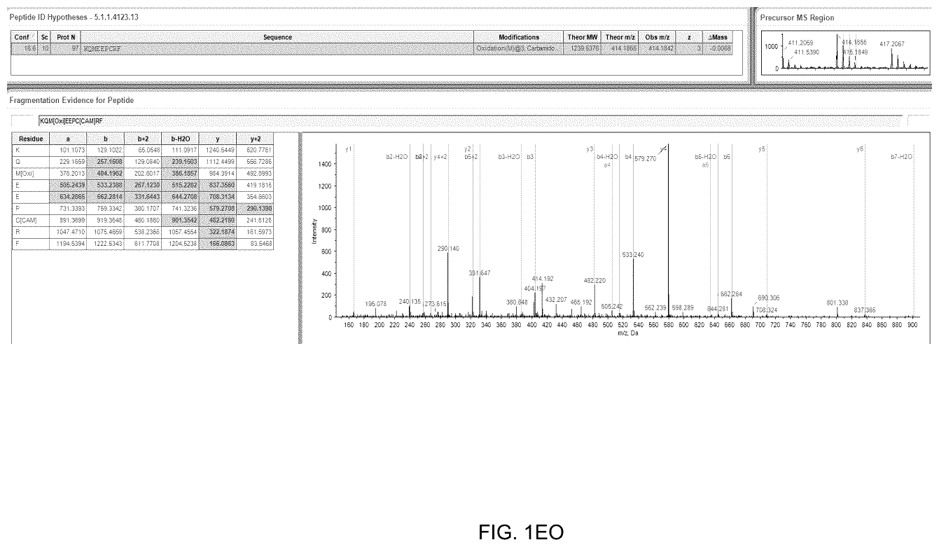

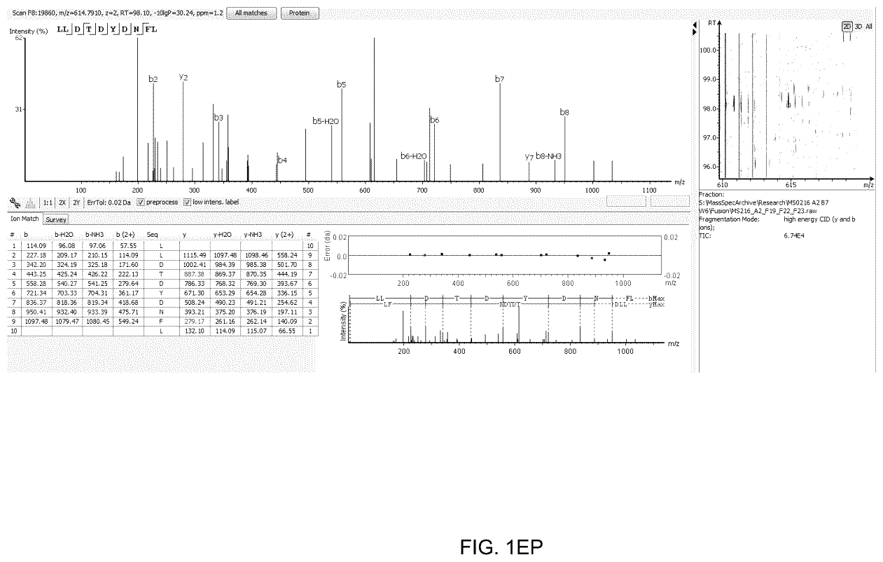

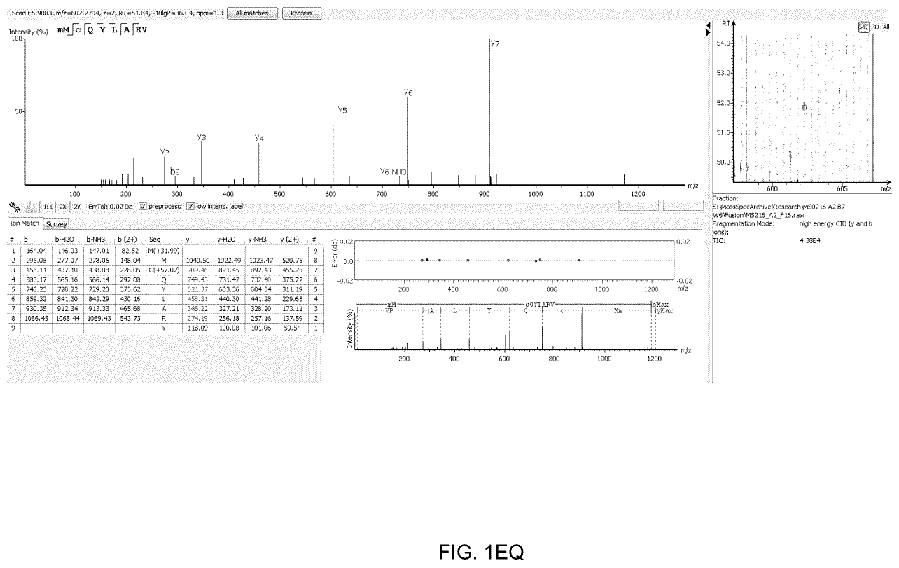

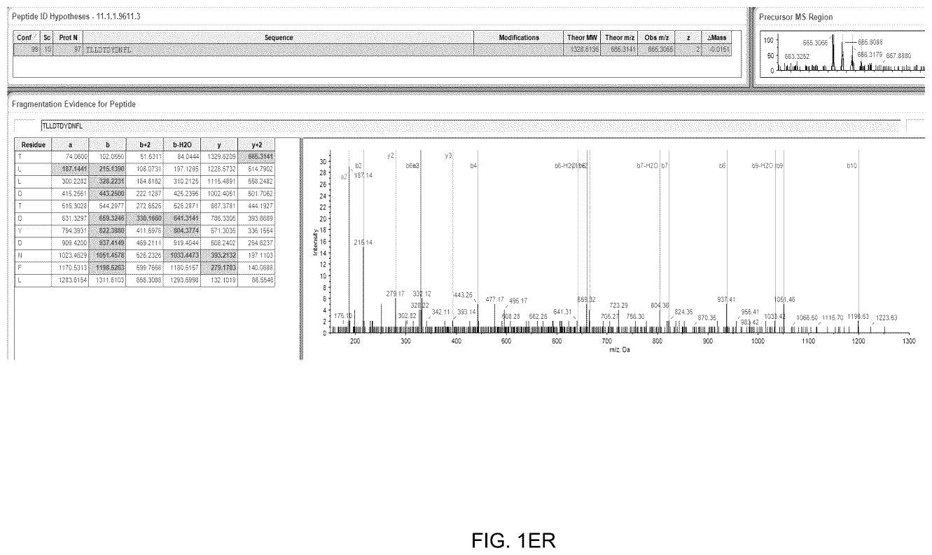

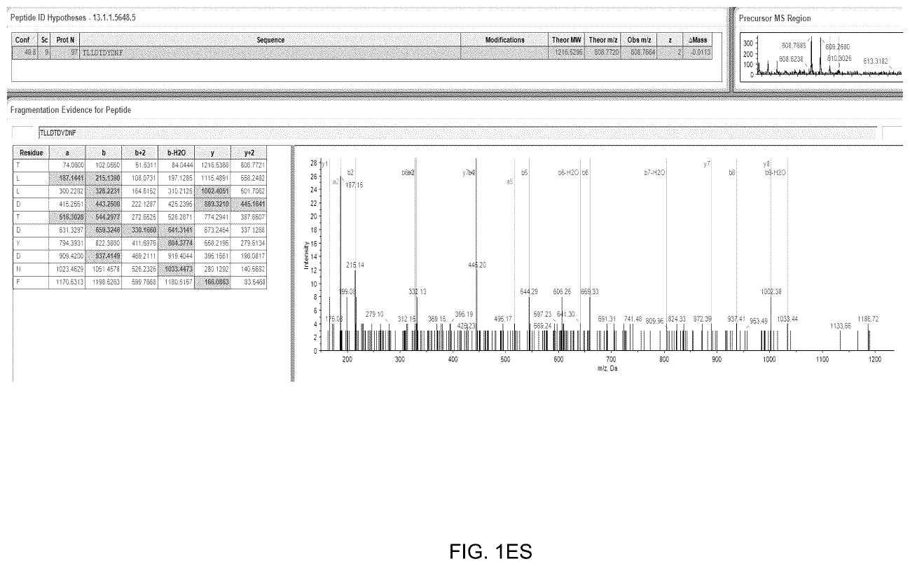

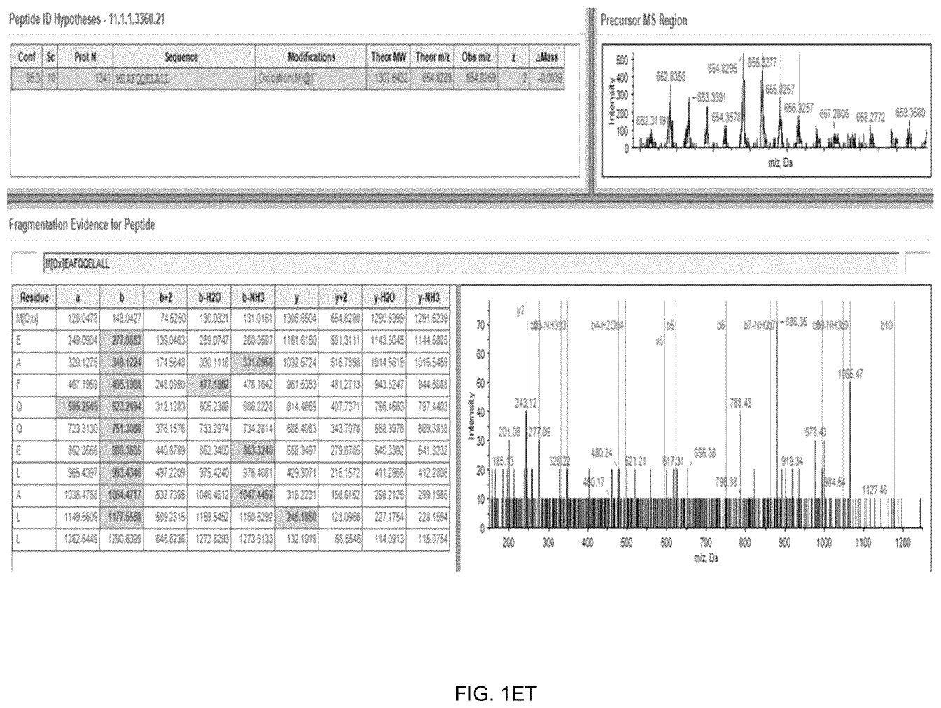

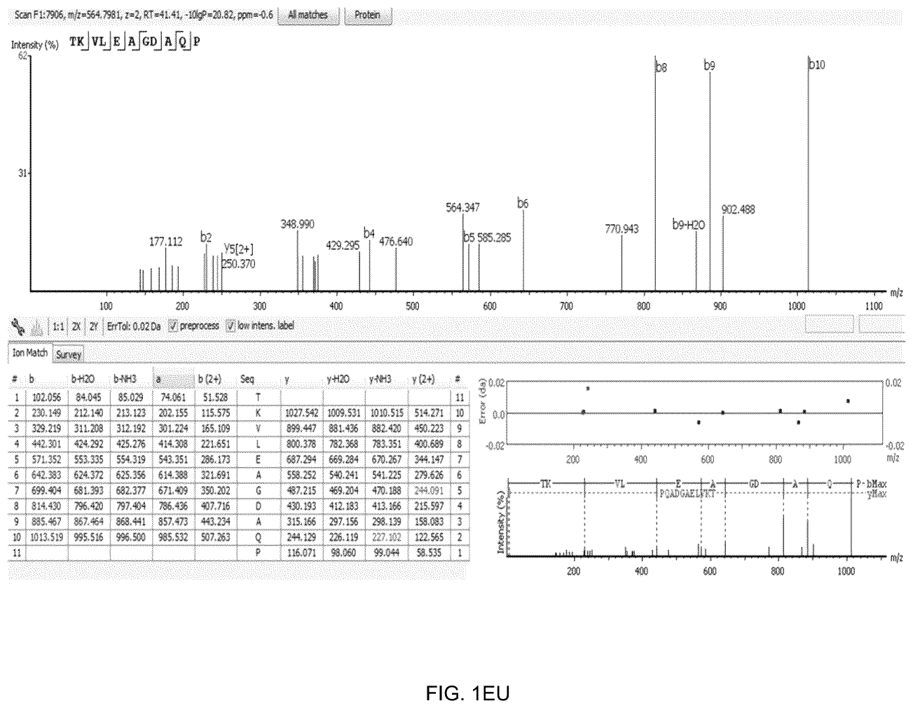

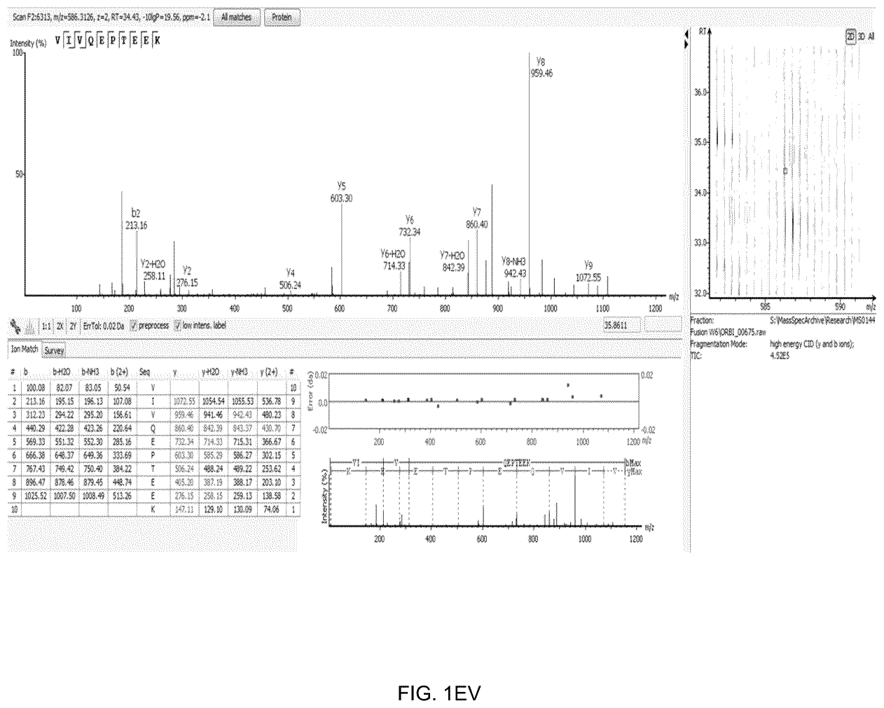

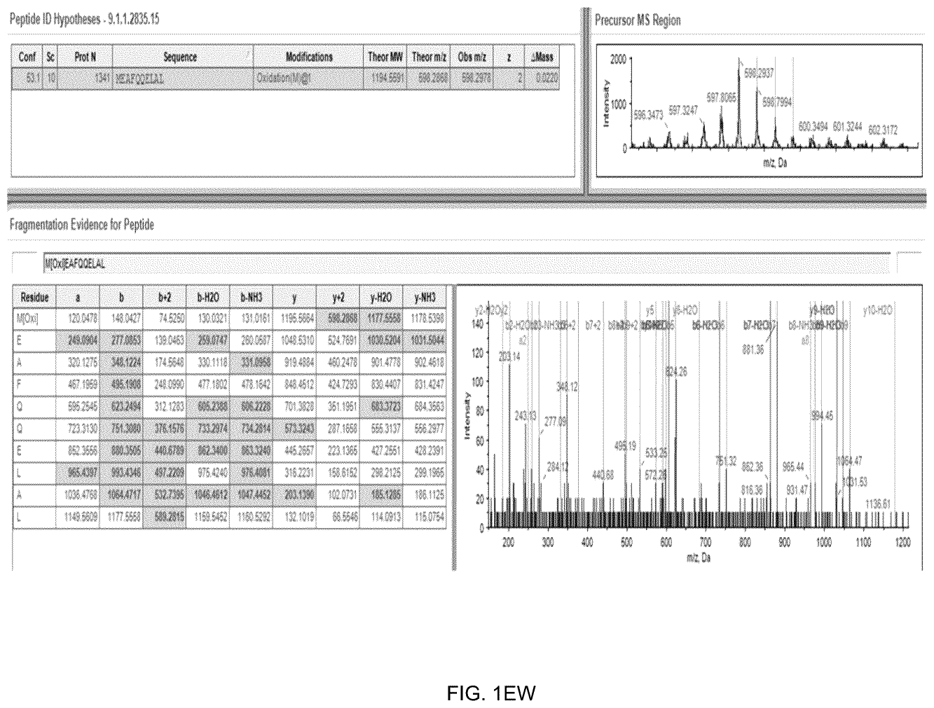

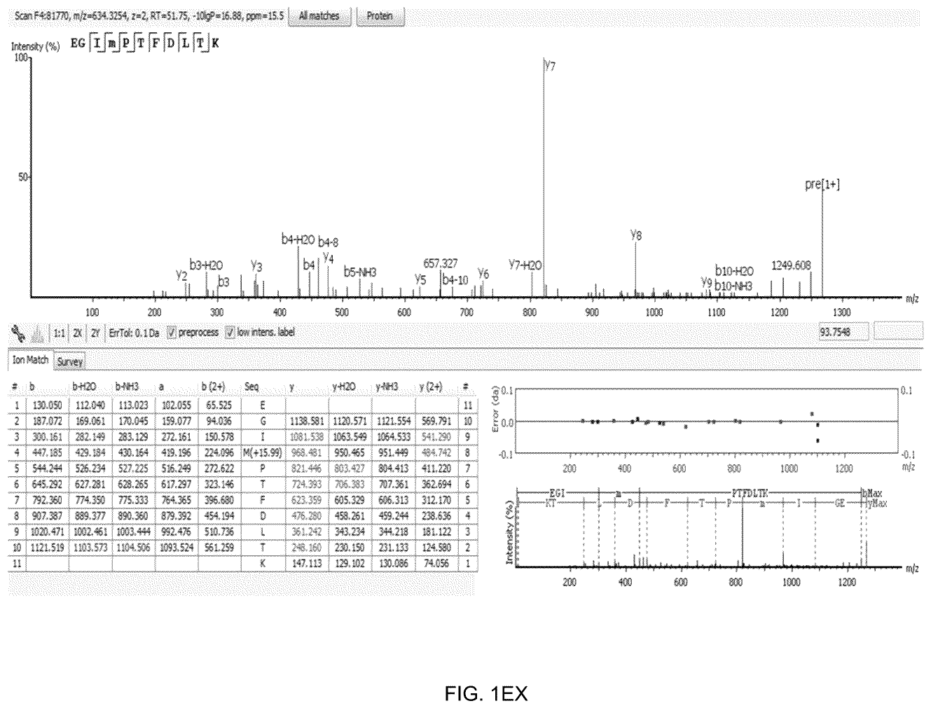

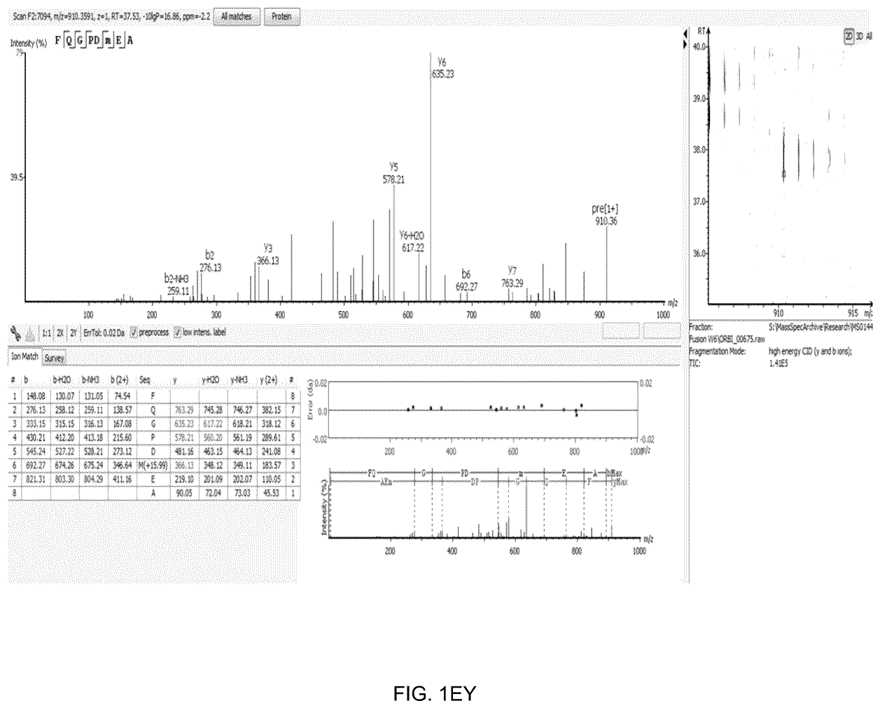

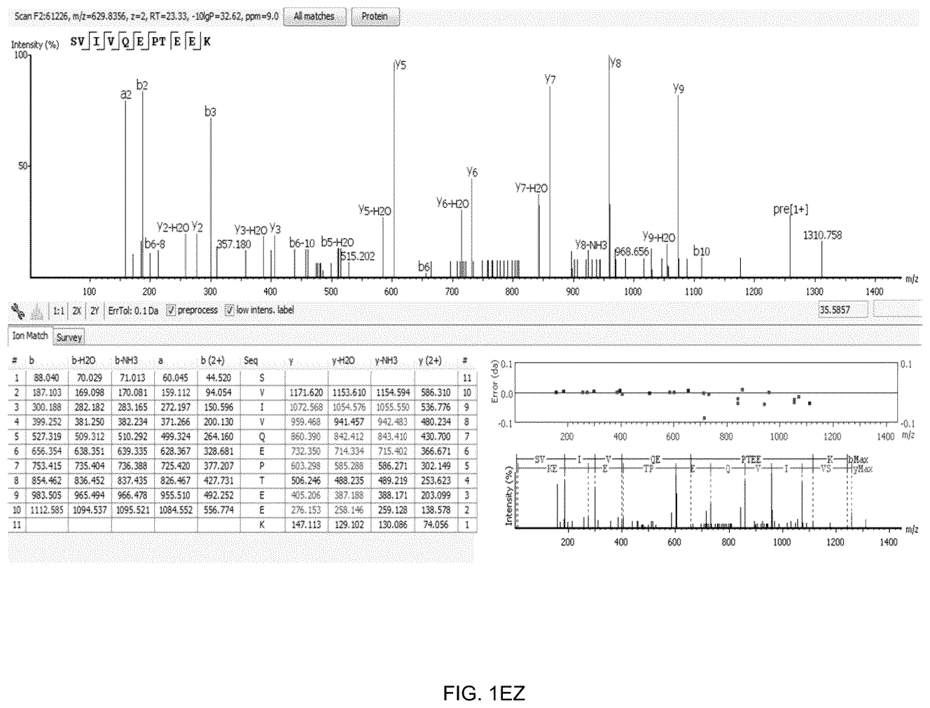

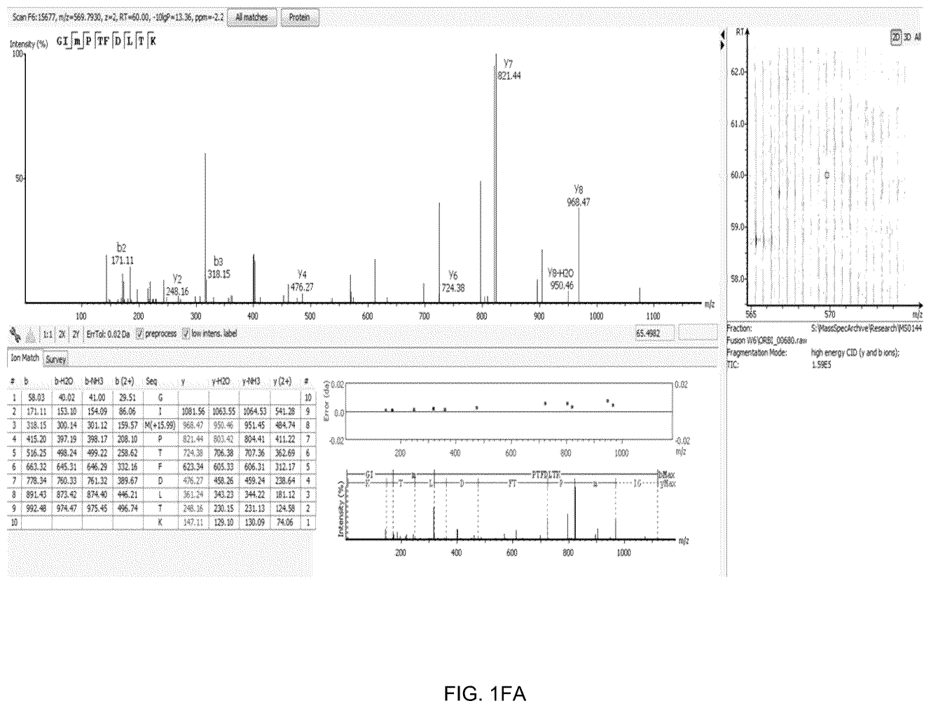

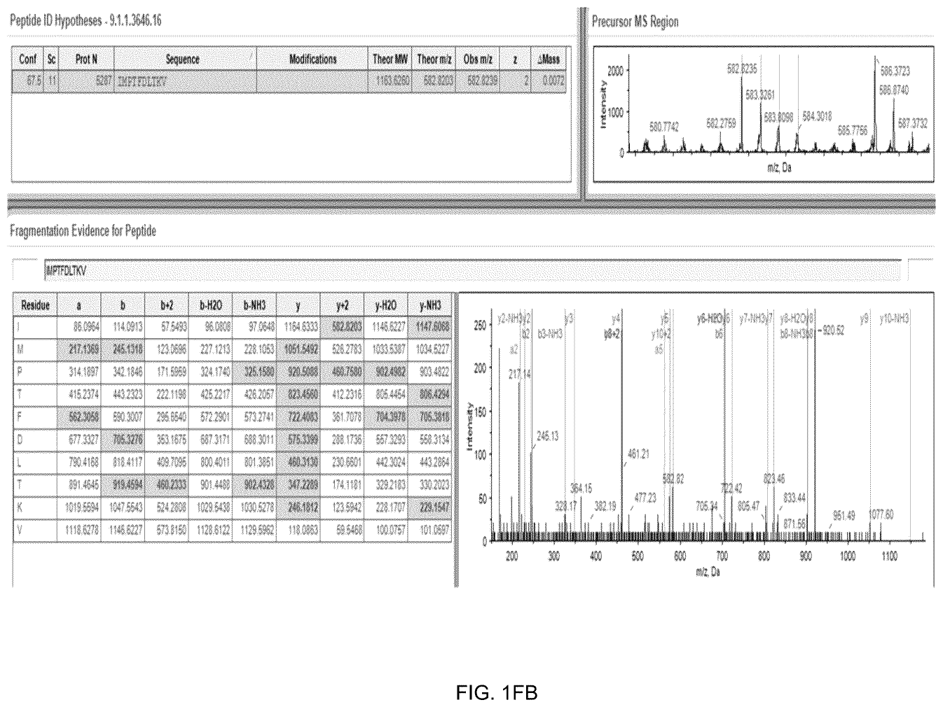

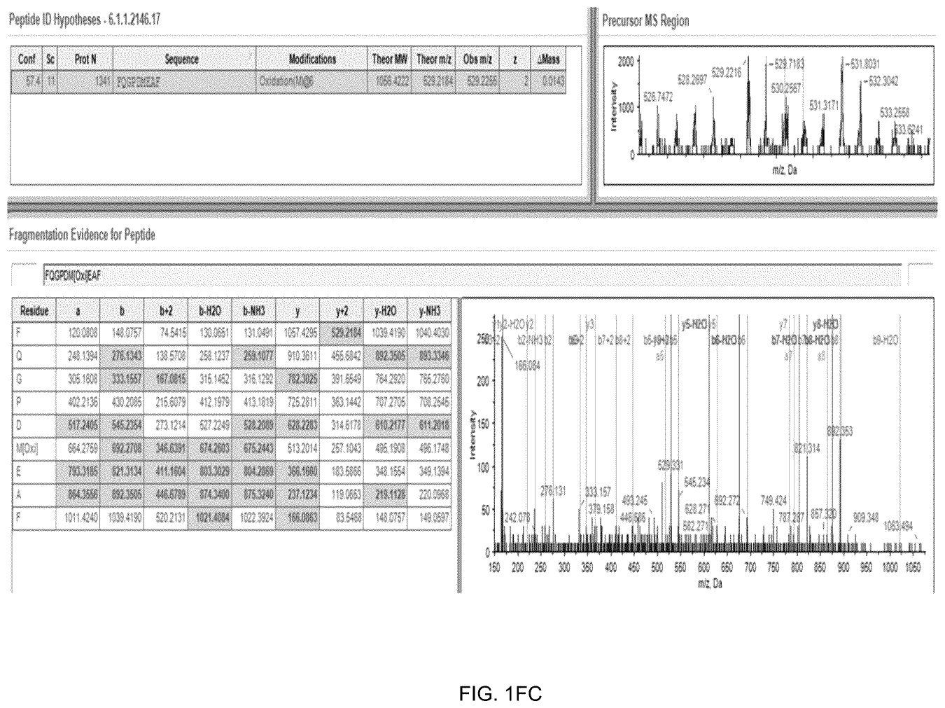

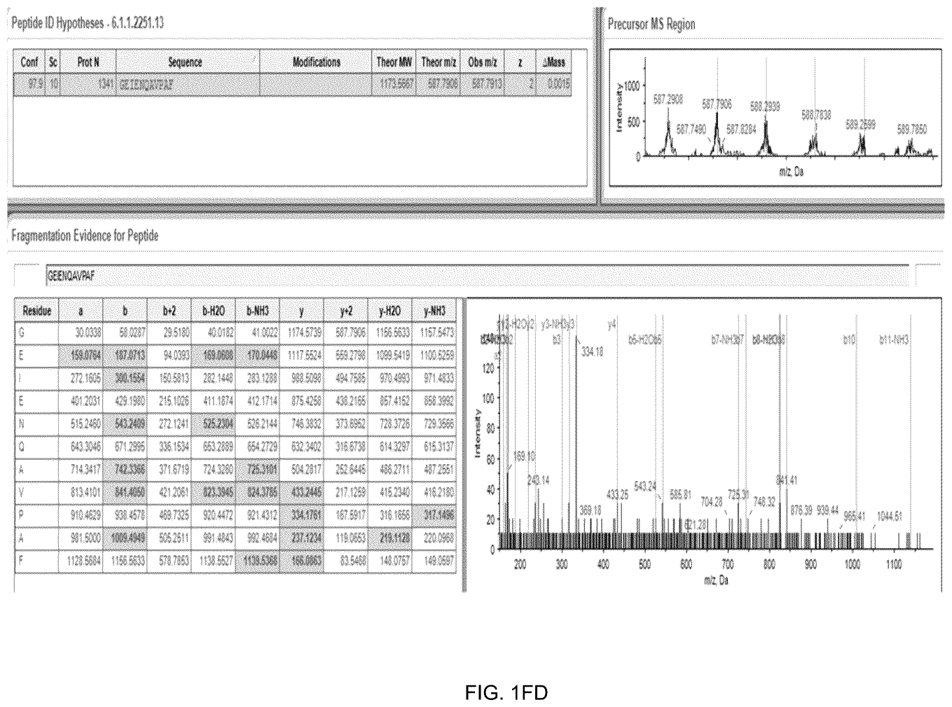

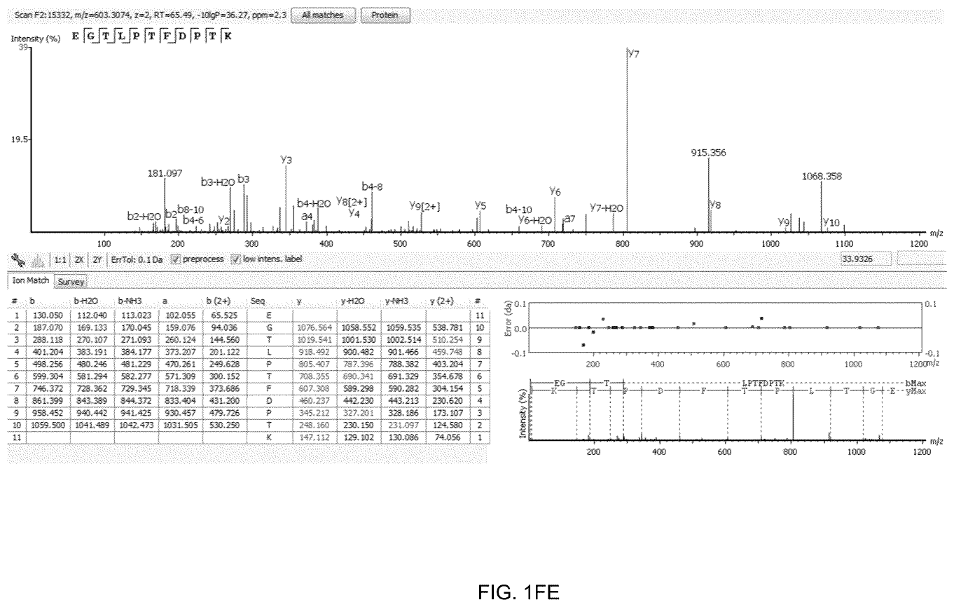

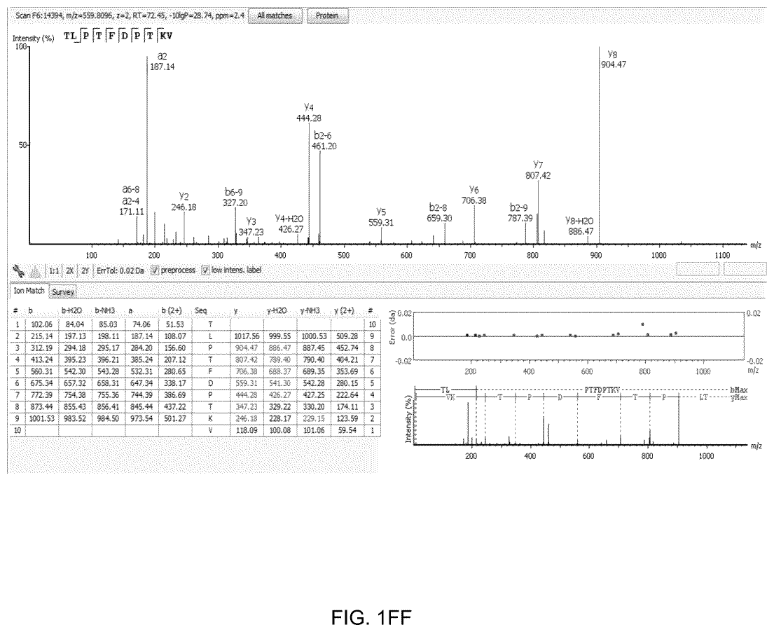

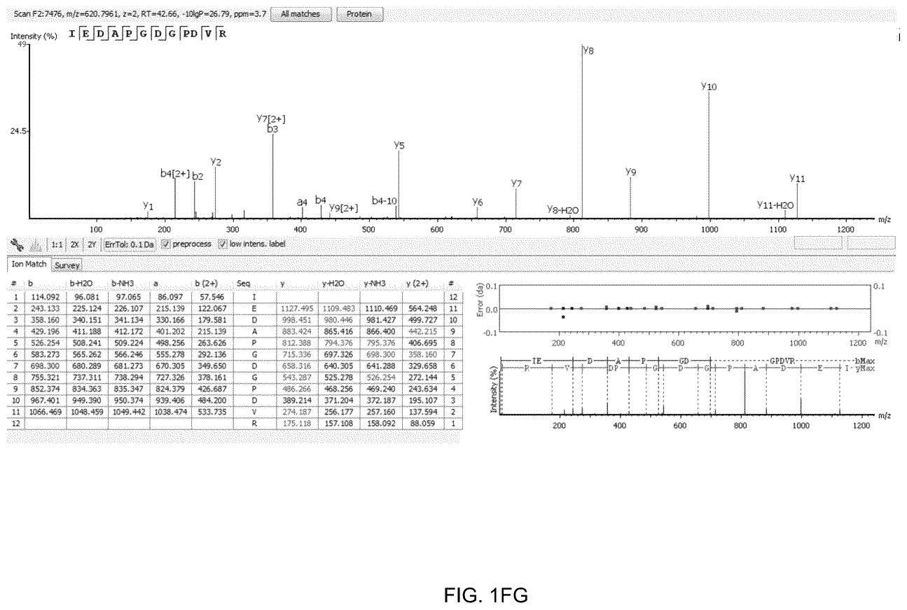

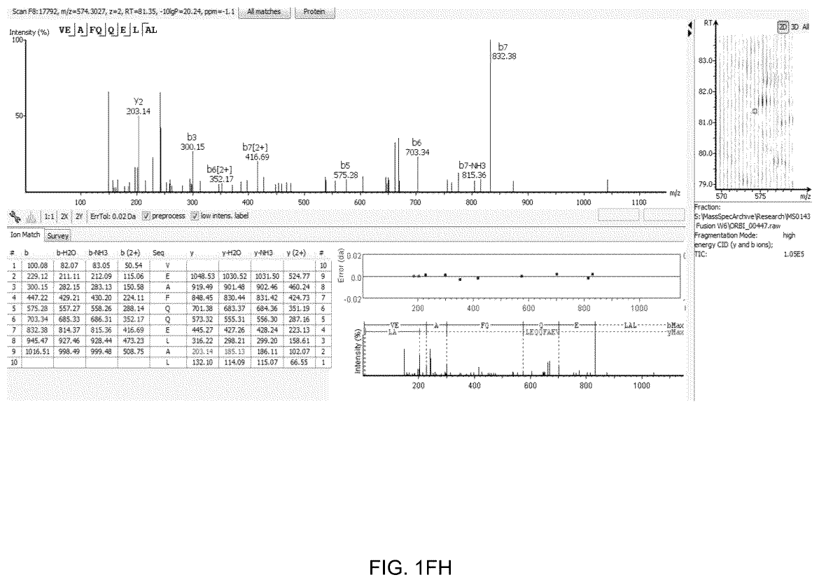

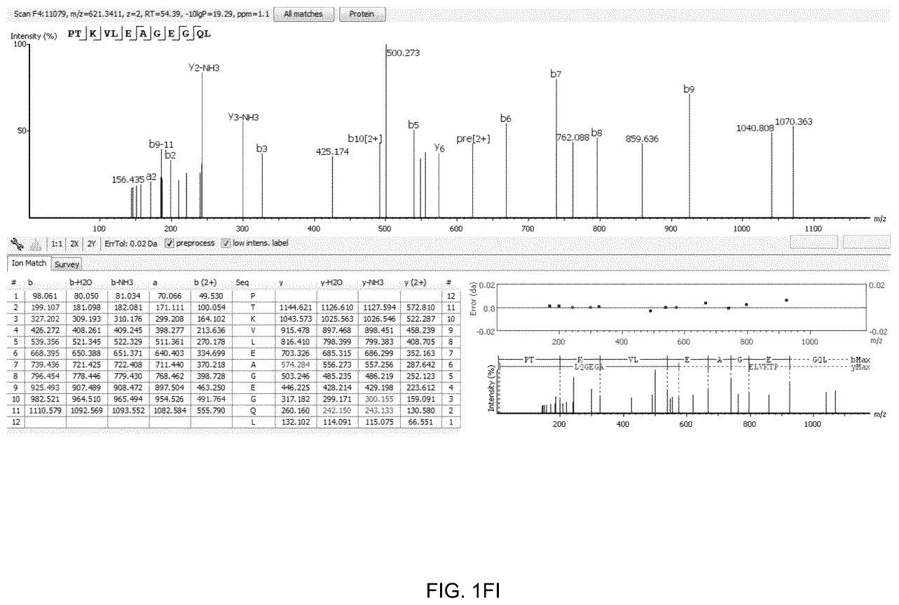

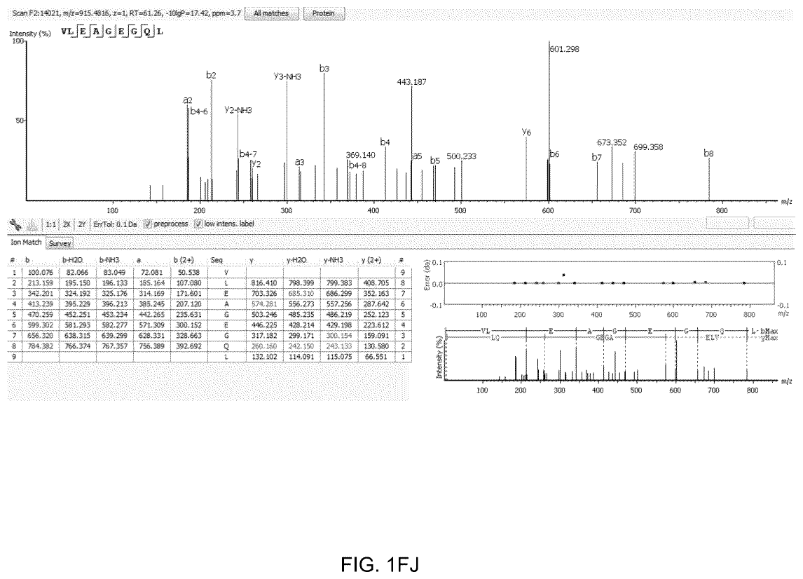

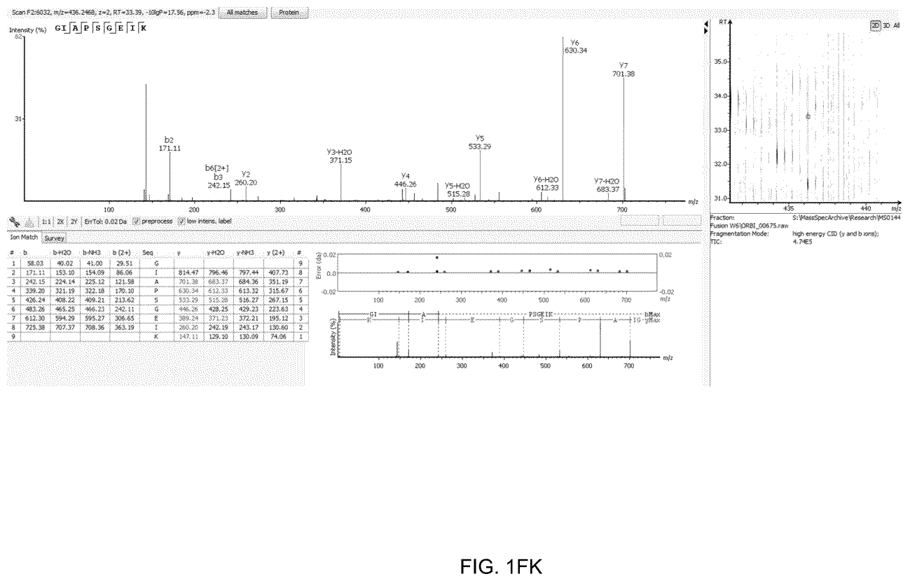

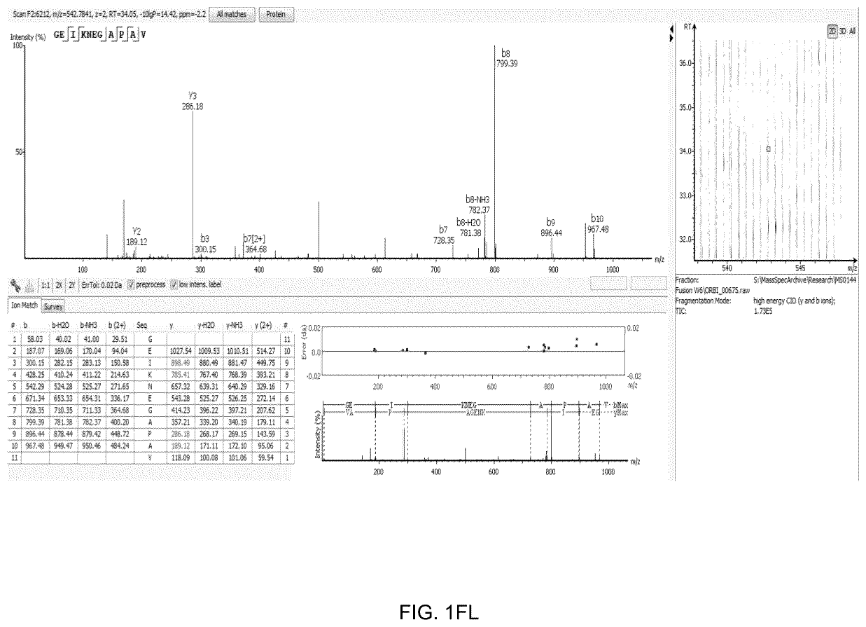

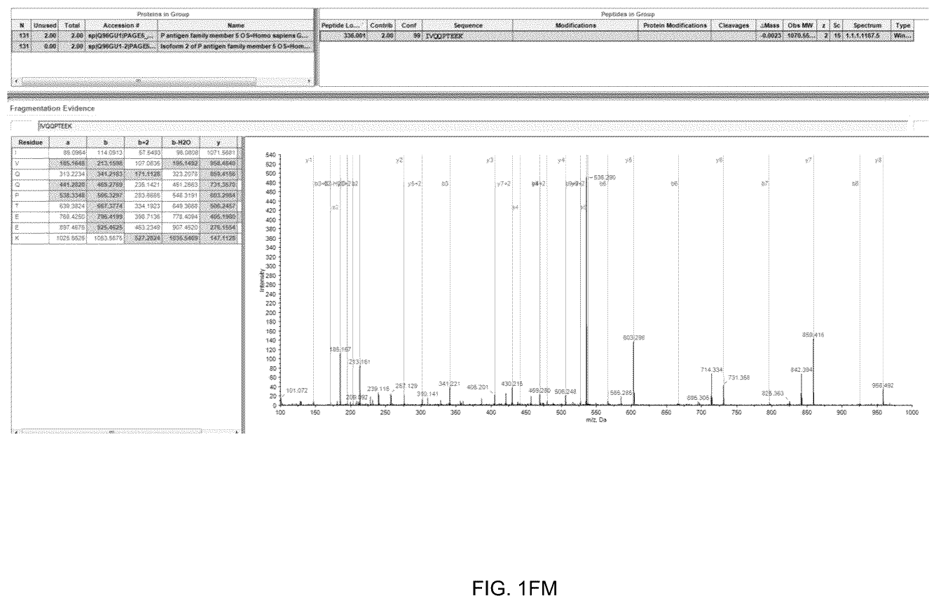

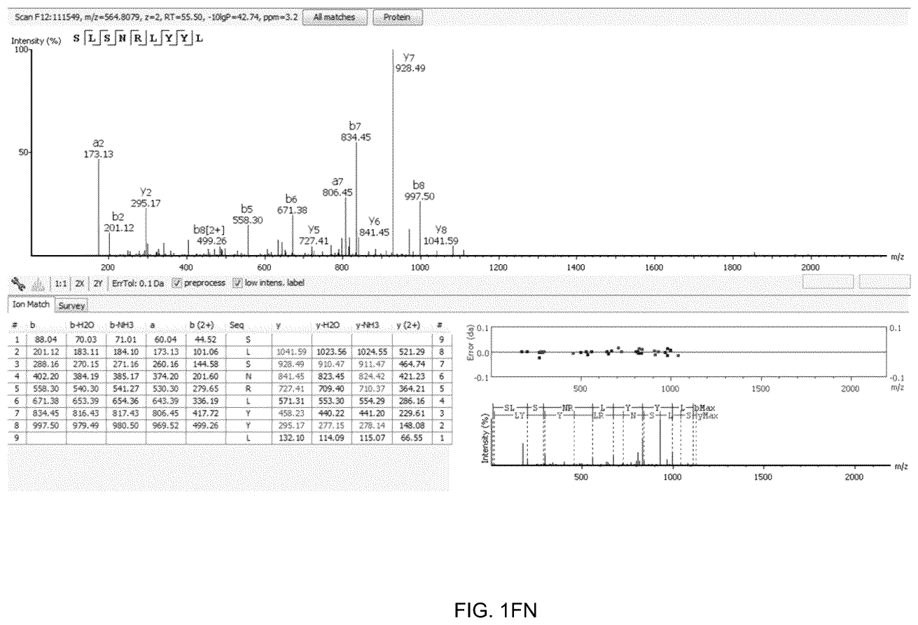

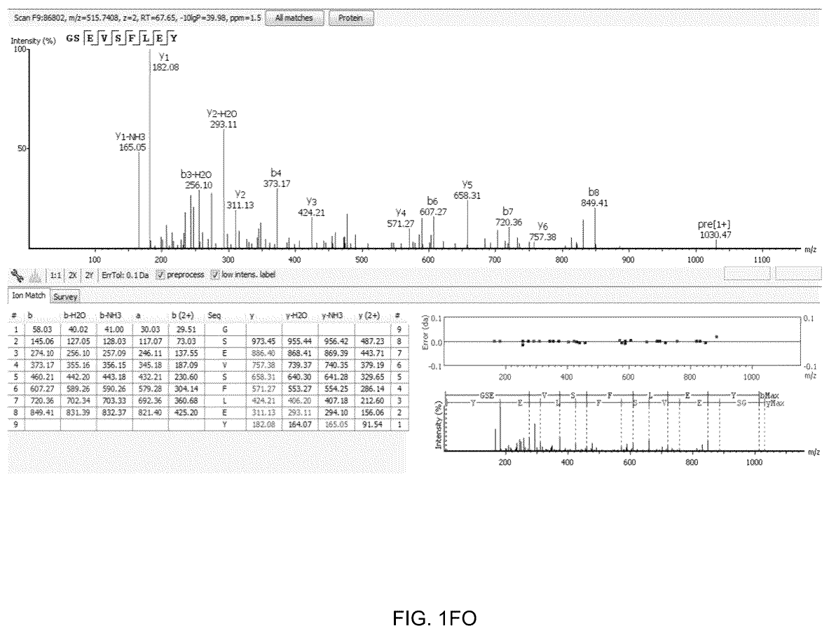

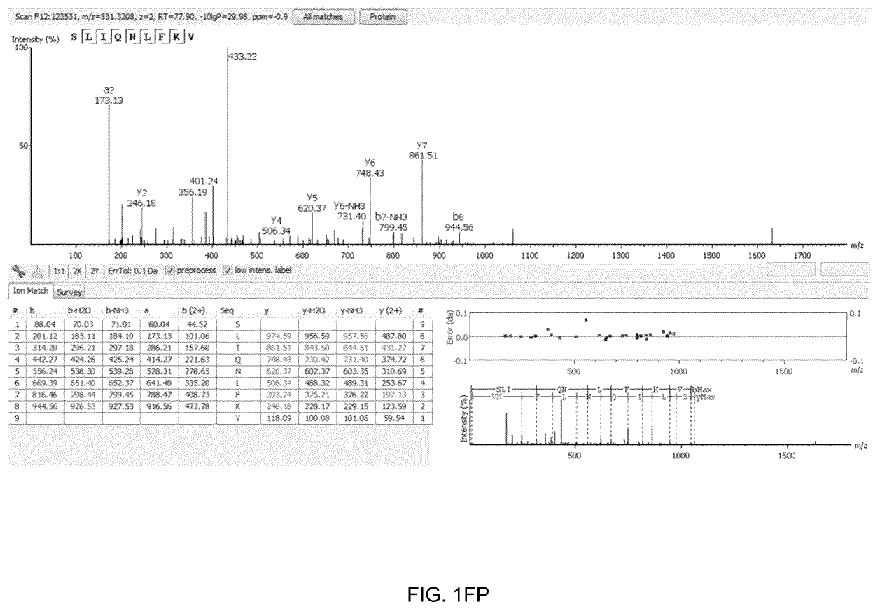

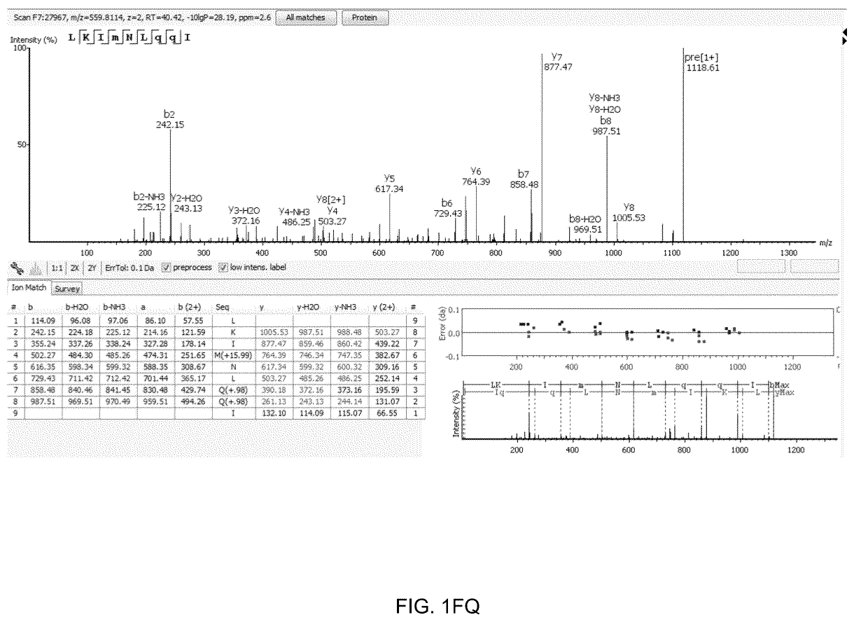

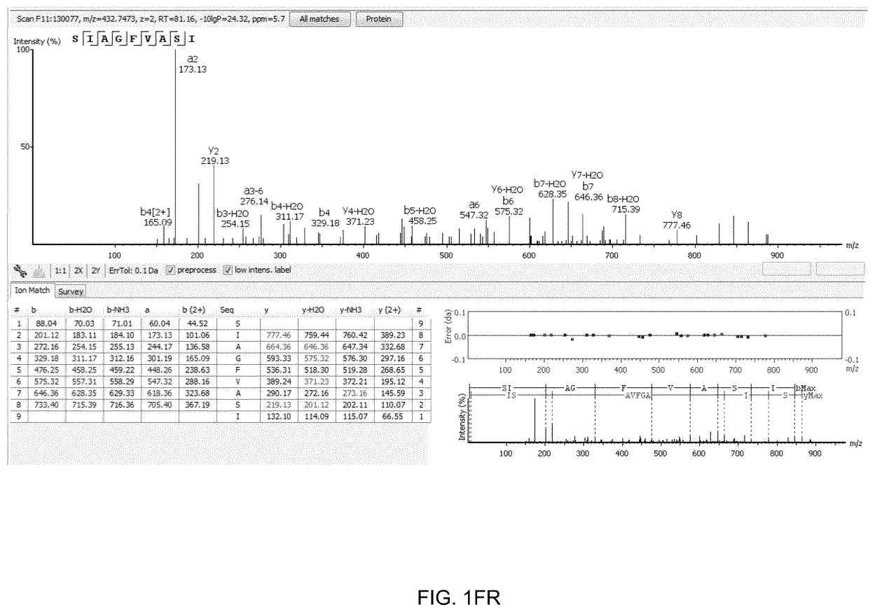

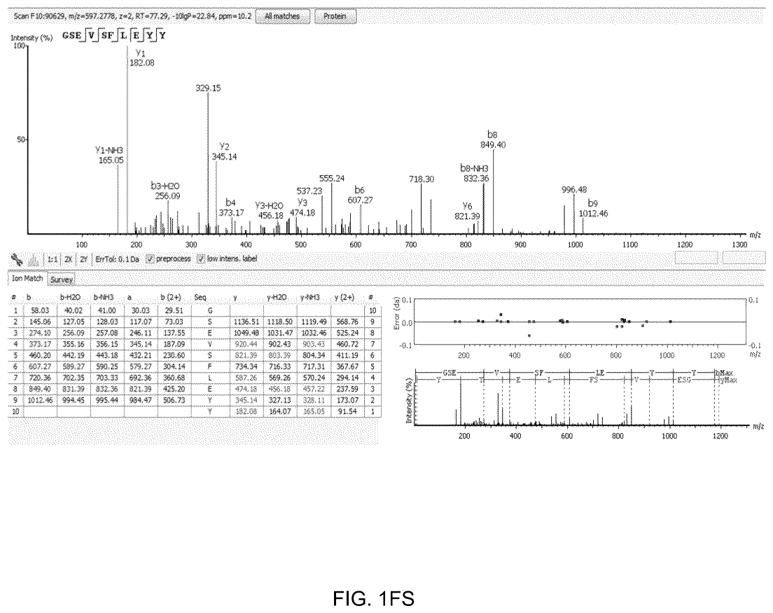

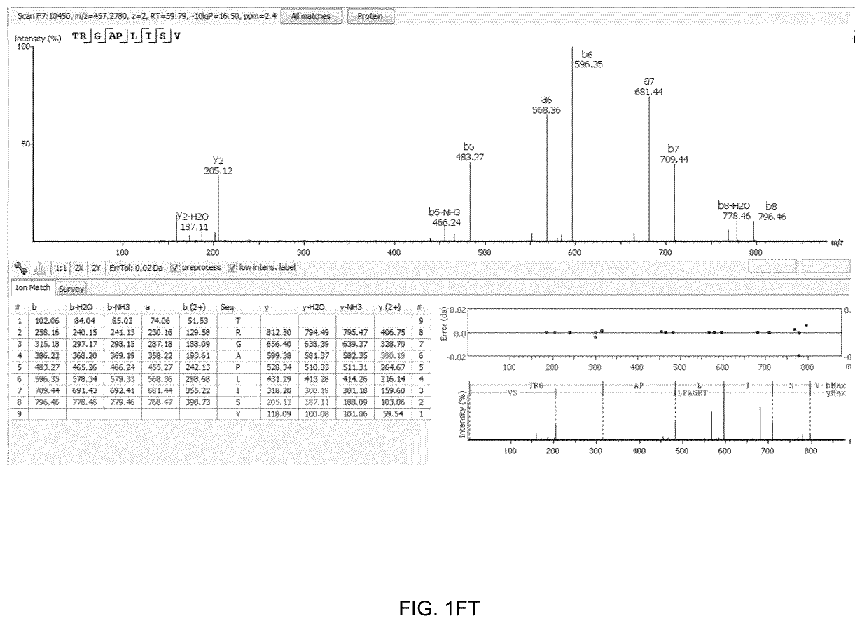

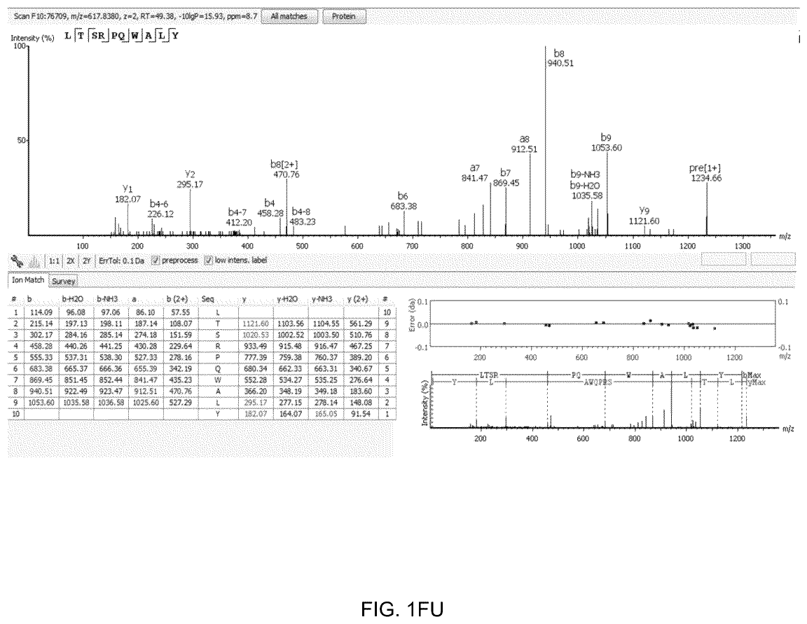

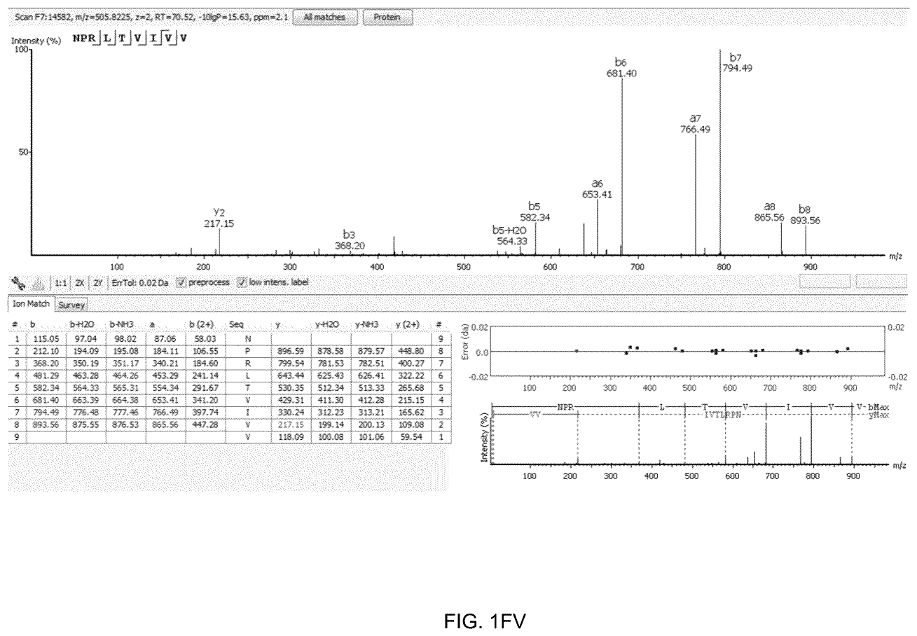

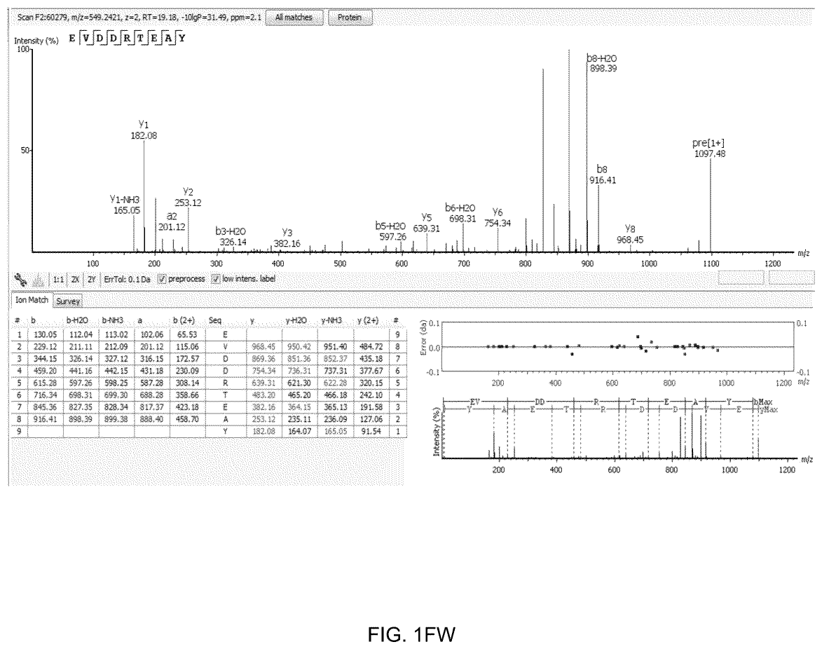

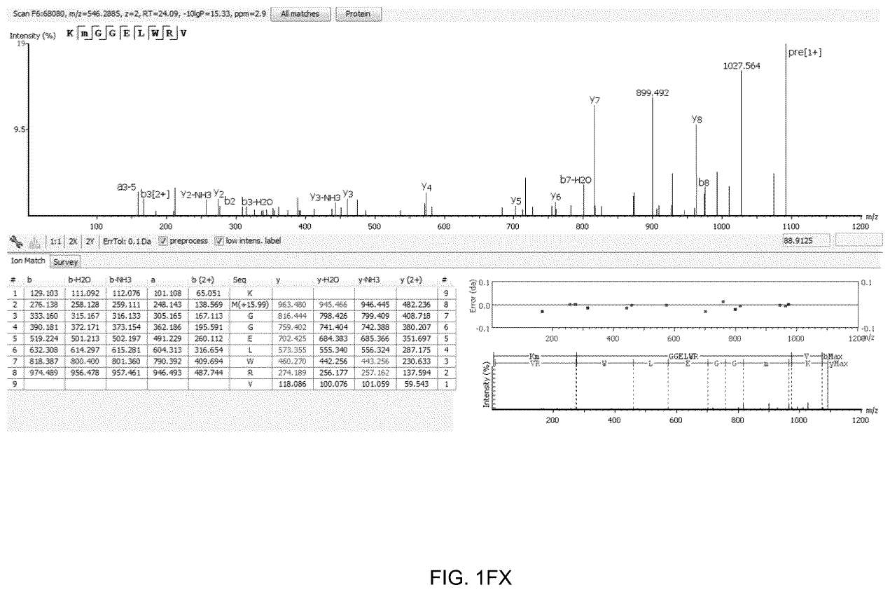

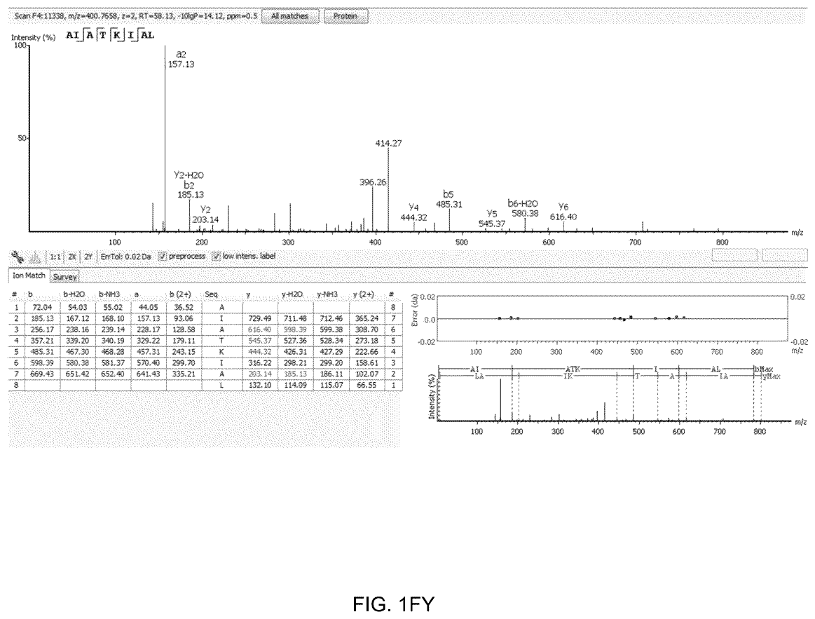

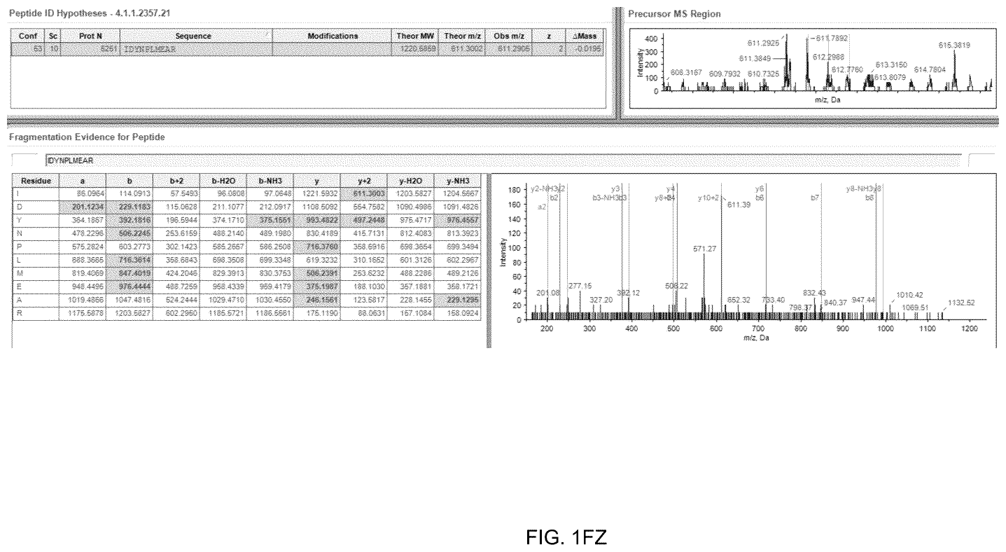

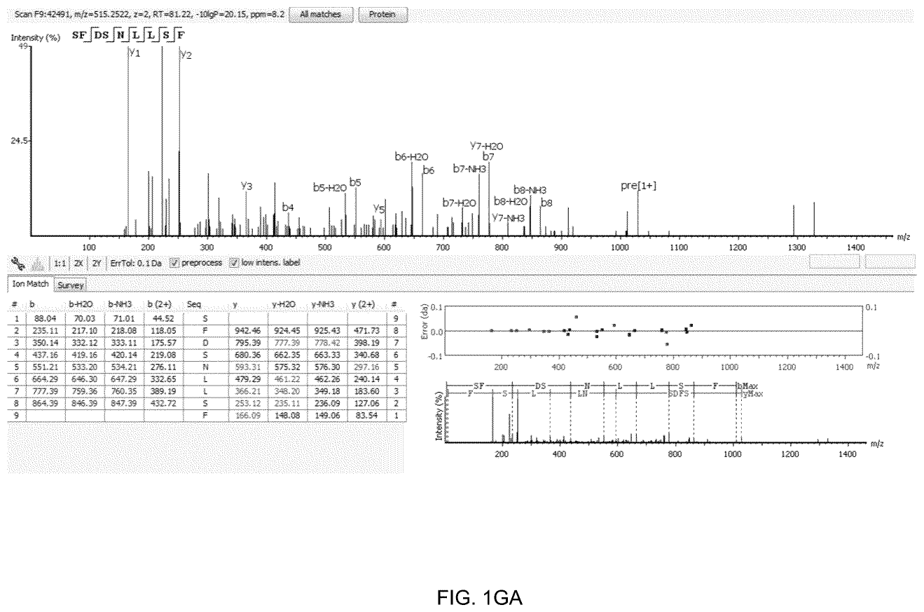

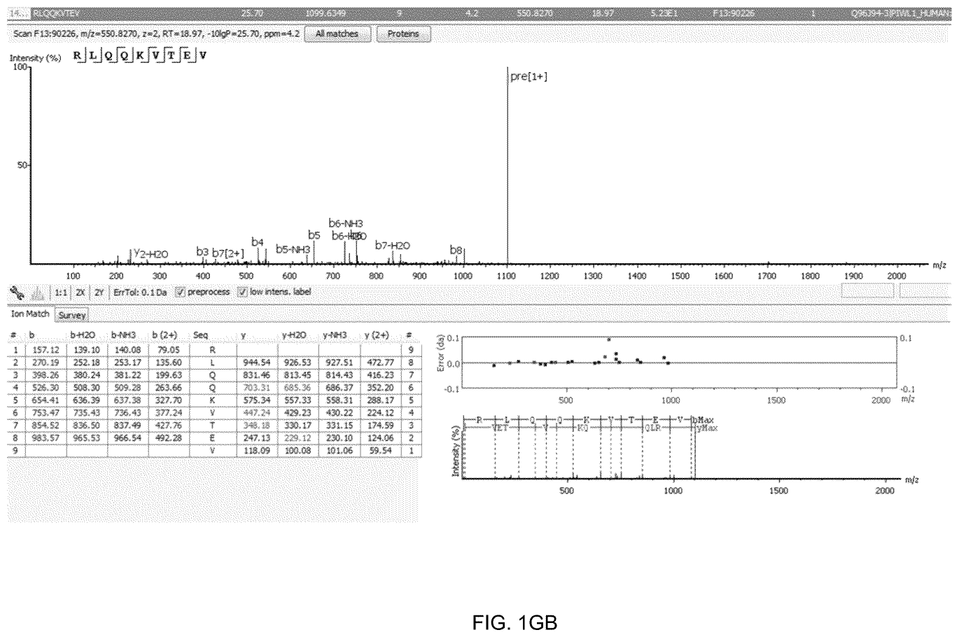

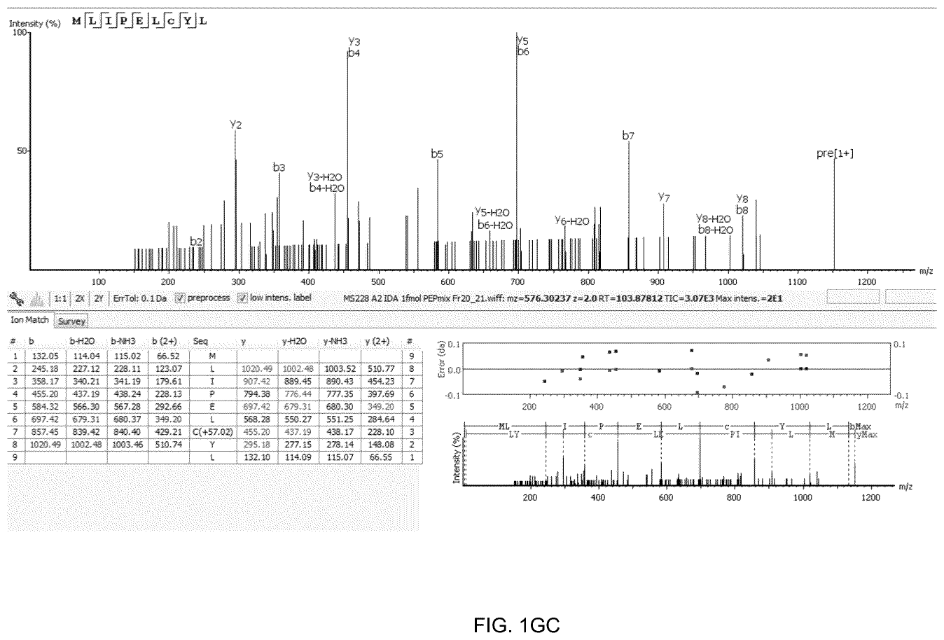

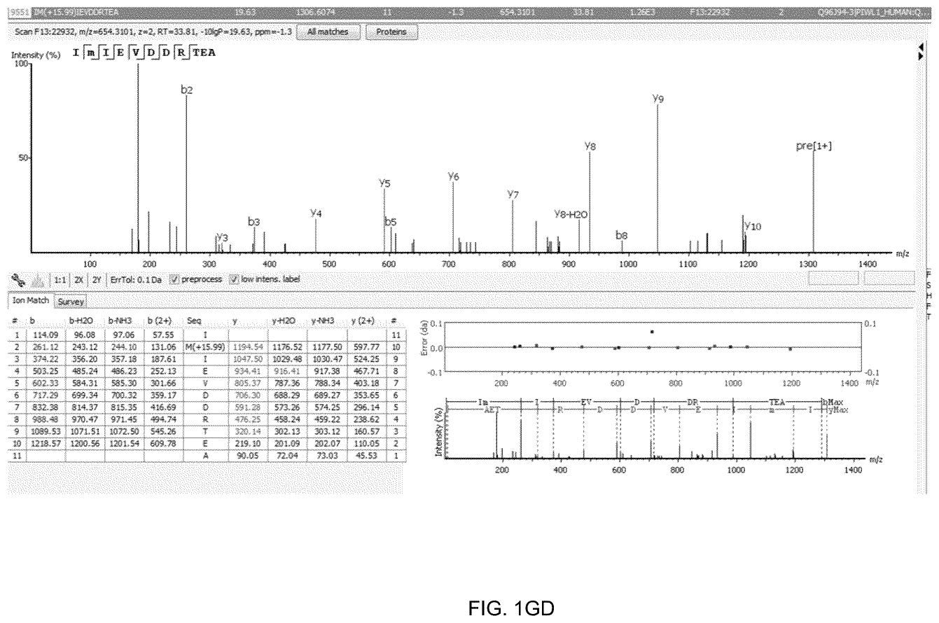

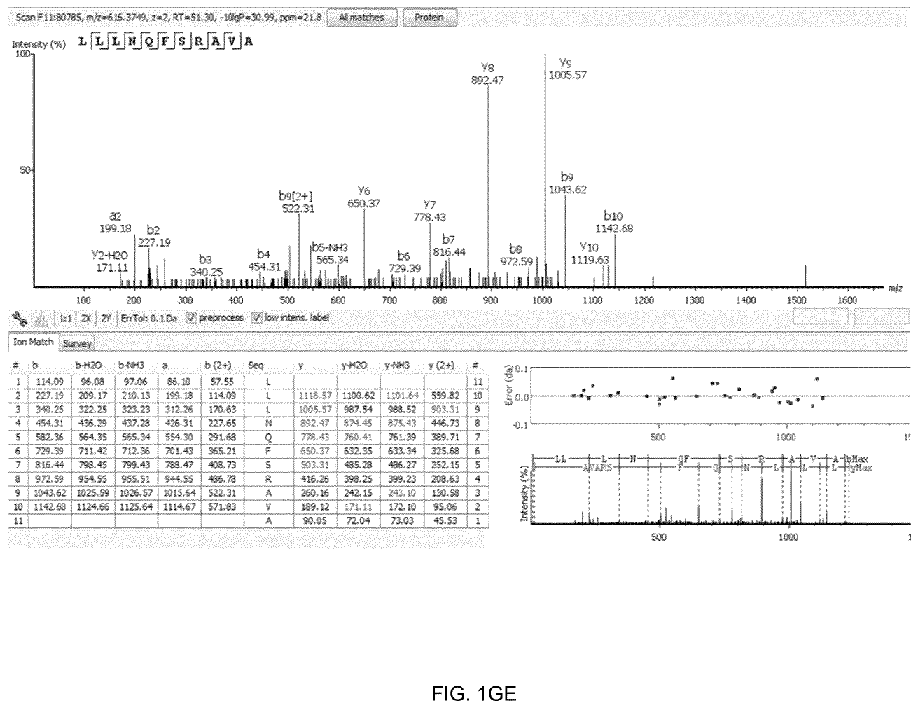

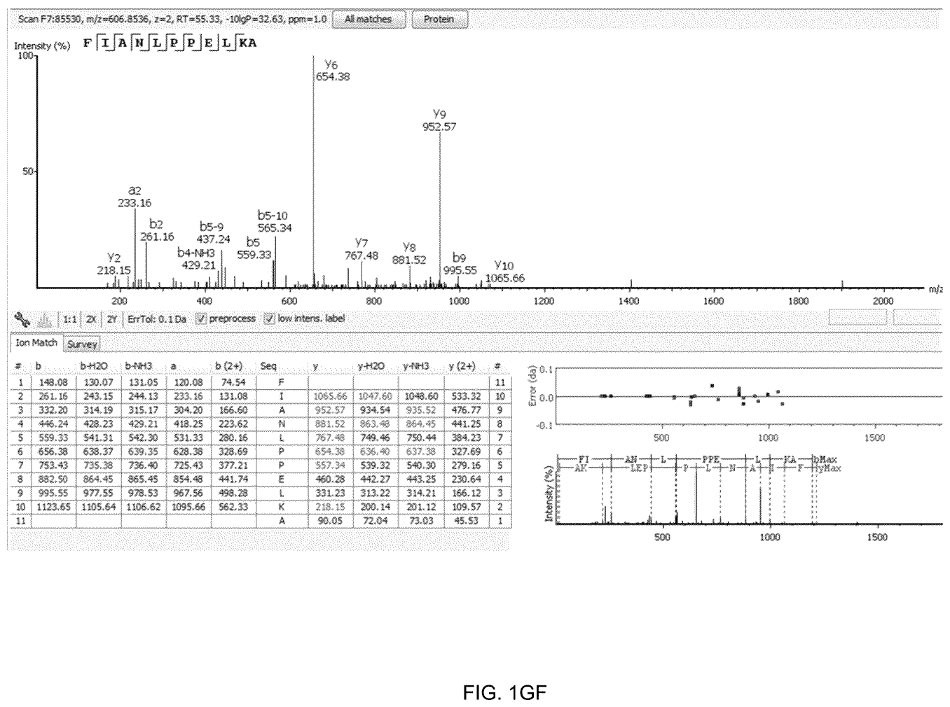

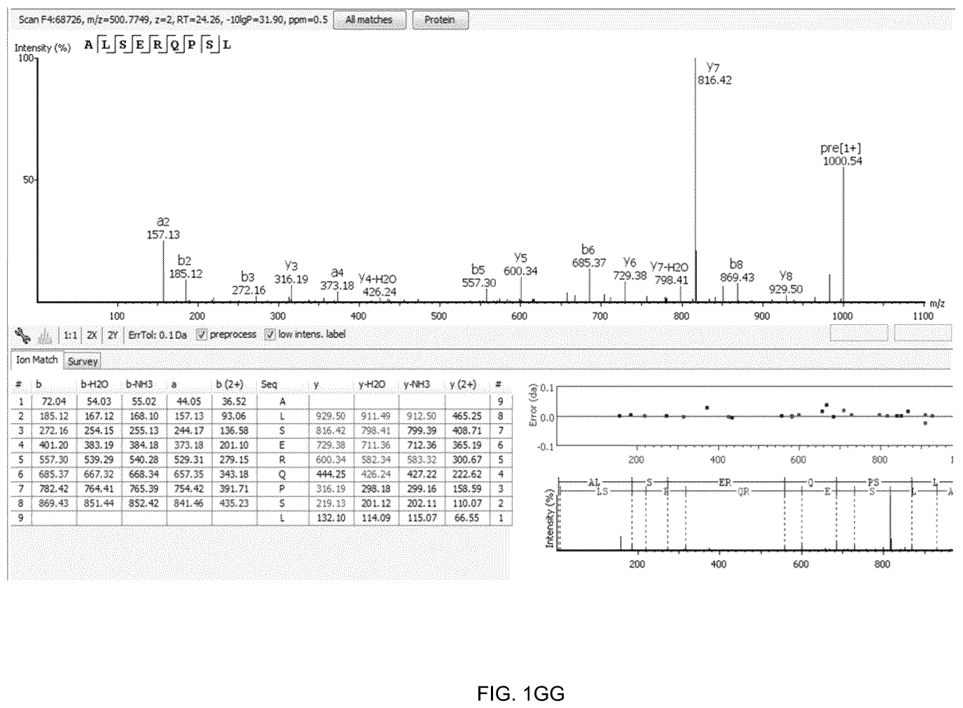

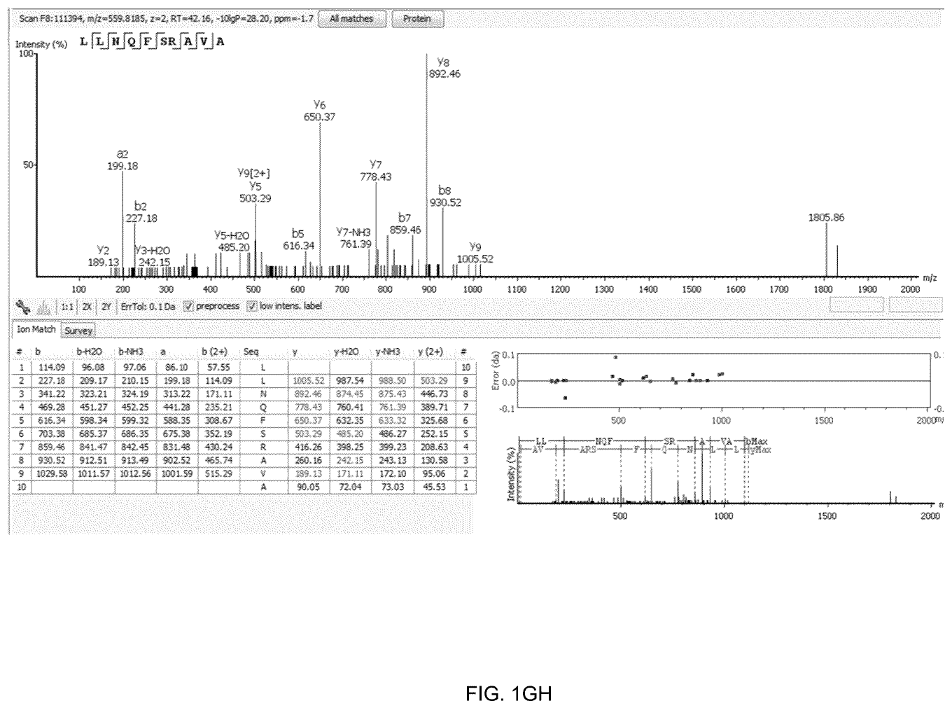

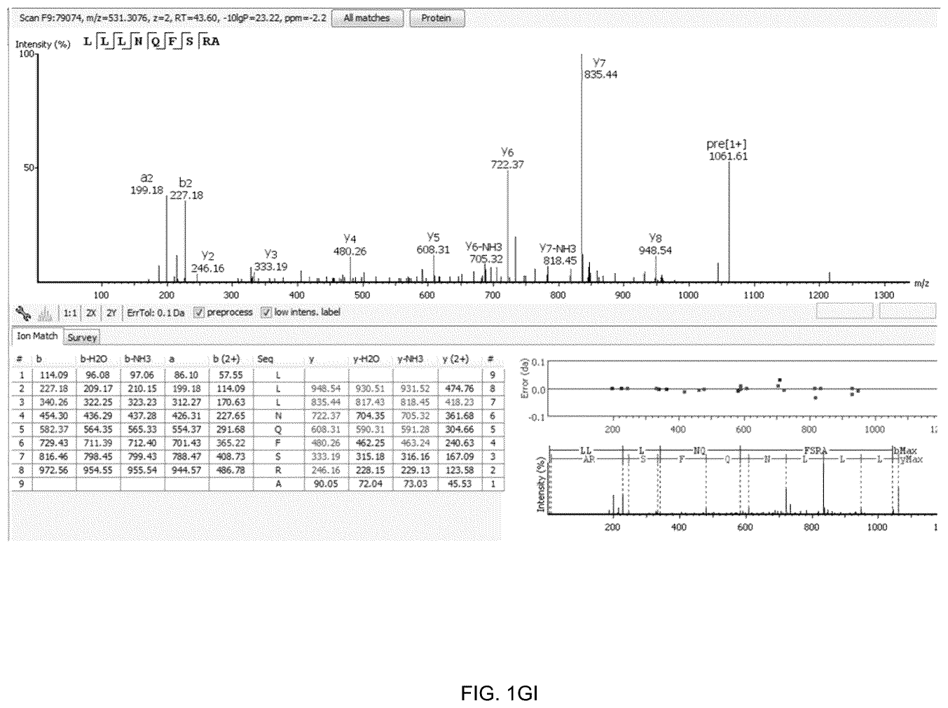

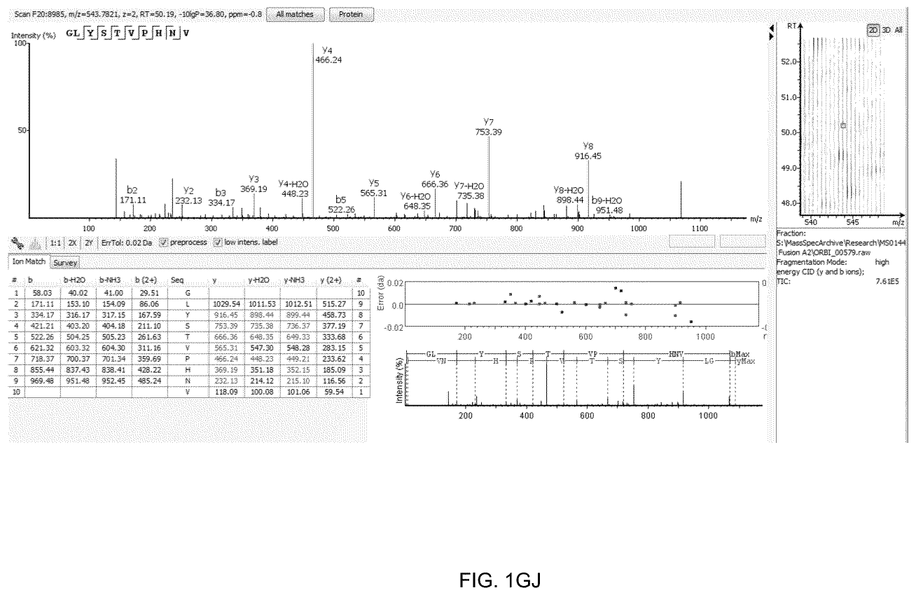

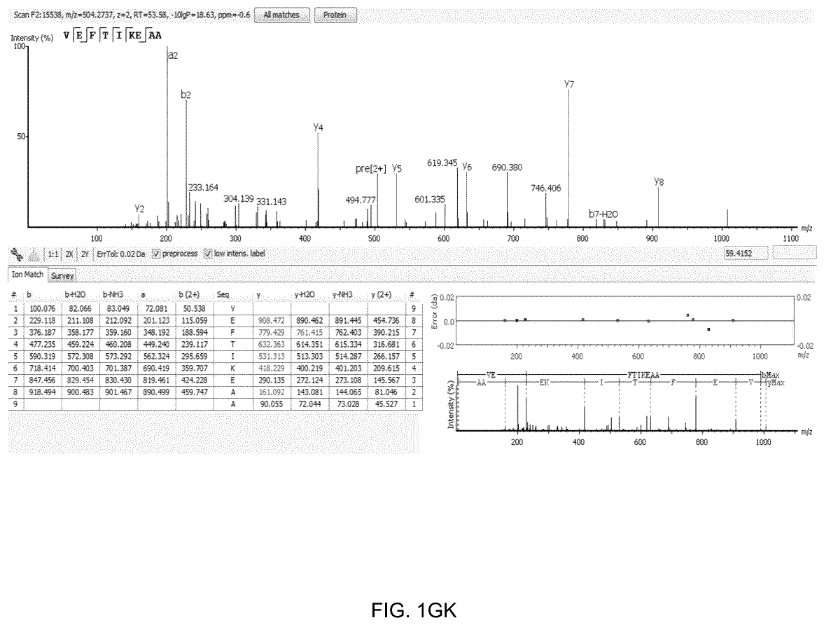

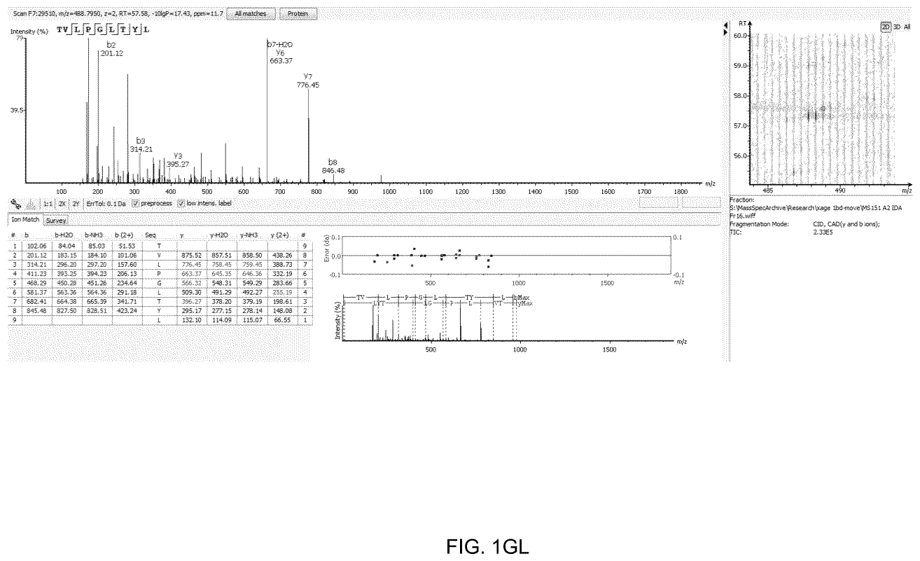

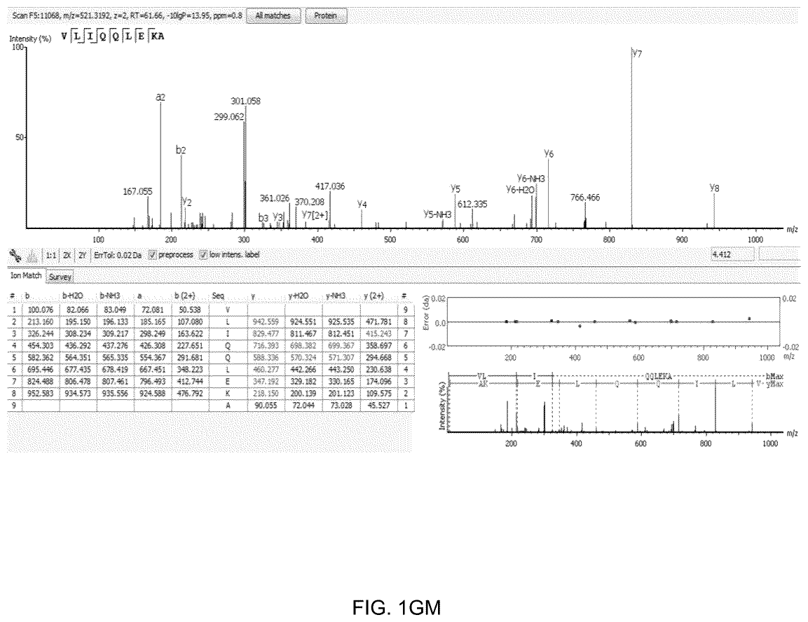

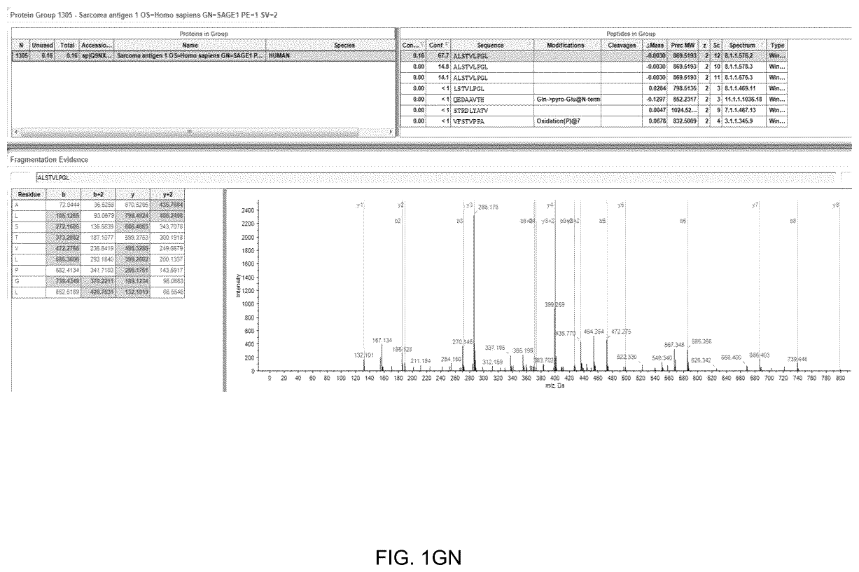

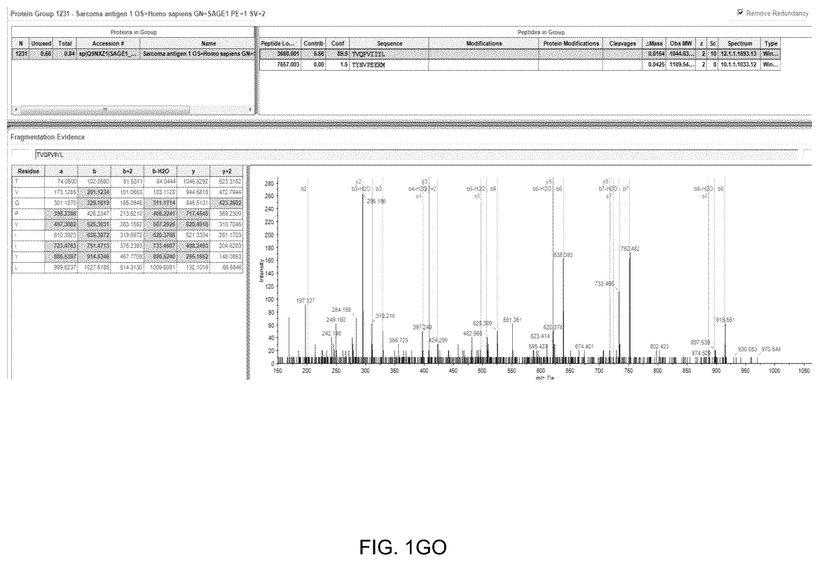

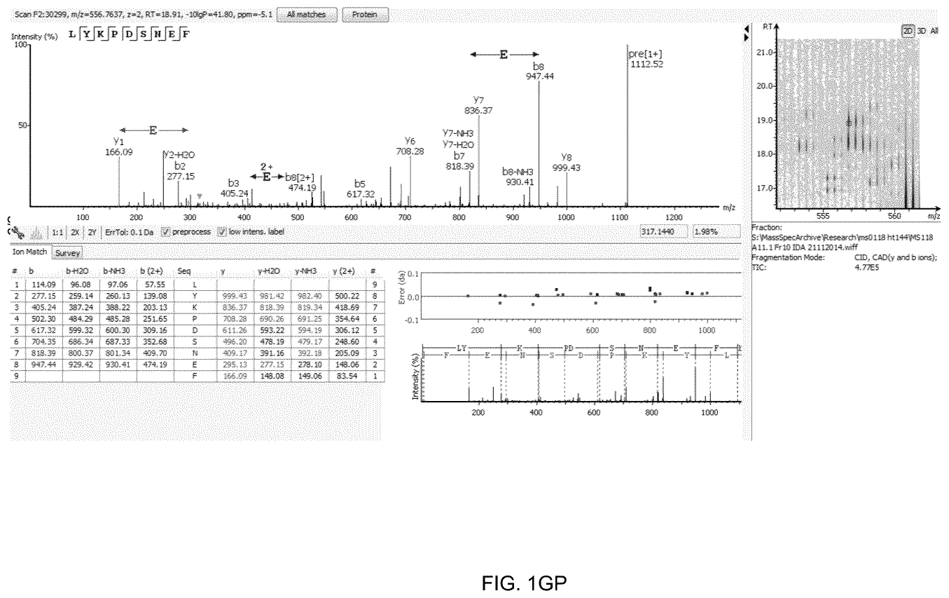

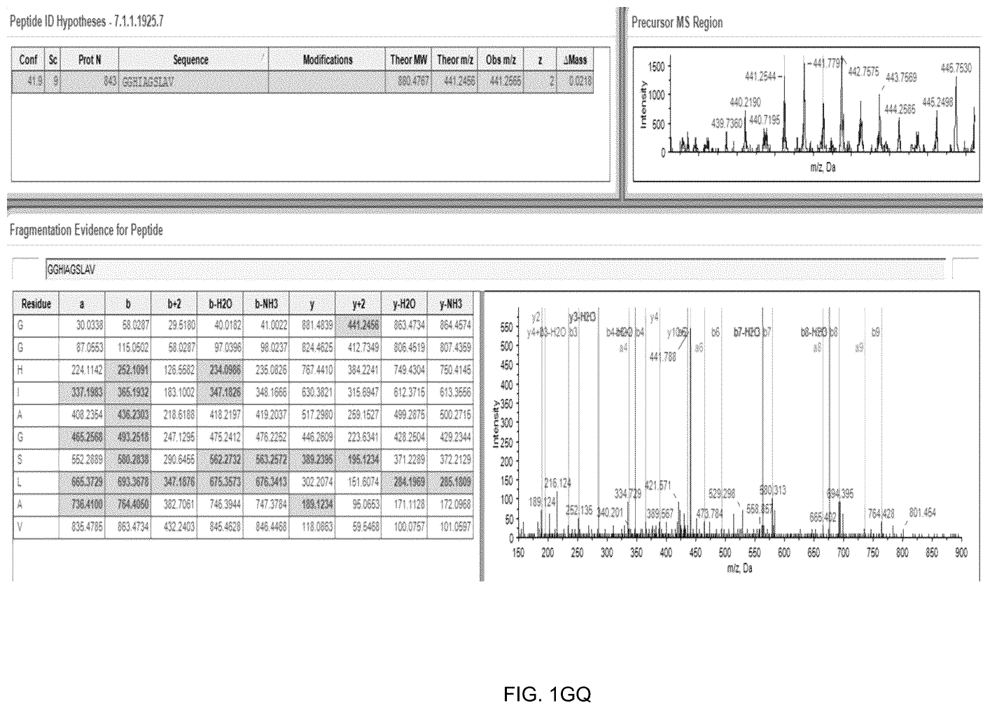

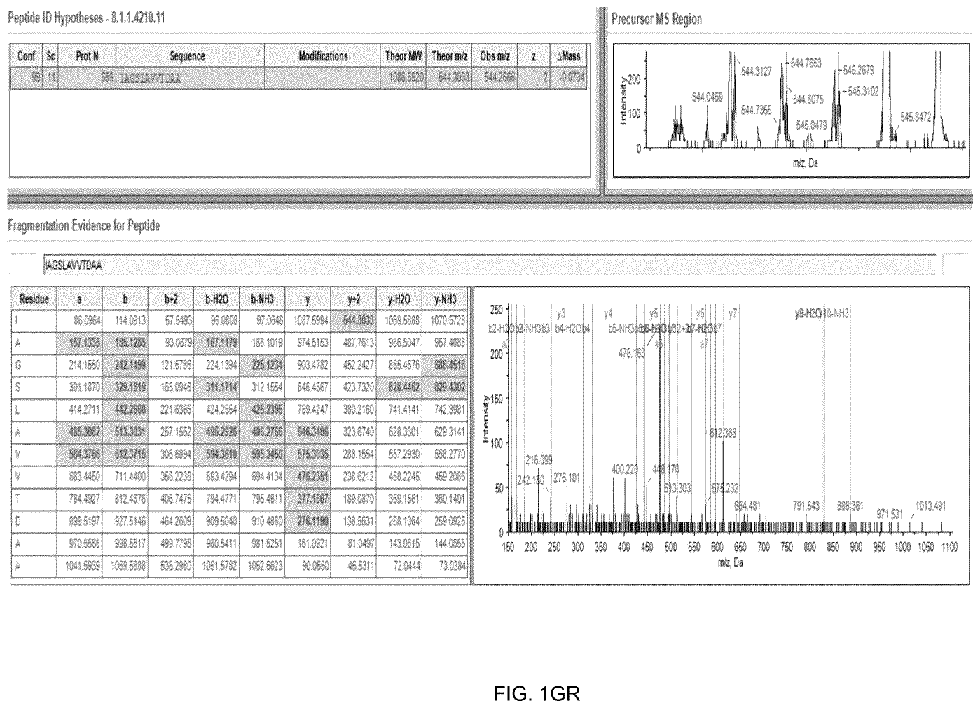

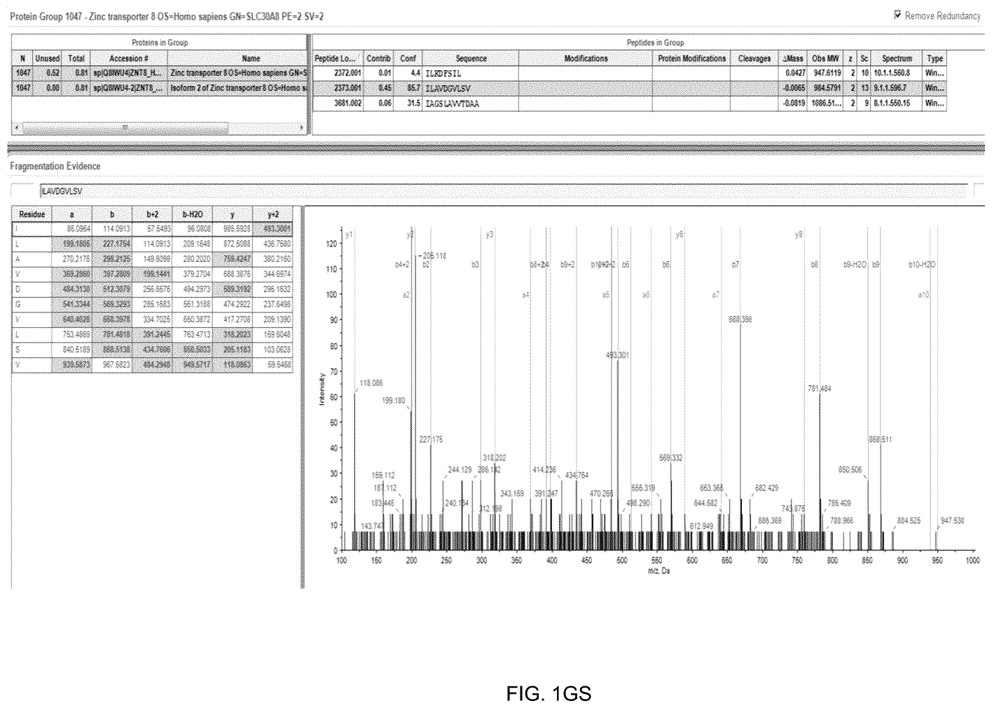

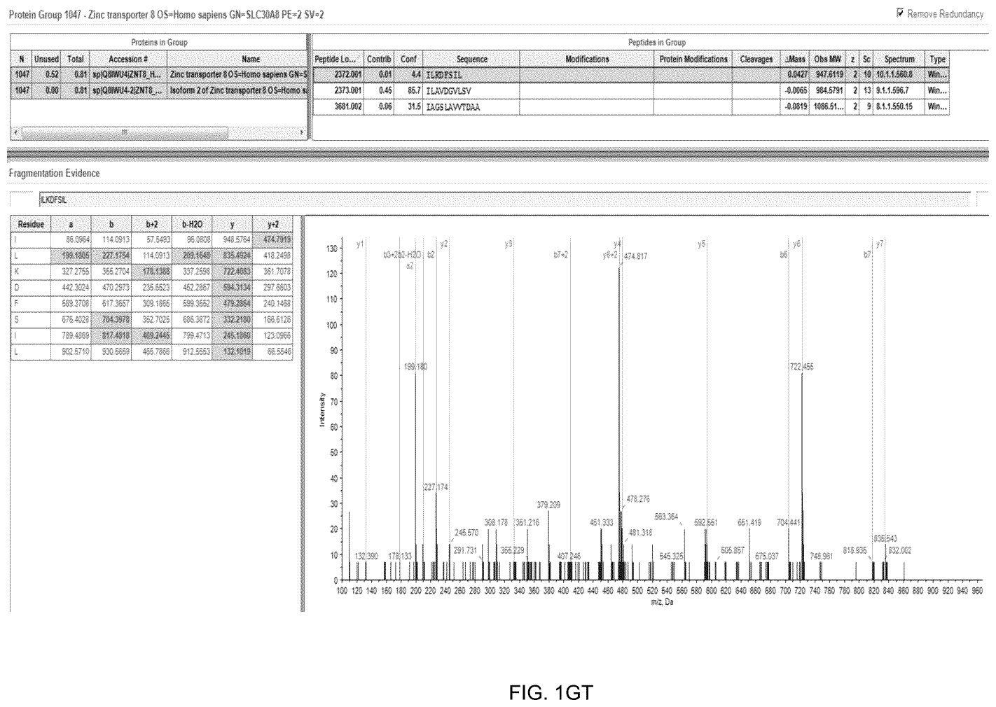

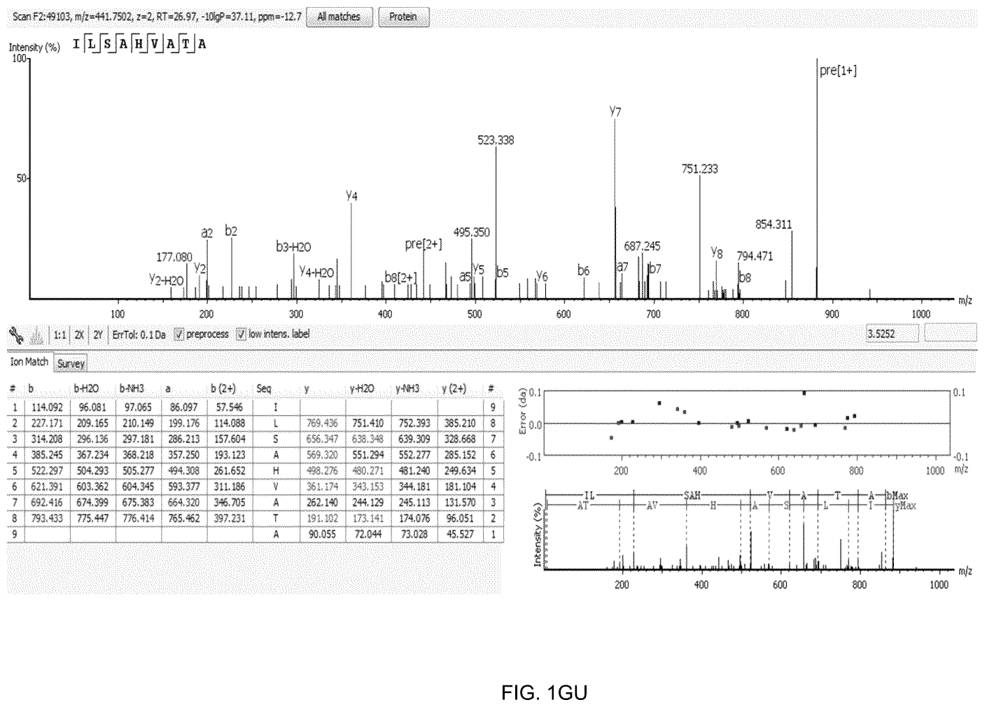

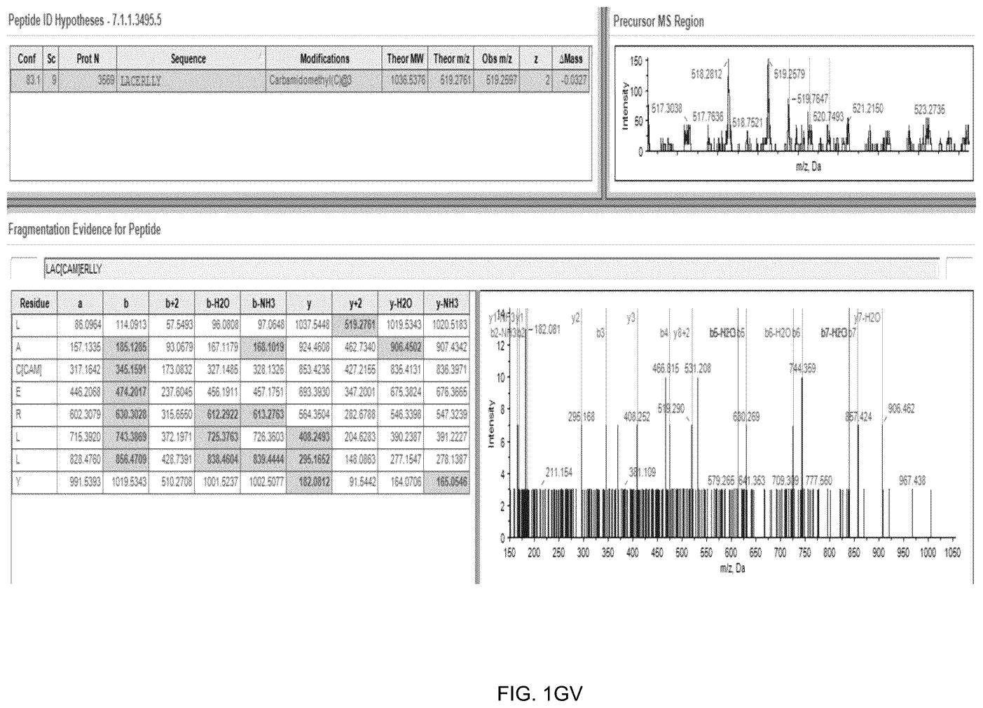

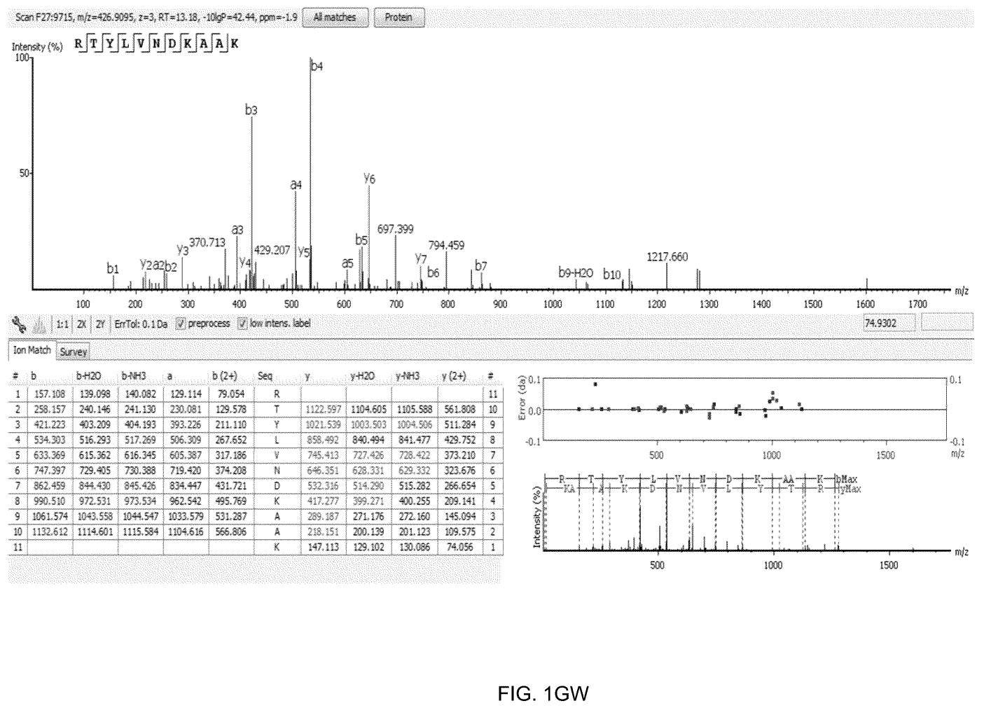

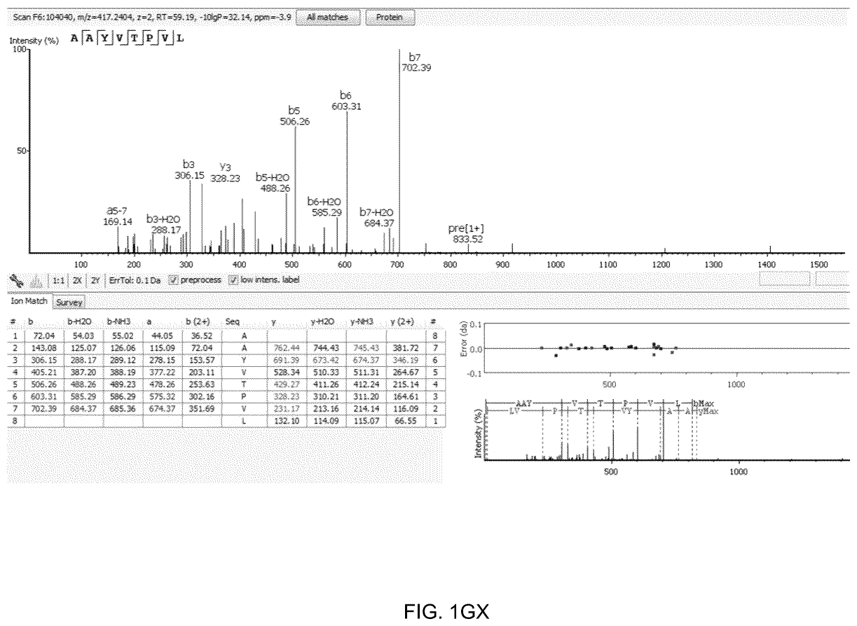

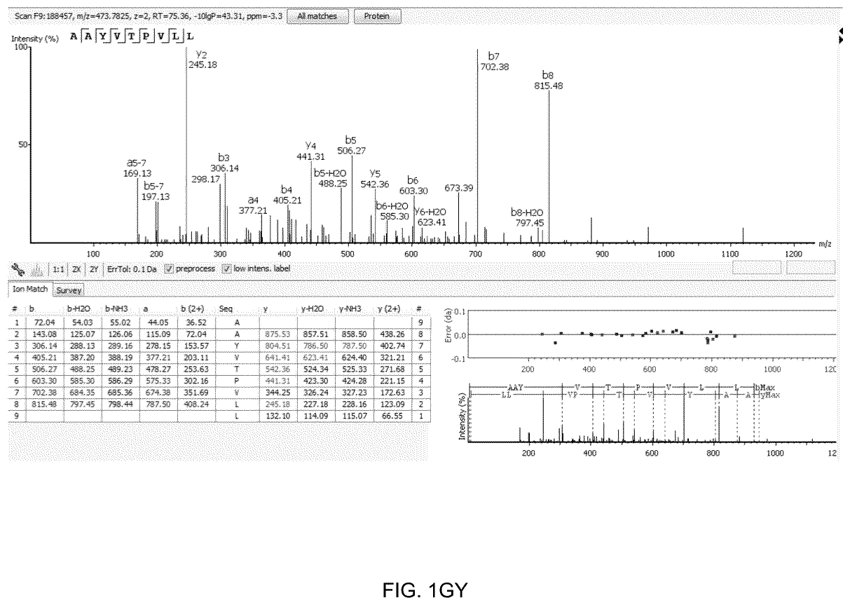

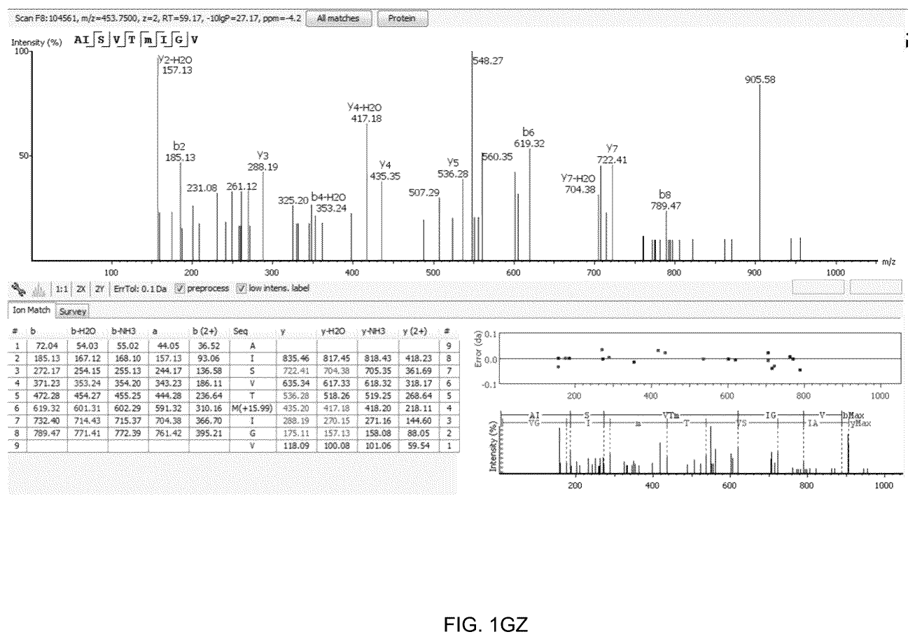

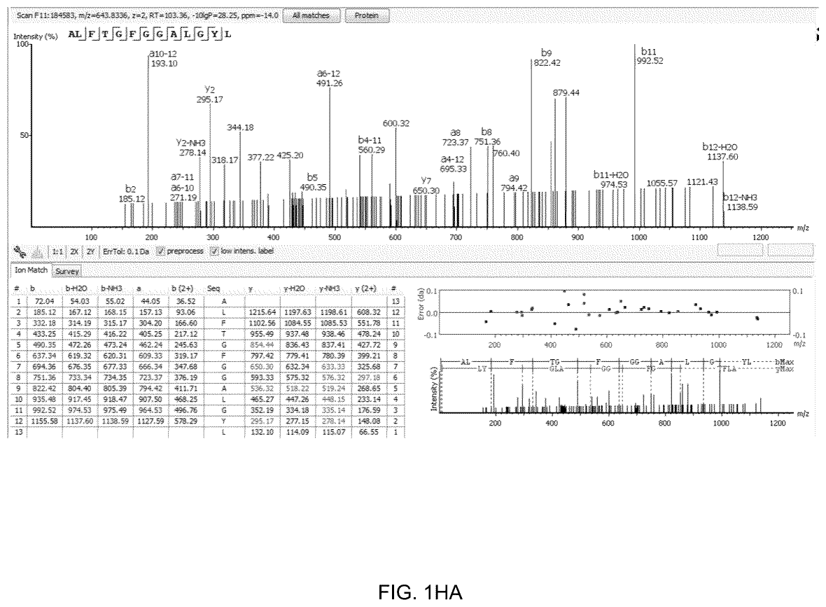

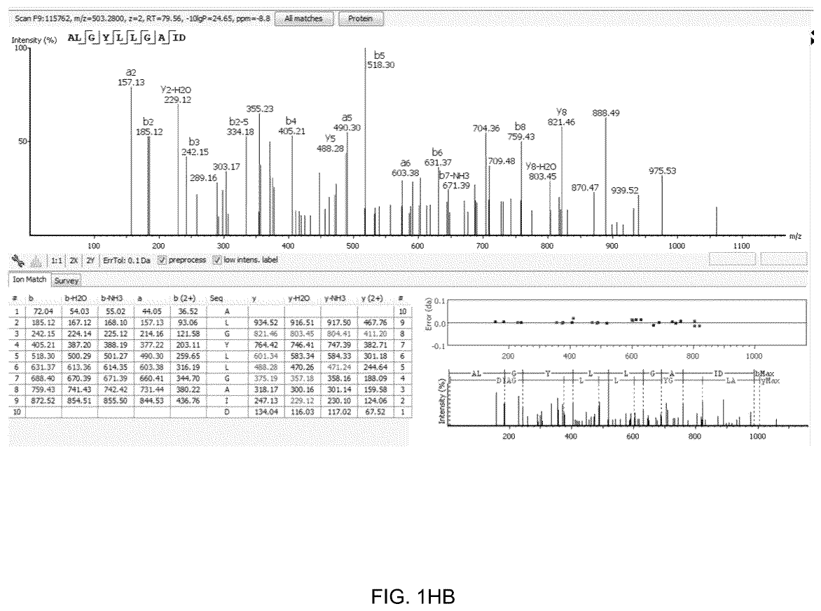

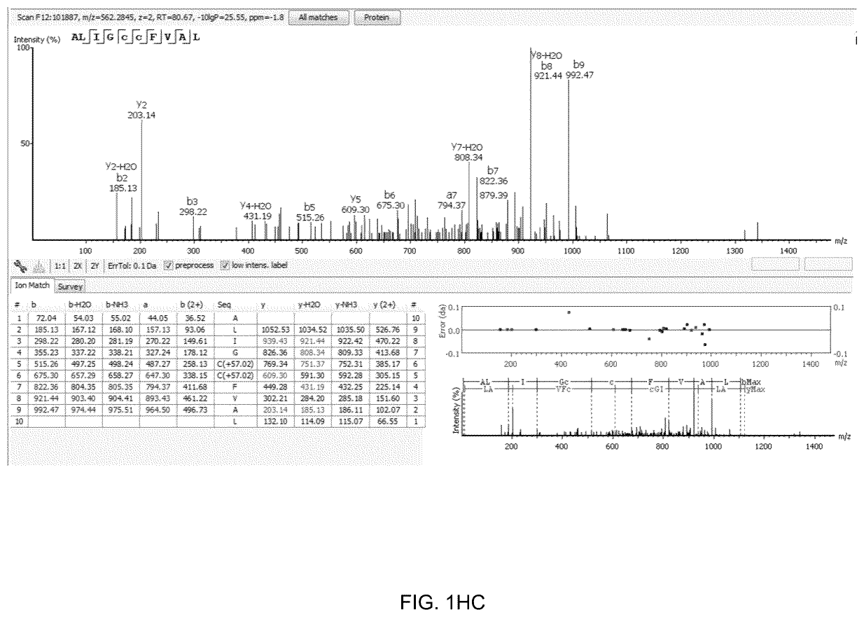

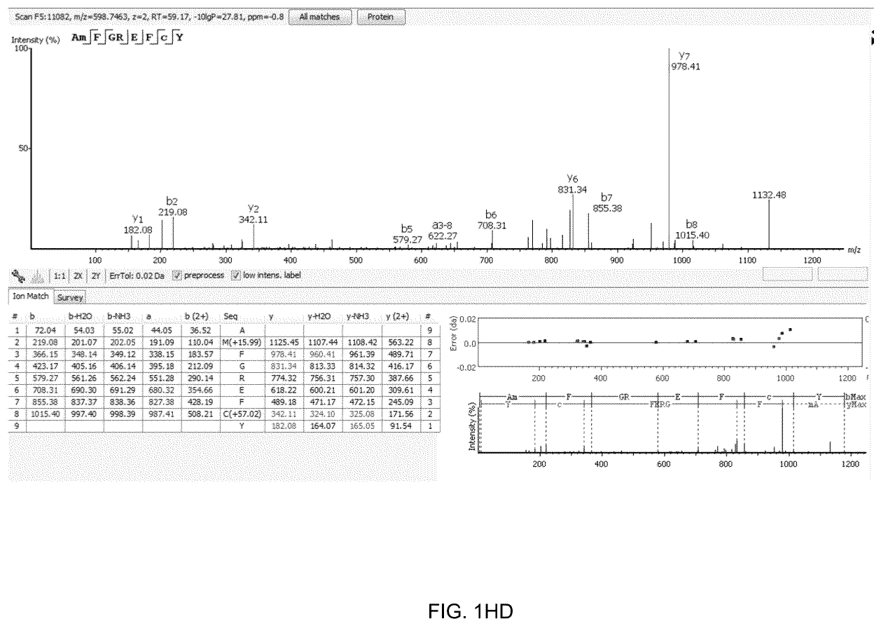

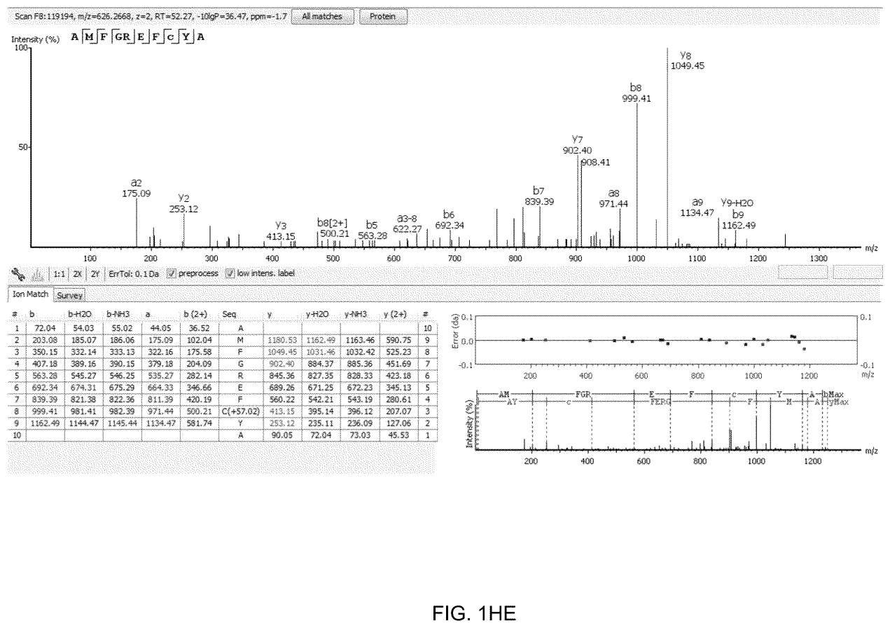

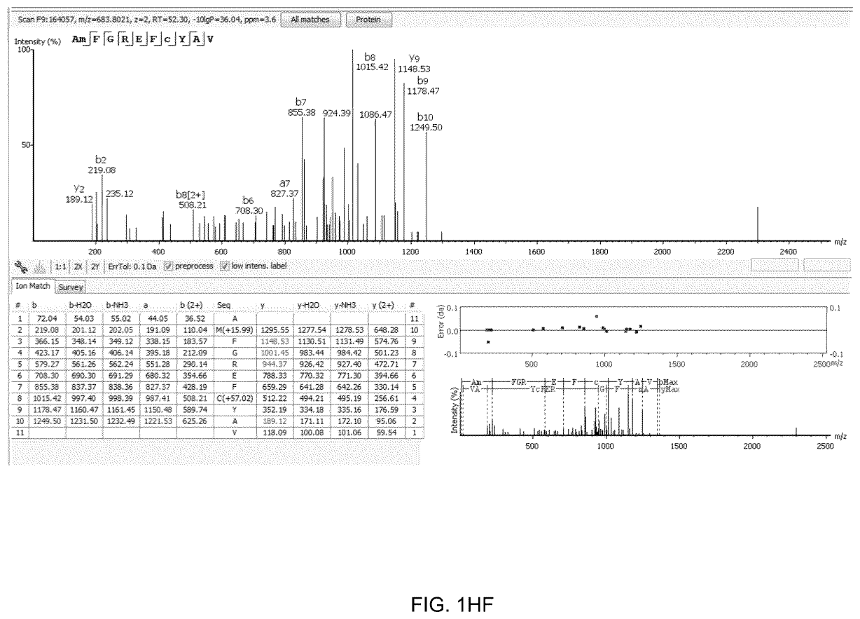

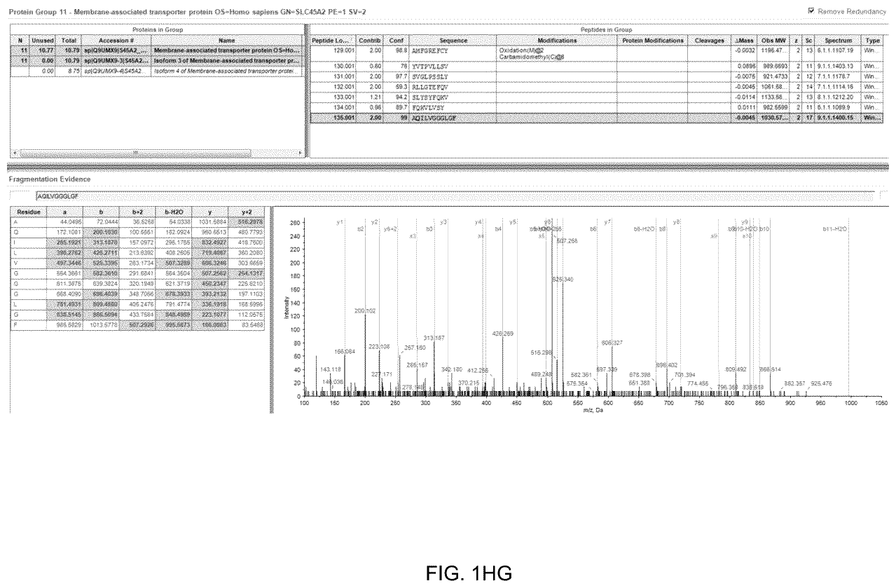

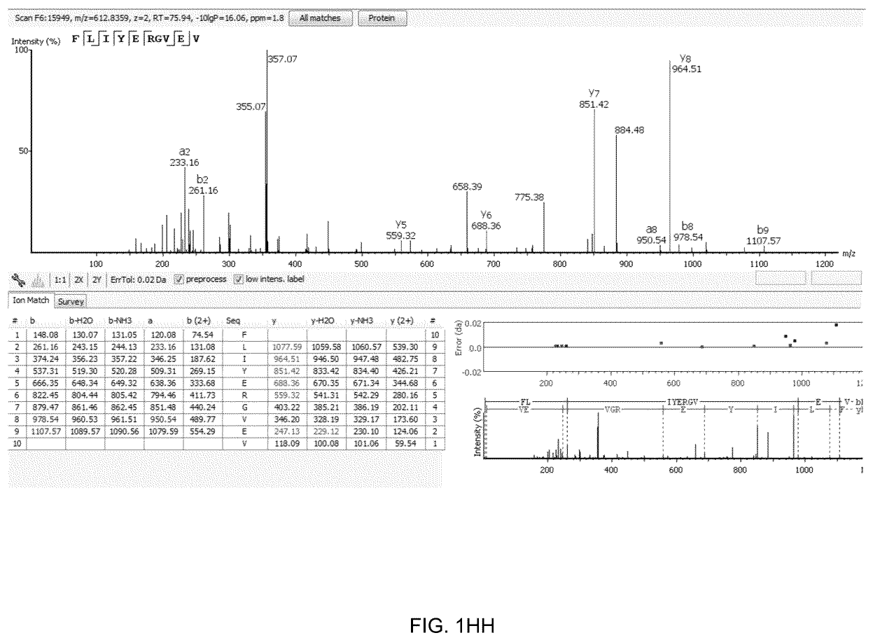

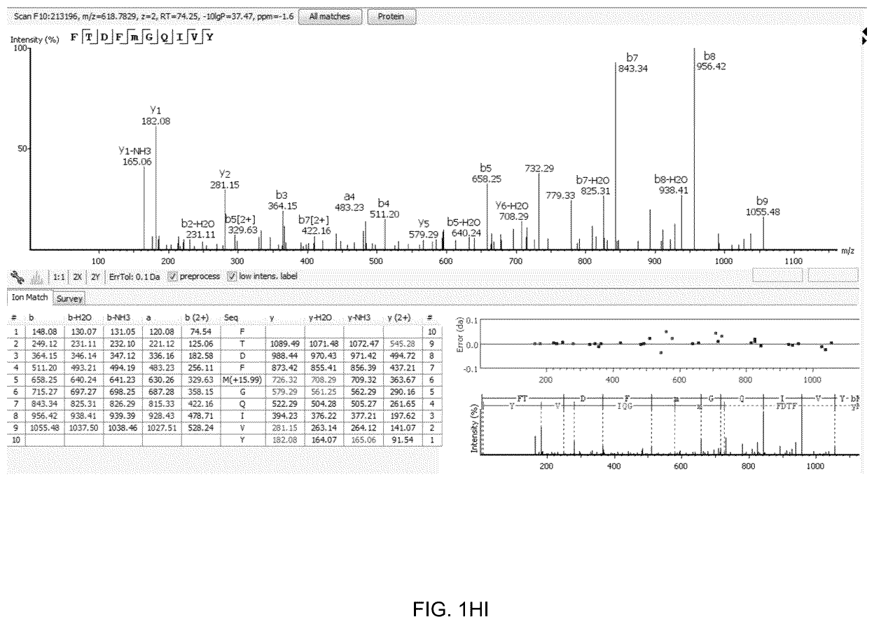

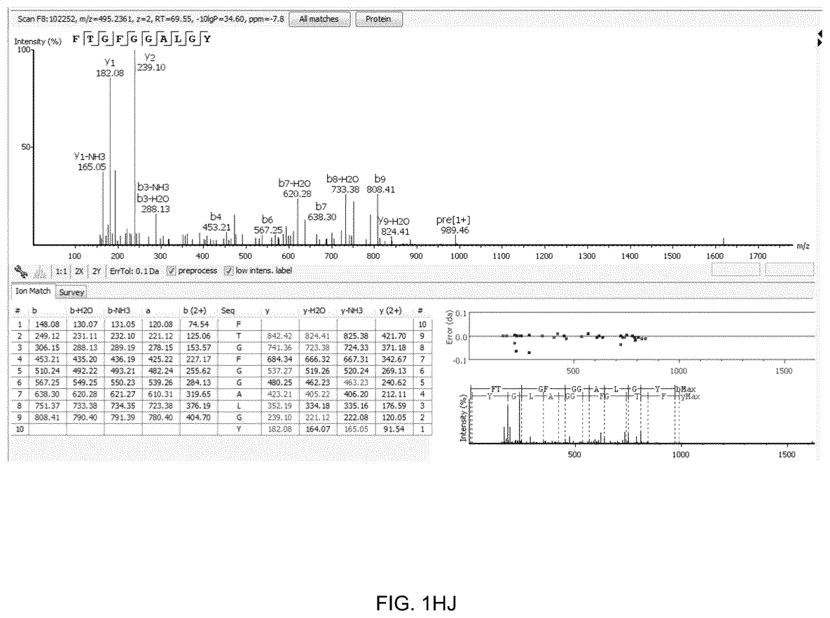

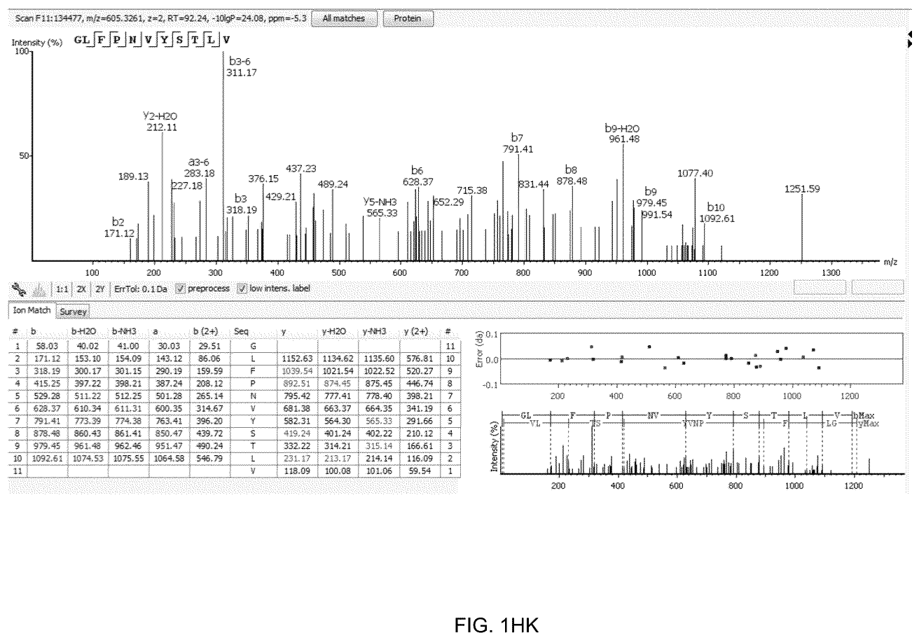

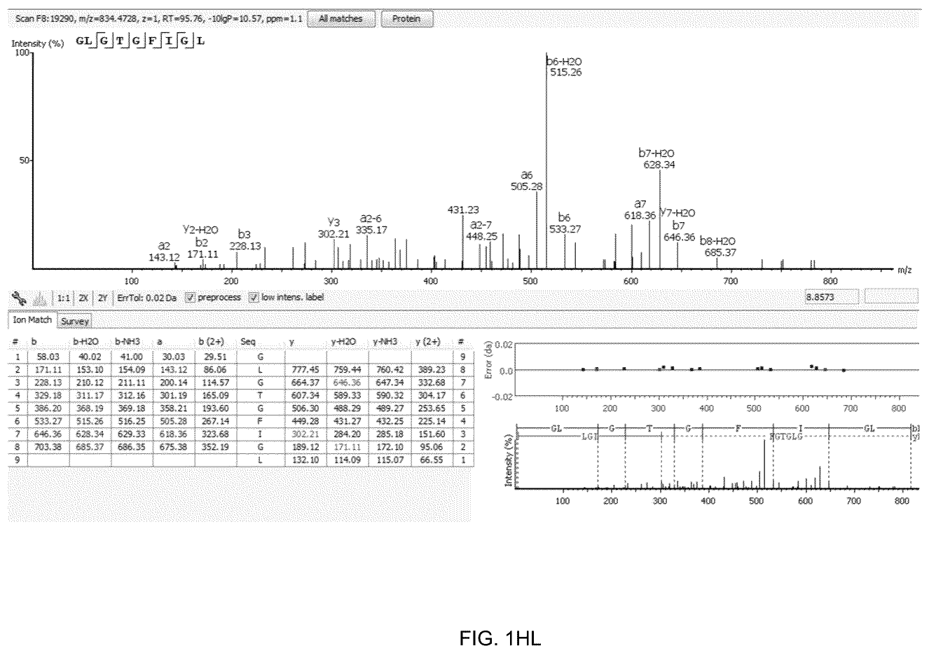

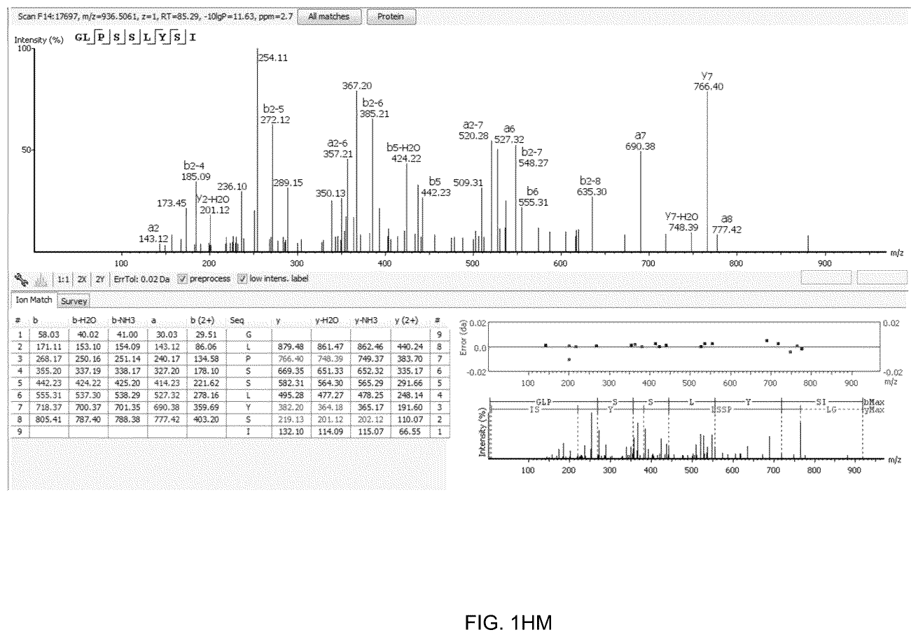

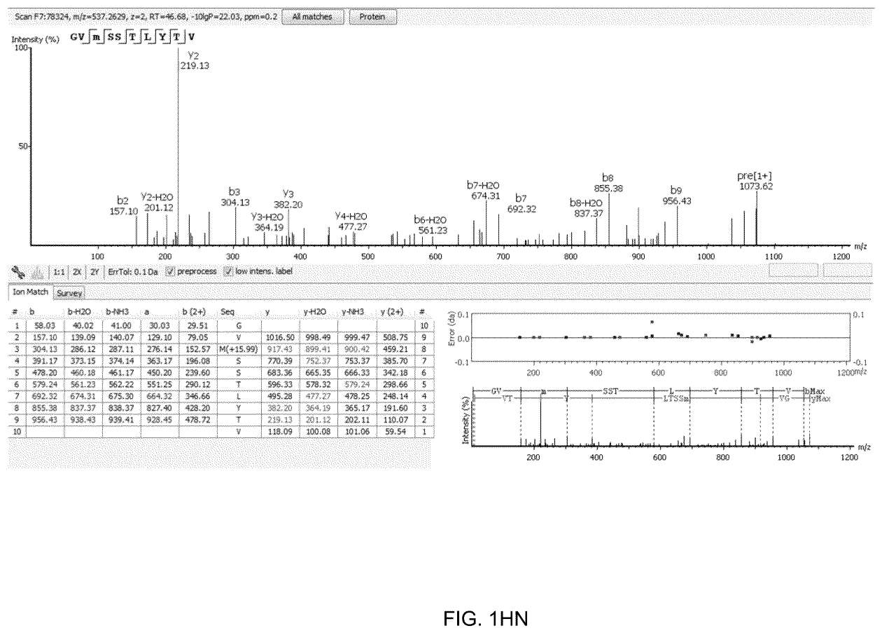

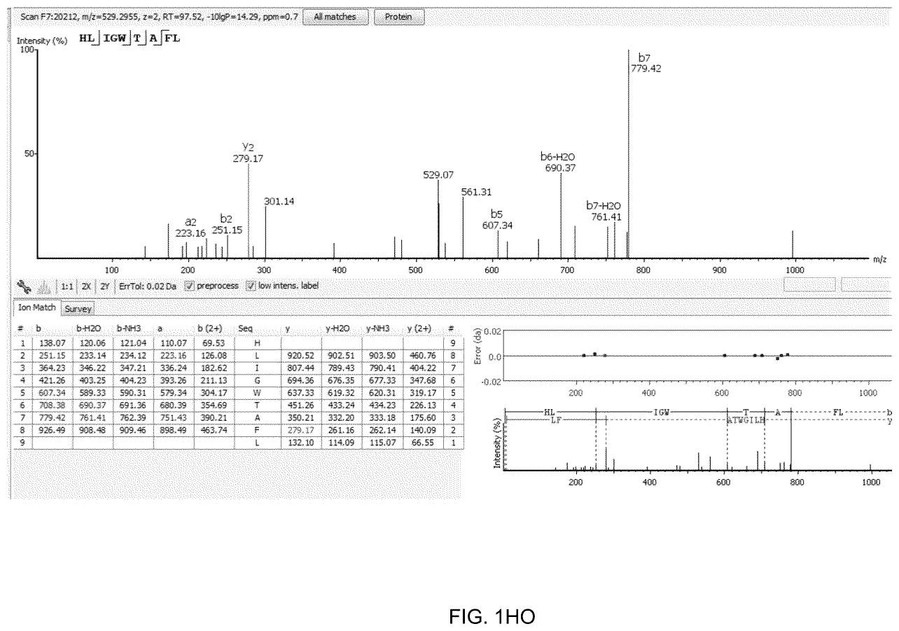

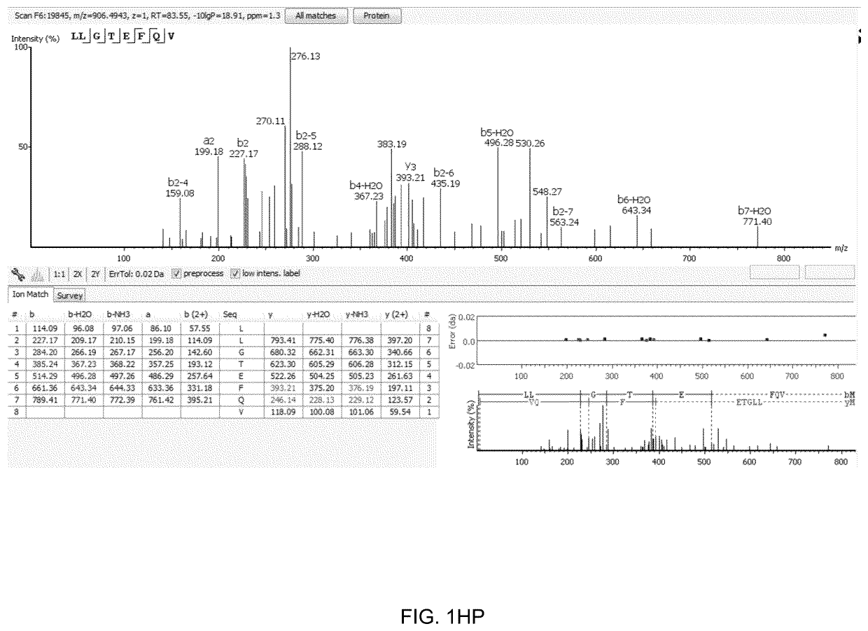

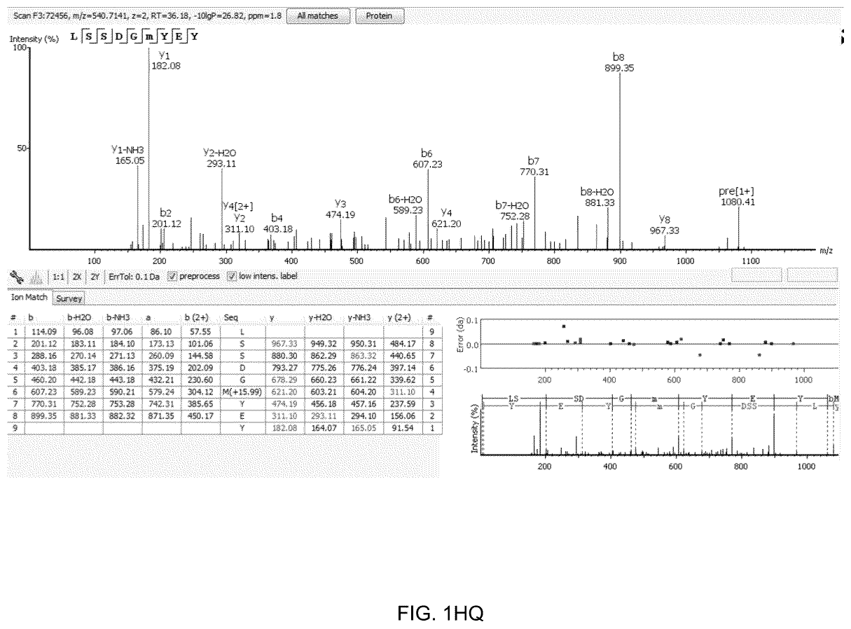

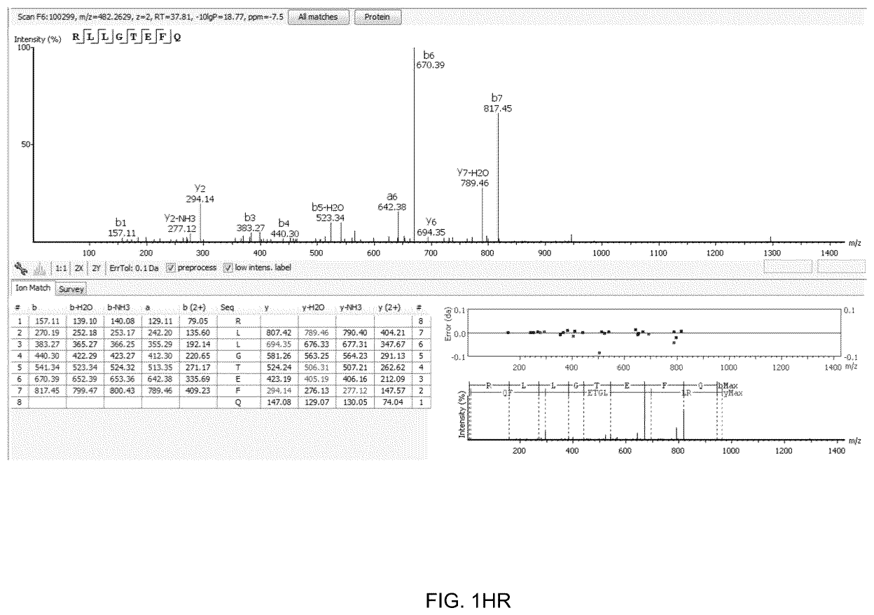

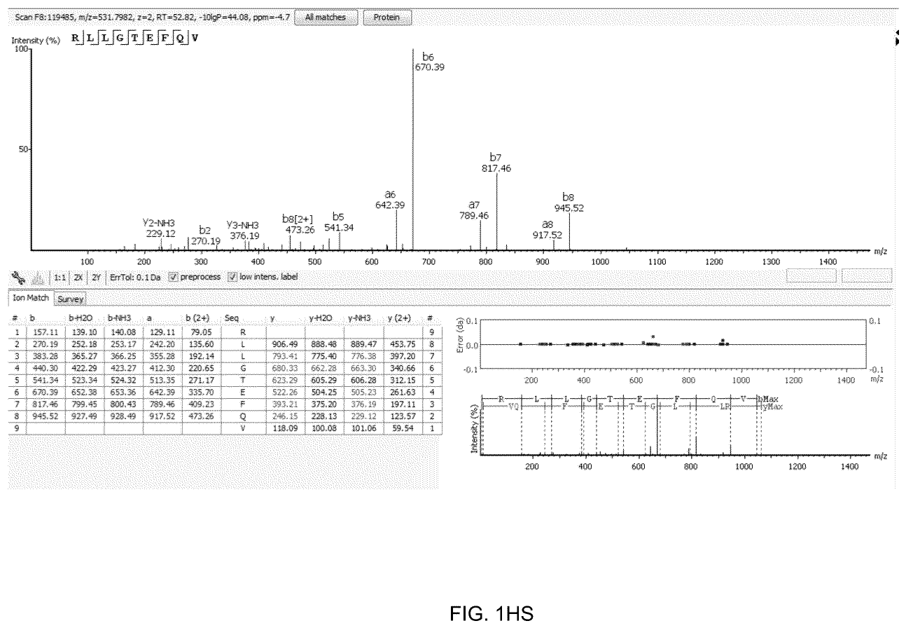

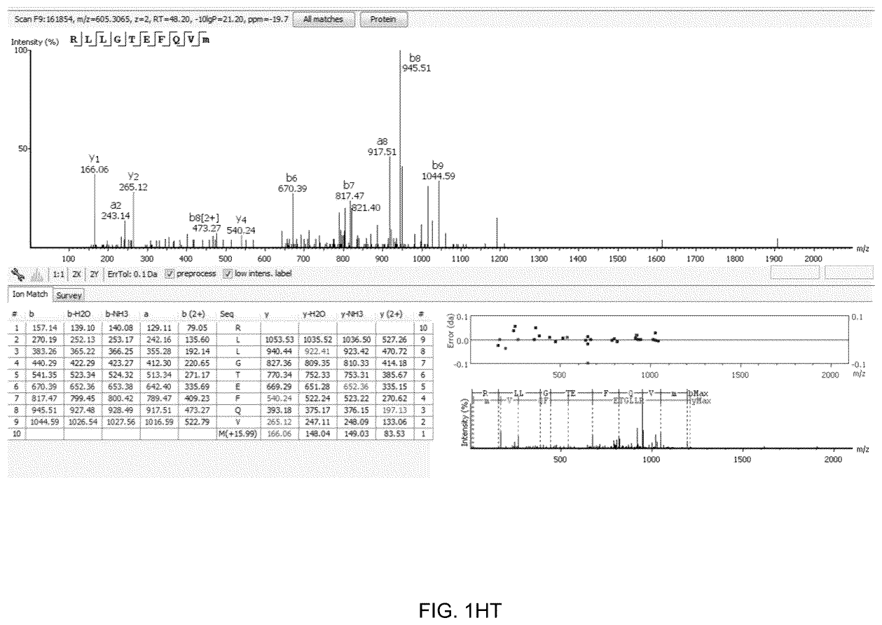

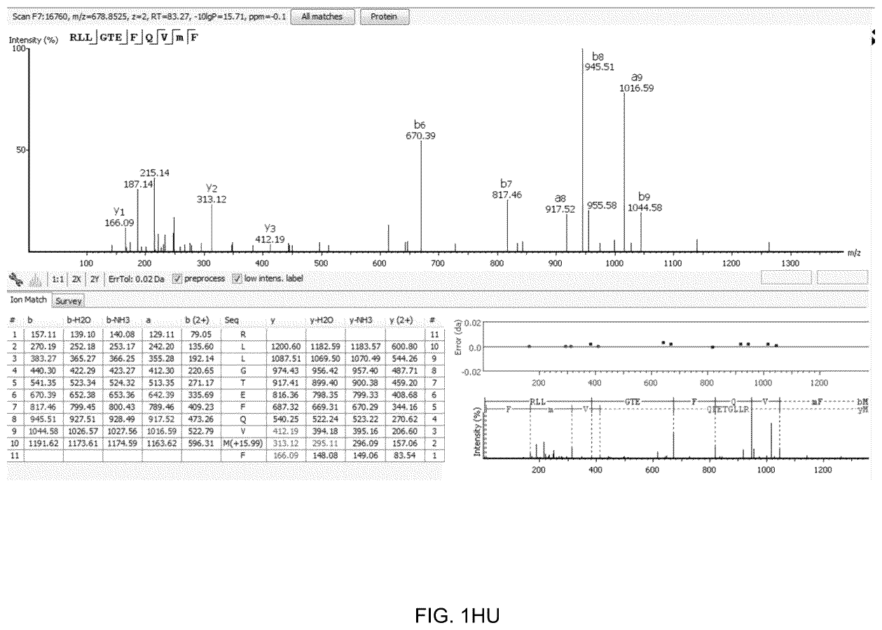

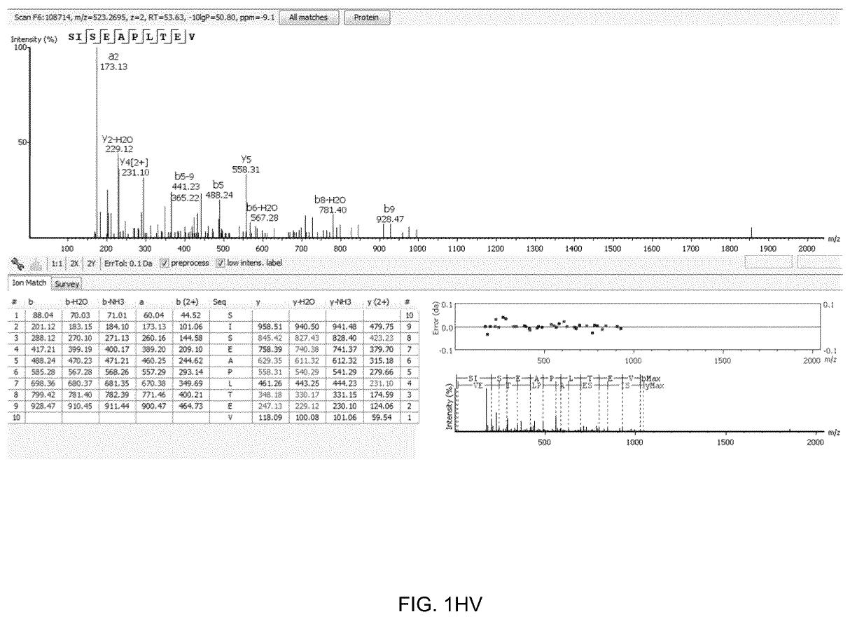

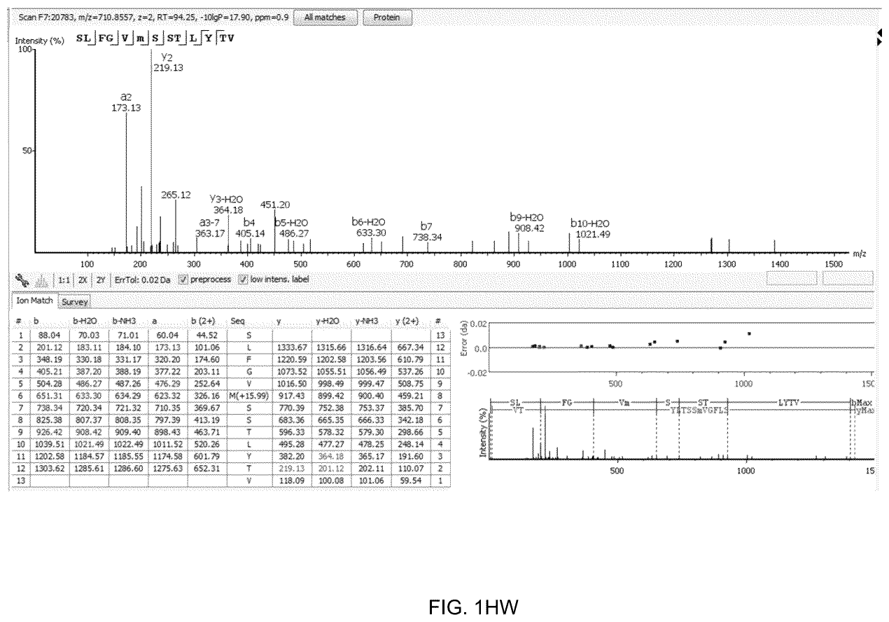

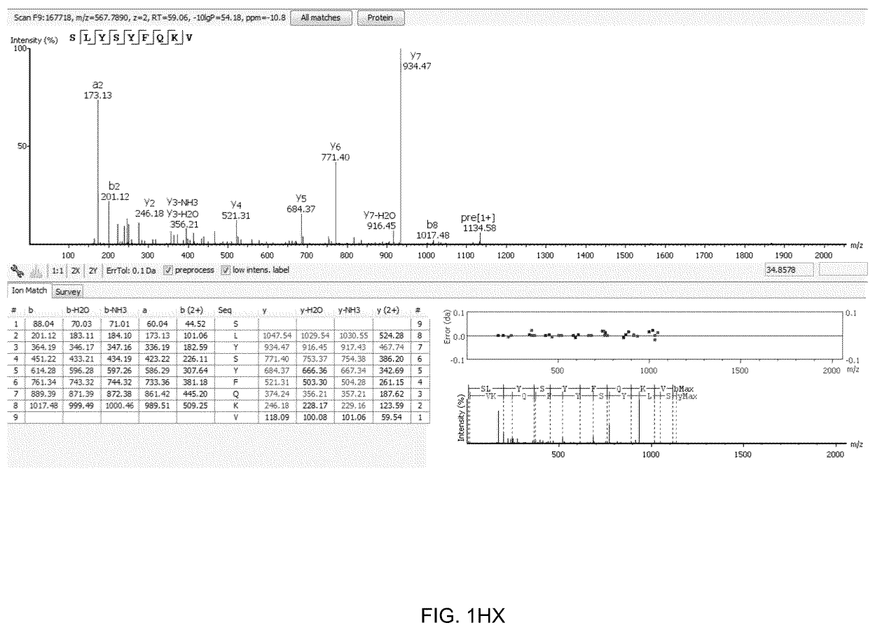

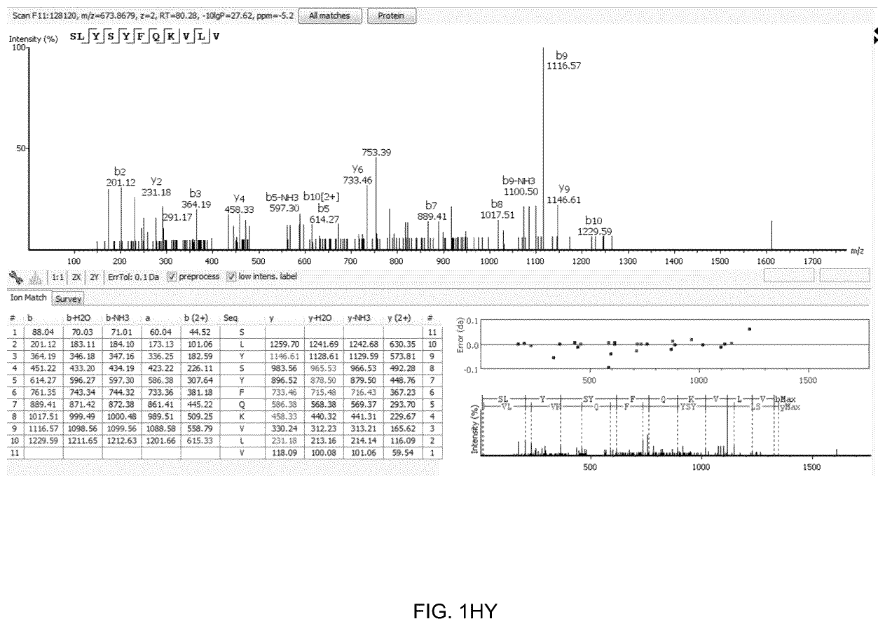

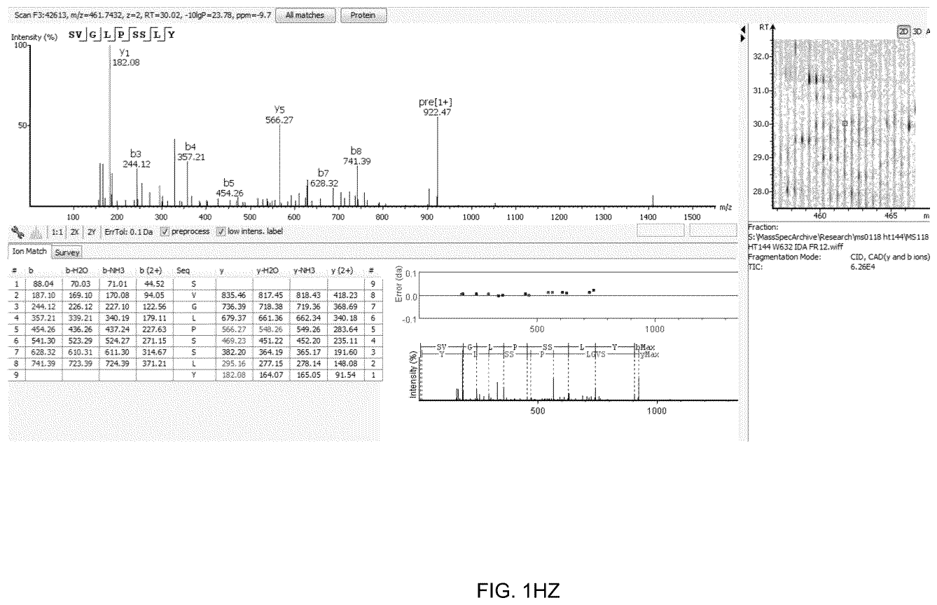

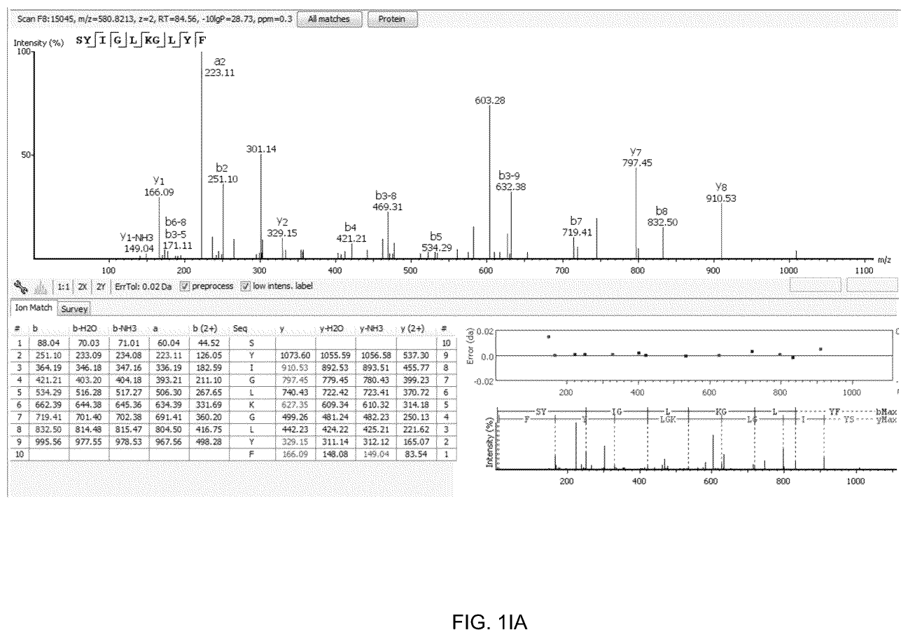

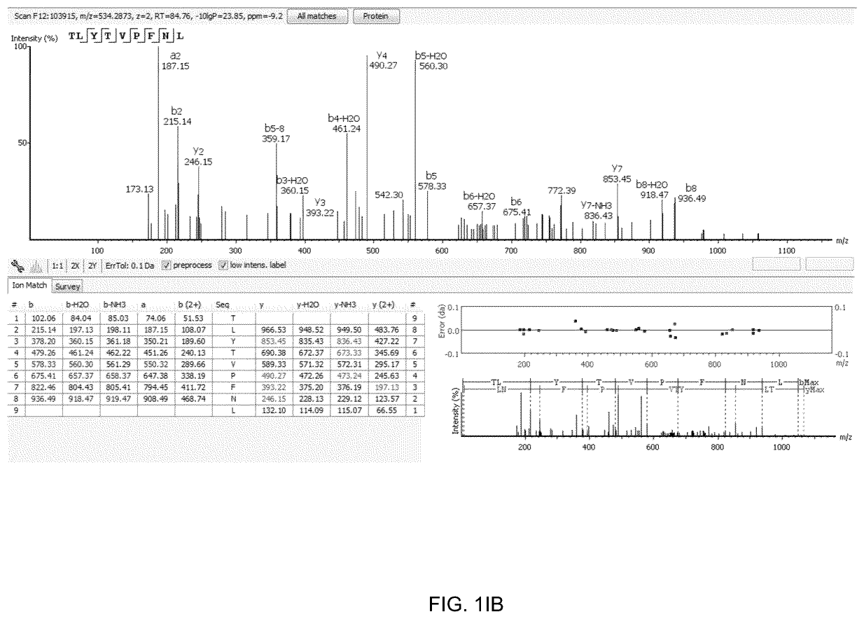

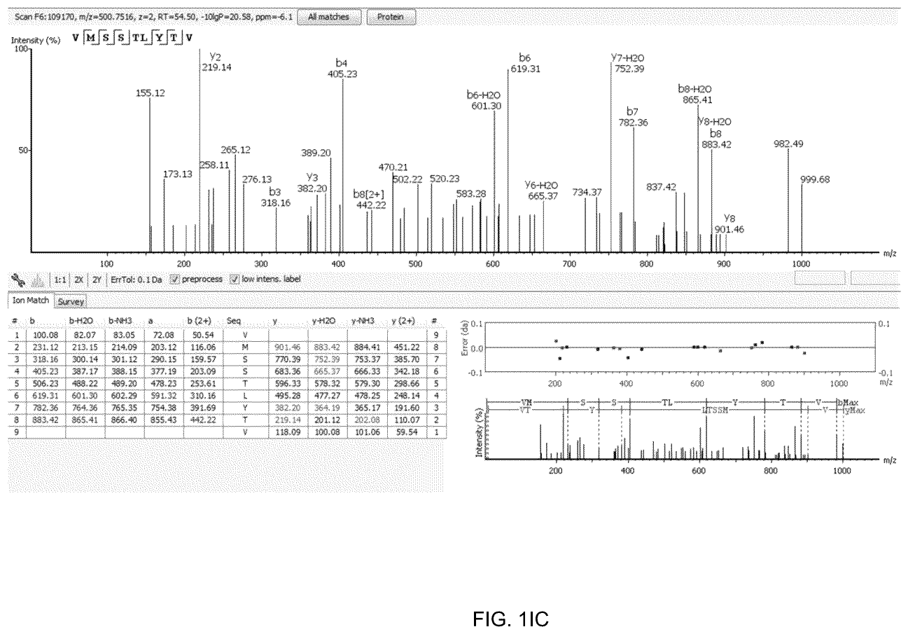

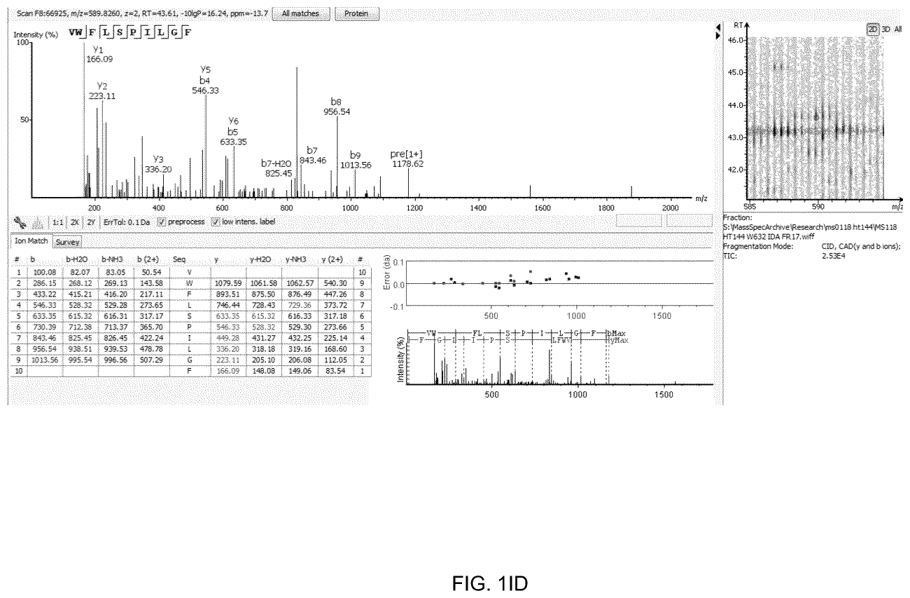

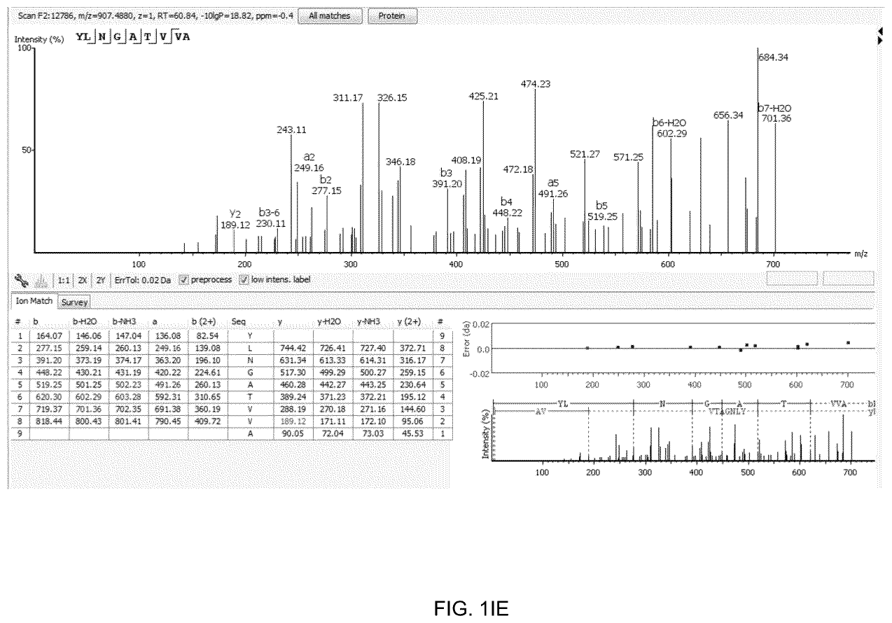

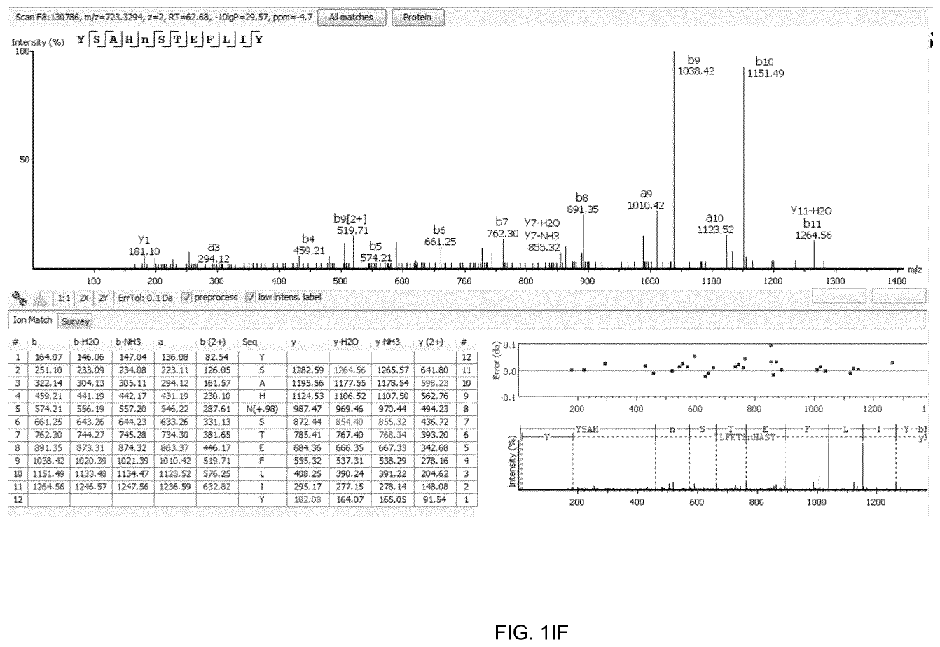

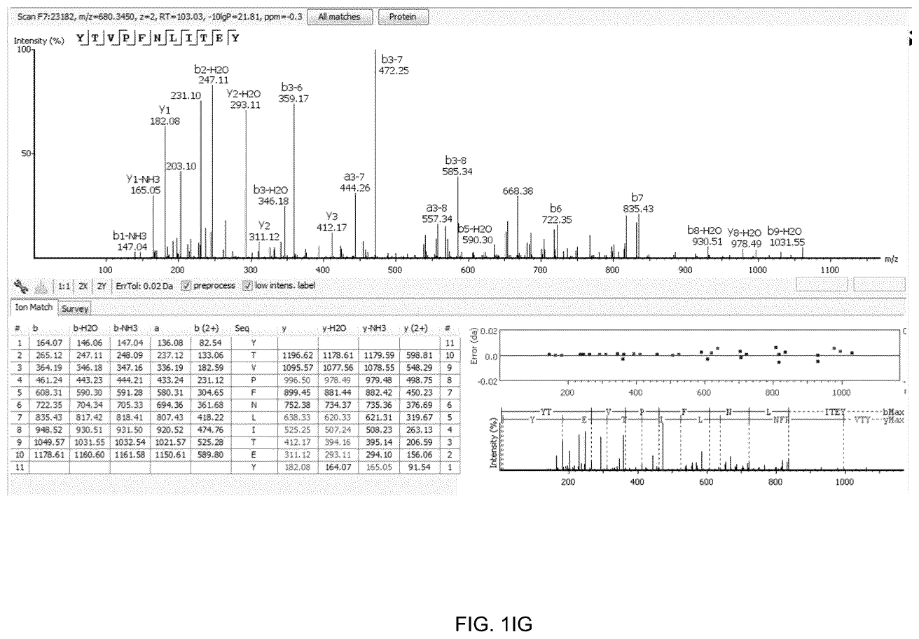

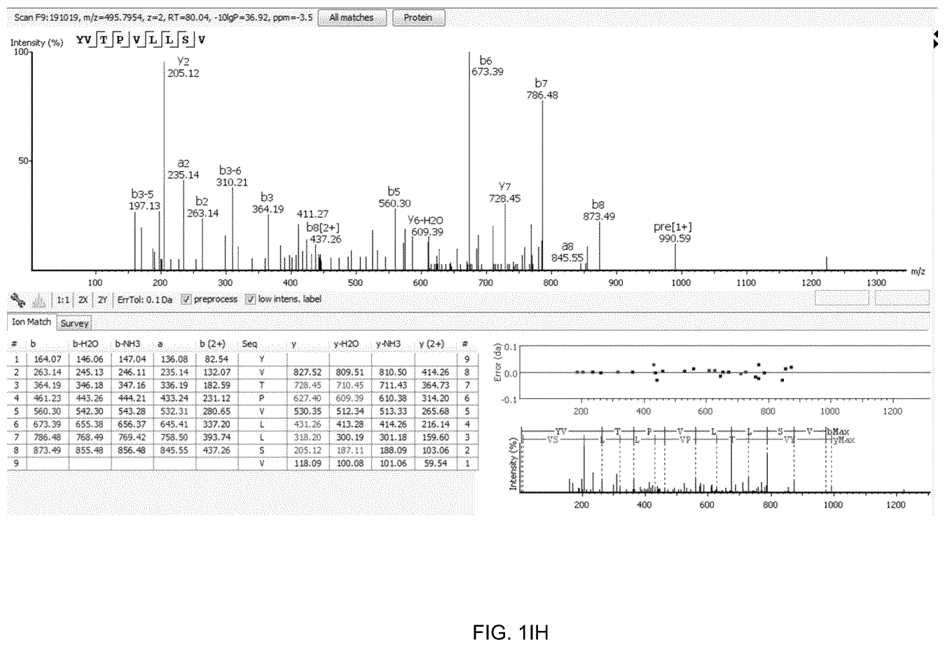

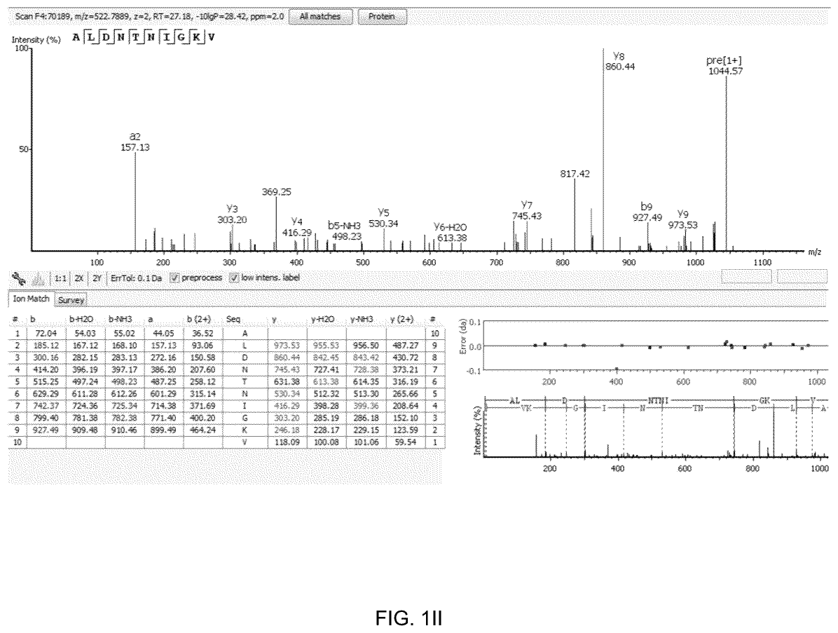

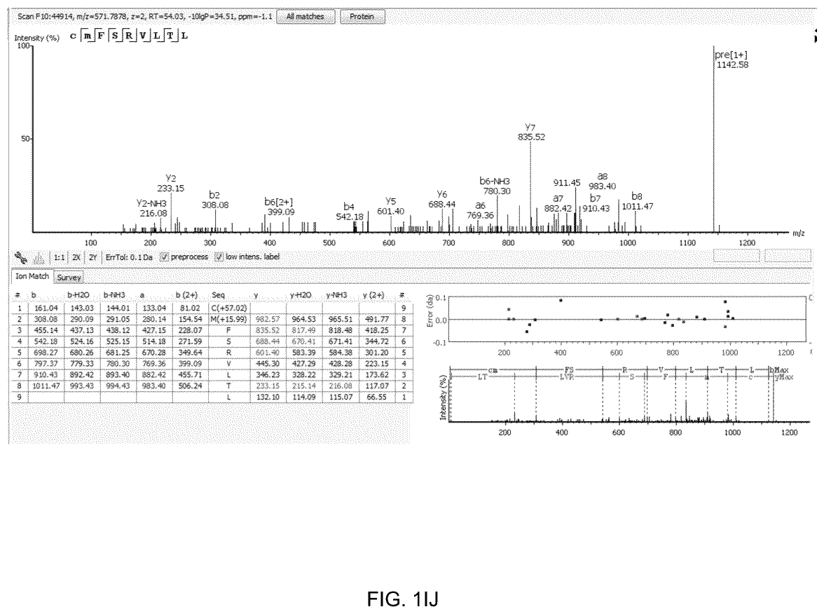

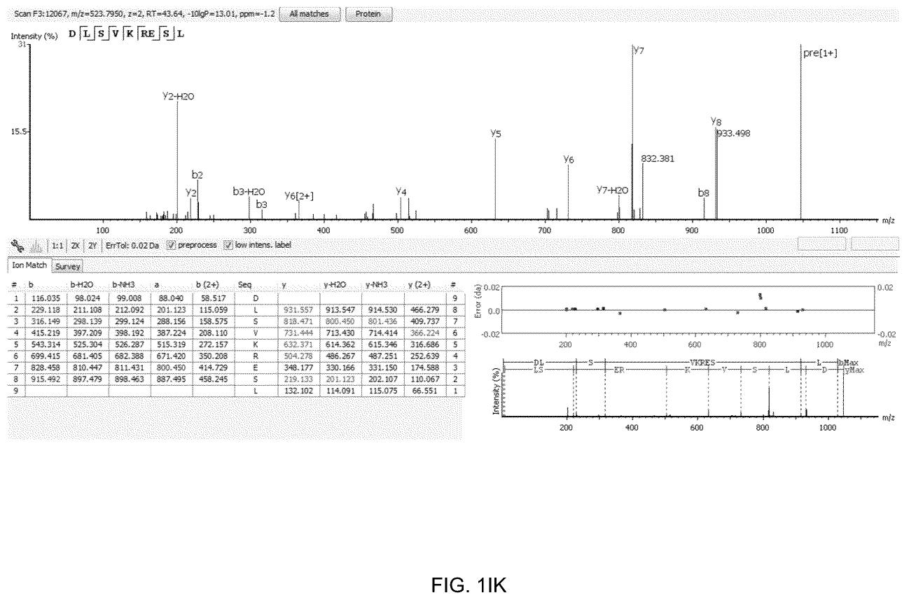

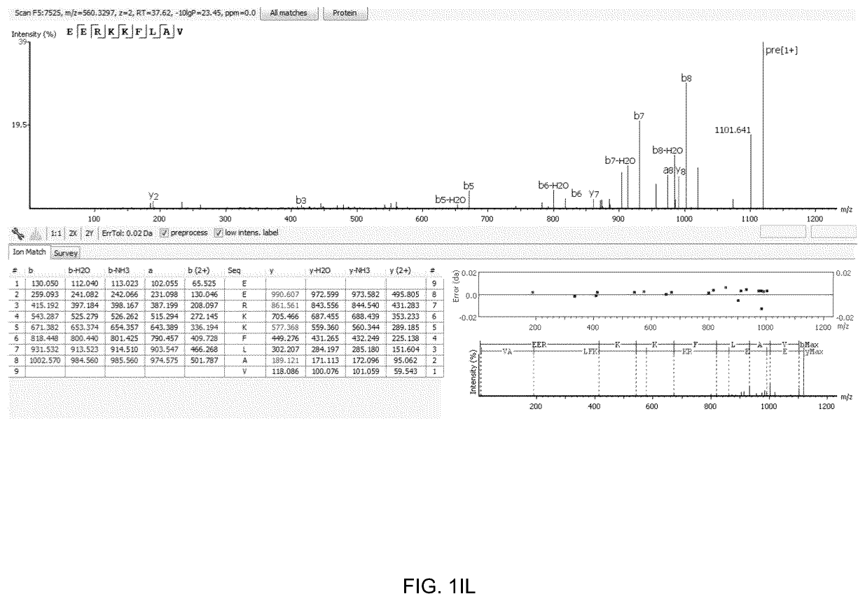

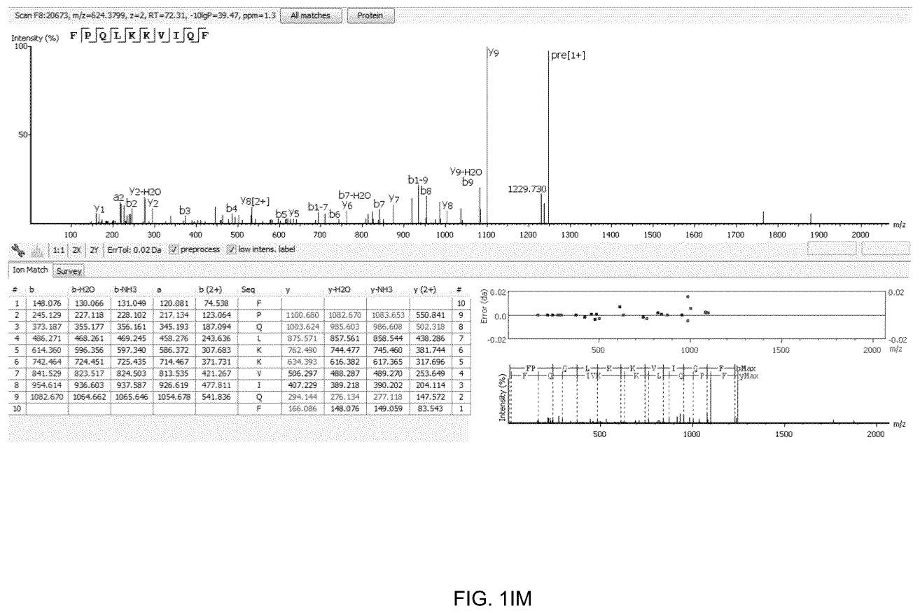

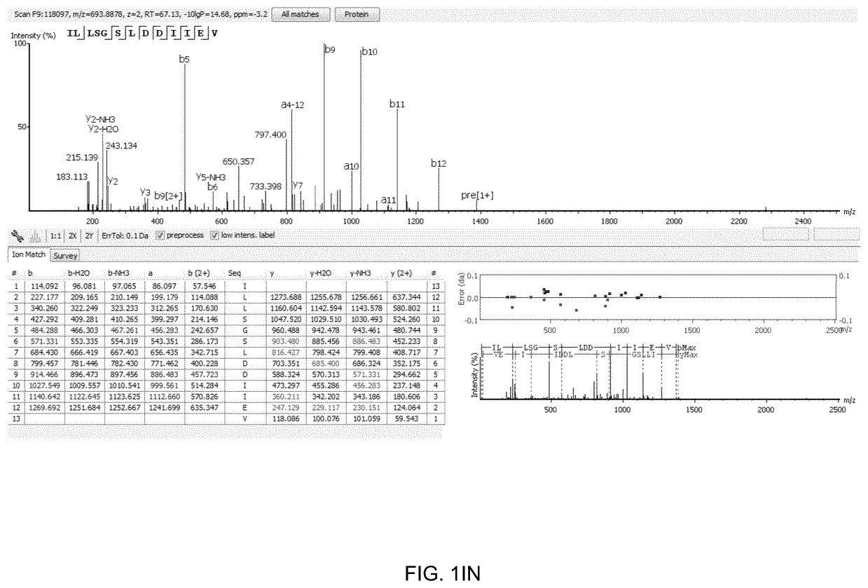

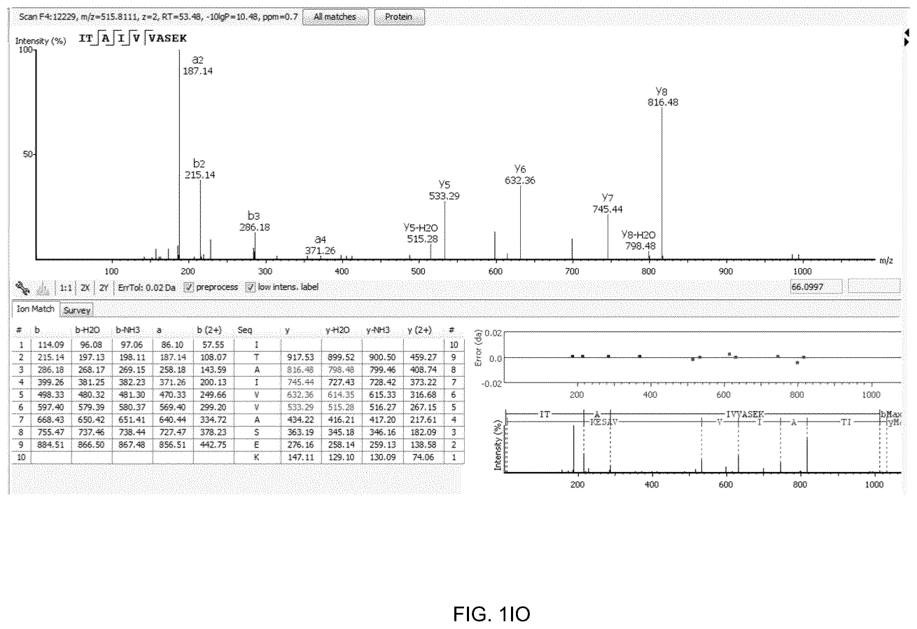

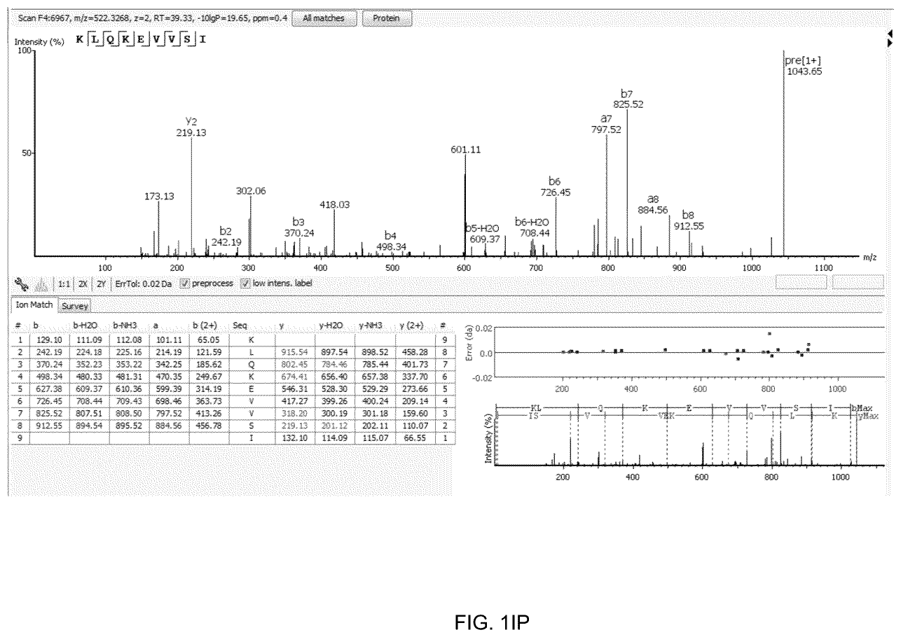

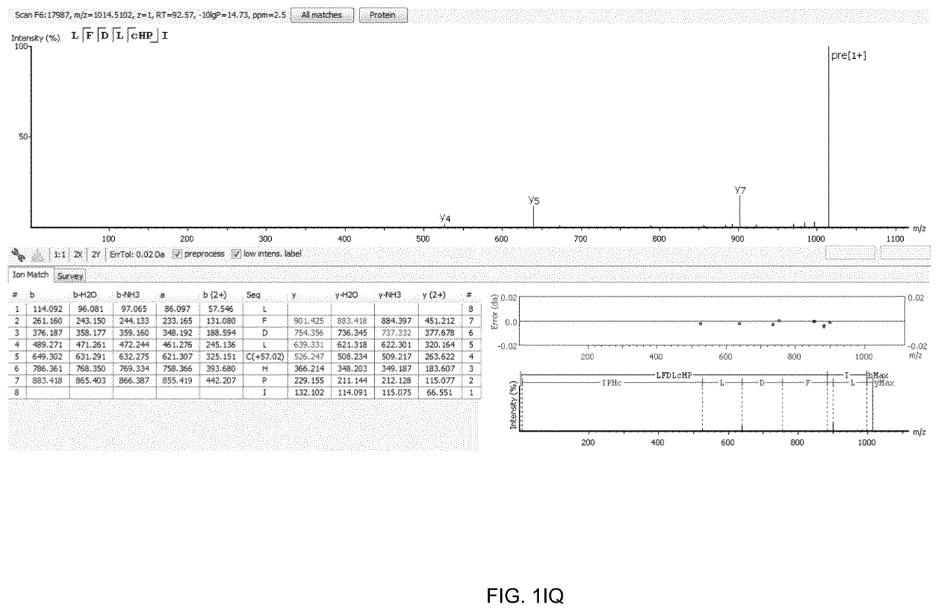

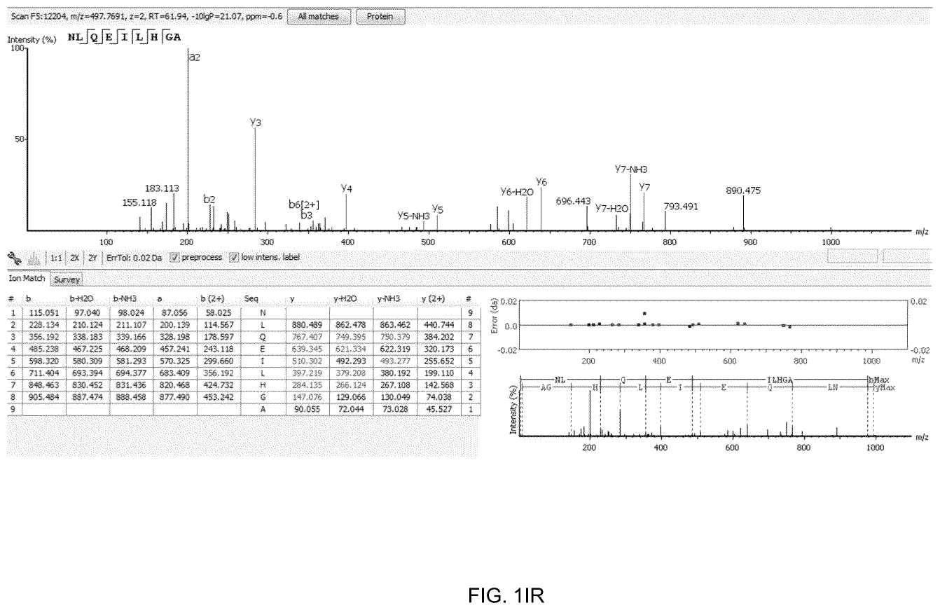

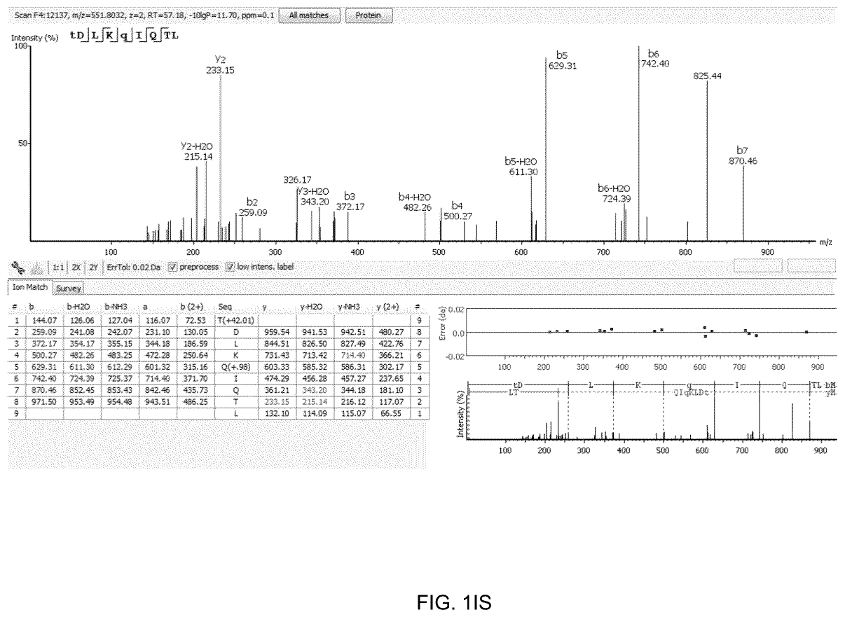

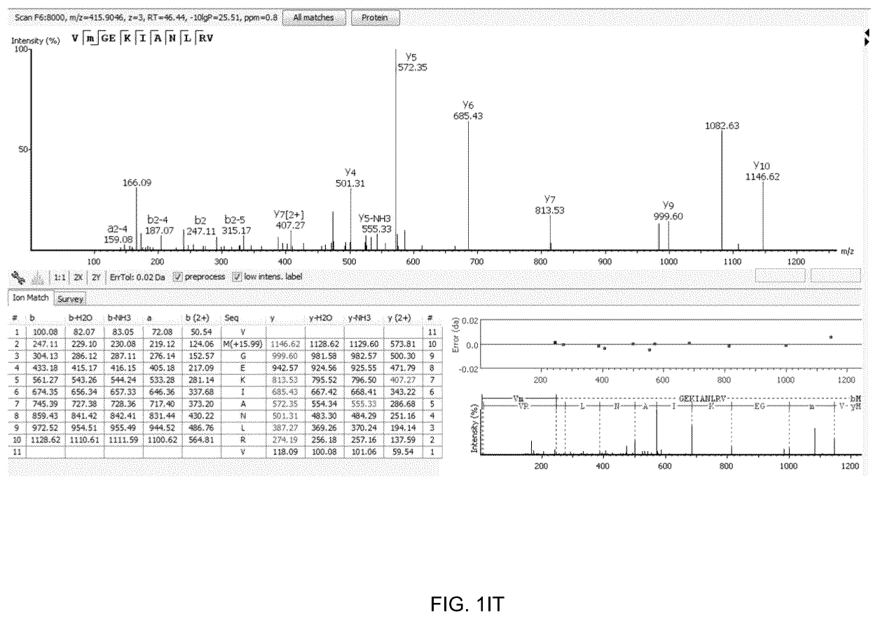

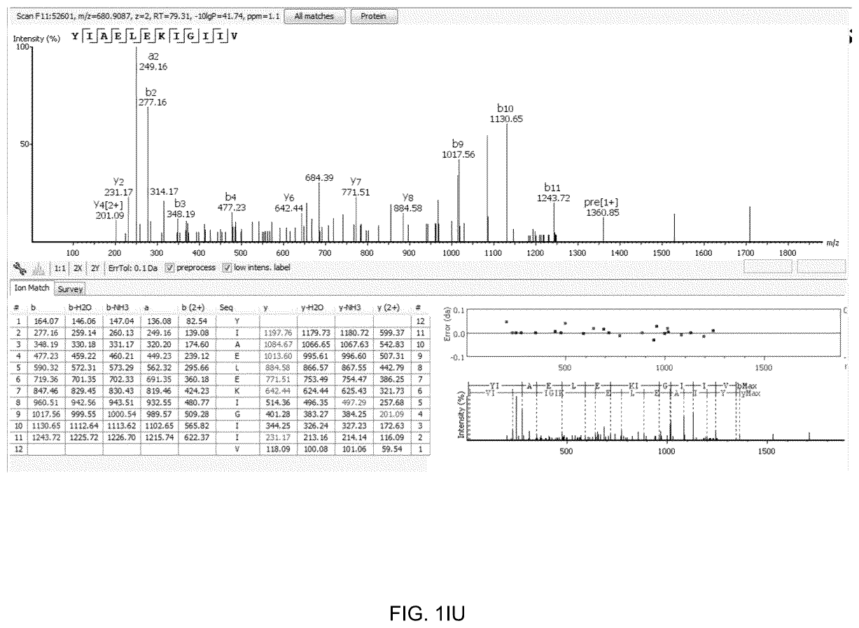

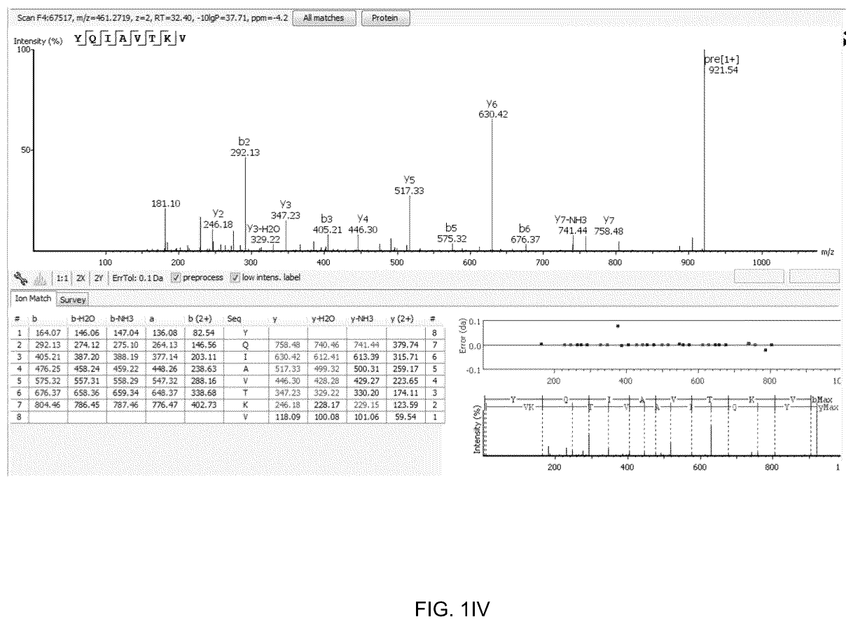

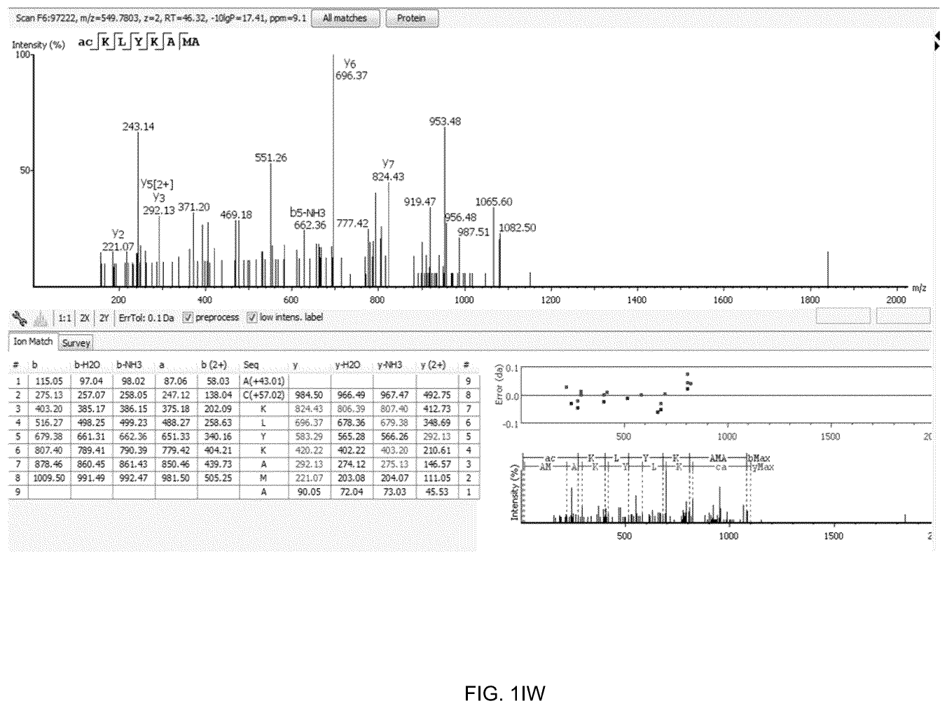

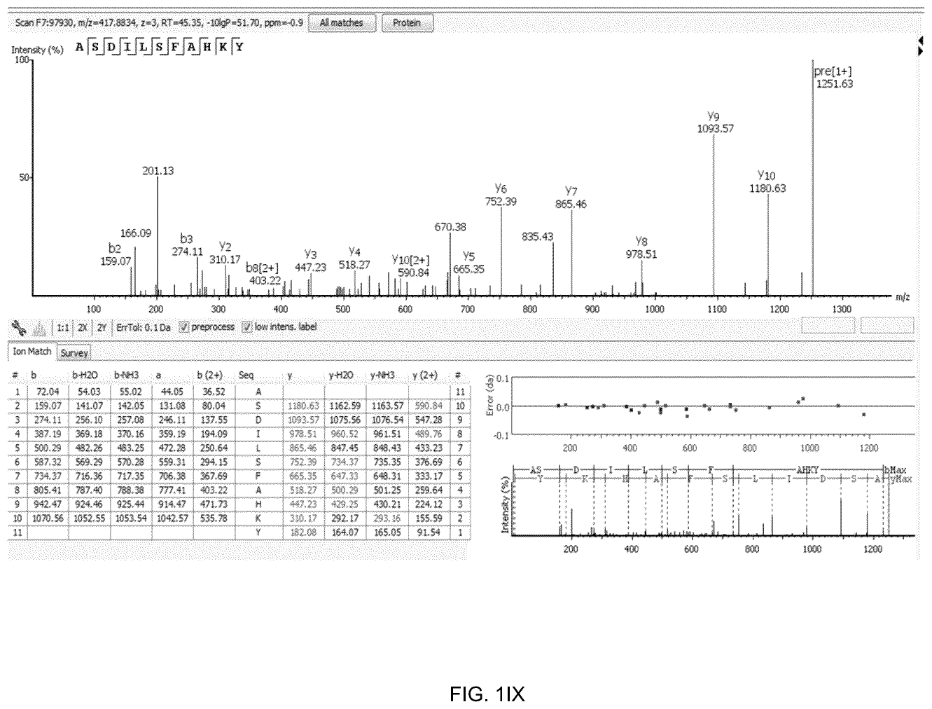

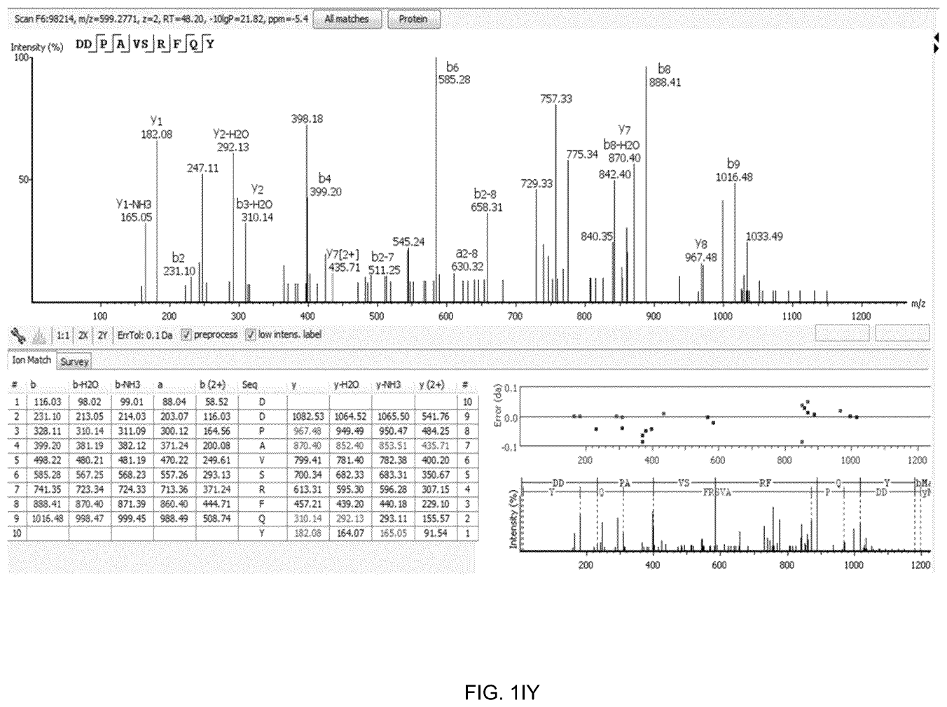

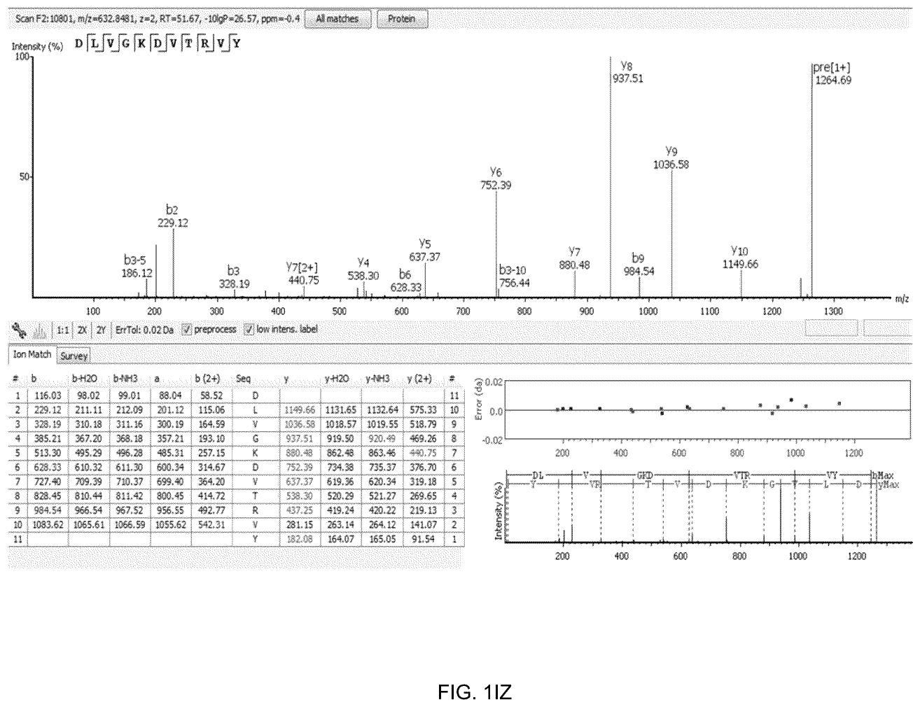

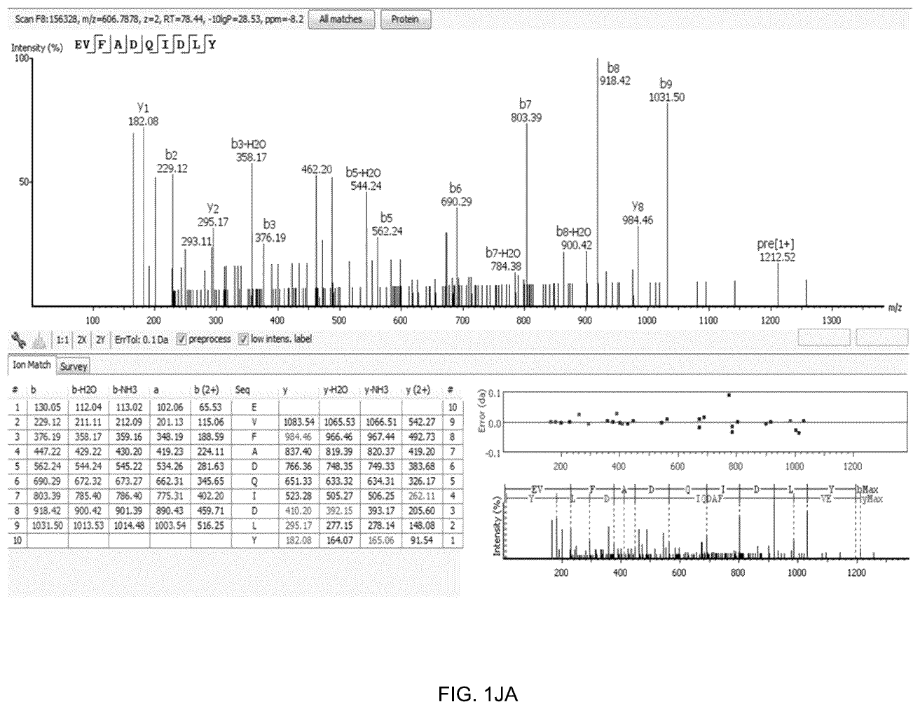

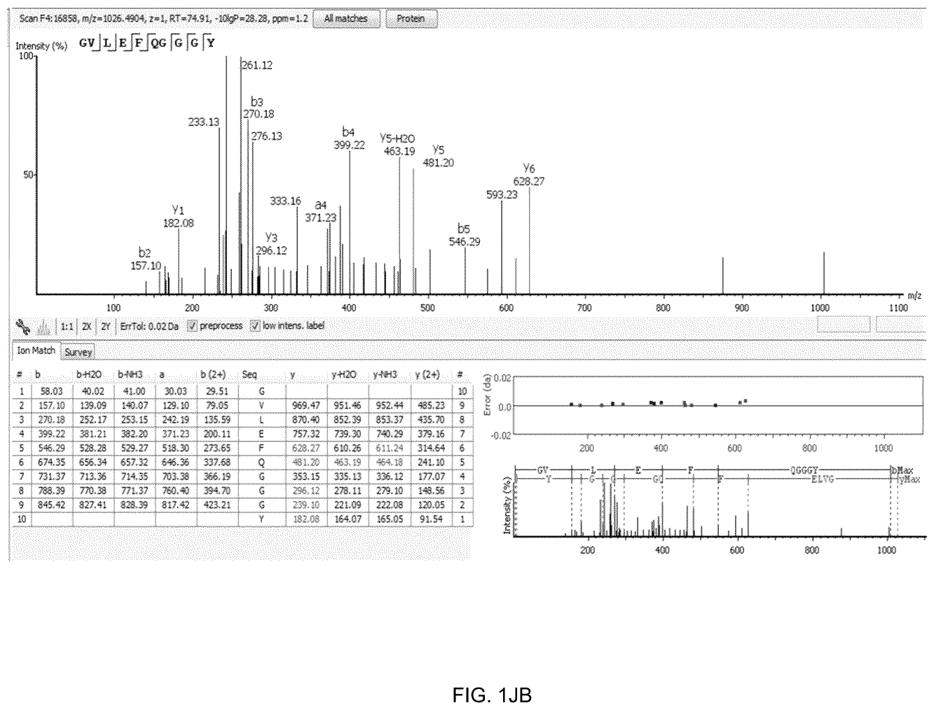

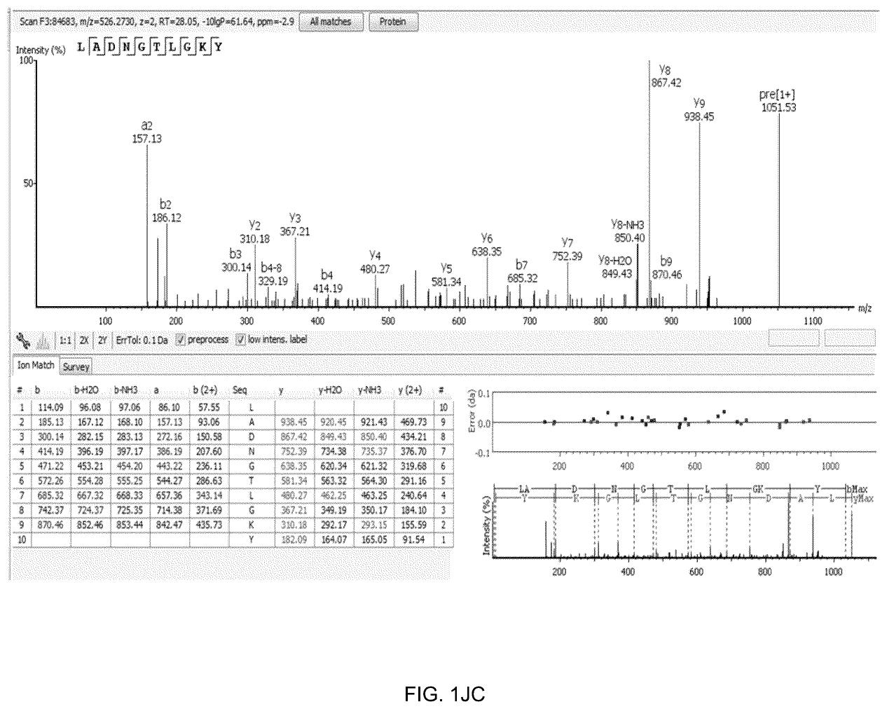

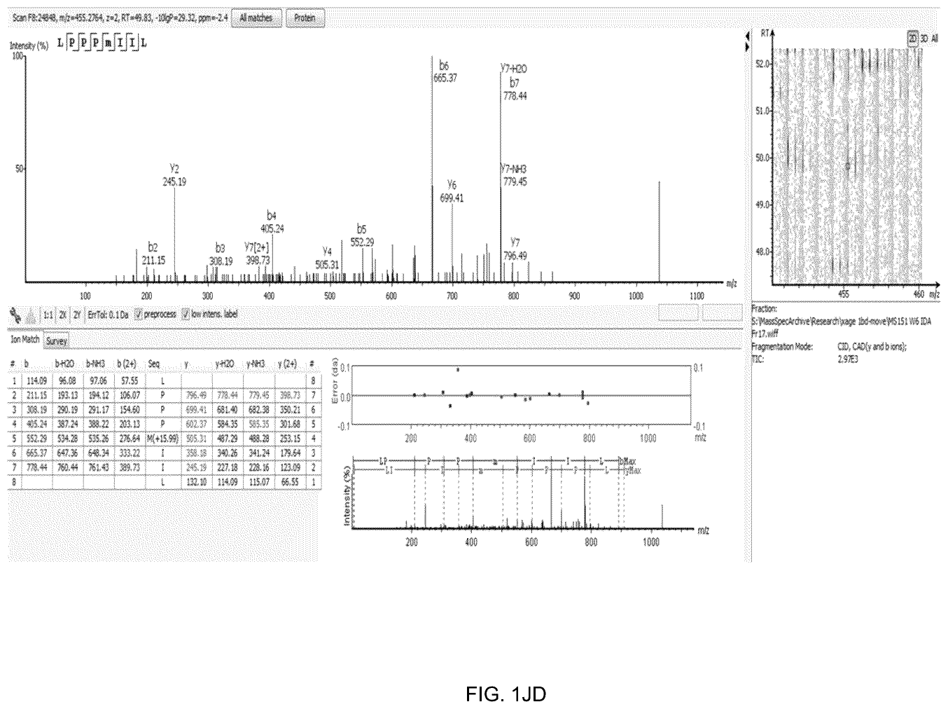

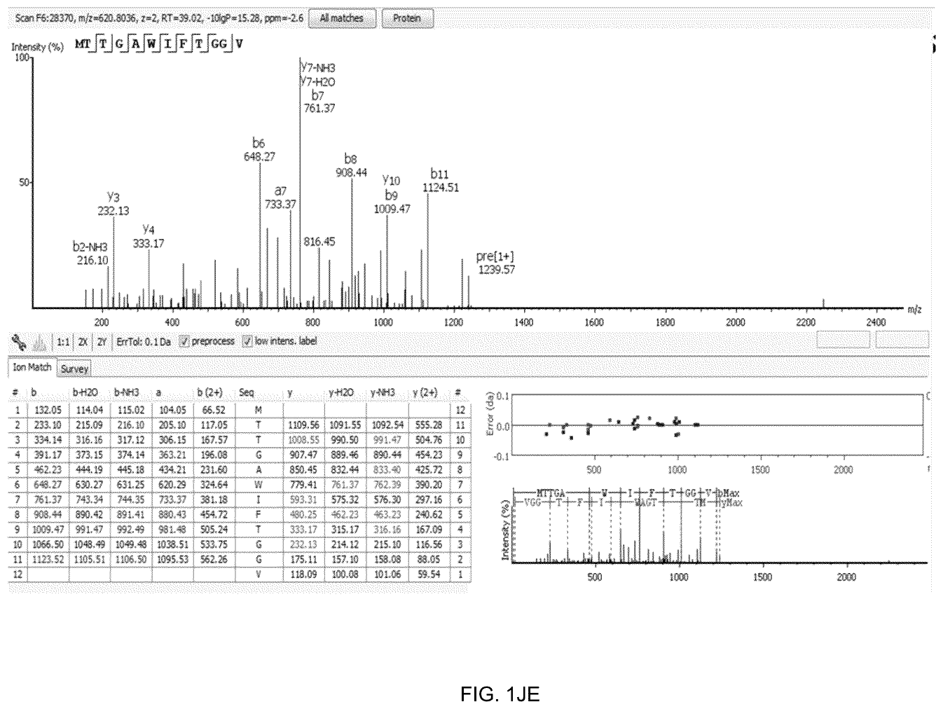

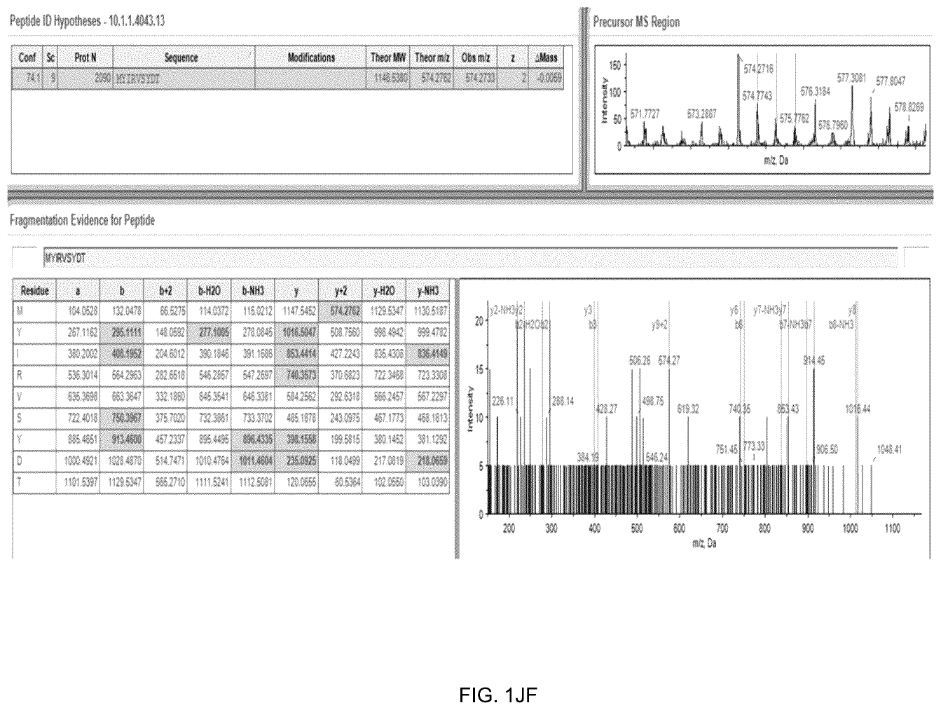

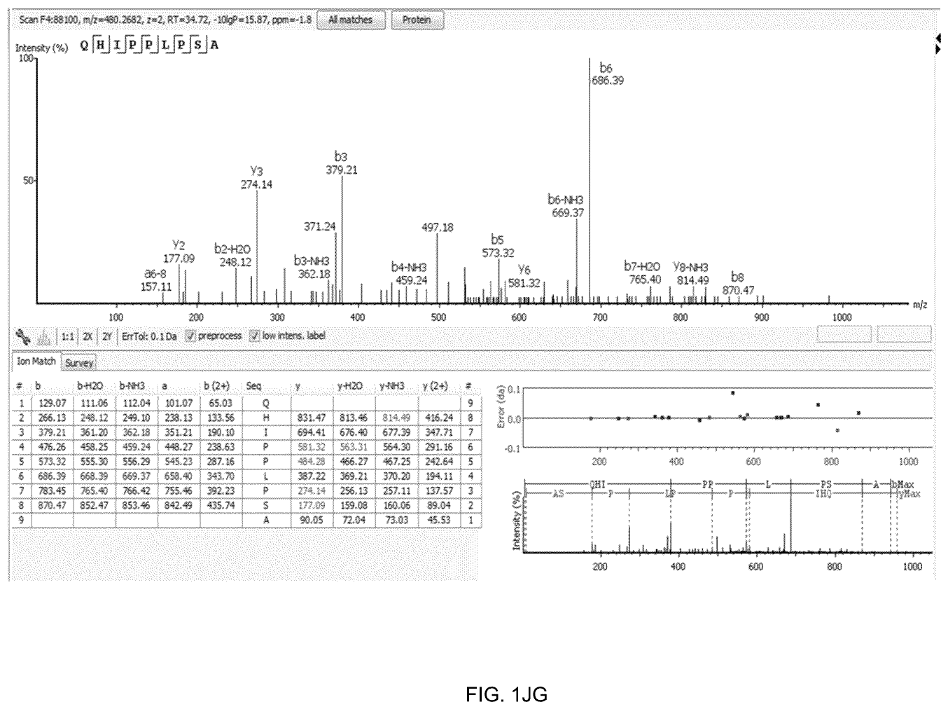

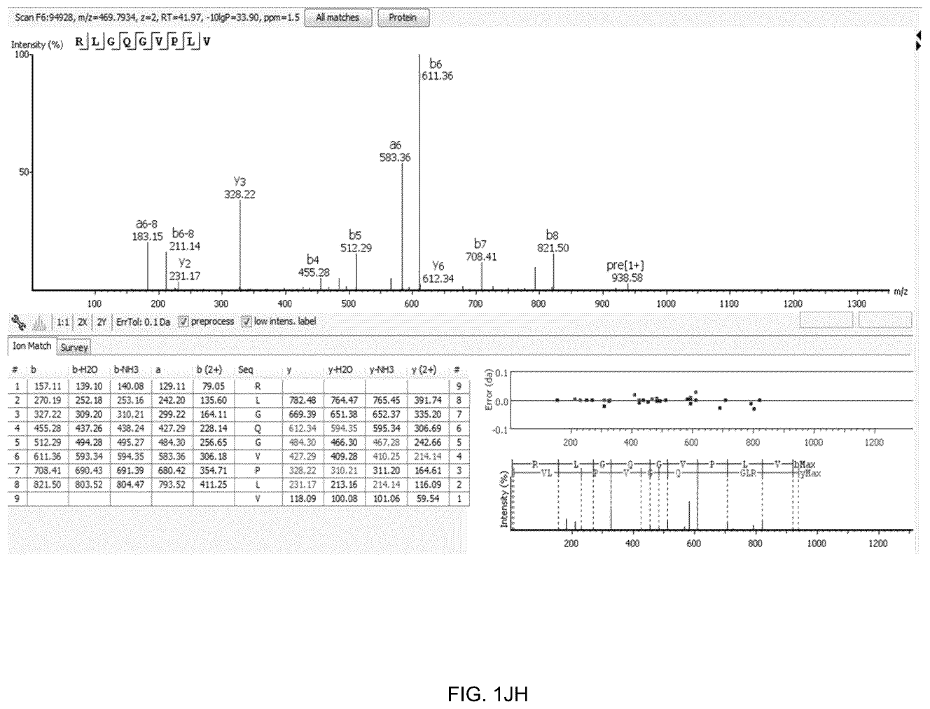

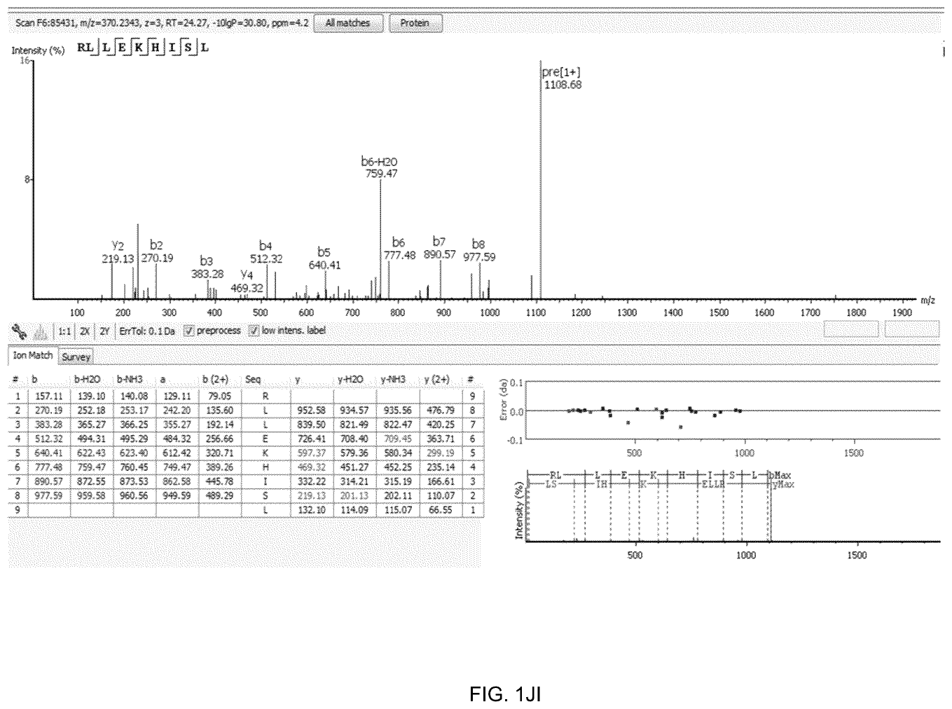

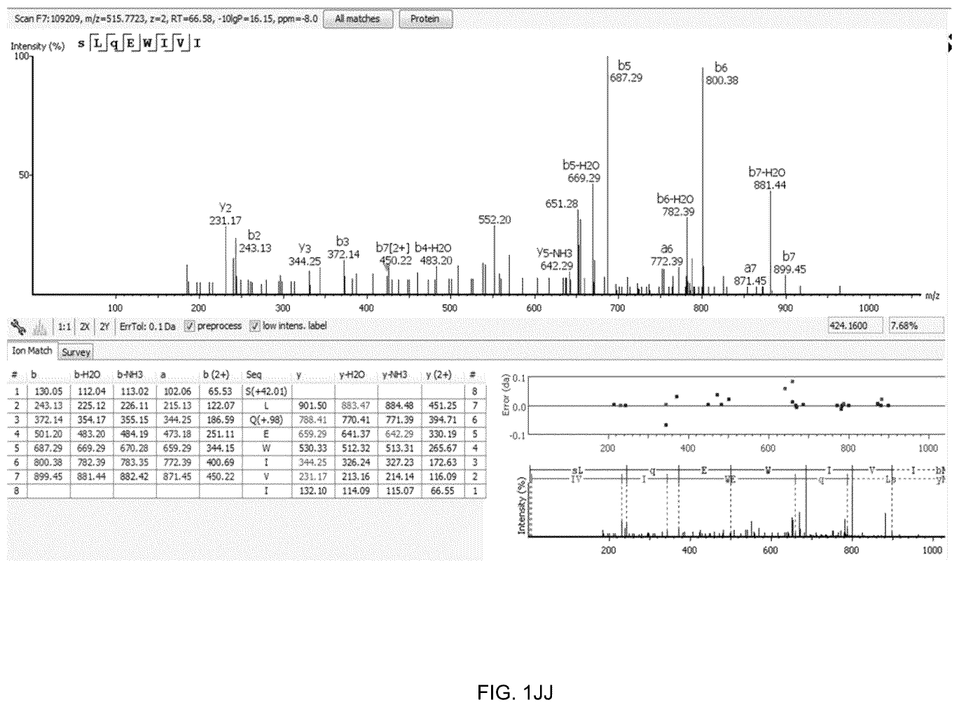

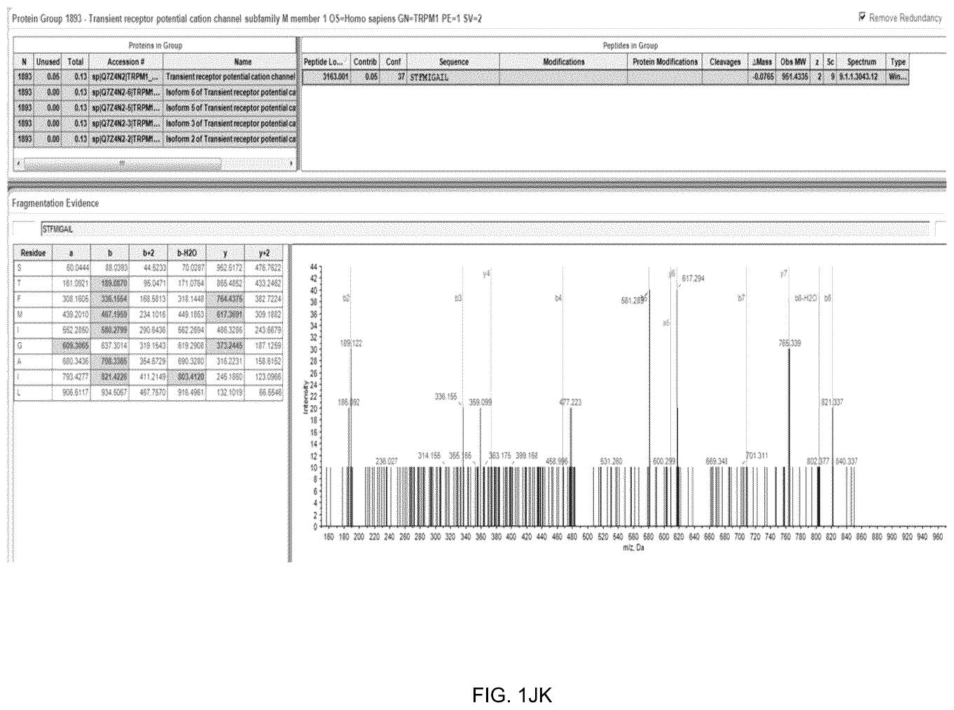

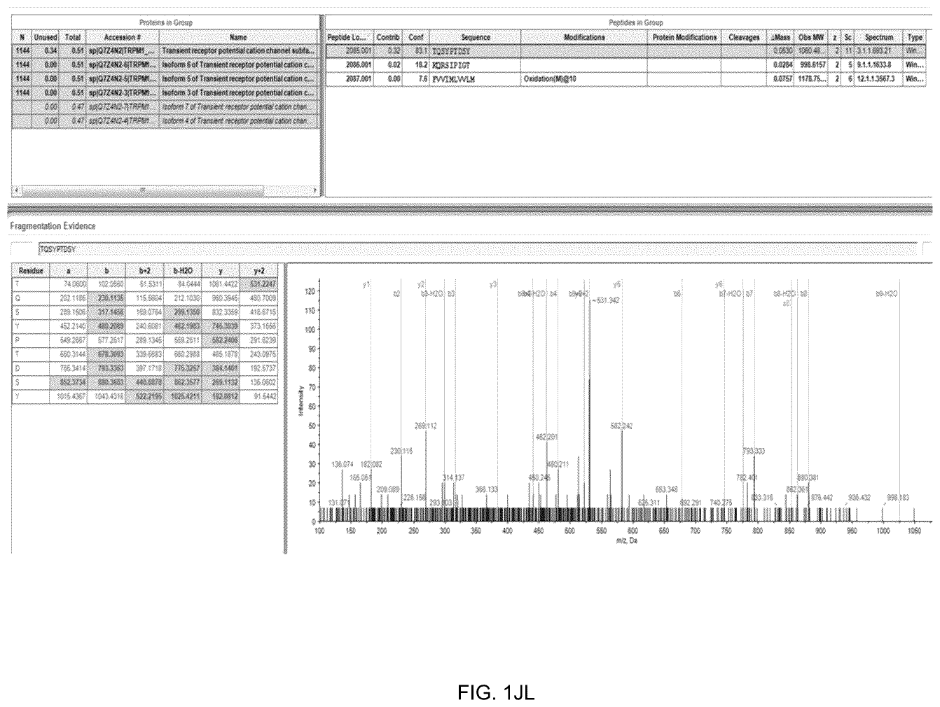

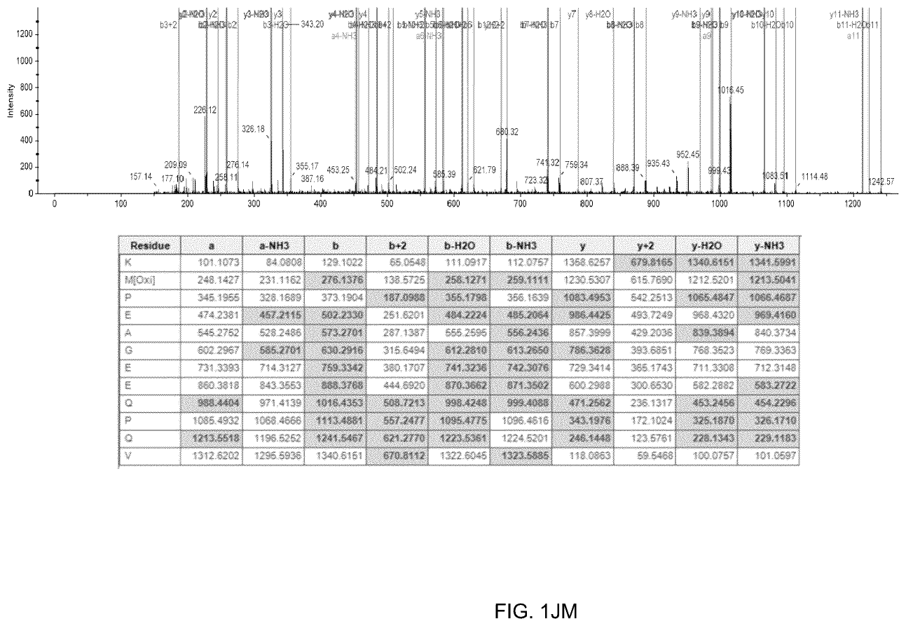

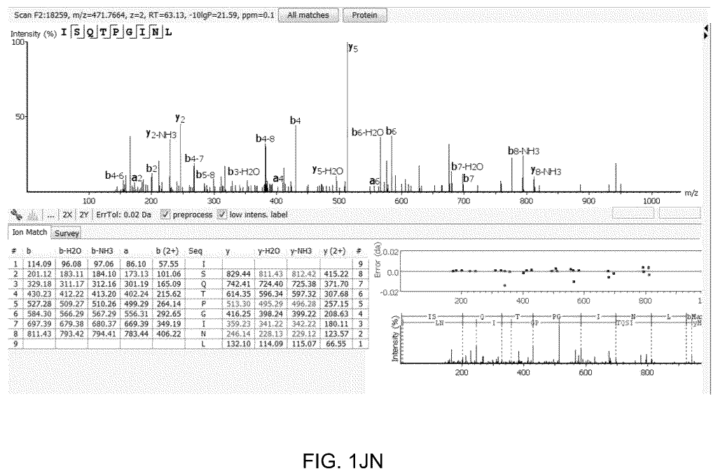

The following peptides were detected by mass spec following extraction from cancer cell lines.

| Target Protein | Peptides | Spectra |
|---|---|---|
| ACTL8 | Seq ID NOs: 1-6 | FIG. 1A-F |
| ASCL2 | Seq ID NOs: 7-21 | FIG. 1G-U |
| BRDT | Seq ID NOs: 22-24 | FIG. 1V-X |
| CALHM3 | Seq ID NOs: 25-29 | FIG. 1Y-AC |
| CLDN6 | Seq ID NOs: 30-34 | FIG. 1AD-AH |
| CT45A1 | Seq ID NOs: 35-39 | FIG. 1AI-AM |
| DCAF4L2 | Seq ID NOs: 40-42 | FIG. 1AN-AP |
| HOXB13 | Seq ID NOs: 43-45 | FIG. 1AQ-AS |
| HTR3A | Seq ID NOs: 46-51 | FIG. 1AT-AY |
| KLK3 | Seq ID NOs: 52 | FIG. 1AZ |
| KLK4 | Seq ID NOs: 53-58 | FIG. 1BA-BF |
| LGSN | Seq ID NOs: 59-61 | FIG. 1BG-BI |
| MAGEB2 | Seq ID NOs: 62-66 | FIG. 1BJ-BN |
| MAGEC1 | Seq ID NOs: 67-112 | FIG. 1BO-DH |
| NPSR1 | Seq ID NOs: 113-122 | FIG. 1DI-DR |
| NR0B1 | Seq ID NOs: 123-141 | FIG. 1DS-EK |
| PAEP | Seq ID NOs: 142-149 | FIG. 1EL-ES |
| PAGE2 | Seq ID NOs: 150-160 | FIG. 1ET-FD |
| PAGE5 | Seq ID NOs: 161-169 | FIG. 1FE-FM |
| PIWIL1 | Seq ID NOs: 170-186 | FIG. 1FN-GD |
| RLN1 | Seq ID NOs: 187-191 | FIG. 1GE-GI |
| SAGE1 | Seq ID NOs: 192-198 | FIG. 1GJ-GP |
| SLC30A8 | Seq ID NOs: 199-205 | FIG. 1GQ-GW |
| SLC45A2 | Seq ID NOs: 206-242 | FIG. 1GX-IH |
| SMC1B | Seq ID NOs: 243-256 | FIG. 1II-IV |
| TRPM1 | Seq ID NOs: 257-272 | FIG. 1IW-JL |
| XAGE1 | Seq ID NOs: 272-274 | FIG. 1JM-JN |

FIG. 1 shows representative fragmentation patterns for each of the peptides of SEQ ID NOs: 1-274 respectively. A table highlighting the matching ions is shown below each spectrum.

### Example 2—Preparation of Recombinant Peptide-HLA Complexes

The following describes a suitable method for the preparation of soluble recombinant HLA loaded with TAA peptide.

Class I HLA molecules (HLA-heavy chain and HLA light-chain (β2m)) were expressed separately in E. coli as inclusion bodies, using appropriate constructs. HLA-heavy chain additionally contained a C-terminal biotinylation tag which replaces the transmembrane and cytoplasmic domains (O'Callaghan et al. (1999) Anal. Biochem. 266: 9-15). E. coli cells were lysed and inclusion bodies processed to approximately 80% purity.

Inclusion bodies of β2m and heavy chain were denatured separately in denaturation buffer (6 M guanidine, 50 mM Tris pH 8.1, 100 mM NaCl, 10 mM DTT, 10 mM EDTA) for 30 mins at 37° C. Refolding buffer was prepared containing 0.4 M L-Arginine, 100 mM Tris pH 8.1, 2 mM EDTA, 3.1 mM cystamine dihydrochloride, 7.2 mM cysteamine hydrochloride. Synthetic peptide was dissolved in DMSO to a final concentration of 4mg/ml and added to the refold buffer at 4 mg/litre (final concentration). Then 30 mg/litre β2m followed by 60 mg/litre heavy chain (final concentrations) are added. Refolding was allowed to reach completion at room temperature for at least 1 hour.

The refold mixture was then dialysed against 20 L of deionised water at 4° C. for 16 h, followed by 10 mM Tris pH 8.1 for a further 16 h. The protein solution was then filtered through a 0.45 pm cellulose acetate filter and loaded onto a POROS HQ anion exchange column (8 ml bed volume) equilibrated with 20 mM Tris pH 8.1. Protein was eluted with a linear 0-500 mM NaCl gradient using an AKTA purifier (GE Healthcare). HLA-peptide complex eluted at approximately 250 mM NaCl, and peak fractions were collected, a cocktail of protease inhibitors (Calbiochem) was added and the fractions were chilled on ice.

Biotinylation tagged pHLA molecules were buffer exchanged into 10 mM Tris pH 8.1, 5 mM NaCl using a GE Healthcare fast desalting column equilibrated in the same buffer. Immediately upon elution, the protein-containing fractions were chilled on ice and protease inhibitor cocktail (Calbiochem) was added. Biotinylation reagents were then added: 1 mM biotin, 5 mM ATP (buffered to pH 8), 7.5 mM MgCl2, and 5 μg/mIBirA enzyme (purified according to O'Callaghan et al., (1999) Anal. Biochem. 266: 9-15). The mixture was then allowed to incubate at room temperature overnight.

The biotinylated pHLA molecules were further purified by gel filtration chromatography using an AKTA purifier with a GE Healthcare Superdex 75 HR 10/30 column pre-equilibrated with filtered PBS. The biotinylated pHLA mixture was concentrated to a final volume of 1 ml loaded onto the column and was developed with PBS at 0.5 ml/min. Biotinylated pHLA molecules eluted as a single peak at approximately 15 ml. Fractions containing protein were pooled, chilled on ice, and protease inhibitor cocktail was added. Protein concentration was determined using a Coomassie-binding assay (PerBio) and aliquots of biotinylated pHLA molecules were stored frozen at −20 ° C.

Such peptide-MHC complexes may be used in soluble form or may be immobilised through their C terminal biotin moiety on to a solid support, to be used for the detection of T cells and T cell receptors which bind said complex. For example, such complexes can be used in panning phage libraries, performing ELISA assays and preparing sensor chips for Biacore measurements.

### Example 3—Identification of TCRs that Bind to a Peptide-MHC Complex of the Invention

### Method

Antigen binding TCRs were obtained using peptides of the invention to pan a TCR phage library. The library was constructed using alpha and beta chain sequences obtained from a natural repertoire (as described in WO2015/136072, PCT/EP2016/071757, PCT/EP2016/071761, PCT/EP2016/071762, PCT/EP2016/071765, PCT/EP2016/071767, PCT/EP2016/071768, PCT/EP2016/071771 or PCT/EP2016/071772). The random combination of these alpha and beta chain sequences, which occurs during library creation, produces a non-natural repertoire of alpha beta chain combinations.

TCRs obtained from the library were assessed by ELISA to confirm specific antigen recognition. ELISA assays were performed as described in WO2015/136072. Briefly, 96 well MaxiSorp ELISA plates were coated with streptavidin and incubated with the biotinylated peptide-HLA complex of the invention. TCR bearing phage clones were added to each well and detection carried out using an anti-Mβ-HRP antibody conjugate. Bound antibody was detected using the KPL labs TMB Microwell peroxidase Substrate System. The appearance of a blue colour in the well indicated binding of the TCR to the antigen. An absence of binding to alternative peptide-HLA complexes indicated the TCR is not highly cross reactive.

Further confirmation that TCRs are able to bind a complex of comprising a peptide HLA complex of the invention can be obtained by surface plasmon reasonance (SPR) using isolated TCRs. In this case alpha and beta chain sequences are expressed in E. coli as soluble TCRs, (WO2003020763; Boulter, et al., Protein Eng, 2003. 16: 707-711). Binding of the soluble TCRs to the complexes is analysed by surface plasmon resonance using a BiaCore 3000 instrument. Biotinylated peptide-HLA monomers are prepared as previously described (Example 2) and immobilized on to a streptavidin-coupled CM-5 sensor chip. All measurements are performed at 25° C. in PBS buffer supplemented with 0.005% Tween at a constant flow rate. To measure affinity, serial dilutions of the soluble TCRs are flowed over the immobilized peptide-MHCs and the response values at equilibrium determined for each concentration. Data are analysed by plotting the specific equilibrium binding against protein concentration followed by a least squares fit to the Langmuir binding equation, assuming a 1:1 interaction.

### Results

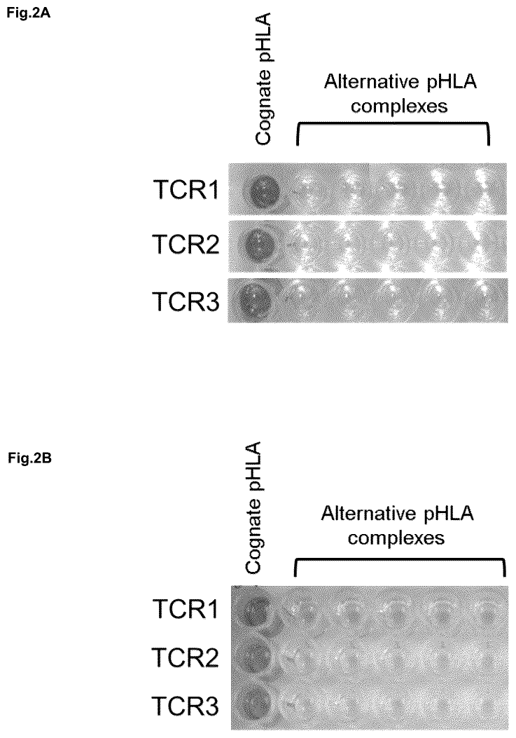

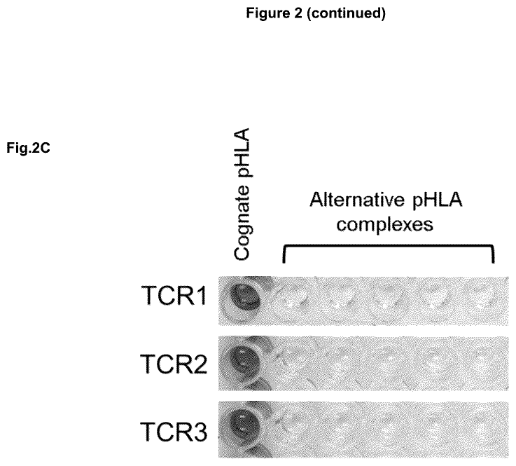

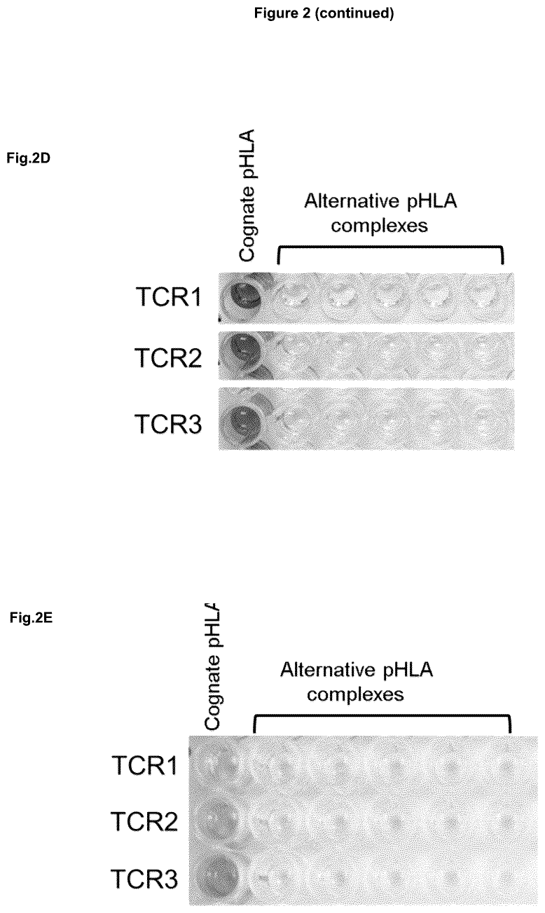

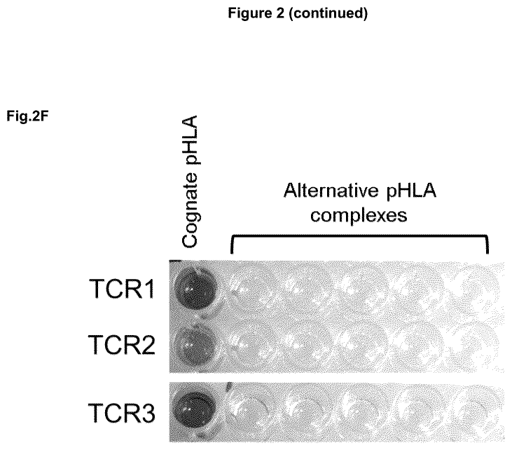

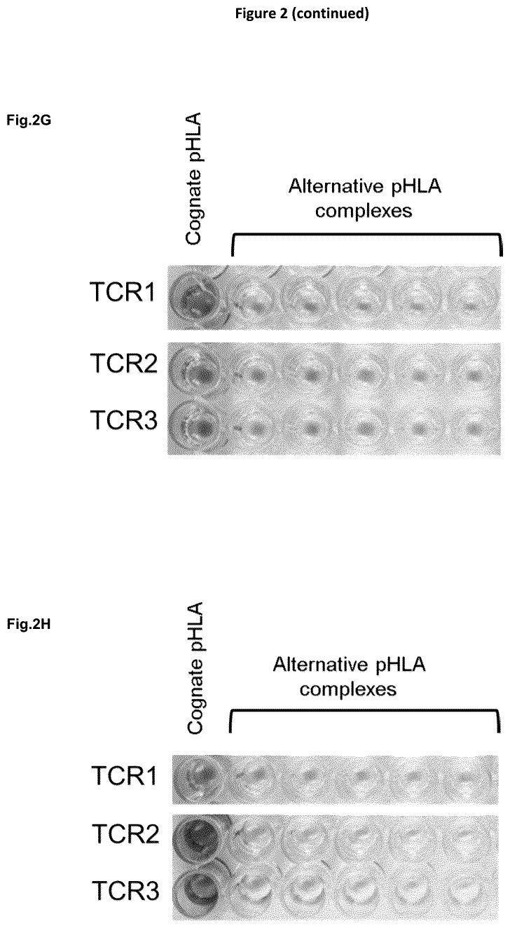

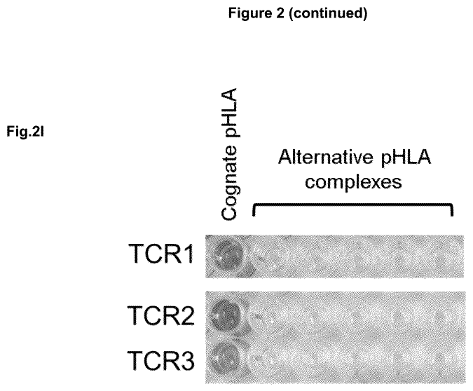

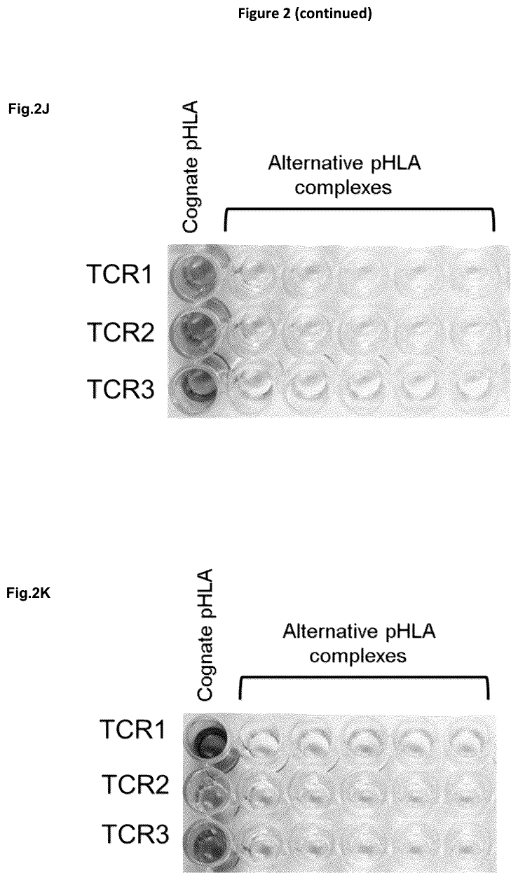

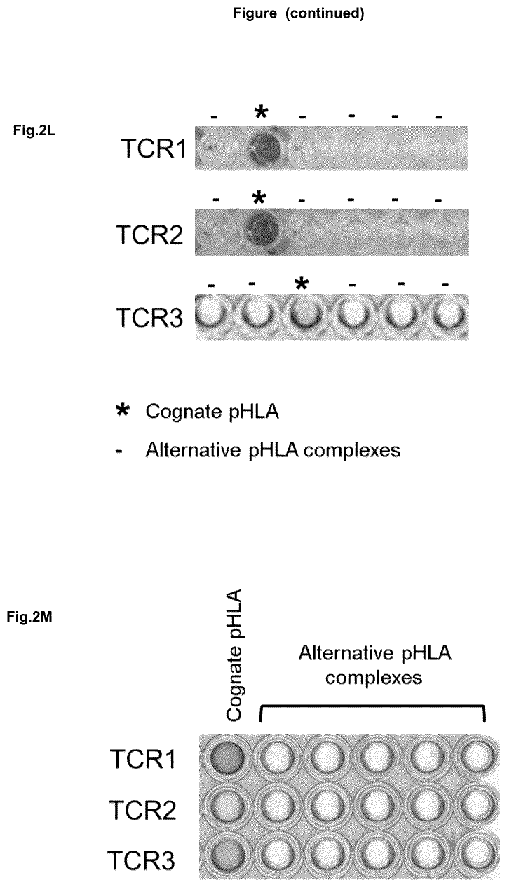

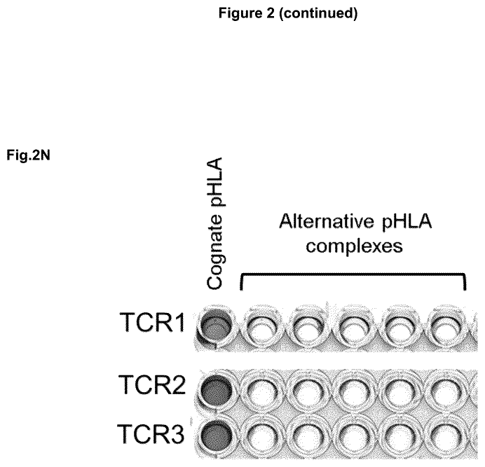

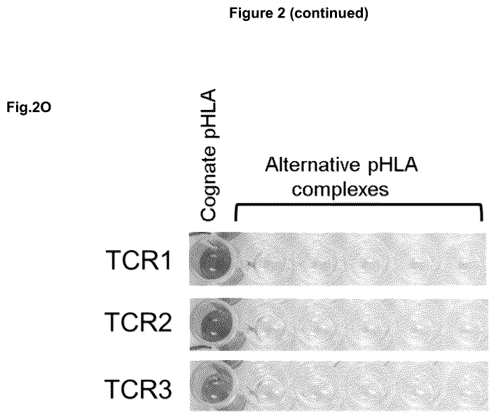

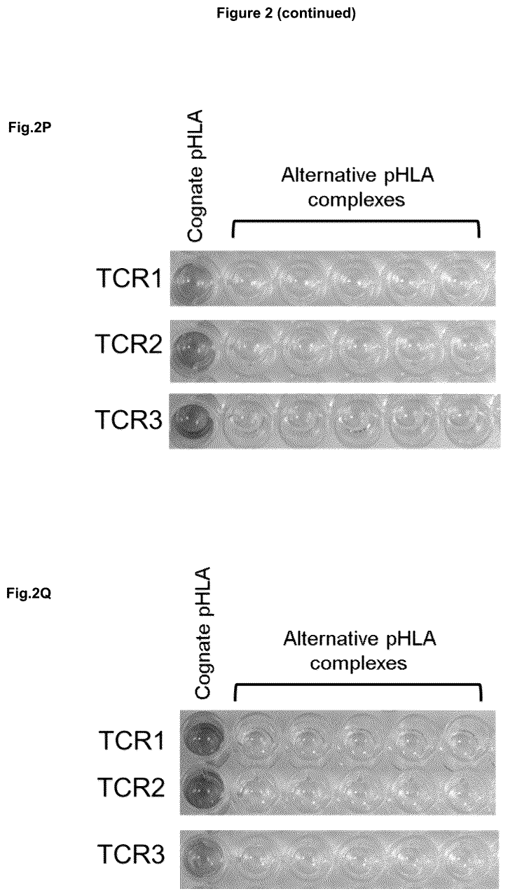

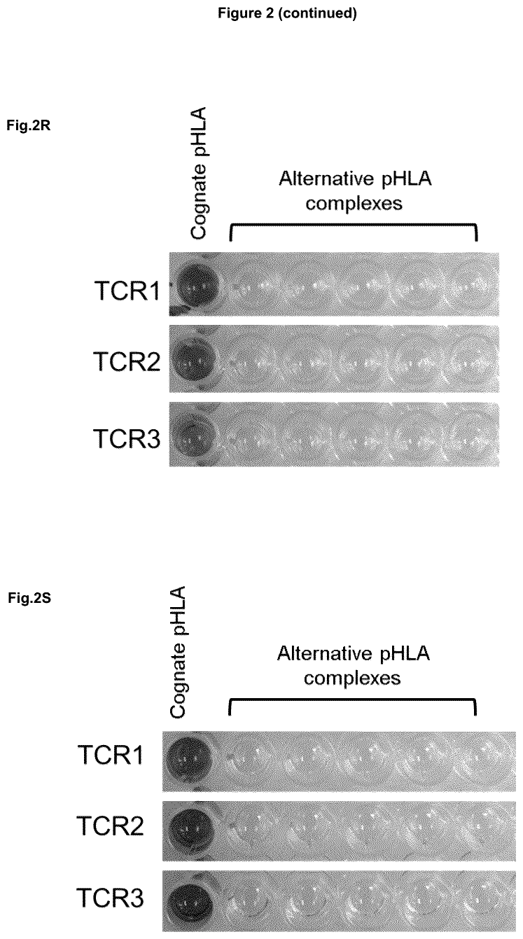

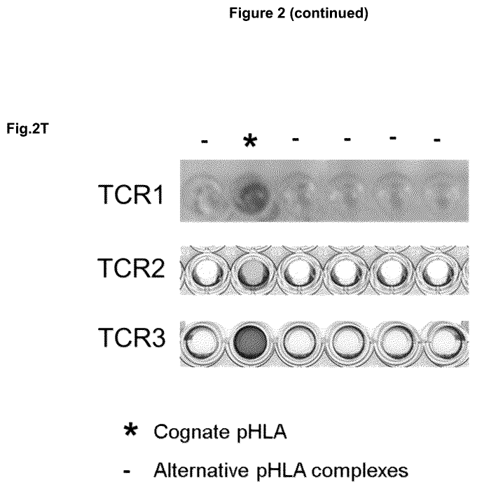

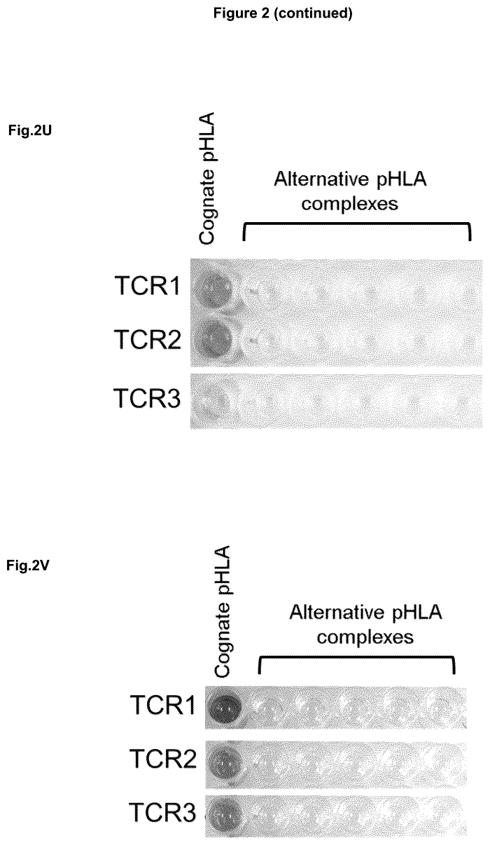

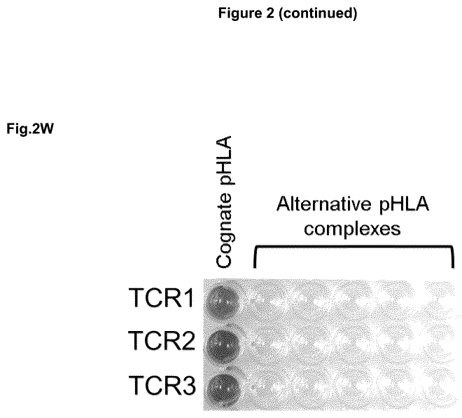

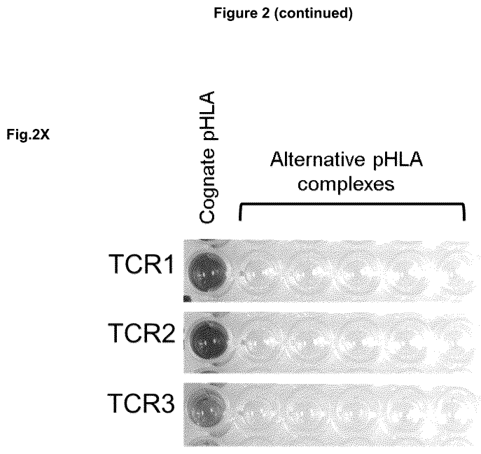

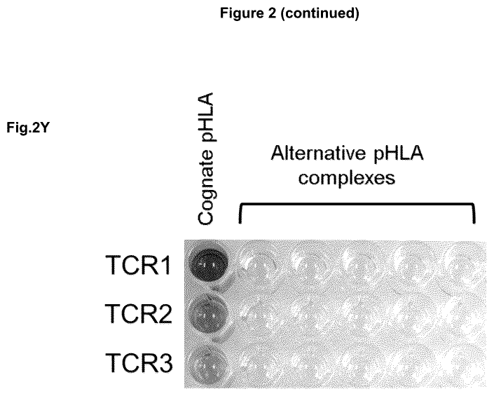

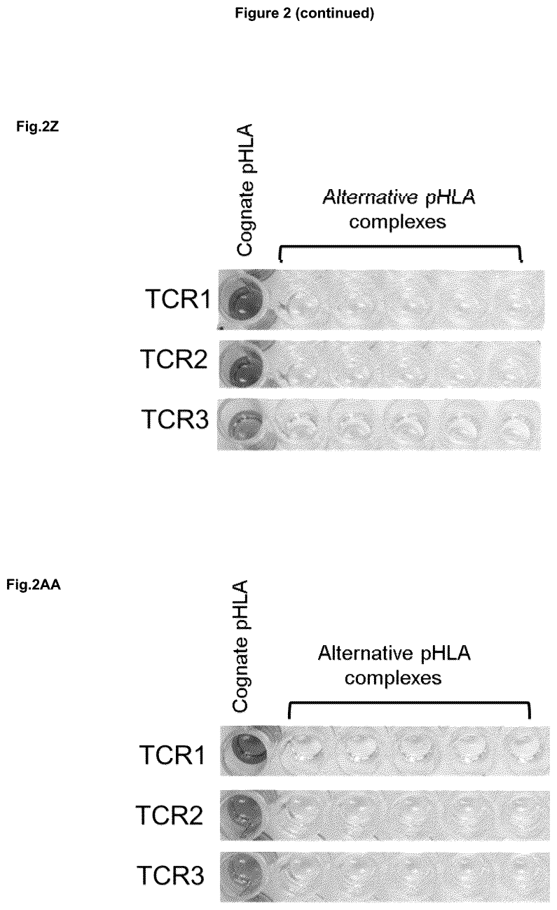

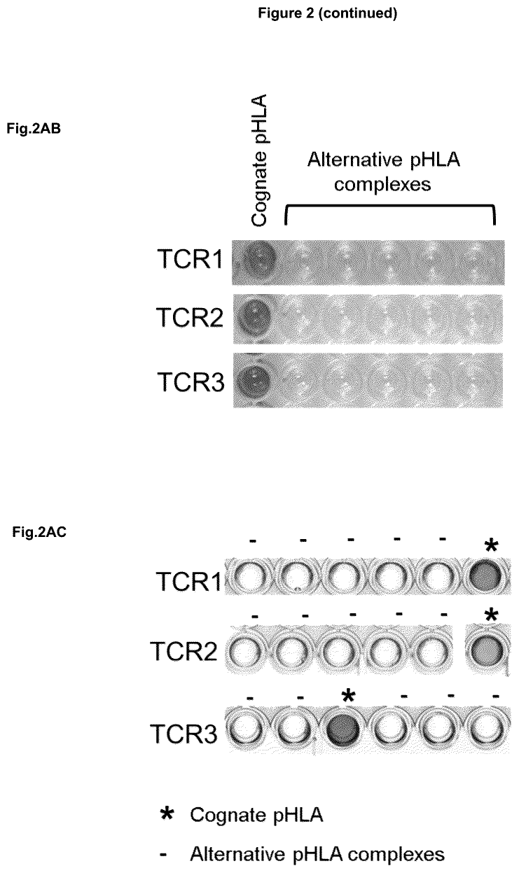

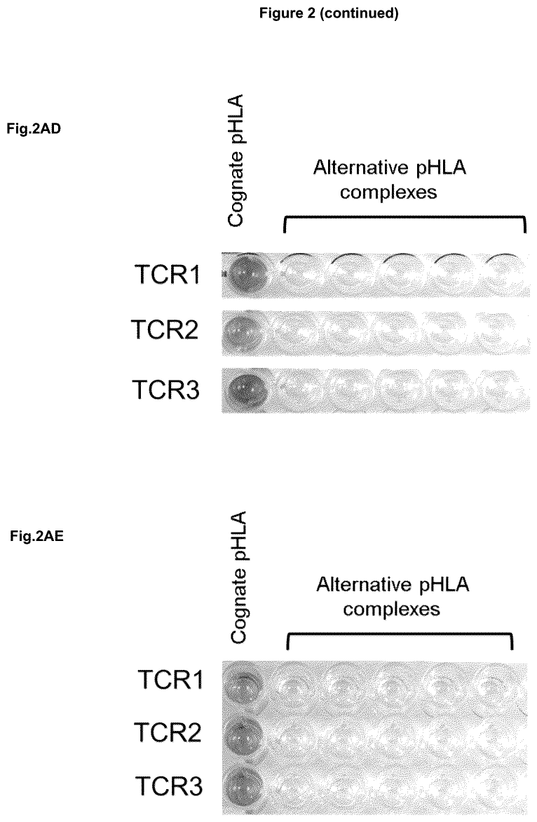

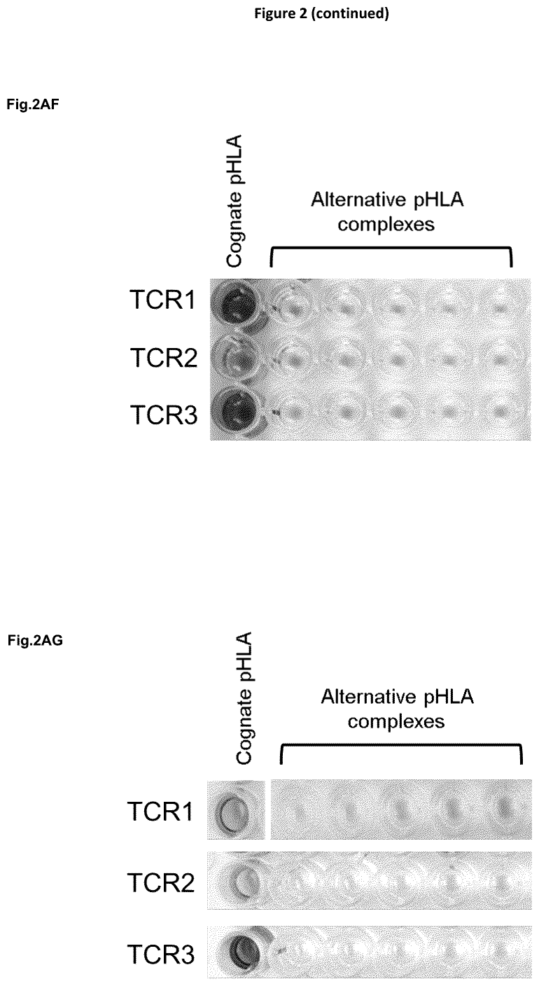

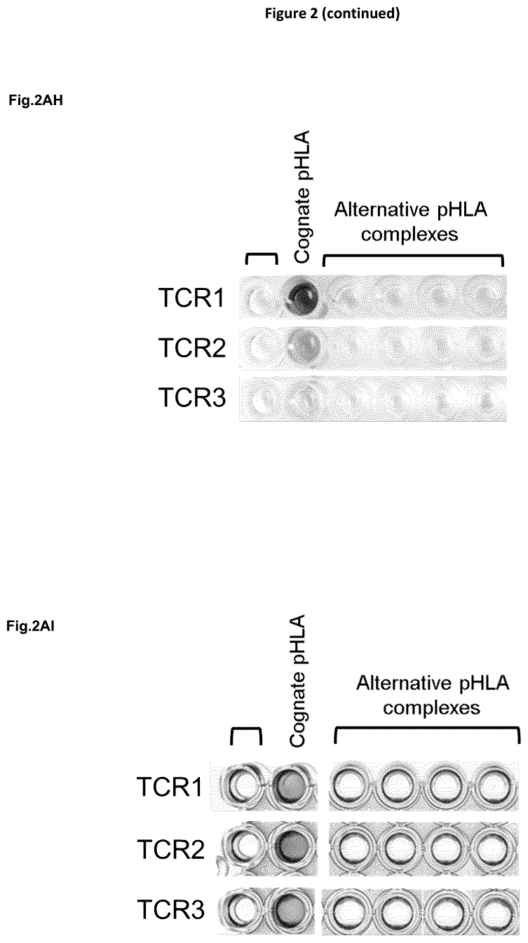

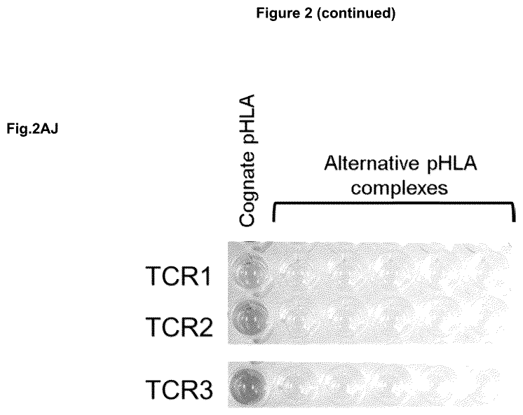

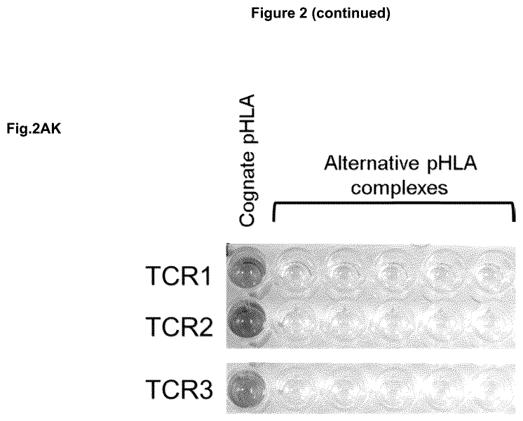

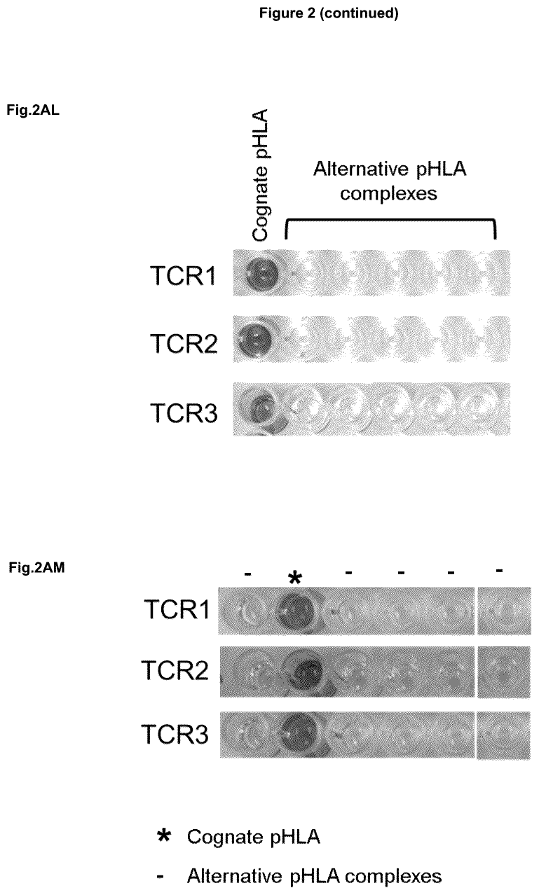

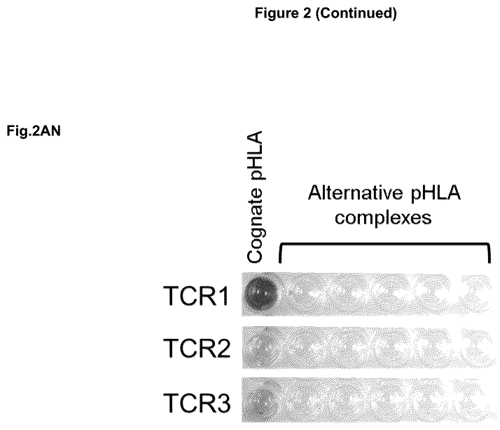

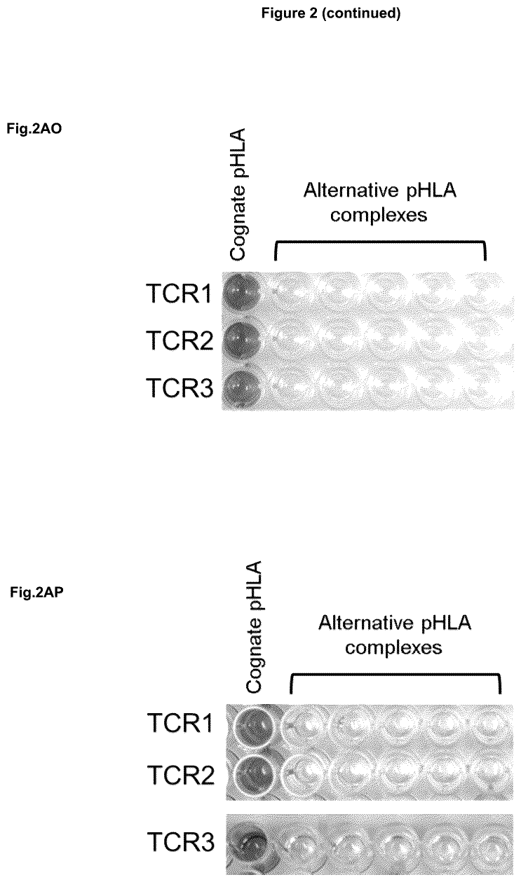

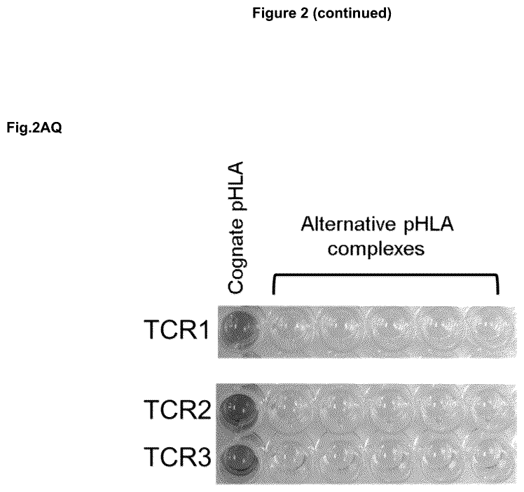

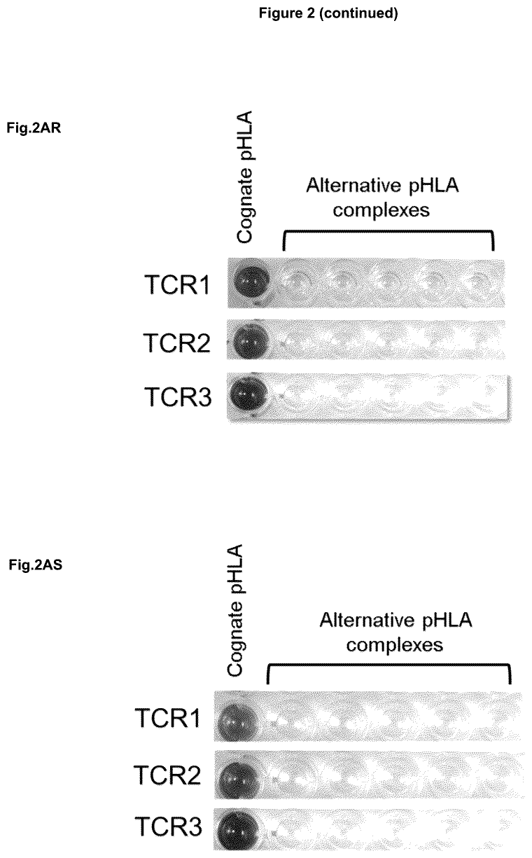

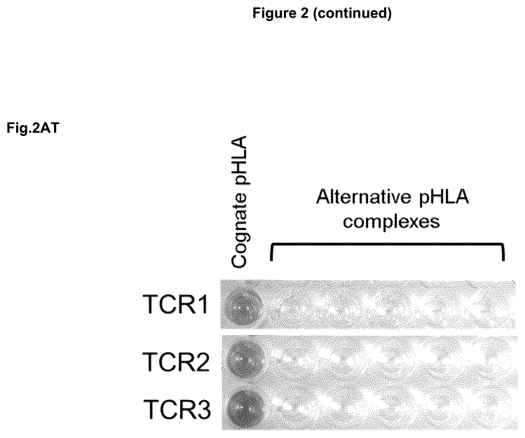

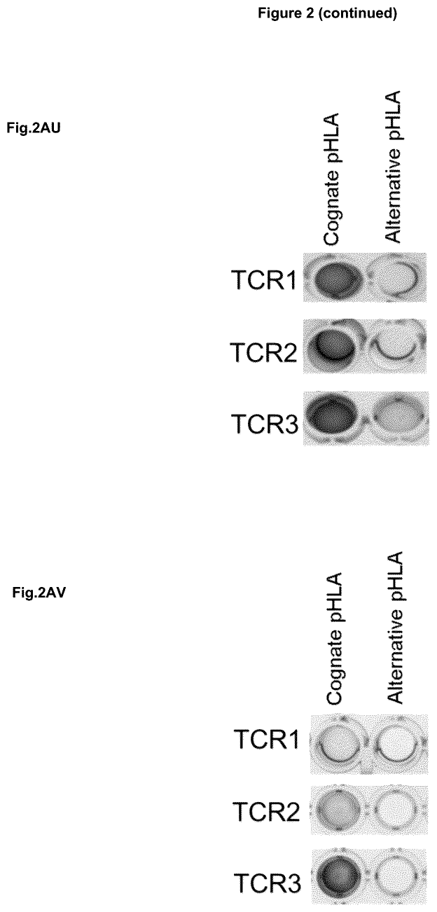

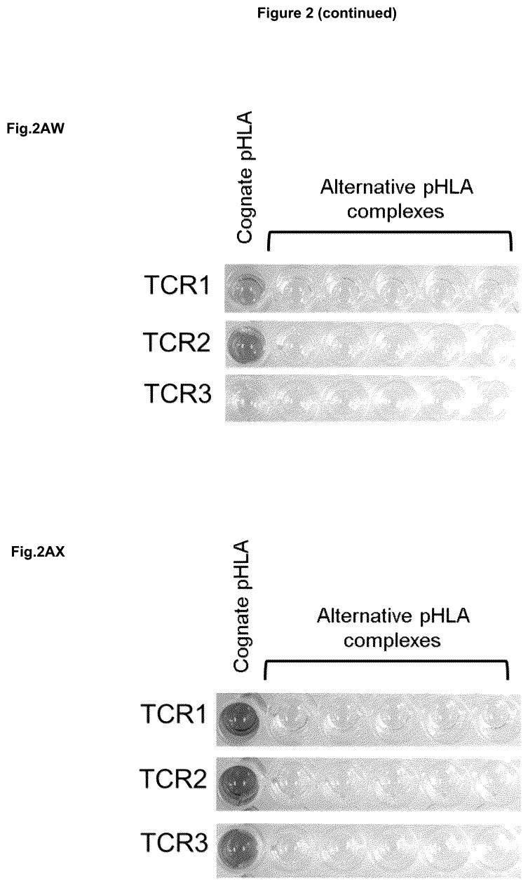

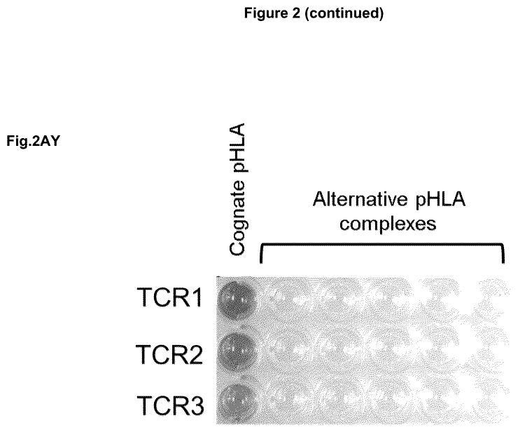

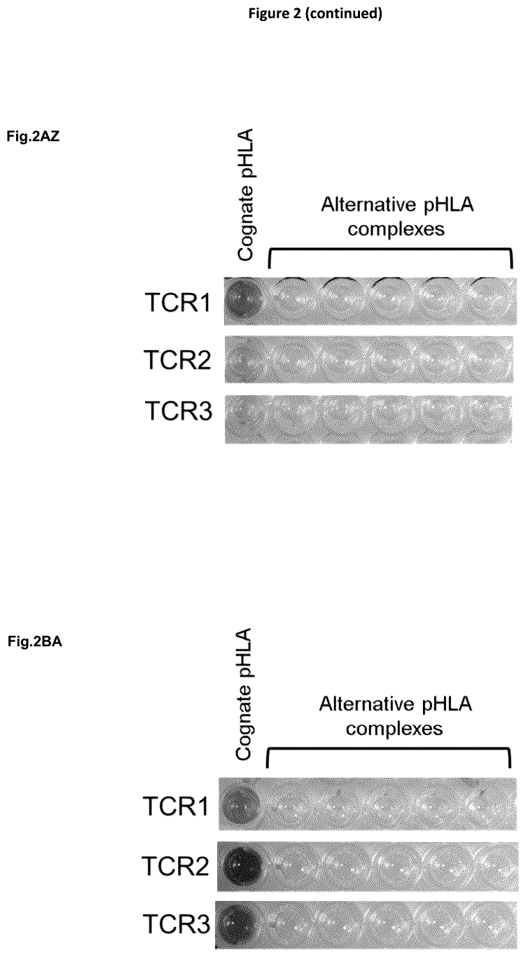

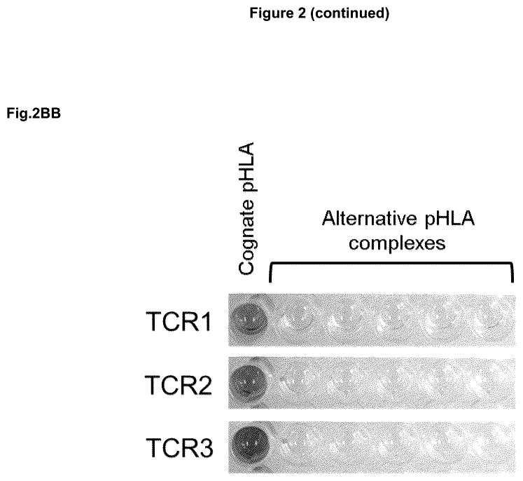

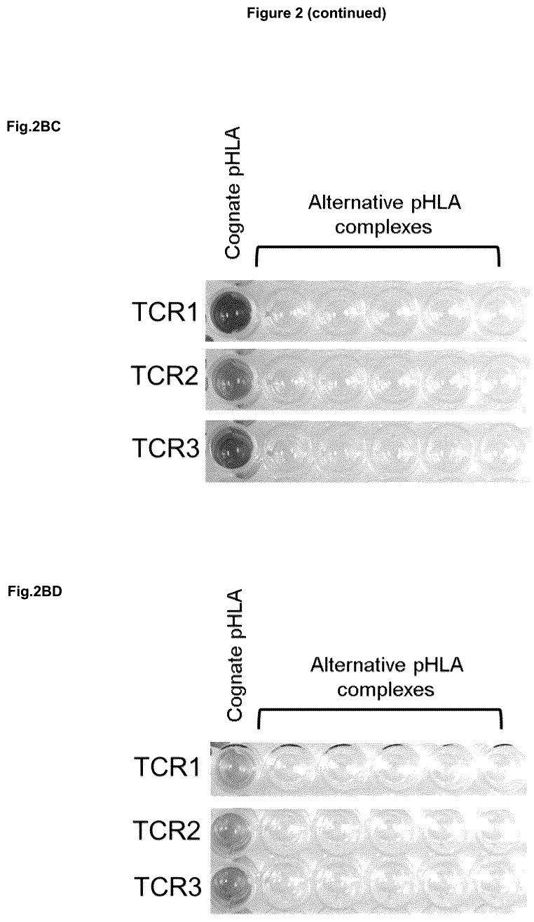

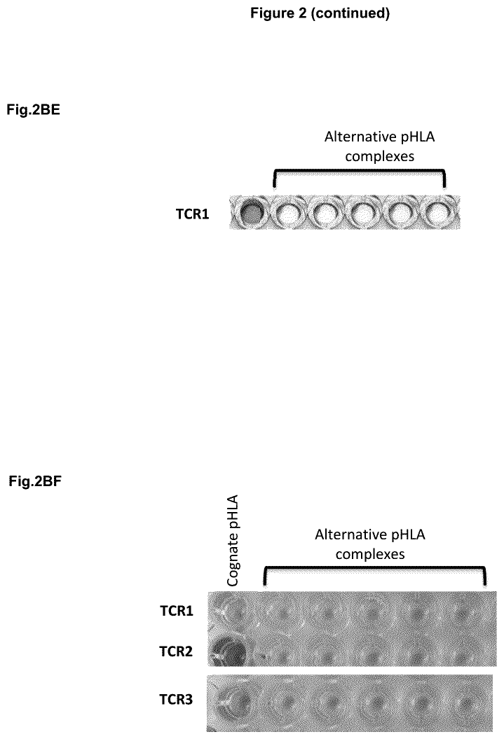

TCRs that specifically recognise the following peptides in complex with HLA complexes were obtained from the library. FIG. 2 shows ELISA data for such TCRs.

| SEQ ID | Peptide | ELISA data |
|---|---|---|
| 1 | ALDESNTYQL | FIG. 2A |
| 2 | SLYASGLLTGV | FIG. 2B |
| 3 | RCLFQLETV | FIG. 2C |
| 9 | LLAEHDAVRNAL | FIG. 2D |
| 11 | ALSPAERELL | FIG. 2E |
| 16 | LLAEHDAVRNA | FIG. 2F |
| 22 | EMFPKFTEV | FIG. 2G |
| 23 | RLLDVNNQL | FIG. 2H |
| 26 | ALLDGKCFV | FIG. 21 |
| 31 | TLIPVCWTA | FIG. 2J |
| 32 | VLTSGIVFV | FIG. 2K |
| 36 | SLIAGSAMSKA | FIG. 2L |
| 38 | KLVKELRCV | FIG. 2M |
| 39 | KIFEMLEGV | FIG. 2N |
| 41 | ILQDGQFLV | FIG. 20 |
| 44 | SLSERQITI | FIG. 2P |
| 45 | YLDVSVVQT | FIG. 2Q |
| 47 | AILNVDEKNQV | FIG. 2R |
| 51 | SLAETIFIV | FIG. 2S |
| 52 | KVMDLPTQEPAL | FIG. 2T |
| 53 | KLYDPLYHPSM | FIG. 2U |
| 55 | SIASQCPTA | FIG. 2V |
| 56 | FQNSYTIGL | FIG. 2W |
| 59 | ALGETFIRYFV | FIG. 2X |
| 66 | KVLEFLAKV | FIG. 2Y |
| 71 | ILQSSPESA | FIG. 2Z |
| 75 | FLAMLKNTV | FIG. 2AA |
| 114 | AINPLIYCV | FIG. 2AB |
| 121 | VIIQNLPAL | FIG. 2AC |
| 128 | GLLKTLRFV | FIG. 2AD |
| 129 | GLPGGRPVAL | FIG. 2AE |
| 142 | AMATNNISL | FIG. 2AF |
| 146 | LLDTDYDNFL | FIG. 2AG |
| 147 | MMCQYLARV | FIG. 2AH |
| 149 | TLLDTDYDNFL | FIG. 2AI |
| 158 | IMPTFDLTKV | FIG. 2AJ |
| 162 | TLPTFDPTKV | FIG. 2AK |
| 170 | SLSNRLYYL | FIG. 2AL |
| 172 | SLIQNLFKV | FIG. 2AM |
| 174 | SIAGFVASI | FIG. 2AN |
| 188 | FIANLPPELKA | FIG. 2AO |
| 189 | ALSERQPSL | FIG. 2AP |
| 191 | LLLNQFSRA | FIG. 2AQ |
| 192 | GLYSTVPHNV | FIG. 2AR |
| 194 | TVLPGLTYL | FIG. 2AS |
| 195 | VLIQQLEKA | FIG. 2AT |
| 201 | ILAVDGVLSV | FIG. 2AU |
| 203 | ILSAHVATA | FIG. 2AV |
| 227 | RLLGTEFQV | FIG. 2AW |
| 232 | SLYSYFQKV | FIG. 2AX |
| 242 | YVTPVLLSV | FIG. 2AY |
| 243 | ALDNTNIGKV | FIG. 2AZ |
| 250 | KLQKEVVSI | FIG. 2BA |
| 252 | NIQELIHGA | FIG. 2BB |
| 268 | RLGQGVPLV | FIG. 2BC |
| 269 | RLLEKHISL | FIG. 2BD |
| 273 | KMPEAGEEQPQV | FIG. 2BE |
| 274 | ISQTPGINL | FIG. 2BF |

Example amino acid sequences of TCR alpha and beta variable region pairs that bind to the indicated peptide are provided below

| SEQ ID | Peptide | TCR alpha | TCR beta |
|---|---|---|---|
| 1 | ALDESNTYQL | 275 | 276 |
| | | 277 | 278 |
| | | 279 | 280 |
| 2 | SLYASGLLTGV | 281 | 282 |
| | | 283 | 284 |
| | | 285 | 286 |
| 3 | RCLFQLETV | 287 | 288 |
| | | 289 | 290 |
| | | 291 | 292 |
| 9 | LLAEHDAVRNAL | 293 | 294 |
| | | 295 | 296 |
| | | 297 | 298 |
| 11 | ALSPAERELL | 299 | 300 |
| | | 301 | 302 |
| | | 303 | 304 |
| 16 | LLAEHDAVRNA | 305 | 306 |
| | | 307 | 308 |
| | | 309 | 310 |
| 22 | EMFPKFTEV | 311 | 312 |
| | | 313 | 314 |
| | | 315 | 316 |
| 23 | RLLDVNNQL | 317 | 318 |
| | | 319 | 320 |
| | | 321 | 322 |
| 26 | ALLDGKCFV | 323 | 324 |
| | | 325 | 326 |
| | | 327 | 328 |
| 31 | TLIPVCWTA | 329 | 330 |
| | | 331 | 332 |
| | | 333 | 334 |
| 32 | VLTSGIVFV | 335 | 336 |
| | | 337 | 338 |
| | | 339 | 340 |
| 36 | SLIAGSAMSKA | 341 | 342 |
| | | 343 | 344 |
| | | 345 | 346 |
| 38 | KLVKELRCV | 347 | 348 |
| | | 349 | 350 |
| | | 351 | 352 |
| 39 | KIFEMLEGV | 353 | 354 |
| | | 355 | 356 |
| | | 357 | 358 |
| 41 | ILQDGQFLV | 359 | 360 |
| | | 361 | 362 |
| | | 363 | 364 |
| 44 | SLSERQITI | 365 | 366 |
| | | 367 | 368 |
| | | 369 | 370 |
| 45 | YLDVSVVQT | 371 | 372 |
| | | 373 | 374 |
| | | 375 | 376 |
| 47 | AILNVDEKNQV | 377 | 378 |
| | | 379 | 380 |
| | | 381 | 382 |
| 51 | SLAETIFIV | 383 | 384 |
| | | 385 | 386 |
| | | 387 | 388 |
| 52 | KVMDLPTQEPAL | 389 | 390 |
| | | 391 | 392 |
| | | 393 | 394 |
| 53 | KLYDPLYHPSM | 395 | 396 |
| | | 397 | 398 |
| | | 399 | 400 |
| 55 | SIASQCPTA | 401 | 402 |
| | | 403 | 404 |
| | | 405 | 406 |
| 56 | FQNSYTIGL | 407 | 408 |
| | | 409 | 410 |
| | | 411 | 412 |
| 59 | ALGETFIRYFV | 413 | 414 |
| | | 415 | 416 |
| | | 417 | 418 |
| 66 | KVLEFLAKV | 419 | 420 |
| | | 421 | 422 |
| | | 423 | 424 |
| 71 | ILQSSPESA | 425 | 426 |
| | | 427 | 428 |
| | | 429 | 430 |
| 75 | FLAMLKNTV | 431 | 432 |
| | | 433 | 434 |
| | | 435 | 436 |
| 114 | AINPLIYCV | 437 | 438 |
| | | 439 | 440 |
| | | 441 | 442 |
| 121 | VIIQNLPAL | 443 | 444 |
| | | 445 | 446 |
| | | 447 | 448 |
| 128 | GLLKTLRFV | 449 | 450 |
| | | 451 | 452 |
| | | 453 | 454 |
| 129 | GLPGGRPVAL | 455 | 456 |
| | | 457 | 458 |
| | | 459 | 460 |
| 142 | AMATNNISL | 461 | 462 |
| | | 463 | 464 |
| | | 465 | 466 |
| 146 | LLDTDYDNFL | 467 | 468 |
| | | 469 | 470 |
| | | 471 | 472 |
| 147 | MMCQYLARV | 473 | 474 |
| | | 475 | 476 |
| | | 477 | 478 |
| 149 | TLLDTDYDNFL | 479 | 480 |
| | | 481 | 482 |
| | | 483 | 484 |
| 158 | IMPTFDLTKV | 485 | 486 |
| | | 487 | 488 |
| | | 489 | 490 |
| 162 | TLPTFDPTKV | 491 | 492 |
| | | 493 | 494 |
| | | 495 | 496 |
| 170 | SLSNRLYYL | 497 | 498 |
| | | 499 | 500 |
| | | 501 | 502 |
| 172 | SLIQNLFKV | 503 | 504 |
| | | 505 | 506 |
| | | 507 | 508 |
| 174 | SIAGFVASI | 509 | 510 |
| | | 511 | 512 |
| | | 513 | 514 |
| 188 | FIANLPPELKA | 515 | 516 |
| | | 517 | 518 |
| | | 519 | 520 |
| 189 | ALSERQPSL | 521 | 522 |
| | | 523 | 524 |
| | | 525 | 526 |
| 191 | LLLNQFSRA | 527 | 528 |
| | | 529 | 530 |
| | | 531 | 532 |
| 192 | GLYSTVPHNV | 533 | 534 |
| | | 535 | 536 |
| | | 537 | 538 |
| 194 | TVLPGLTYL | 539 | 540 |
| | | 541 | 542 |
| | | 543 | 544 |
| 195 | VLIQQLEKA | 545 | 546 |
| | | 547 | 548 |
| | | 549 | 550 |
| 201 | ILAVDGVLSV | 551 | 552 |
| | | 553 | 554 |
| | | 555 | 556 |
| 203 | ILSAHVATA | 557 | 558 |
| | | 559 | 560 |
| | | 561 | 562 |
| 227 | RLLGTEFQV | 563 | 564 |
| | | 565 | 566 |
| | | 567 | 568 |
| 232 | SLYSYFQKV | 569 | 570 |
| | | 571 | 572 |
| | | 573 | 574 |
| 242 | YVTPVLLSV | 575 | 576 |
| | | 577 | 578 |
| | | 579 | 580 |
| 243 | ALDNTNIGKV | 581 | 582 |
| | | 583 | 584 |
| | | 585 | 586 |
| 250 | KLQKEVVSI | 587 | 588 |
| | | 589 | 590 |
| | | 591 | 592 |
| 252 | NIQELIHGA | 593 | 594 |
| | | 595 | 596 |
| | | 597 | 598 |
| 268 | RLGQGVPLV | 599 | 600 |
| | | 601 | 602 |
| | | 603 | 604 |
| 269 | RLLEKHISL | 605 | 606 |
| | | 607 | 608 |
| | | 609 | 610 |
| 273 | KMPEAGEEQPQV | 611 | 612 |
| 274 | ISQTPGINL | 613 | 614 |
| | | 615 | 616 |
| | | 617 | 618 |

These data confirm that antigen specific TCRs can be isolated.

### Example 4—Expression in Tumour Tissues

### Method

XAGE1 expression was analysed by Quantitative real-time PCR using a lung cancer array panel (Origene TissueScan HLRT503). The PCR assay was performed with an internal fluorescent probe 5′-CAGCAGCTGAAAGTCGGGATCCTACACC-3′ (SEQ ID NO: 619) synthesized by IDT Integrated DNA Technologies. Primers were designed in-house (forward 5′-AACACAGAACCACACAGCCAGTC-3′ (SEQ ID NO: 620) and reverse 5′-CAGCTGTATCCTGATCTTCTTCTGTC-3′ (SEQ ID NO: 621)) and synthesized by Eurofins MWG Operon. The assay spans over introns to avoid any genomic DNA amplification, and its specificity was validated by resolution on agarose gel and sequencing.

PCR reactions were performed on the lyophilised cDNA for the cancer panel with 500 nM of each primer, the fluorescent probe, and 2× Quantitect Probe Mastermix (Qiagen). PCR cycling conditions consisted of: 15 min at 95° C.; then 40 cycles of 15 s at 95° C., 60s at 60° C.; and was performed using a QuantStudio 6 instrument (Life Technologies). Purified PCR products were previously cloned into a pCR®4-TOPO plasmid to produce a standard template of a known copy number. Serial 1:10 dilutions were used to generate a standard curve from 101 to 106 transcripts/reaction and run in parallel, thus allowing the calculation of absolute transcript number in the cancer samples.

### Results

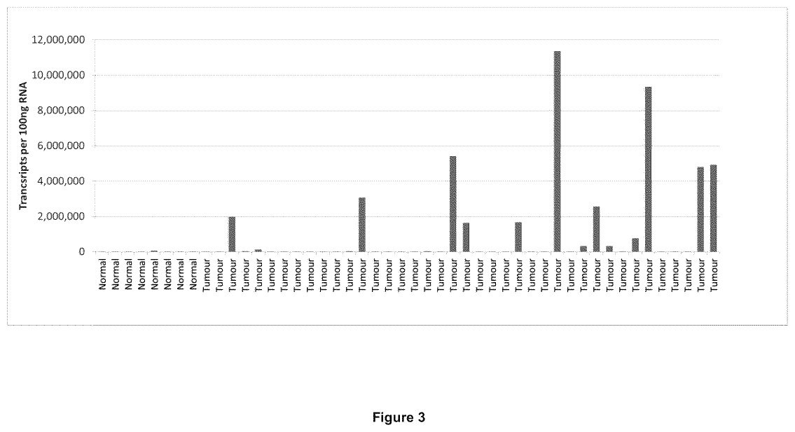

FIG. 3 shows mRNA transcript levels of XAGE1 are elevated in lung tissue compared to normal tissues, indicating that XAGE1 is a valid TAA.

1. A polypeptide comprising, consisting essentially of or consisting of:
(a) the amino acid sequence of any one of SEQ ID NOS: 1-274, or
(b) the amino acid sequence of any one of SEQ ID NOs: 1-274 with the exception of 1, 2 or 3 amino acid substitutions and/or 1, 2 or 3 amino acid insertions, and/or 1, 2 or 3 amino acid deletions,
wherein the polypeptide forms a complex with a Major Histocompatibility Complex (MHC) molecule.

2. The polypeptide of claim 1, wherein the polypeptide consists of from 8 to 16 amino acids.

3. The polypeptide of claim 1, wherein the polypeptide consists of the amino acid sequence of SEQ ID NOs 1-274.

4. A complex of the polypeptide of any preceding claim and a Major Histocompatibility Complex (MHC) molecule.

5. The complex of claim 4, wherein the MHC molecule is MHC class I

6. A nucleic acid molecule comprising a nucleic acid sequence encoding the polypeptide as defined in claim 1.

7. A vector comprising a nucleic acid sequence as defined in claim 6.

8. A cell comprising a vector as claimed in claim 7.

9. A binding moiety that binds the polypeptide of claim 1.

10. The binding moiety of claim 9, which binds the polypeptide when it is in complex with WIC.

11. The binding moiety of claim 10, wherein the binding moiety is a T cell receptor (TCR) or an antibody.

12. The binding moiety of claim 11, wherein the TCR is on the surface of a cell.

13. A polypeptide as defined in claim 1, a complex as defined in claim 4, a nucleic acid molecule as defined in claim 6, a vector as defined in claim 7, a cell as defined in claim 8 or a binding moiety as defined in claim 9 for use in medicine.

14. The polypeptide, complex, nucleic acid, vector or cell for use as defined in claim 13 for use in treating or preventing cancer.

15. A pharmaceutical composition comprising a polypeptide as defined in claim 1, a complex as defined in claim 4, a nucleic acid molecule as defined in claim 6, a vector as defined in claim 7, a cell as defined in claim 8 or a binding moiety as defined in claim 9 together with a pharmaceutically acceptable carrier.

16. A method of identifying a binding moiety that binds a complex as claimed in claim 4, the method comprising contacting a candidate binding moiety with the complex and determining whether the candidate binding moiety binds the complex.


SEQUENCE LISTING (621 sequences)

SEQ ID NO: 1 - protein, 10 aa, Homo sapiens
ALDESNTYQL

SEQ ID NO: 2 - protein, 11 aa, Homo sapiens
SLYASGLLTGV

SEQ ID NO: 3 - protein, 9 aa, Homo sapiens
RCLFQLETV

SEQ ID NO: 4 - protein, 9 aa, Homo sapiens
SLYASGLLT

SEQ ID NO: 5 - protein, 9 aa, Homo sapiens
YASGLLTGV

SEQ ID NO: 6 - protein, 9 aa, Homo sapiens
LPDGSRVEL

SEQ ID NO: 7 - protein, 13 aa, Homo sapiens
APRGPPGTTPVAA

SEQ ID NO: 8 - protein, 9 aa, Homo sapiens
SAVEYIRAL

SEQ ID NO: 9 - protein, 12 aa, Homo sapiens
LLAEHDAVRNAL

SEQ ID NO: 10 - protein, 9 aa, Homo sapiens
AGGLRPQAV

SEQ ID NO: 11 - protein, 10 aa, Homo sapiens
ALSPAERELL

SEQ ID NO: 12 - protein, 12 aa, Homo sapiens
APRGPPGTTPVA

SEQ ID NO: 13 - protein, 11 aa, Homo sapiens
APRGPPGTTPV

SEQ ID NO: 14 - protein, 10 aa, Homo sapiens
PGTTPVAASP

SEQ ID NO: 15 - protein, 10 aa, Homo sapiens
APRGPPGTTP

SEQ ID NO: 16 - protein, 11 aa, Homo sapiens
LLAEHDAVRNA

SEQ ID NO: 17 - protein, 12 aa, Homo sapiens
TAETGGGAAAVA

SEQ ID NO: 18 - protein, 9 aa, Homo sapiens
ETLRSAVEY

SEQ ID NO: 19 - protein, 11 aa, Homo sapiens
SSDDSGCEGAL

SEQ ID NO: 20 - protein, 9 aa, Homo sapiens
ALSPAEREL

SEQ ID NO: 21 - protein, 10 aa, Homo sapiens
LAGGLRPQAV

SEQ ID NO: 22 - protein, 9 aa, Homo sapiens
EMFPKFTEV

SEQ ID NO: 23 - protein, 9 aa, Homo sapiens
RLLDVNNQL

SEQ ID NO: 24 - protein, 9 aa, Homo sapiens
RELEKYVSA

SEQ ID NO: 25 - protein, 9 aa, Homo sapiens
SVLQRALAA

SEQ ID NO: 26 - protein, 9 aa, Homo sapiens
ALLDGKCFV

SEQ ID NO: 27 - protein, 9 aa, Homo sapiens
SHRAPTLAL

SEQ ID NO: 28 - protein, 10 aa, Homo sapiens
LFQHFQSSSE

SEQ ID NO: 29 - protein, 10 aa, Homo sapiens
EPPAVPEPPE

SEQ ID NO: 30 - protein, 11 aa, Homo sapiens
SLLALPQDLQA

SEQ ID NO: 31 - protein, 9 aa, Homo sapiens
TLIPVCWTA

SEQ ID NO: 32 - protein, 9 aa, Homo sapiens
VLTSGIVFV

SEQ ID NO: 33 - protein, 15 aa, Homo sapiens
AISRGPSEYPTKNYV

SEQ ID NO: 34 - protein, 9 aa, Homo sapiens
GLLLLGGGL

SEQ ID NO: 35 - protein, 9 aa, Homo sapiens
KYEKIFEML

SEQ ID NO: 36 - protein, 11 aa, Homo sapiens
SLIAGSAMSKA

SEQ ID NO: 37 - protein, 11 aa, Homo sapiens
REINADIKRKL

SEQ ID NO: 38 - protein, 9 aa, Homo sapiens
KLVKELRCV

SEQ ID NO: 39 - protein, 9 aa, Homo sapiens
KIFEMLEGV

SEQ ID NO: 40 - protein, 10 aa, Homo sapiens
APSMLRKNQL

SEQ ID NO: 41 - protein, 9 aa, Homo sapiens
ILQDGQFLV

SEQ ID NO: 42 - protein, 9 aa, Homo sapiens
NHLDSHLLL

SEQ ID NO: 43 - protein, 11 aa, Homo sapiens
HSPLTSHPAAP

SEQ ID NO: 44 - protein, 9 aa, Homo sapiens
SLSERQITI

SEQ ID NO: 45 - protein, 9 aa, Homo sapiens
YLDVSVVQT

SEQ ID NO: 46 - protein, 9 aa, Homo sapiens
AIGTPLIGV

SEQ ID NO: 47 - protein, 11 aa, Homo sapiens
AILNVDEKNQV

SEQ ID NO: 48 - protein, 10 aa, Homo sapiens
EASLAVCGLL

SEQ ID NO: 49 - protein, 9 aa, Homo sapiens
FLIIVSDTL

SEQ ID NO: 50 - protein, 14 aa, Homo sapiens
LGYSVFLIIVSDTL

SEQ ID NO: 51 - protein, 9 aa, Homo sapiens
SLAETIFIV

SEQ ID NO: 52 - protein, 12 aa, Homo sapiens
KVMDLPTQEPAL

SEQ ID NO: 53 - protein, 11 aa, Homo sapiens
KLYDPLYHPSM

SEQ ID NO: 54 - protein, 9 aa, Homo sapiens
GYLQGLVSF

SEQ ID NO: 55 - protein, 9 aa, Homo sapiens
SIASQCPTA

SEQ ID NO: 56 - protein, 9 aa, Homo sapiens
FQNSYTIGL

SEQ ID NO: 57 - protein, 11 aa, Homo sapiens
SVSESDTIRSI

SEQ ID NO: 58 - protein, 11 aa, Homo sapiens
LVSGWGLLANG

SEQ ID NO: 59 - protein, 11 aa, Homo sapiens
ALGETFIRYFV

SEQ ID NO: 60 - protein, 9 aa, Homo sapiens
LSCLMAPSV

SEQ ID NO: 61 - protein, 8 aa, Homo sapiens
QILTPPQL

SEQ ID NO: 62 - protein, 10 aa, Homo sapiens
EEIWEFLNML

SEQ ID NO: 63 - protein, 12 aa, Homo sapiens
SVFGEPWKLITK

SEQ ID NO: 64 - protein, 11 aa, Homo sapiens
REHFPEILKKA

SEQ ID NO: 65 - protein, 9 aa, Homo sapiens
SVFGEPWKL

SEQ ID NO: 66 - protein, 9 aa, Homo sapiens
KVLEFLAKV

SEQ ID NO: 67 - protein, 9 aa, Homo sapiens
RTHSTFEGF

SEQ ID NO: 68 - protein, 11 aa, Homo sapiens
TLLSILQSSPE

SEQ ID NO: 69 - protein, 11 aa, Homo sapiens
SIFQSSPESAQ

SEQ ID NO: 70 - protein, 11 aa, Homo sapiens
SIFQSSPERTQ

SEQ ID NO: 71 - protein, 9 aa, Homo sapiens
ILQSSPESA

SEQ ID NO: 72 - protein, 11 aa, Homo sapiens
STFEGFPQSPL

SEQ ID NO: 73 - protein, 9 aa, Homo sapiens
IESEPLFTY

SEQ ID NO: 74 - protein, 11 aa, Homo sapiens
SPSFSSTLLSL

SEQ ID NO: 75 - protein, 9 aa, Homo sapiens
FLAMLKNTV

SEQ ID NO: 76 - protein, 9 aa, Homo sapiens
LQIPVSPSF

SEQ ID NO: 77 - protein, 9 aa, Homo sapiens
MTSSFSSTL

SEQ ID NO: 78 - protein, 11 aa, Homo sapiens
REVDPDDSYVF

SEQ ID NO: 79 - protein, 9 aa, Homo sapiens
RAGREHFAF

SEQ ID NO: 80 - protein, 10 aa, Homo sapiens
SPLYFPQSPL

SEQ ID NO: 81 - protein, 11 aa, Homo sapiens
QSPPEGPVQSP

SEQ ID NO: 82 - protein, 9 aa, Homo sapiens
SICSSSTSL

SEQ ID NO: 83 - protein, 9 aa, Homo sapiens
VIKRKVVEF

SEQ ID NO: 84 - protein, 14 aa, Homo sapiens
GDKDMPTAGMPSLL

SEQ ID NO: 85 - protein, 10 aa, Homo sapiens
SLLQIPMTSS

SEQ ID NO: 86 - protein, 8 aa, Homo sapiens
SPVSICSS

SEQ ID NO: 87 - protein, 9 aa, Homo sapiens
QPITKAEML

SEQ ID NO: 88 - protein, 9 aa, Homo sapiens
DELARFLLL

SEQ ID NO: 89 - protein, 9 aa, Homo sapiens
NSSPPRYEF

SEQ ID NO: 90 - protein, 12 aa, Homo sapiens
REVDPDDSYVFV

SEQ ID NO: 91 - protein, 9 aa, Homo sapiens
RYTGYFPVI

SEQ ID NO: 92 - protein, 11 aa, Homo sapiens
SILQSSPESAQ

SEQ ID NO: 93 - protein, 8 aa, Homo sapiens
FSYTLLSL

SEQ ID NO: 94 - protein, 8 aa, Homo sapiens
SPLQIPQS

SEQ ID NO: 95 - protein, 9 aa, Homo sapiens
LQIPVSSSF

SEQ ID NO: 96 - protein, 9 aa, Homo sapiens
SFFSSALLS

SEQ ID NO: 97 - protein, 9 aa, Homo sapiens
QVKQPITKA

SEQ ID NO: 98 - protein, 11 aa, Homo sapiens
SPSFSSTLVSL

SEQ ID NO: 99 - protein, 11 aa, Homo sapiens
EGFAQSPLQIP

SEQ ID NO: 100 - protein, 9 aa, Homo sapiens
FEGFPQSLL

SEQ ID NO: 101 - protein, 9 aa, Homo sapiens
ISFSSSTSL

SEQ ID NO: 102 - protein, 10 aa, Homo sapiens
MSQNRLLILI

SEQ ID NO: 103 - protein, 12 aa, Homo sapiens
PEGENTHSPLQI

SEQ ID NO: 104 - protein, 10 aa, Homo sapiens
PSFSSTLVSL

SEQ ID NO: 105 - protein, 9 aa, Homo sapiens
QEHYLEYRE

SEQ ID NO: 106 - protein, 9 aa, Homo sapiens
QIPQSPLEG

SEQ ID NO: 107 - protein, 11 aa, Homo sapiens
SAFEGFPQSPL

SEQ ID NO: 108 - protein, 9 aa, Homo sapiens
SEEVIWDVL

SEQ ID NO: 109 - protein, 9 aa, Homo sapiens
SEPLFTYTL

SEQ ID NO: 110 - protein, 9 aa, Homo sapiens
SPFEGFPQS

SEQ ID NO: 111 - protein, 9 aa, Homo sapiens
SPLQIVPSL

SEQ ID NO: 112 - protein, 9 aa, Homo sapiens
VSSSFSYTL

SEQ ID NO: 113 - protein, 10 aa, Homo sapiens
CSDGKLCSSY

SEQ ID NO: 114 - protein, 9 aa, Homo sapiens
AINPLIYCV

SEQ ID NO: 115 - protein, 9 aa, Homo sapiens
FTEVVEGKE

SEQ ID NO: 116 - protein, 9 aa, Homo sapiens
NSAINPLIY

SEQ ID NO: 117 - protein, 11 aa, Homo sapiens
VVEGKEWGSFY

SEQ ID NO: 118 - protein, 14 aa, Homo sapiens
FTEVVEGKEWGSFY

SEQ ID NO: 119 - protein, 12 aa, Homo sapiens
VVEGKEWGSFYY

SEQ ID NO: 120 - protein, 8 aa, Homo sapiens
GVPQHRQI

SEQ ID NO: 121 - protein, 9 aa, Homo sapiens
VIIQNLPAL

SEQ ID NO: 122 - protein, 9 aa, Homo sapiens
QILSKPEFI

SEQ ID NO: 123 - protein, 9 aa, Homo sapiens
APKAPEATL

SEQ ID NO: 124 - protein, 9 aa, Homo sapiens
ASAGLLKTL

SEQ ID NO: 125 - protein, 9 aa, Homo sapiens
AYLKGTVLF

SEQ ID NO: 126 - protein, 11 aa, Homo sapiens
EYAYLKGTVLF

SEQ ID NO: 127 - protein, 11 aa, Homo sapiens
GEDHPQQGSTL

SEQ ID NO: 128 - protein, 9 aa, Homo sapiens
GLLKTLRFV

SEQ ID NO: 129 - protein, 10 aa, Homo sapiens
GLPGGRPVAL

SEQ ID NO: 130 - protein, 9 aa, Homo sapiens
GPHDRFIEL

SEQ ID NO: 131 - protein, 9 aa, Homo sapiens
KYLPCFQVL

SEQ ID NO: 132 - protein, 9 aa, Homo sapiens
LPGGRATAL

SEQ ID NO: 133 - protein, 9 aa, Homo sapiens
LPGGRPVAL

SEQ ID NO: 134 - protein, 9 aa, Homo sapiens
RFIELNSTL

SEQ ID NO: 135 - protein, 10 aa, Homo sapiens
RFIELNSTLF

SEQ ID NO: 136 - protein, 9 aa, Homo sapiens
RPIIGTVSM

SEQ ID NO: 137 - protein, 9 aa, Homo sapiens
SASQVQAIK

SEQ ID NO: 138 - protein, 9 aa, Homo sapiens
SGALRPVAL

SEQ ID NO: 139 - protein, 9 aa, Homo sapiens
YAAPKAPEA

SEQ ID NO: 140 - protein, 9 aa, Homo sapiens
YAYLKGTVL

SEQ ID NO: 141 - protein, 8 aa, Homo sapiens
YLKGTVLF

SEQ ID NO: 142 - protein, 9 aa, Homo sapiens
AMATNNISL

SEQ ID NO: 143 - protein, 9 aa, Homo sapiens
DTDYDNFLF

SEQ ID NO: 144 - protein, 10 aa, Homo sapiens
DTDYDNFLFL

SEQ ID NO: 145 - protein, 9 aa, Homo sapiens
KQMEEPCRF

SEQ ID NO: 146 - protein, 10 aa, Homo sapiens
LLDTDYDNFL

SEQ ID NO: 147 - protein, 9 aa, Homo sapiens
MMCQYLARV

SEQ ID NO: 148 - protein, 10 aa, Homo sapiens
TLLDTDYDNF

SEQ ID NO: 149 - protein, 11 aa, Homo sapiens
TLLDTDYDNFL

SEQ ID NO: 150 - protein, 11 aa, Homo sapiens
MEAFQQELALL

SEQ ID NO: 151 - protein, 11 aa, Homo sapiens
TKVLEAGDAQP

SEQ ID NO: 152 - protein, 10 aa, Homo sapiens
VIVQEPTEEK

SEQ ID NO: 153 - protein, 10 aa, Homo sapiens
MEAFQQELAL

SEQ ID NO: 154 - protein, 11 aa, Homo sapiens
EGIMPTFDLTK

SEQ ID NO: 155 - protein, 8 aa, Homo sapiens
FQGPDMEA

SEQ ID NO: 156 - protein, 11 aa, Homo sapiens
SVIVQEPTEEK

SEQ ID NO: 157 - protein, 10 aa, Homo sapiens
GIMPTFDLTK

SEQ ID NO: 158 - protein, 10 aa, Homo sapiens
IMPTFDLTKV

SEQ ID NO: 159 - protein, 9 aa, Homo sapiens
FQGPDMEAF

SEQ ID NO: 160 - protein, 11 aa, Homo sapiens
GEIENQAVPAF

SEQ ID NO: 161 - protein, 11 aa, Homo sapiens
EGTLPTFDPTK

SEQ ID NO: 162 - protein, 10 aa, Homo sapiens
TLPTFDPTKV

SEQ ID NO: 163 - protein, 12 aa, Homo sapiens
IEDAPGDGPDVR

SEQ ID NO: 164 - protein, 10 aa, Homo sapiens
VEAFQQELAL

SEQ ID NO: 165 - protein, 12 aa, Homo sapiens
PTKVLEAGEGQL

SEQ ID NO: 166 - protein, 9 aa, Homo sapiens
VLEAGEGQL

SEQ ID NO: 167 - protein, 9 aa, Homo sapiens
GIAPSGEIK

SEQ ID NO: 168 - protein, 11 aa, Homo sapiens
GEIKNEGAPAV

SEQ ID NO: 169 - protein, 9 aa, Homo sapiens
IVQQPTEEK

SEQ ID NO: 170 - protein, 9 aa, Homo sapiens
SLSNRLYYL

SEQ ID NO: 171 - protein, 9 aa, Homo sapiens
GSEVSFLEY

SEQ ID NO: 172 - protein, 9 aa, Homo sapiens
SLIQNLFKV

SEQ ID NO: 173 - protein, 9 aa, Homo sapiens
LKIMNLQQI

SEQ ID NO: 174 - protein, 9 aa, Homo sapiens
SIAGFVASI

SEQ ID NO: 175 - protein, 10 aa, Homo sapiens
GSEVSFLEYY

SEQ ID NO: 176 - protein, 9 aa, Homo sapiens
TRGAPLISV

SEQ ID NO: 177 - protein, 10 aa, Homo sapiens
LTSRPQWALY

SEQ ID NO: 178 - protein, 9 aa, Homo sapiens
NPRLTVIVV

SEQ ID NO: 179 - protein, 9 aa, Homo sapiens
EVDDRTEAY

SEQ ID NO: 180 - protein, 9 aa, Homo sapiens
KMGGELWRV

SEQ ID NO: 181 - protein, 8 aa, Homo sapiens
AIATKIAL

SEQ ID NO: 182 - protein, 10 aa, Homo sapiens
IDYNPLMEAR

SEQ ID NO: 183 - protein, 9 aa, Homo sapiens
SFDSNLLSF

SEQ ID NO: 184 - protein, 9 aa, Homo sapiens
RLQQKVTEV

SEQ ID NO: 185 - protein, 9 aa, Homo sapiens
MLIPELCYL

SEQ ID NO: 186 - protein, 11 aa, Homo sapiens
IMIEVDDRTEA

SEQ ID NO: 187 - protein, 11 aa, Homo sapiens
LLLNQFSRAVA

SEQ ID NO: 188 - protein, 11 aa, Homo sapiens
FIANLPPELKA

SEQ ID NO: 189 - protein, 9 aa, Homo sapiens
ALSERQPSL

SEQ ID NO: 190 - protein, 10 aa, Homo sapiens
LLNQFSRAVA

SEQ ID NO: 191 - protein, 9 aa, Homo sapiens
LLLNQFSRA

SEQ ID NO: 192 - protein, 10 aa, Homo sapiens
GLYSTVPHNV

SEQ ID NO: 193 - protein, 9 aa, Homo sapiens
VEFTIKEAA

SEQ ID NO: 194 - protein, 9 aa, Homo sapiens
TVLPGLTYL

SEQ ID NO: 195 - protein, 9 aa, Homo sapiens
VLIQQLEKA

SEQ ID NO: 196 - protein, 9 aa, Homo sapiens
ALSTVLPGL

SEQ ID NO: 197 - protein, 9 aa, Homo sapiens
TVQPVIIYL

SEQ ID NO: 198 - protein, 9 aa, Homo sapiens
LYKPDSNEF

SEQ ID NO: 199 - protein, 10 aa, Homo sapiens
GGHIAGSLAV

SEQ ID NO: 200 - protein, 12 aa, Homo sapiens
IAGSLAVVTDAA

SEQ ID NO: 201 - protein, 10 aa, Homo sapiens
ILAVDGVLSV

SEQ ID NO: 202 - protein, 8 aa, Homo sapiens
ILKDFSIL

SEQ ID NO: 203 - protein, 9 aa, Homo sapiens
ILSAHVATA

SEQ ID NO: 204 - protein, 8 aa, Homo sapiens
LACERLLY

SEQ ID NO: 205 - protein, 11 aa, Homo sapiens
RTYLVNDKAAK

SEQ ID NO: 206 - protein, 8 aa, Homo sapiens
AAYVTPVL

SEQ ID NO: 207 - protein, 9 aa, Homo sapiens
AAYVTPVLL

SEQ ID NO: 208 - protein, 9 aa, Homo sapiens
AISVTMIGV

SEQ ID NO: 209 - protein, 13 aa, Homo sapiens
ALFTGFGGALGYL

SEQ ID NO: 210 - protein, 10 aa, Homo sapiens
ALGYLLGAID

SEQ ID NO: 211 - protein, 10 aa, Homo sapiens
ALIGCCFVAL

SEQ ID NO: 212 - protein, 9 aa, Homo sapiens
AMFGREFCY

SEQ ID NO: 213 - protein, 10 aa, Homo sapiens
AMFGREFCYA

SEQ ID NO: 214 - protein, 11 aa, Homo sapiens
AMFGREFCYAV

SEQ ID NO: 215 - protein, 11 aa, Homo sapiens
AQILVGGGLGF

SEQ ID NO: 216 - protein, 10 aa, Homo sapiens
FLIYERGVEV

SEQ ID NO: 217 - protein, 10 aa, Homo sapiens
FTDFMGQIVY

SEQ ID NO: 218 - protein, 10 aa, Homo sapiens
FTGFGGALGY

SEQ ID NO: 219 - protein, 11 aa, Homo sapiens
GLFPNVYSTLV

SEQ ID NO: 220 - protein, 9 aa, Homo sapiens
GLGTGFIGL

SEQ ID NO: 221 - protein, 9 aa, Homo sapiens
GLPSSLYSI

SEQ ID NO: 222 - protein, 10 aa, Homo sapiens
GVMSSTLYTV

SEQ ID NO: 223 - protein, 9 aa, Homo sapiens
HLIGWTAFL

SEQ ID NO: 224 - protein, 8 aa, Homo sapiens
LLGTEFQV

SEQ ID NO: 225 - protein, 9 aa, Homo sapiens
LSSDGMYEY

SEQ ID NO: 226 - protein, 8 aa, Homo sapiens
RLLGTEFQ

SEQ ID NO: 227 - protein, 9 aa, Homo sapiens
RLLGTEFQV

SEQ ID NO: 228 - protein, 10 aa, Homo sapiens
RLLGTEFQVM

SEQ ID NO: 229 - protein, 11 aa, Homo sapiens
RLLGTEFQVMF

SEQ ID NO: 230 - protein, 10 aa, Homo sapiens
SISEAPLTEV

SEQ ID NO: 231 - protein, 13 aa, Homo sapiens
SLFGVMSSTLYTV

SEQ ID NO: 232 - protein, 9 aa, Homo sapiens
SLYSYFQKV

SEQ ID NO: 233 - protein, 11 aa, Homo sapiens
SLYSYFQKVLV

SEQ ID NO: 234 - protein, 9 aa, Homo sapiens
SVGLPSSLY

SEQ ID NO: 235 - protein, 10 aa, Homo sapiens
SYIGLKGLYF

SEQ ID NO: 236 - protein, 9 aa, Homo sapiens
TLYTVPFNL

SEQ ID NO: 237 - protein, 9 aa, Homo sapiens
VMSSTLYTV

SEQ ID NO: 238 - protein, 10 aa, Homo sapiens
VWFLSPILGF

SEQ ID NO: 239 - protein, 9 aa, Homo sapiens
YLNGATVVA

SEQ ID NO: 240 - protein, 12 aa, Homo sapiens
YSAHNSTEFLIY

SEQ ID NO: 241 - protein, 11 aa, Homo sapiens
YTVPFNLITEY

SEQ ID NO: 242 - protein, 9 aa, Homo sapiens
YVTPVLLSV

SEQ ID NO: 243 - protein, 10 aa, Homo sapiens
ALDNTNIGKV

SEQ ID NO: 244 - protein, 9 aa, Homo sapiens
CMFSRVLTL

SEQ ID NO: 245 - protein, 9 aa, Homo sapiens
DLSVKRESL

SEQ ID NO: 246 - protein, 9 aa, Homo sapiens
EERKKFLAV

SEQ ID NO: 247 - protein, 10 aa, Homo sapiens
FPQLKKVIQF

SEQ ID NO: 248 - protein, 13 aa, Homo sapiens
ILLSGSLDDIIEV

SEQ ID NO: 249 - protein, 10 aa, Homo sapiens
ITAIVVASEK

SEQ ID NO: 250 - protein, 9 aa, Homo sapiens
KLQKEVVSI

SEQ ID NO: 251 - protein, 8 aa, Homo sapiens
LFDLCHPI

SEQ ID NO: 252 - protein, 9 aa, Homo sapiens
NIQELIHGA

SEQ ID NO: 253 - protein, 9 aa, Homo sapiens
TDLKQIQTL

SEQ ID NO: 254 - protein, 11 aa, Homo sapiens
VMGEKIANLRV

SEQ ID NO: 255 - protein, 12 aa, Homo sapiens
YIAELEKIGIIV

SEQ ID NO: 256 - protein, 8 aa, Homo sapiens
YQLAVTKV

SEQ ID NO: 257 - protein, 9 aa, Homo sapiens
ACKLYKAMA

SEQ ID NO: 258 - protein, 11 aa, Homo sapiens
ASDILSFAHKY

SEQ ID NO: 259 - protein, 10 aa, Homo sapiens
DDPAVSRFQY

SEQ ID NO: 260 - protein, 11 aa, Homo sapiens
DLVGKDVTRVY

SEQ ID NO: 261 - protein, 10 aa, Homo sapiens
EVFADQIDLY

SEQ ID NO: 262 - protein, 10 aa, Homo sapiens
GVLEFQGGGY

SEQ ID NO: 263 - protein, 10 aa, Homo sapiens
LADNGTLGKY

SEQ ID NO: 264 - protein, 8 aa, Homo sapiens
LPPPMIIL

SEQ ID NO: 265 - protein, 12 aa, Homo sapiens
MTTGAWIFTGGV

SEQ ID NO: 266 - protein, 9 aa, Homo sapiens
MYIRVSYDT

SEQ ID NO: 267 - protein, 9 aa, Homo sapiens
QHIPPLPSA

SEQ ID NO: 268 - protein, 9 aa, Homo sapiens
RLGQGVPLV

SEQ ID NO: 269 - protein, 9 aa, Homo sapiens
RLLEKHISL

SEQ ID NO: 270 - protein, 8 aa, Homo sapiens
SLQEWIVI

SEQ ID NO: 271 - protein, 9 aa, Homo sapiens
STFMIGAIL

SEQ ID NO: 272 - protein, 9 aa, Homo sapiens
TQSYPTDSY

SEQ ID NO: 273 - protein, 12 aa, Homo sapiens
KMPEAGEEQPQV

SEQ ID NO: 274 - protein, 9 aa, Homo sapiens
ISQTPGINL

SEQ ID NO: 275 - protein, 110 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRGYNKLIFGAGTRLAVHP

SEQ ID NO: 276 - protein, 112 aa, Homo sapiens
NAGVTQTPKFRILKIGQSMTLQCTQDMNHNYMYWYRQDPGMGLKLIYYSVGAGITDKGEVPNGYNVSRSTTEDFPLRLLSAAPSQTSVYFCASSYSPIVGAFFGQGTRLTVV

SEQ ID NO: 277 - protein, 110 aa, Homo sapiens; identical to SEQ ID NO: 275

SEQ ID NO: 278 - protein, 112 aa, Homo sapiens
NAGVTQTPKFRILKIGQSMTLQCAQDMNHNYMYWYRQDPGMGLKLIYYSVGAGITDKGEVPNGYNVSRSTTEDFPLXLELAAPSQTSVYFCASTRDTYYGYTFGSGTXLTVV

SEQ ID NO: 279 - protein, 109 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCATGFQKLVFGTGTRLLVSP

SEQ ID NO: 280 - protein, 113 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASTLGRLLTEAFFGQGTRLTVV

SEQ ID NO: 281 - protein, 110 aa, Homo sapiens; identical to SEQ ID NO: 275

SEQ ID NO: 282 - protein, 111 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSFDVTEAFFGQGTRLTVV

SEQ ID NO: 283 - protein, 112 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVKKDAGNKLTFGGGTRVLVKP

SEQ ID NO: 284 - protein, 119 aa, Homo sapiens
NAGVTQTPKFRVLKTGQSMTLLCAQDMNHEYMYWYRQDPGMGLRLIYYSASEGTTDKGEVPNGYNVSRLNKREFSLRLESAAPSQTSVYFCASSDSRRGGLLLYNSPLHFGNGTRLTVT

SEQ ID NO: 285 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRAAGNKLTFGGGTRVLVKP

SEQ ID NO: 286 - protein, 119 aa, Homo sapiens; identical to SEQ ID NO: 284

SEQ ID NO: 287 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVNYYNQGGKLIFGQGTELSVKP

SEQ ID NO: 288 - protein, 114 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASTPTYWDSYEQYFGPGTRLTVT

SEQ ID NO: 289 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVDSSGSRLTFGEGTQLTVNP

SEQ ID NO: 290 - protein, 112 aa, Homo sapiens
NAGVTQTPKFQVLKTGQSMTLQCAQDMNHEYMYWYRQDPGMGLRLIHYSVGAGITDQGEVPNGYNVSRSTTEDFPLRLLSAAPSQTSVYFCASRLLGGSGYTFGSGTRLTVV

SEQ ID NO: 291 - protein, 109 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVDADKLIFGTGTRLQVFP

SEQ ID NO: 292 - protein, 112 aa, Homo sapiens
NAGVTQTPKFRILKIGQSMTLQCTQDMNHEYMYWYRQDPGMGLRLIHYSVGEGTTAKGEVPDGYNVSRLKKQNFLLGLESAAPSQTSVYFCASREVGRDEHFFGPGTRLTVL

SEQ ID NO: 293 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRQHTGRRALTFGSGTRLQVQP

SEQ ID NO: 294 - protein, 113 aa, Homo sapiens
NAGVTQTPKFRILKIGQSMTLQCTQDMNHNYMYWYRQDPGMGLKLIYYSVGAGITDKGEVPNGYNVSRSTTEDFPLRLELAAPSQTSVYFCASSYSPENTEAFFGQGTRLTVV

SEQ ID NO: 295 - protein, 113 aa, Homo sapiens; identical to SEQ ID NO: 293

SEQ ID NO: 296 - protein, 113 aa, Homo sapiens
NAGVTQTPKFRILKIGQSMTLQCTQDMNHNYMYWYRQDPGMGLRLIHYSVGAGITDQGEVPNGYNVSRSTTEDFPLRLLSAAPSQTSVYFCASSYSPEADIQYFGAGTRLSVL

SEQ ID NO: 297 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVLTNDYKLSFGAGTTVTVRA

SEQ ID NO: 298 - protein, 113 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSYSGFDSPLHFGNGTRLTVT

SEQ ID NO: 299 - protein, 110 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCAVDTGRRALTFGSGTRLQVQP

SEQ ID NO: 300 - protein, 112 aa, Homo sapiens
NAGVTQTPKFQVLKTGQSMTLLCAQDMNHNSMYWYRQDPGMGLRLIYYSASEGTTDKGEVPNGYNVSRLNKREFSLRLESAAPSQTSVYFCASSVGGTYGYTFGSGTRLTVV

SEQ ID NO: 301 - protein, 110 aa, Homo sapiens; identical to SEQ ID NO: 299

SEQ ID NO: 302 - protein, 112 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASSTGGSYGYTFGSGTRLTVV

SEQ ID NO: 303 - protein, 110 aa, Homo sapiens; identical to SEQ ID NO: 299

SEQ ID NO: 304 - protein, 112 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASSVGGTYYDYFFSSTTLLAV

SEQ ID NO: 305 - protein, 114 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRSSTDSWGKFQFGAGTQVVVTP

SEQ ID NO: 306 - protein, 114 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYSAGLTGELFFGEGSRLTVL

SEQ ID NO: 307 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVTDSWGKLQFGAGTQVVVTP

SEQ ID NO: 308 - protein, 113 aa, Homo sapiens
NAGVTQTPKFQVLKTGQSMTLQCAQDMNHNSMYWYRQDPGMGLRLIYYSVGAGITDQGEVPNGYNVSRSTTEDFPLRLLSAAPSQTSVYFCASSRGPINYGYTFGSGTRLTVV

SEQ ID NO: 309 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRPAGQNFVFGPGTRLSVLP

SEQ ID NO: 310 - protein, 115 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASSEALWGGNTEAFFGQGTRLPVV

SEQ ID NO: 311 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVTGGTSYGKLTFGQGTILTVHP

SEQ ID NO: 312 - protein, 114 aa, Homo sapiens
NAGVTQTPKFRILKIGQSMTLQCTQDMNHNYMYWYRQDPGMGLKLIYYSVGAGITDKGEVPNGYNVSRSTTEDFPLRLELAAPSQTSVYFCASSYWAGVLTEAFFGQGTRLTVV

SEQ ID NO: 313 - protein, 114 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVSNYGGSQGNLIFGKGTKLSVKP

SEQ ID NO: 314 - protein, 113 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASTHWGRDSPLHFGNGTRLTVT

SEQ ID NO: 315 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVSPRSNDYKLSFGAGTTVTVRA

SEQ ID NO: 316 - protein, 114 aa, Homo sapiens
NAGVTQTPKFRILKIGQSMTLQCTQDMNHNYMYWYRQDPGMGLKLIYYSVGAGTTDKGEVPNGYNVSRLNKREFSLRLESAAPSQTSVYFCASSATRRGDYGYTFGSGTRLTVV

SEQ ID NO: 317 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVKGSQGNLIFGKGTKLSVKP

SEQ ID NO: 318 - protein, 113 aa, Homo sapiens
NAGVTQTPKFRILKIGQSMTLQCTQDMNHEYMSWYRQDPGMGLRLIHYSVGAGITDQGEVPNGYNVSRSTTEDFPLRLLSAAPSQTSVYFCASRTLGDHYGYTFGSGTRLTVV

SEQ ID NO: 319 - protein, 111 aa, Homo sapiens
KQEVTQIPVALSVPEGENLVLNCSFTDSAIYNLQWFRQDPGKGLTSLLLIQSSQREQTSGRLNASLDKSSGRSTLYIAASQPGDSATYLCAVKGGANNLFFGTGTRLTVIP

SEQ ID NO: 320 - protein, 113 aa, Homo sapiens
NAGVTQTPKFRILKIGQSMTLQCTQDMNHNYMYWYRQDPGMGLKLIYYSVGAGITDKGEVPNGYNVSRSTTEDFPLRLLSAAPSQTSVYFCASRGDGDRSPLHFGNGTRLTVT

SEQ ID NO: 321 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRGGYQKVTFGTGTKLQVIP

SEQ ID NO: 322 - protein, 113 aa, Homo sapiens
NAGVTQTPKFQVLKTGQSMTLQCAQDMNHNSMYWYRQDPGMGLRLIHYSVGAGITDQGEVPNGYNVSRSTTEDFPLRLLSAAPSQTSVYFCASRGDGDRSPLHFGNGTRLTVT

SEQ ID NO: 323 - protein, 114 aa, Homo sapiens
KQEVTQIPAALSVPEGENLVLNCSFTDSAIYNFQWFRQDPGKGLTSLLLIQSSQREQTSGRLNASLDKSSGRSTLYIAASQPGDSATYLCAAHPATGANSKLTFGKGITLSVRP

SEQ ID NO: 324 - protein, 114 aa, Homo sapiens
NAGVTQTPKFQVLKTGQGMTLQCAQDMNHNSMYWYRQDPGMGLRLIYYSASEGTTDKGEVPNGYNVSRLNKREFSLRLESAAPSQTSVYFCASSEASGSNSPLHFGNGTRLTVT

SEQ ID NO: 325 - protein, 113 aa, Homo sapiens
QKEVEQNSGPLSVPEGAIASLNCTYSDRGSQSFFWYRQYSGKSPELIMFIYSNGDKEDGRFTAQLNKASQYVSLLIRDSQPSDSATYLCAVRTPSGSARQLTFGSGTQLTVLP

SEQ ID NO: 326 - protein, 117 aa, Homo sapiens
DAGVTQSPTHLIKTRGQHVTLRCSPKSGHDTVSWYQQALGQGPQFIFQYYGEEERQRGNFPDRFSGHQFPNYSSELNVNALLLGDSALYLCASSLGAGGGHSGNTIYFGEGSWLTVV

SEQ ID NO: 327 - protein, 109 aa, Homo sapiens
GNSVTQMEGPVTLSEEAFLTINCTYTATGYPSLFWYVQYPGEGLQLLLKATKADDKGSNKGFEATYRKETTSFHLEKGSVQVSDSAVYFCAPHTDKLIFGTGTRLQVFP

SEQ ID NO: 328 - protein, 112 aa, Homo sapiens
DSGVTQTPKHLITATGQRVTLRCSPRSGDLSVYWYQQSLDQGLQFLIQYYNGEERAKGNILERFSAQQFPDLHSELNLSSLELGDSALYFCASSGGTGDGYTFGSGTRLTVV

SEQ ID NO: 329 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVQGYGTALIFGKGTTLSVSS

SEQ ID NO: 330 - protein, 114 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSQGPYMNTEAFFGQGTRLTVV

SEQ ID NO: 331 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAAKAAGNKLTFGGGTRVLVKP

SEQ ID NO: 332 - protein, 114 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASSESLGVTGTAFFGQGTRLTVV

SEQ ID NO: 333 - protein, 112 aa, Homo sapiens; identical to SEQ ID NO: 283

SEQ ID NO: 334 - protein, 114 aa, Homo sapiens
NAGVTQTPKFQVLKTGQSMTLQCAQDMNHNSMYWYRQDPGMGLRLIYYSASEGTTDKGEVPNGYNVSRLNKREFPLRLLSAAPSQTSVYFCASSRRLGASSEAFFGQGTRLTVV

SEQ ID NO: 335 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRHLSNDYKLSFGAGTTVTVRA

SEQ ID NO: 336 - protein, 114 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASKRGFDEGSPLHFGNGTRLTVT

SEQ ID NO: 337 - protein, 112 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRHNQGGKLIFGQGTELSVKP

SEQ ID NO: 338 - protein, 112 aa, Homo sapiens
NAGVTQTPKFRVLKTGQSMTLLCAQDMNHEYMYWYRQDPGMGLRLIHYSVGAGITDQGEVPNGYNVSRLNKREFSLRLESAAPSQTSVYFCASSDRGVYEQYFGPGTRLTVT

SEQ ID NO: 339 - protein, 114 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRPLGGGYQKVTFGTGTKLQVIP

SEQ ID NO: 340 - protein, 115 aa, Homo sapiens
NAGVTQTPKFRVLKTGQSMTLQCAQDMNHNSMYWYRQDPGMGLRLIYYSASEGTTDKGEVPNGYNVSRLNKREFSLRLESAAPSQTSVYFCASSDTGTSVTGELFFGEGSRLTVL

SEQ ID NO: 341 - protein, 110 aa, Homo sapiens
QKEVEQNSGPLSVPEGAIASLNCTYSDRGSQSFFWYRQYSGKSPELIMFIYSNGDKEDGRFTAQLNKASQYVSLLIRDSQPSDSATYLCAVGTSYDKVIFGPGTSLSVIP

SEQ ID NO: 342 - protein, 112 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYGGGSGYTFGSGTRLTVV

SEQ ID NO: 343 - protein, 112 aa, Homo sapiens
QKEVEQNSGPLSVPEGAIASLNCTYSDRGSQSFFWYRQYSGKSPELIMFIYSNGDKEDGRFTAQLNKASQYVSLLIRDSQPSDSATYLCAVGGAGGYQKVTFGTGTKLQVIP

SEQ ID NO: 344 - protein, 111 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYSGNEQYFGPGTRLTVT

SEQ ID NO: 345 - protein, 110 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRGYQKVTFGTGTKLQVIP

SEQ ID NO: 346 - protein, 114 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASSEASGLRSPLHFGNGTRLTVT

SEQ ID NO: 347 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRTTDSWGKLQFGAGTPVVVTP

SEQ ID NO: 348 - protein, 111 aa, Homo sapiens
NAGVTQTPKFQVLKIGQSMTLQCTQDMNHNYMYWYRQDPGMGLKLIYYSVGAGITDKGEVPNGYNVSRSTTEDFPLRLELAAPSQTSVYFCASSSPLGKQYFGPGTRLTVL

SEQ ID NO: 349 - protein, 110 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVTNAGKSTFGDGTTLTVKP

SEQ ID NO: 350 - protein, 113 aa, Homo sapiens
NAGVTQTPKFQVLKTGQSMTLQCAQDMNHEYMSWYRQDPGMGLRLIHYSVGAGITDQGEVPNGYNVSRSTTEDFPLRLESAAPSQTSVYFCASSEVPGPYGYTFGSGTRLTVV

SEQ ID NO: 351 - protein, 114 aa, Homo sapiens
KQEVTQIPVALSVPEGENLVLNCSFTDSAIYNLQWFRQDPGKGLTSLLLIQSSQREQTSGRLNASLDKSSGRSTLYIAASQPGDSATYLCALRGEADSWGKLQFGAGTQVVVTP

SEQ ID NO: 352 - protein, 111 aa, Homo sapiens
NAGVTQTPKFQVLKTGQSMTLQCAQDMNHNYMYWYRQDPGMGLKLIYYSVGAGITDKGEVPNGYNVSRSTTEDFPLRLELAAPSQTSVYFCASSSPLGEQYFGPGTRLTVT

SEQ ID NO: 353 - protein, 109 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCAVNYRGALTFGSGTRLQVQP

SEQ ID NO: 354 - protein, 115 aa, Homo sapiens
NAGVTQTPKFRILKIGQSMTLQCTQDMNHNYMYWYRQDPGMGLKLIYYSVGAGITDQGEVPNGYNVSRSTTEDFPLRLLSAAPSQTSVYFCASSYLRTGGNYGYTFGSGTRLTVV

SEQ ID NO: 355 - protein, 114 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVWRTTDSWGKLQFGAGTQVVVTP

SEQ ID NO: 356 - protein, 112 aa, Homo sapiens
NAGVTQTPKFRILKIGQSMTLQCTQDMNHNYMYWYRQDPGMGLKLIYYSVGAGITDKGEVPNGYNVSRSTTEDFPLRLLSAAPSQTSVYFCASSYGQGYGYTFGSGTRLTVV

SEQ ID NO: 357 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVKVWSNTGKLIFGQGTTLQVKP

SEQ ID NO: 358 - protein, 114 aa, Homo sapiens
NAGVTQTPKFRILKIGQSMTLQCTQDMNHNYMYWYRQDPGMGLKLIYYSVGAGITDKGEVPNGYNVSRSTTEDFPLRLELAAPSQTSVYFCASSYEGDISYGYTFGFGTRLTVV

SEQ ID NO: 359 - protein, 115 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRPTRGDSWGKLQFGAGTQVVVTP

SEQ ID NO: 360 - protein, 113 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASSYSLLGNEAFFGQGTRLTVV

SEQ ID NO: 361 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVDTGRRALTFGSGTRLQVQP

SEQ ID NO: 362 - protein, 112 aa, Homo sapiens
NAGVTQTPKFQVLKTGQSMTLQCAQDMNHEYMYWYRQDPGMGLRLIHYSVGEGTTAKGEVPDGYNVSRLKKQNFLLGLESAAPSQTSVYFCASRDQINEKLFFGSGTQLSVL

SEQ ID NO: 363 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVNLGAQKLVFGQGTRLTINP

SEQ ID NO: 364 - protein, 112 aa, Homo sapiens
NAGVTQTPKFRILKIGQSMTLQCTQDMNHNYMYWYRQDPGMGLKLIYYSVGAGITDKGEVPNGYNVSRSTTEDFPLRLELAAPSQTSVYFCASSSFDRDEAFFGQGTKLTVV

SEQ ID NO: 365 - protein, 110 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVGGYNKLIFGAGTRLAVHP

SEQ ID NO: 366 - protein, 112 aa, Homo sapiens
NAGVTQTPKFQVLKTGQSMTLQCAQDMNHNSMYWYRQDPGMGLRLIYYSASEGTTDKGEVPNGYNVSRSTTEDFPLRLLSAAPSQTSVYFCASSYQGTGELFFGEGSRLTVL

SEQ ID NO: 367 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRPHGQNFVFGPGTRLSVLP

SEQ ID NO: 368 - protein, 112 aa, Homo sapiens
NAGVTQTPKFRVLKTGQSMTLLCAQDMNHEYMYWYRQDPGMGLRLIHYSVGEGTTDKGEVPNGYNVSRLNKREFSLRLESAAPSQTSVYFCASSSLARETQYFGPGTRLLVL

SEQ ID NO: 369 - protein, 110 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVGGYQKVTFGTGTKLQVIP

SEQ ID NO: 370 - protein, 113 aa, Homo sapiens
NAGVTQTPKFQVLKTGQSMTLQCAQDMNHNSMYWYRQDPGMGLRLIYYSASEGTTDKGEVPNGYNVSRSTTEDFPLRLLSAAPSQTSVYFCASSYQGGNEKLFFGSGTQLSVL

SEQ ID NO: 371 - protein, 110 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRHDYQLSFGAGTTVTVRA

SEQ ID NO: 372 - protein, 112 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSRIGQRASFFGSGTRLTVV

SEQ ID NO: 373 - protein, 114 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAANDPMDSSYKLIFGSGTRLLVRP

SEQ ID NO: 374 - protein, 114 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSPLDGWNYGYTFGSGTRLTVV

SEQ ID NO: 375 - protein, 110 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRHDYKLSFGAGTTVTVRA

SEQ ID NO: 376 - protein, 112 aa, Homo sapiens; identical to SEQ ID NO: 372

SEQ ID NO: 377 - protein, 111 aa, Homo sapiens
KQEVTQIPVALSVPEGENLVLNCSFTDSAIYNLQWFRQDPGKGLTSLLLIQSSQREQTSGRLNASLDKSSGRSTLYIAASQPGDSATYLCAVTDSWGKLQFGAGTQVVVTP

SEQ ID NO: 378 - protein, 114 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSLYFCASSEGPADTYGYTFGSGTRLTVV

SEQ ID NO: 379 - protein, 114 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVSPFDLYGQNFVFGPGTRLSVLP

SEQ ID NO: 380 - protein, 112 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASRGTLFDPQHFGDGTRLSIL

SEQ ID NO: 381 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRPADSWGKLQFGAGTQVVVTP

SEQ ID NO: 382 - protein, 111 aa, Homo sapiens
NAGVTQTPKFRILKIGQSMTLQCTQDMNHNYMYWYRQDPGMGLKLIYYSVGAGITDKGEVPNGYNVSRSTTEDFPLRLELAAPSQTSVYFCASSYSLGELFFGEGSRLTVL

SEQ ID NO: 383 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVLTTDSWGKLQFGAGTQVVVTP

SEQ ID NO: 384 - protein, 118 aa, Homo sapiens
NAGVTQTPKFRILKIGQSMTLQCTQDMNHNSMYWYRQDPGMGLRLIYYSASEGTTDKGEVPNGYNVSRLNKREFSLRLESAAPSQTSVYFCASSELIGTVDYSNQPQHFGDGTRLSIL

SEQ ID NO: 385 - protein, 112 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVLTDSWGKLQFGAGTQVVVTP

SEQ ID NO: 386 - protein, 113 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSYWGGNTEAFFGQGTRLTVV

SEQ ID NO: 387 - protein, 112 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVSGDSWGKLQFGAGTQVVVTP

SEQ ID NO: 388 - protein, 113 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASSYWGPNTEAFFGQGTRLTVV

SEQ ID NO: 389 - protein, 113 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCAVWGKSGGYNKLIFGAGTRLAVHP

SEQ ID NO: 390 - protein, 117 aa, Homo sapiens
NAGVTQTPKFGILKTGQSMTLQCAQDMNHEYMSWYRQDPGMGLRLIHYSVGAGITDKGEVPNGYNVSRSTTEDFPLRLELAAPSQTSVYFCASSYSSPLRQDNSPLHFGNGTRLTVT

SEQ ID NO: 391 - protein, 111 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCAVNSPGNARLMFGDGTQLVVKP

SEQ ID NO: 392 - protein, 113 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYWRGGLEQYFGPGTRLTVT

SEQ ID NO: 393 - protein, 112 aa, Homo sapiens
AQSVTQLDSHVSVSEGTPVLLRCNYSSSVPPYLFWYVQYPNQGLQLLLKYTTGATLVKGINGFEAEFKKSETSFHLTKPSAHMSDAAEYFCAVSEDGNKLVFGAGTILRVKS

SEQ ID NO: 394 - protein, 117 aa, Homo sapiens
AAGVIQSPRHLIKEKRETATLKCYPIPRHDTVYWYQQGPGQDPQFLISFYEKMQSDKGSIPDRFSAQQFSDYHSELNMSSLELGDSALYFCASRLTGVSGEGDYGYTFGSGTRLTVV

SEQ ID NO: 395 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVLDSWGKLQFGAGTQVVVTP

SEQ ID NO: 396 - protein, 113 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSGLLSPDTQYFGPGTRLKVL

SEQ ID NO: 397 - protein, 110 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAAGGYQKVTFGTGTKLQVIP

SEQ ID NO: 398 - protein, 111 aa, Homo sapiens
NAGVTQTPKFRILKIGQSMTLQCTQDMNHNYMYWYRQDPGMGLKLIYYSVGAGITDKGEVPNGYNVSRSTTEDFPLRLLSAAPSQTSVYFCASRRPTGELFFGEGSRLTVL

SEQ ID NO: 399 - protein, 114 aa, Homo sapiens
KQEVTQIPAALSVPEGENLVLNCSFTDSAIYNLQWFRQDPGKGLTSLLLIRSSQREQTSGRLNASLDKSSGRSTLYIAASQPGDSATYLCAVMSSQGGSEKLVFGKGTKLTVNP

SEQ ID NO: 400 - protein, 112 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASKGLVADTIYFGEGSWLTVV

SEQ ID NO: 401 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVSPPAAGNKLTFGGGTRVLVKP

SEQ ID NO: 402 - protein, 114 aa, Homo sapiens
NAGVTQTPKFRILKIGQSMTLQCAQDMNHNYMYWYRQDPGMGLKLIYYSVGAGITDKGEVPNGYNVSRSTTEDFPLRLELAAPSQTSVYFCASSLTWTGGFGYTFGSGTRLTVV

SEQ ID NO: 403 - protein, 114 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRPRGDSWGKLQFGAGTQVVVTP

SEQ ID NO: 404 - protein, 112 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSPTGYGEAFFGQGTRLTVV

SEQ ID NO: 405 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRASGGSYIPTFGRGTSLIVHP

SEQ ID NO: 406 - protein, 112 aa, Homo sapiens
NAGVTQTPKFQVLKTGQSMTLQCAQDMNHEYMSWYRQDPGMGLRLIHYSVGAGITYQGEVPNGYNVSRSTTEDFPLRLLSAAPSQTSVYFCASSYSPWGEAFFGQGTRLTVV

SEQ ID NO: 407 - protein, 112 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRSYNTDKLIFGTGTRLQVFP

SEQ ID NO: 408 - protein, 112 aa, Homo sapiens
NAGVTQTPKFRILKIGQSMTLQCTQDMNHNYMYWYRQDPGMGLKLIYYSVGAGITDKGEVPNGYNVSRSTTEDFPLRLELAAPSQTSVYFCASSYSSWTEAFFGQGTRLTVV

SEQ ID NO: 409 - protein, 112 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVTAYNTDKLIFGTGTRLQVFP

SEQ ID NO: 410 - protein, 114 aa, Homo sapiens
NAGVTQTPKFRILKIGQSMTLQCTQDMNHNYMYWYRQDPGMGLKLIYYSVGAGITDKGEVPNGYNVSRSTTEDFPLRLLSAAPSQTSVYFCASSYYPGTSTEAFFGQGTRLTVV

SEQ ID NO: 411 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVSGGSYIPTFGRGTSLIVHP

SEQ ID NO: 412 - protein, 111 aa, Homo sapiens
NAGVTQTPKFRVLKTGQSMTLLCAQDMNHEYMYWYRQDPGMGLRLIHYSVGEGTTDKGEVPDGYNVSRSTTEDFPLRLLSAAPSQTSVYFCASRKAGYGYTFGSGTRLTVV

SEQ ID NO: 413 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVMDSSYKLIFGSGTRLLVRP

SEQ ID NO: 414 - protein, 109 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASGFYGYTFGSGTRLTVV

SEQ ID NO: 415 - protein, 110 aa, Homo sapiens; identical to SEQ ID NO: 345

SEQ ID NO: 416 - protein, 111 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSPGIGELFFGEGSRLTVL

SEQ ID NO: 417 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRAQGGSEKLVFGKGTKLTVNP

SEQ ID NO: 418 - protein, 116 aa, Homo sapiens
NAGVTQTPKFRILKIGQSMTLQCTQDMNHNYMYWYRQDPGMGLKLIYYSVGAGITDKGEVPNGYNVSRSTTEDFPLRLELAAPSQTSVYFCASSYLGQDPEGAEAFFGQGTRLTVV

SEQ ID NO: 419 - protein, 115 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVSGGTTDSWGKLQFGAGTQVVVTP

SEQ ID NO: 420 - protein, 112 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSFTPYEGYTFGSGTRLTVV

SEQ ID NO: 421 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVESDDYKLSFGAGTTVTVRA

SEQ ID NO: 422 - protein, 115 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASSEALGGVTDTQYFGPGTRLTVL

SEQ ID NO: 423 - protein, 115 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVSGGATDSWGKLQFGAGTQVVVTP

SEQ ID NO: 424 - protein, 111 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYSPWTEAFFGQGTRLTV

SEQ ID NO: 425 - protein, 110 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRDDYKLSFGAGTTVTVRA

SEQ ID NO: 426 - protein, 114 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSYGPGQIYGYTFGSGTRLTVV

SEQ ID NO: 427 - protein, 110 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRNDYKLSFGAGTTVTVRA

SEQ ID NO: 428 - protein, 114 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSYGPGQIYGYTFGPGTRLTVL

SEQ ID NO: 429 - protein, 114 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRPFSGGYNKLIFGAGTRLAVHP

SEQ ID NO: 430 - protein, 113 aa, Homo sapiens
NAGVTQTPKFQVLKTGQSMTLQCTQDMNHNYMYWYRQDPGMGLKLIYYSVGAGITDQGEVPNGYNVSRSTTEDFPLRLLSAAPSQTSVYFCASSPEYGYYEQYFGPGTRLTVT

SEQ ID NO: 431 - protein, 110 aa, Homo sapiens; identical to SEQ ID NO: 365

SEQ ID NO: 432 - protein, 112 aa, Homo sapiens
NAGVTQTPKFQVLKTGQSMTLQCAQDMNHNSMYWYRQDPGMGLRLIYYSASEGTTDKGEVPNGYNVSRSTTEDFPLRLLSAAPSQTSVYFCASSLDPLAEAFFGQGTRLTVV

SEQ ID NO: 433 - protein, 110 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVESGNQFYFGTGTSLTVIP

SEQ ID NO: 434 - protein, 113 aa, Homo sapiens
NAGVTQTPKFQVLKTGQSMTLQCAQDMNHEYMSWYRQDPGMGLRLIHYSVGVGITDQGEVPNGYNVSRSTTEDFPLRLLSAAPSQTSVYFCASSFGQGSSPLHFGNGTRLTVT

SEQ ID NO: 435 - protein, 112 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAALSYNTDKLIFGTGTRLQVFP

SEQ ID NO: 436 - protein, 112 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASKDIPYTEAFFGQGTRLTVV

SEQ ID NO: 437 - protein, 110 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCAVGGGADGLTFGKGTHLIIQP

SEQ ID NO: 438 - protein, 113 aa, Homo sapiens
NAGVTQTPKFRILKIGQSMTLQCTQDMNHNYMYWYRQDPGMGLKLIYYSVGAGITDKGEVPNGYNVSRSTTEDFPLRLELAAPSQTSVYFCASSYGNLGTEAFFGQGTRLTVV

SEQ ID NO: 439 - protein, 111 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCAVSGGSQGNLIFGKGTKLSVKP

SEQ ID NO: 440 - protein, 113 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYGLGGTEAFFGQGTRLTVV

SEQ ID NO: 441 - protein, 112 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCAVNDSGGYQKVTFGTGTKLQVIP

SEQ ID NO: 442 - protein, 112 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYSPGNGYTFGSGTRLTVV

SEQ ID NO: 443 - protein, 110 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCAVNDSWGKLQFGAGTQVVVTP

SEQ ID NO: 444 - protein, 113 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSMVPGFSEQFFGPGTRLTVL

SEQ ID NO: 445 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVSPVTSYDKVIFGPGTSLSVIP

SEQ ID NO: 446 - protein, 115 aa, Homo sapiens
NAGVTQTPKFRVLKTGQSMTLLCAQDMNHEYMYWYRQDPGMGLRLIYYSASEGTTDKGEVPNGYNVSRLNKREFSLRLESAAPSQTSVYFCASSYSWFGDGTEAFFGQGTRLTVV

SEQ ID NO: 447 - protein, 110 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCAVSAAGNKLTFGGGTRVLVKP

SEQ ID NO: 448 - protein, 110 aa, Homo sapiens
NAGVTQTPKFRILKIGQSMTLQCTQDMNHNYMYWYRQDPGMGLKLIYYSAGAGITDKGEVPNGYNVSRSTTEDFPLRLELAAPSQTSVYFCASSYFGEQYFGPGTRLTVT

SEQ ID NO: 449 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRPGDSWGKLQFGAGTQVVVTP

SEQ ID NO: 450 - protein, 111 aa, Homo sapiens
NAGVTQTPKFQVLKTGQSMTLQCAQDMNHEYMSWYRQDPGMGLKLIYYSVGAGITDKGEVPNGYNVSRSTTEDFPLRLLSAAPSQTSVYFCASSSPVGTAFFGQGTRLTVV

SEQ ID NO: 451 - protein, 112 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVGTDSWGKFQFGAGTQVVVTP

SEQ ID NO: 452 - protein, 112 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYSVDSPLHFGNGTRLSVL

SEQ ID NO: 453 - protein, 112 aa, Homo sapiens; identical to SEQ ID NO: 451

SEQ ID NO: 454 - protein, 112 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSFSPNTEAFFGQGTRLTVV

SEQ ID NO: 455 - protein, 110 aa, Homo sapiens; identical to SEQ ID NO: 345

SEQ ID NO: 456 - protein, 111 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCATSGLNTEAFFGQGTRLTVV

SEQ ID NO: 457 - protein, 110 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVSGYSTLTFGKGTMLLVSP

SEQ ID NO: 458 - protein, 115 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASTGGGPPLNTEAFFGQGTRLTVV

SEQ ID NO: 459 - protein, 110 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAASGYSTLTFGKGTMLLVSP

SEQ ID NO: 460 - protein, 112 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRLGQFGQAFFGQGTRLTVV

SEQ ID NO: 461 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVSRSWGKFQFGAGTQVVVTP

SEQ ID NO: 462 - protein, 113 aa, Homo sapiens
NAGVTQTPKFQVLKTGQSMTLQCAQDMNHEYMSWYRQDPGMGLRLIHYSVGAGITDQGEVPNGYNVSRLNKREFSLRLESAAPSQTSVYFCASSLTGGAEYGYTFGSGTRLTV

SEQ ID NO: 463 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVQGGSEKLVFGKGTKLTVNP

SEQ ID NO: 464 - protein, 111 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASSEEWSYGYTFGSGTRLTV

SEQ ID NO: 465 - protein, 110 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRSYNQFYFGTGTSLTVIP

SEQ ID NO: 466 - protein, 112 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSPGGPSTEAFFGQGTRLTV

SEQ ID NO: 467 - protein, 111 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCAVKGTGRRALTFGSGTRLQVQP

SEQ ID NO: 468 - protein, 112 aa, Homo sapiens
NAGVTQTPKFQVLKTGQSMTLQCAQDMNHNSMYWYRQDPGMGLRLIHYSVGEGTTAKGEVPDGYNVSRLKKQNFLLGLESAAPSQTSVYFCATAGPNPYEQYFGPGTRLTVT

SEQ ID NO: 469 - protein, 110 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCAVRRNTDKLIFGTGTRLQVFP

SEQ ID NO: 470 - protein, 115 aa, Homo sapiens
DTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSPRDRSPYGYTFGSGTRLTVV

SEQ ID NO: 471 - protein, 112 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVSPINDYKLSFGAGTTVTVRA

SEQ ID NO: 472 - protein, 114 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSLVGTGGSPLHFGNGTRLTVT

SEQ ID NO: 473 - protein, 110 aa, Homo sapiens
QKEVEQNSGPLSVPEGAIASLNCIYSDRGSQSFFWYRQYSGKSPELIMSIYSNGDKEDGRFTAQLNKASQYVSLLIRDSQPSDSATYLCAVTNSGYALNFGKGTSLLVTP

SEQ ID NO: 474 - protein, 111 aa, Homo sapiens
NAGVTQTPKFQVLKTGQSMTLQCAQDMNHNSMYWYRQDPGMGLRLIYYSASEGTTDKGEVPNGYNVSRSTTEDFPLRLLSAAPSQTSVYFCASSYLGGEAFFGQGTRLTVV

SEQ ID NO: 475 - protein, 109 aa, Homo sapiens
QKEVEQNSGPLSVPEGAIASLNCTYSDRGSQSFFWYRQYSGKSPELIMFIYSNGDKEDGRFTAQLNKASQYVSLLIRDSQPSDSATYLCAVDGSYIPTFGRGTSLIVHP

SEQ ID NO: 476 - protein, 114 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSSGGADTGELFFGEGSRLTVL

SEQ ID NO: 477 - protein, 110 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCAVSGSARQLTFGSGTQLTVLP

SEQ ID NO: 478 - protein, 114 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSPFADDSYGYTFGSGTRLTVV

SEQ ID NO: 479 - protein, 109 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCAVNNNARLMFGDGTQLVVKP

SEQ ID NO: 480 - protein, 115 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASNRLAGVYYNEQFFGPGTRLTVL

SEQ ID NO: 481 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAATDSWGKLQFGAGTQVVVTP

SEQ ID NO: 482 - protein, 116 aa, Homo sapiens
DTGVSQNPRHKITKRGQNVTFRCDPISEHNRLYWYRQTLGQGPEFLTYFRNEAQLEKSRLLSDRFSAERPKGSFSTLEIQRTEQGDSAMYLCASSLFSRGTNYGYTFGSGTRLTVV

SEQ ID NO: 483 - protein, 114 aa, Homo sapiens
ELKVEQNPLFLSMQEGKNYTIYCNYSTTSDRLYWYRQDPGKSLESLFVLLSNGAVKQEGRLMASLDTKARLSTLHITAAVHDLSATYFCAVGAYSGAGSYQLTFGKGTKLSVIP

SEQ ID NO: 484 - protein, 117 aa, Homo sapiens
NAGVIQNPRHLVRRRGQEARLRCSPMKGHSHVYWYRQLPEEGLKFMVYLQKENIIDESGMPKERFSAEFPKEGPSILRIQQVVRGDSAAYFCASSKYPAGEANEKLFFGSGTQLSVL

SEQ ID NO: 485 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRLRGSARQLTFGSGTQLTVLP

SEQ ID NO: 486 - protein, 113 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSYSIEFTEAFFGQGTRLTVV

SEQ ID NO: 487 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCALGYRGSARQLTFGSGTQLTVLP

SEQ ID NO: 488 - protein, 112 aa, Homo sapiens
NAGVTQTPKFRVLKTGQSMTLLCAQDMNHEYMYWYRQDPGMGLRLIHYSVGEGTTAKGEVPDGYNVSRLNKREFSLRLESAAPSQTSVYFCASSVGYPGELFFGEGSRLTVL

SEQ ID NO: 489 - protein, 110 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRGDYKLSFGAGTTVTVRA

SEQ ID NO: 490 - protein, 114 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASSDPLGGLYGYTFGSGTRLTVV

SEQ ID NO: 491 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVSNSGGYQKVTFGTGTKLQVIP

SEQ ID NO: 492 - protein, 113 aa, Homo sapiens
NAGVTQTPKFQVLKTGQSMTLQCAQDMNHEYMSWYRQDPGMGLRLIHYSVGAGITDQGEVPDGYNVSRSTTEDFPLRLLSAAPSQTSVYFCASSYSTGSYEQYFGPGTRLTVT

SEQ ID NO: 493 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVGNSGGYQKVTFGTGTKLQVIP

SEQ ID NO: 494 - protein, 113 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYSTGSYEQYFGPGTRLTVT

SEQ ID NO: 495 - protein, 113 aa, Homo sapiens; identical to SEQ ID NO: 493

SEQ ID NO: 496 - protein, 113 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYSTGSYGYTFGSGTRLTVV

SEQ ID NO: 497 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAARYNFNKFYFGSGTKLNVKP

SEQ ID NO: 498 - protein, 114 aa, Homo sapiens
NAGVTQTPKFQVLKTGQSMTLLCAQDMNHEYMYWYRQDPGMGLRLIHYSVGEGTTAKGEVPDGYNVSRSTTEDFPLRLLSAAPSQTSVYFCASSYTPGPSHEQYFGPGTRLTVT

SEQ ID NO: 499 - protein, 117 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVIDRAYSGAGSYQLTFGKGTKLSVIP

SEQ ID NO: 500 - protein, 113 aa, Homo sapiens
NAGVTQTPKFRILKIGQSMTLQCTQDMNHNYMYWYRQDPGMGLKLIHYSVGAGITDQGEVPNGYNVSRSTTEDFPLRLLSAAPSQTSVYFCASSLGVSYNEQFFGPGTRLTVL

SEQ ID NO: 501 - protein, 109 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCAVNDGNKLVFGAGTILRVKS

SEQ ID NO: 502 - protein, 110 aa, Homo sapiens
NAGVTQTPKFRILKIGQSMTLQCTQDMNHNYMYWYRQDPGMGLKLIYYSVGAGITDKGEVPNGYNVSRSTTEDFPLRLELAAPSQTSVYFCASAAGQPQHFGDGTRLSIL

SEQ ID NO: 503 - protein, 110 aa, Homo sapiens
QKEVEQNSGPLSVPEGAIASLNCTYSDRGSQSFFWYRQYSGKSPELIMFIYSNGDKEDGRFTAQLNKASQYVSLLIRDSQPSDSATYLCAVMGNTDKLIFGTGTRLQVFP

SEQ ID NO: 504 - protein, 112 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYGVGSPLHFGNGTRLTVT

SEQ ID NO: 505 - protein, 110 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCAVMDSSYKLIFGSGTRLLVRP

SEQ ID NO: 506 - protein, 112 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYGGGAEAFFGQGTRLTVV

SEQ ID NO: 507 - protein, 110 aa, Homo sapiens
QKEVEQNSGPLSVPEGAIASLNCTYSDRGSQSFFWYRQYSGKSPELIMFIYSNGDKEDGRFTAQLNKASQYVSLLIRDSQPSDSATYLCAVKTSYDKVIFGPGTSLSVIP

SEQ ID NO: 508 - protein, 112 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYGAGAFYTFGSGTRLTVV

SEQ ID NO: 509 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVPDNNAGNMLTFGGGTRLMVKP

SEQ ID NO: 510 - protein, 110 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSSGAGYTFGSGTRLTVV

SEQ ID NO: 511 - protein, 112 aa, Homo sapiens
KQEVTQIPVALSVPEGENLVLNCSFTDSAIYNLQWFRQDPGKGLTSLLLIQSSQREQTSGRLNASLDKSSGRSTLYIAASQPGDSATYLCAVSPYNNNDMRFGAGTRLTVKP

SEQ ID NO: 512 - protein, 112 aa, Homo sapiens
NAGVTQTPKFQVLKTGQSMTLLCAQDMNHEYMYWYRQDPGMGLRLIHYSVGEGTTDKGEVPNGYNVSRSTTEDFPLRLLSAAPSQTSVYFCASSFTGDSPLHFGNGTRLTVT

SEQ ID NO: 513 - protein, 110 aa, Homo sapiens; identical to SEQ ID NO: 365

SEQ ID NO: 514 - protein, 112 aa, Homo sapiens
NAGVTQTPKFRVLKTGQSMTLLCAQDMNHEYMYWYRQDPGMGLRLIHYSVGAGITDQGEVPNGYNVSRSTTEDFPLRLLSAAPSQTSVYFCASRATGLTEAFFGQGTRLTVV

SEQ ID NO: 515 - protein, 110 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVVSNNDMRFGAGTRLTVKP

SEQ ID NO: 516 - protein, 116 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSLHSPVQGDYGYTFGSGTRLTVV

SEQ ID NO: 517 - protein, 110 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAPGGYQKVTFGTGTKLQVIP

SEQ ID NO: 518 - protein, 112 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRRDPLGGYTFGSGTRLTVV

SEQ ID NO: 519 - protein, 114 aa, Homo sapiens
KQEVTQIPAALSVPEGENLALNCSFTDSAIYNLQWFRQDPGKGLTSLLLIQSSQREQTSGRLNASLDKSSGRSTLYIAASQPGDSATYLCAVNAGGTSYGKLTFGQGTILTVHP

SEQ ID NO: 520 - protein, 113 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASSYWREYYEQYFGPGTRLTVT

SEQ ID NO: 521 - protein, 110 aa, Homo sapiens
QKEVEQNSGPLSVPEGAIASLNCTYSDRGSQSFFWYRQYSGKSPELIMFIYSNGDKEDGRFTAQLNKASQYVSLLIRDSQPSDSATYLCAVKNSSYKLIFGSGTRLLVRP

SEQ ID NO: 522 - protein, 113 aa, Homo sapiens
NAGVTQTPKFRILKIGQSMTLQCTQDMNHNYMYWYRQDPGMGLKLIYYSVGAGITDKGEVPNGYNVSRSTTEDFPLRLELAAPSQTSVYFCASSYEQVYQPQHFGDGTRLSIL

SEQ ID NO: 523 - protein, 113 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCAVNAGGTSYGKLTFGQGTILTVHP

SEQ ID NO: 524 - protein, 115 aa, Homo sapiens
NAGVTQTPKFRVLKTGQSMTLQCAQDMNHNSMYWYRQDPGMGLRLIYYSASEGTTDKGEVPNGYNVSRLNKREFSLRLESAAPSQTSVYFCASSEIDVWYNSPLHFGNGTRLTVT

SEQ ID NO: 525 - protein, 109 aa, Homo sapiens
QKEVEQNSGPLSVPEGAIASLNCTYSDRGSQSFFWYRQYSGKSPELIMFIYSNGDKEDGRFTAQLNKASQYVSLLIRDSQPSDSATYLCAVGGYQKVTFGTGTKLQVIP

SEQ ID NO: 526 - protein, 112 aa, Homo sapiens
NAGVTQTPKFGILKIGQSMTLQCAQDMNHNYMYWYRQDPGMGLKLIYYSVGAGITDKGEVPNGYNVSRSTTEDFPLRLELAAPSQTSVYFCASRVDSYNEQFFGPGTRLTVL

SEQ ID NO: 527 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRPSGSARQLTFGSGTQLTVLP

SEQ ID NO: 528 - protein, 112 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYGVYTEAFFGQGTRLTVV

SEQ ID NO: 529 - protein, 114 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRPSTDSWGKLQFGAGTQVVVTP

SEQ ID NO: 530 - protein, 112 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSPTGYYGYTFGSGTRLTVV

SEQ ID NO: 531 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVLSNFGNEKLTFGTGTRLTIIP

SEQ ID NO: 532 - protein, 114 aa, Homo sapiens
NAGVTQTPKFRILKIGQSMTLQCTQDMNHNYMYWYRQDPGMGLKLIYYSVGAGITDKGEVPNGYNVSRSTTEDFPLRLLSAAPSQTSVYFCASTREAGLDYGYTFGSGTRLTVV

SEQ ID NO: 533 - protein, 110 aa, Homo sapiens; identical to SEQ ID NO: 457

SEQ ID NO: 534 - protein, 112 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSEGPFSEAFFGQGTRLTVV

SEQ ID NO: 535 - protein, 110 aa, Homo sapiens; identical to SEQ ID NO: 369

SEQ ID NO: 536 - protein, 112 aa, Homo sapiens
NAGVTQTPKFRVLKTGQSMTLLCAQDMNHNYMYWYRQDPGMGLKLIYYSVGAGITDKGEVPNGYNVSRSTTEDFPLRLELAAPSQTSVYFCASRVDTLGEAFFGQGTRLTVV

SEQ ID NO: 537 - protein, 110 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVGGYDKVIFGPGTSLSVIP

SEQ ID NO: 538 - protein, 112 aa, Homo sapiens; identical to SEQ ID NO: 460

SEQ ID NO: 539 - protein, 112 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRVTGANNLFFGTGTRLTVIP

SEQ ID NO: 540 - protein, 115 aa, Homo sapiens
NAGVTQTPKFQVLKTGQSMTLQCAQDMNHEYMSWYRQDPGMGLRLIHYSVGAGITDQGEVPNGYNVSRLNKREFSLRLESAAPSQTSVYFCASRPVGGDYTGELFFGEGSRLTVL

SEQ ID NO: 541 - protein, 110 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVDSWGKLQFGAGTQVVVTP

SEQ ID NO: 542 - protein, 114 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSSLGLGDYGYTFGSGTRLTVV

SEQ ID NO: 543 - protein, 110 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVGDDYKLSFGAGTTVTVRA

SEQ ID NO: 544 - protein, 114 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASSEGWGGRTEAFFGQGTRLTVV

SEQ ID NO: 545 - protein, 110 aa, Homo sapiens; identical to SEQ ID NO: 425

SEQ ID NO: 546 - protein, 114 aa, Homo sapiens; identical to SEQ ID NO: 426

SEQ ID NO: 547 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRQGYSTLTFGKGTMLLVSP

SEQ ID NO: 548 - protein, 115 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASSYSYPLAVGELFFGEGSRLTVL

SEQ ID NO: 549 - protein, 110 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVSNDYKLSFGAGTTVTVRA

SEQ ID NO: 550 - protein, 114 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSYSSRIYNEQFFGPGTRLTVL

SEQ ID NO: 551 - protein, 109 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVTGNQFYFGTGTSLTVIP

SEQ ID NO: 552 - protein, 115 aa, Homo sapiens
NAGVTQTPKFRVLKTGQSMTLQCAQDMNHEYMYWYRQDPGMGLRLIHYSVGAGITDQGEVPNGYNVSRSTTEDFPLRLLSAAPSQTSVYFCASSTYPVWGGEKMFFGSGTQLSVL

SEQ ID NO: 553 - protein, 110 aa, Homo sapiens
QKEVEQNSEPLSVPEGAIASLNCTYSDRGSQSFFWYRQYSGKSPELIMSIYSNGDKEDGRFTAQLNKASQYVSLLIRDSQPSDSATYLCAVNSQGGKLMFGQGTELSVKP

SEQ ID NO: 554 - protein, 115 aa, Homo sapiens
NAGVTQTPKFRVLKTGQSMTLQCAQDMNHNYMYWYRQDPGMGLRLIHYSVGEGTTAKGEVPDGYNVSRLKKQNFLLGLESAAPSQTSVYFCASSYAPWPGPYEQYVGPGTRLTVT

SEQ ID NO: 555 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVQTGANNLFFGTGTRLTVIP

SEQ ID NO: 556 - protein, 115 aa, Homo sapiens
NAGVTQTPKFRILKIGQSMTLQCTQDMNHNYMYWYRQDPGMGLRLIHYSVGAGITDQGEVPNGYNVSRSTTEDFPLRLLSAAPSQTSVYFCASSTYPVWGGEKLFFGSGTQLSVL

SEQ ID NO: 557 - protein, 112 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCASDSGGGADGLTFGKGTHLIIQP

SEQ ID NO: 558 - protein, 111 aa, Homo sapiens
NAGVTQTPKFQVLKTGQSMTLQCAQDMNHEYMSWYRQDPGMGLRLIHYSVGAGITDQGEVPNGYNVSRSTTEDFPLRLESAAPSQTSVYFCASSYGGDGYTFGSGTRLTVV

SEQ ID NO: 559 - protein, 112 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCAADSGGGADGLTFGKGTHLIIQP

SEQ ID NO: 560 - protein, 112 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASTSPGGDGYTFGSGTRLTVV

SEQ ID NO: 561 - protein, 112 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCAVDSGGGADGLTFGKGTHLIIQP

SEQ ID NO: 562 - protein, 111 aa, Homo sapiens
NAGVTQTPKFRILKTGQSMTLQCAQDMNHEYMSWYRQDPGMGLRLIHYSVGEGTTAKGEVPDGYNVSRLKKQNFLLGLESAAPSQTSVYFCASSYEGYGYTFGSGTRLTVV

SEQ ID NO: 563 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRPTEYGNKLVFGAGTILRVKS

SEQ ID NO: 564 - protein, 111 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSNEGGGYTFGSGTRLTVV

SEQ ID NO: 565 - protein, 112 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVDKAAGNKLTFGGGTRVLVKP

SEQ ID NO: 566 - protein, 111 aa, Homo sapiens
NAGVTQTPKFQVLKTGQSMTLQCAQDMNHNYMYWYRQDPGMGLRLIHYSVGAGITDQGEVPNGYNVSRSTTEDFPLRLLSAAPSQTSVYFCASKNWGTEAFFGQGTRLTVV

SEQ ID NO: 567 - protein, 112 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVSGGGADGLTFGKGTHLIIQP

SEQ ID NO: 568 - protein, 114 aa, Homo sapiens
NAGVTQTPKFRILKIGQSMTLQCTQDMNHNYMYWYRQDPGMGLKLIYYSVGAGITDQGEVPNGYNVSRLNKREFSLRLESAAPSQTSVYFCASSEEGWSNSPLHFGNGTRLTVT

SEQ ID NO: 569 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRDSWGKFQFGAGTQVVVTP

SEQ ID NO: 570 - protein, 117 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSLGQGYGLAYDEQFFGPGTRLTVL

SEQ ID NO: 571 - protein, 112 aa, Homo sapiens; identical to SEQ ID NO: 435

SEQ ID NO: 572 - protein, 112 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASRYSPYTEAFFGQGTRLTVV

SEQ ID NO: 573 - protein, 110 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRGYSTLTFGKGTMLLVSP

SEQ ID NO: 574 - protein, 112 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSRGQFTEAFFGQGTRLTVV

SEQ ID NO: 575 - protein, 112 aa, Homo sapiens; identical to SEQ ID NO: 435

SEQ ID NO: 576 - protein, 111 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASSEAYLAAFFGQGTRLTVV

SEQ ID NO: 577 - protein, 112 aa, Homo sapiens; identical to SEQ ID NO: 283

SEQ ID NO: 578 - protein, 110 aa, Homo sapiens
NAGVTQTPKFRILKIGQSMTLQCTQDMNHNYMYWYRQDPGMGLKLIYYSVGAGITDKGEVPNGYNVSRSTTEDFPLRLESAAPSQTSVYFCASSSYNEQFFGPGTRLTVL

SEQ ID NO: 579 - protein, 114 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRLTQGGSEKLVFGKGTKLTVNP

SEQ ID NO: 580 - protein, 110 aa, Homo sapiens
NAGVTQTPKFRILKIGQSMTLQCTQDMNHNYMYWYRQDPGMGLKLIYYSVGAGITDKGEVPNGYNVSRSTTEDFPLRLLSAAPSQTSVYFCASSSYNEQFFGPGTRLTVL

SEQ ID NO: 581 - protein, 110 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVFNDYKLSFGAGTTVTVRA

SEQ ID NO: 582 - protein, 112 aa, Homo sapiens
NAGVTQTPKFQVLKTGQSMTLQCAQDMNHEYMSWYRQDPGMGLRLIHYSVGAGITDQGEVPNGYNVSRLNKREFSLRLESAAPSQTSVYFCASSEWFDTEAFFGQGTRLTVV

SEQ ID NO: 583 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRGGAGSYQLTFGKGTKLSVIP

SEQ ID NO: 584 - protein, 114 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASSDESYGYYGYTFGSGTRLTVV

SEQ ID NO: 585 - protein, 115 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRGAGGTSYGKLTFGQGTILTVHP

SEQ ID NO: 586 - protein, 114 aa, Homo sapiens; identical to SEQ ID NO: 584

SEQ ID NO: 587 - protein, 113 aa, Homo sapiens; identical to SEQ ID NO: 315

SEQ ID NO: 588 - protein, 114 aa, Homo sapiens
NAGVTQTPKFRILKIGQSMTLQCAQDMNHNYMYWYRQDPGMGLKLIYYSVGAGITDKGEVPNGYNVSRSTTEDFPLRLELAAPSQTSVYFCASSTGSSYNSPLHFGNGTRLTVT

SEQ ID NO: 589 - protein, 110 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCAVGGRDDKIIFGKGTRLHILP

SEQ ID NO: 590 - protein, 114 aa, Homo sapiens
NAGVTQTPKFQVLKTGQSMTLQCAQDMNHNSMYWYRQDPGMGLRLIYYSASEGTTDKGEVPNGYNVSRSTTEDFPLRLLSAAPSQTSVYFCASSYTDGVNSPLHFGNGTRLTVT

SEQ ID NO: 591 - protein, 112 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVDQDSGYALNFGKGTSLLVTP

SEQ ID NO: 592 - protein, 113 aa, Homo sapiens
DTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSLYRSYEQYFGPGTRLTVT

SEQ ID NO: 593 - protein, 115 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRPWGYNQGGKLIFGQGTELSVKP

SEQ ID NO: 594 - protein, 112 aa, Homo sapiens
NAGVTQTPKFQVLKTGQSMTLQCAQDMNHNSMYWYRQDPGMGLRLIYYSASEGTTDKGEVPNGYNVSRSTTEDFPLRLLSAAPSQTSVYFCASSYWGDTEAFFGQGTRLTVV

SEQ ID NO: 595 - protein, 114 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVKEGTDSWGKFQFGAGTQVVVTP

SEQ ID NO: 596 - protein, 111 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSYPSREAFFGQGTRLTVV

SEQ ID NO: 597 - protein, 110 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVWSWGKLQFGAGTQVVVTP

SEQ ID NO: 598 - protein, 111 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSRGVPEVFFGQGTRLTVV

SEQ ID NO: 599 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVSGSWGKLQFGAGTQVVVTP

SEQ ID NO: 600 - protein, 113 aa, Homo sapiens
NAGVTQTPKFRVLKTGQSMTLLCAQDMNHEYMYWYRQDPGMGLRLIHYSVGEGTTAKGEVPDGYNVSRSTTEDFPLRLLSAAPSQTSVYFCASSLMDRPGEQFFGPGTRLTVL

SEQ ID NO: 601 - protein, 111 aa, Homo sapiens; identical to SEQ ID NO: 307

SEQ ID NO: 602 - protein, 112 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSYADTYEQYFGPGTRLTVT

SEQ ID NO: 603 - protein, 111 aa, Homo sapiens; identical to SEQ ID NO: 569

SEQ ID NO: 604 - protein, 115 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASSEWAWGLGTEAFFGQGTRLTVV

SEQ ID NO: 605 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAAATRNNTGKLIFGQGTTLQVKP

SEQ ID NO: 606 - protein, 112 aa, Homo sapiens
NAGVTQTPKFRILKIGQSMTLQCTQDMNHNYMYWYRQDPGMGLKLIYYSVGAGITDKGEVPNGYNVSRSTTEDFPLRLELAAPSQTSVYFCASSKSDLGGYTFGSGTRLTVV

SEQ ID NO: 607 - protein, 114 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRGMNTGFQKLVFGTGTRLLVSP

SEQ ID NO: 608 - protein, 113 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSPGVTSTEAFFGQGTRLTVV

SEQ ID NO: 609 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVPAQGGSEKLVFGKGTKLTVNP

SEQ ID NO: 610 - protein, 112 aa, Homo sapiens
NAGVTQTPKFQVLKTGQSMTLQCAQDMNHEYMSWYRQDPGMGLKLIYYSVGAGITDKGEVPNGYNVSRSTTEDFPLRLESAAPSQTSVYFCASTESPYYGYTFGSGTRLTVV

SEQ ID NO: 611 - protein, 114 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRPRGDSSYKLIFGSGTRLLVRP

SEQ ID NO: 612 - protein, 114 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSASGSIYNEQFFGPGTRLTVL

SEQ ID NO: 613 - protein, 110 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCAVNEGYQKVTFGIGTKLQVIP

SEQ ID NO: 614 - protein, 112 aa, Homo sapiens
NAGVTQTPKFQVLKTGQSMTLQCAQDMNHEYMYWYRQDPGMGLRLIHYSVGAGITDQGEVPNGYNVSRSTTEDFPLRLLSAAPSQTSVYFCASSRDYDYGQYFGPGTRLTVT

SEQ ID NO: 615 - protein, 111 aa, Homo sapiens
QKEVEQNSGPLSVPEGAIASLSCTYSDRGSQSFFWYRQYSGKSPELIMSIYSNGDKEDGRFTAQLNKASQYVSLLIRDSQPSDSATYLCAVGAGGYNKLIFGAGTRLAVHP

SEQ ID NO: 616 - protein, 112 aa, Homo sapiens
NAGVTQTPKFRVLKTGQSMTLQCTQDMNHNYMYWYRQDPGMETGLRLIHYSVGEGTTAKGEVPDGYNVSRSTTEDFPLRLLSAAPSQTSVYFCASRREGPLHFGNGTRLTVT

SEQ ID NO: 617 - protein, 112 aa, Homo sapiens
QKEVEQNSGPLSVPEGAIASLNCTYSDRGSQSFFWYRQYSGKSPELIMFIYSNGDKEDGRFTAQLNKASQYVSLLIRDSQPSDSATYLCAVGERGSQGNLIFGKGTKLSVKP

SEQ ID NO: 618 - protein, 112 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRRDSDYGYTFGSGTRLTVV

SEQ ID NO: 619 - DNA, 28 bp, synthetic construct
  source 1..28: mol_type = other DNA
cagcagctgaaagtcgggatcctacacc

SEQ ID NO: 620 - DNA, 23 bp, synthetic construct
  source 1..23: mol_type = other DNA
aacacagaaccacacagccagtc

SEQ ID NO: 621 - DNA, 26 bp, synthetic construct
  source 1..26: mol_type = other DNA
cagctgtatcctgatcttcttctgtc